(19) United States
(12) Patent Application Publication
(10) Pub. No.: US 2023/0372409 A1
(43) Pub. Date: Nov. 23, 2023

(54) SOLID DOSAGE FORMS OF BACTERIA

(71) Applicant: Evelo Biosciences, Inc, Cambridge, MA (US)

(72) Inventors: Syed Altaf, Cambridge, MA (US); Ruiming Huang, Cambridge, MA (US); Robert Ward, Llandudno (GB); Ryan Wilson, Flintshire (GB); Chun Zhang, Sudbury, MA (US)

(21) Appl. No.: 18/027,003

(22) PCT Filed: Sep. 17, 2021
(86) PCT No.: PCT/US21/50844

§ 371 Date: Mar. 17, 2023

Related U.S. Application Data
(60) Provisional application No. 63/234,483, filed on Aug. 18, 2021.
(60) Provisional application No. 63/161,617, filed on Mar. 16, 2021.
(60) Provisional application No. 63/157,153, filed on Mar. 5, 2021.
(60) Provisional application No. 63/145,786, filed on Feb. 4, 2021.
(60) Provisional application No. 63/110,090, filed on Nov. 5, 2020.
(60) Provisional application No. 63/089,799, filed on Oct. 9, 2020.
(60) Provisional application No. 63/080,263, filed on Sep. 18, 2020.

(51) Int. Cl. A61K 35/74; A61K 47/26; A61K 47/02; A61K 9/48

(52) U.S. Cl. CPC A61K 35/74; A61K 47/26; A61K 47/02; A61K 9/4816

(57) ABSTRACT

Methods and compositions related to solid dosage forms that facilitate the oral delivery of bacteria and/or agents of bacterial origin are provided herein.

### CROSS-REFERENCE TO RELATED APPLICATIONS

This application claims the benefit of the following U.S. Provisional Application Ser. No. 63/080,263, filed Sep. 18, 2020, 63/089,799, filed Oct. 9, 2020, 63/157,153, filed Mar. 5, 2021, 63/145,786, filed Feb. 4, 2021, 63/161,617, filed Mar. 16, 2021, 63/234,483, filed Aug. 18, 2021, and 63/110,090, filed Nov. 5, 2020, the entire contents of each of which are incorporated herein by reference.

### BACKGROUND

The formulation of the solid dosage form of a pharmaceutical product can have a significant impact on the bioavailability of its active pharmaceutical ingredients.

### SUMMARY

In certain aspects provided herein are solid dosage forms of pharmaceutical compositions. The pharmaceutical composition is also referred to as drug product. In certain embodiments, the solid dosage form comprises a pharmaceutical agent, wherein the pharmaceutical agent comprises bacteria and/or an agent of bacterial origin, such as mEVs, or a powder comprising bacteria and/or an agent of bacterial origin, such as mEVs and a diluent.

In certain aspects, provided herein is a solid dosage form of a pharmaceutical composition comprising a pharmaceutical agent having a total pharmaceutical agent mass that is at least 5% and no more than 95% of the total mass of the pharmaceutical composition, wherein the pharmaceutical agent comprises bacteria, a diluent having a total mass that is at least 1% and no more than 95% of the total mass of the pharmaceutical composition, a lubricant having a total mass that is at least 0.1% and no more than 5% of the total mass of the pharmaceutical composition, and a glidant having a total mass that is at least 0.01% and no more than 2% of the total mass of the pharmaceutical composition. In some embodiments, the solid dosage form is a capsule.

In certain aspects, provided herein is a solid dosage form of a pharmaceutical composition comprising a pharmaceutical agent having a total pharmaceutical agent mass that is at least 5% and no more than 95% of the total mass of the pharmaceutical composition, wherein the pharmaceutical agent comprises Prevotella histicola bacteria, a diluent having a total mass that is at least 1% and no more than 95% of the total mass of the pharmaceutical composition, a lubricant having a total mass that is at least 0.1% and no more than 5% of the total mass of the pharmaceutical composition, and a glidant having a total mass that is at least 0.01% and no more than 2% of the total mass of the pharmaceutical composition. In some embodiments, the solid dosage form is a capsule.

In some aspects, provided herein is a solid dosage form of a pharmaceutical composition comprising a pharmaceutical agent having a total pharmaceutical agent mass that is at least 2.5% and no more than 70% of the total mass of the pharmaceutical composition, wherein the pharmaceutical agent comprises bacteria, a diluent having a total mass that is at least 30% and no more than 98% of the total mass of the pharmaceutical composition, a lubricant having a total mass that is at least 0.5% and no more than 2.5% of the total mass of the pharmaceutical composition, and a glidant having a total mass that is at least 0.1% and no more than 1% of the total mass of the pharmaceutical composition. In some embodiments, the solid dosage form is a capsule.

In some aspects, provided herein is a solid dosage form of a pharmaceutical composition comprising a pharmaceutical agent having a total pharmaceutical agent mass that is at least 2.5% and no more than 70% of the total mass of the pharmaceutical composition, wherein the pharmaceutical agent comprises Veillonella parvula bacteria, a diluent having a total mass that is at least 30% and no more than 98% of the total mass of the pharmaceutical composition, a lubricant having a total mass that is at least 0.5% and no more than 2.5% of the total mass of the pharmaceutical composition, and a glidant having a total mass that is at least 0.1% and no more than 1% of the total mass of the pharmaceutical composition. In some embodiments, the solid dosage form is a capsule.

In certain aspects, provided herein is a solid dosage form of a pharmaceutical composition comprising a pharmaceutical agent having a total pharmaceutical agent mass that is at least 10% and no more than 90% of the total mass of the pharmaceutical composition, wherein the pharmaceutical agent comprises microbial extracellular vesicles (mEVs), a diluent having a total mass that is at least 7.5% and no more than 87.5% of the total mass of the pharmaceutical composition, a lubricant having a total mass that is about 1.5% of the total mass of the pharmaceutical composition, and a glidant having a total mass that is about 1% of the total mass of the pharmaceutical composition. In some embodiments, the solid dosage form is a capsule.

In certain aspects, provided herein is a solid dosage form of a pharmaceutical composition comprising a pharmaceutical agent having a total pharmaceutical agent mass that is at least 10% and no more than 90% of the total mass of the pharmaceutical composition, wherein the pharmaceutical agent comprises Prevotella histicola microbial extracellular vesicles (mEVs), a diluent having a total mass that is at least 7.5% and no more than 87.5% of the total mass of the pharmaceutical composition, a lubricant having a total mass that is about 1.5% of the total mass of the pharmaceutical composition, and a glidant having a total mass that is about 1% of the total mass of the pharmaceutical composition. In some embodiments, the solid dosage form is a capsule.

In certain embodiments, the total pharmaceutical agent mass is at least 2.5%, 5%, 10%, 15%, 20%, 25%, 30%, 35%, 40%, 45%, 50%, 55%, 60%, 65%, 70%, 75%, 80%, 85%, 90%, or 95% of the total mass of the pharmaceutical composition. In some embodiments, the total pharmaceutical agent mass is no more than 95%, 90%, 85%, 80%, 75%, 70%, 65%, 60%, 55%, 50%, 45%, 40%, 35%, 30%, 25%, 20%, 15%, 10%, 5%, or 2.5% of the total mass of the pharmaceutical composition. In some embodiments, the pharmaceutical agent has a total pharmaceutical agent mass that is at least 2.5% and no more than 95% of the total mass of the pharmaceutical composition. In some embodiments, the total pharmaceutical agent mass is about 5% to about 90% of the total mass of the pharmaceutical composition.

In some embodiments, the total mass of the diluent is at least 1%, 5%, 10%, 15%, 20%, 25%, 30%, 35%, 40%, 45%, 50%, 55%, 60%, 65%, 70%, 75%, 80%, 85%, 90%, 95%, or 98% of the total mass of the pharmaceutical composition. In some embodiments, the total mass of the diluent is no more than 98%, 95%, 90%, 85%, 80%, 75%, 70%, 65%, 60%, 55%, 50%, 45%, 40%, 35%, 30%, 25%, 20%, 15%, 10%, 5%, or 1%. In some embodiments, the diluent has a total mass that is at least 1% and no more than 98% of the total mass of the pharmaceutical composition. In some embodiments, the diluent has a total mass that is at least 35% and no more than 95% of the total mass of the pharmaceutical composition. In some embodiments, the diluent has a total mass that is about 38% to 93% of the total mass of the pharmaceutical composition. In some embodiments, the diluent comprises mannitol. In some embodiments, the diluent comprises microcrystalline cellulose.

In certain embodiments, the solid dosage form provided herein comprises a lubricant. In certain embodiments, the total lubricant mass is at least 0.1%, 0.5%, 1%, 2%, 3%, 4%, or 5% of the total mass of the pharmaceutical composition. In certain embodiments, the total lubricant mass is no more than 0.1%, 0.5%, 1%, 2%, 3%, 4%, or 5% of the total mass of the pharmaceutical composition. In certain embodiments, the total lubricant mass is about 0.1%, 0.5%, 1%, 2%, 3%, 4%, or 5% of the total mass of the pharmaceutical composition. In certain embodiments, the total lubricant mass is about 0.5% to about 1.5% of the total mass of the pharmaceutical composition. In certain embodiments, the total lubricant mass is about 1% of the total mass of the pharmaceutical composition. In certain embodiments, the total lubricant mass is about 1% to about 2% of the total mass of the pharmaceutical composition. In certain embodiments, the total lubricant mass is about 1.5% of the total mass of the pharmaceutical composition. In some embodiments, the lubricant comprises magnesium stearate.

In certain embodiments, the solid dosage forms provided herein comprise a glidant. In some embodiments, the glidant is colloidal silicon dioxide. In certain embodiments, the total glidant mass is at least 0.01%, 0.1%, 0.2%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is no more than 0.01%, 0.1%, 0.2%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is about 0.01%, 0.1%, 0.2%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is about 0.25% to about 0.75% of the total mass of the pharmaceutical composition. In certain embodiments, the total glidant mass is about 0.5% to about 1.5% of the total mass of the pharmaceutical composition. In certain embodiments, the total glidant mass is about 0.5% of the total mass of the pharmaceutical composition. In certain embodiments, the total glidant mass is about 1% of the total mass of the pharmaceutical composition.

In certain embodiments, the solid dosage forms provided herein comprise: (i) a pharmaceutical agent having a total pharmaceutical agent mass that is at least 4% and no more than 65% of the total mass of the pharmaceutical composition; (ii) a diluent (e.g., mannitol) having a total mass that is at least 35% and no more than 95% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is at least 5% and no more than 60% of the total mass of the pharmaceutical composition; (ii) a diluent (e.g., mannitol) having a total mass that is at least 38% and no more than 93% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is at least 20% and no more than 55% of the total mass of the pharmaceutical composition; (ii) a diluent (e.g., mannitol) having a total mass that is at least 45% and no more than 80% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is about 20% to about 50% of the total mass of the pharmaceutical composition; (ii) a diluent (e.g., mannitol) having a total mass that is about 50% to 80% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 20% to about 50% of the total mass of the pharmaceutical composition; (ii) a diluent (e.g., mannitol) having a total mass that is about 50% to 80% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is at least 5% and no more than 95% of the total mass of the pharmaceutical composition; (ii) a diluent (e.g., mannitol) having a total mass that is at least 1% and no more than 95% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is about 8% to about 92% the total mass of the pharmaceutical composition; (ii) a diluent (e.g., mannitol) having a total mass that is about 5% to 90% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 10% to about 90% of the total mass of the pharmaceutical composition; (ii) a diluent (e.g., mannitol) having a total mass that is about 7% to about 88% of the total mass of the pharmaceutical composition; (iii) a lubricant (e.g., magnesium stearate) having a total mass that is about 1.5% of the total mass of the pharmaceutical composition; and (iv) a glidant (e.g., colloidal silicon dioxide) having a total mass that is about 1% of the total mass of the pharmaceutical composition.

In certain embodiments, the solid dosage forms provided herein comprise: (i) a pharmaceutical agent having a total pharmaceutical agent mass that is at least 30% and no more than 50% of the total mass of the pharmaceutical composition; (ii) a diluent (e.g., mannitol) having a total mass that is at least 45% and no more than 70% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is about 30% to about 50% of the total mass of the pharmaceutical composition; (ii) a diluent (e.g., mannitol) having a total mass that is about 45% to 70% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 50% of the total mass of the pharmaceutical composition; (ii) a diluent (e.g., mannitol) having a total mass that is about 48.5% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is at least 8% and no more than 92% of the total mass of the pharmaceutical composition; (ii) a diluent (e.g., mannitol) having a total mass that is at least 5% and no more than 90% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is at least 10% and no more than 90% of the total mass of the pharmaceutical composition; (ii) a diluent (e.g., mannitol) having a total mass that is at least 8.5% and no more than 88.5% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is about 13.5% of the total mass of the pharmaceutical composition; (ii) a diluent (e.g., mannitol) having a total mass that is about 85.% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 90.2% of the total mass of the pharmaceutical composition; (ii) a diluent (e.g., mannitol) having a total mass that is about 8.3% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is at least 5% and no more than 50% of the total mass of the pharmaceutical composition; (ii) a diluent (e.g., mannitol) having a total mass that is at least 50% and no more than 95% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is at least 8% and no more than 45% of the total mass of the pharmaceutical composition; (ii) a diluent (e.g., mannitol) having a total mass that is at least 55% and no more than 90% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is about 40% of the total mass of the pharmaceutical composition; (ii) a diluent (e.g., mannitol) having a total mass that is about 58% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 10.6% of the total mass of the pharmaceutical composition; (ii) a diluent (e.g., mannitol) having a total mass that is about 87.4% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is at least 30% and no more than 50% of the total mass of the pharmaceutical composition; (ii) a diluent (e.g., mannitol) having a total mass that is at least 50% and no more than 70% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is at least 30% and no more than 50% of the total mass of the pharmaceutical composition; (ii) a diluent (e.g., mannitol) having a total mass that is at least 45% and no more than 70% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 50% of the total mass of the pharmaceutical composition; (ii) a diluent (e.g., mannitol) having a total mass that is about 48.5% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is at least 10% and no more than 90% of the total mass of the pharmaceutical composition; (ii) a diluent (e.g., mannitol) having a total mass that is at least 8.5% and no more than 88.5% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 13.51% of the total mass of the pharmaceutical composition; (ii) a diluent (e.g., mannitol) having a total mass that is about 84.99% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 90.22% of the total mass of the pharmaceutical composition; (ii) a diluent (e.g., mannitol) having a total mass that is about 8.28% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 50% of the total mass of the pharmaceutical composition; (ii) a diluent (e.g., mannitol) having a total mass that is about 48.5% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 5% of the total mass of the pharmaceutical composition; (ii) a diluent (e.g., mannitol) having a total mass that is about 93% of the total mass of the pharmaceutical composition; (iii) a lubricant (e.g., magnesium stearate) having a total mass that is about 1.5% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 60% of the total mass of the pharmaceutical composition; (ii) a diluent (e.g., mannitol) having a total mass that is about 38% of the total mass of the pharmaceutical composition; (iii) a lubricant (e.g., magnesium stearate) having a total mass that is about 1.5% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 10.6% of the total mass of the pharmaceutical composition; (ii) a diluent (e.g., mannitol) having a total mass that is about 87.4% of the total mass of the pharmaceutical composition; (iii) a lubricant (e.g., magnesium stearate) having a total mass that is about 1.5% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 40% of the total mass of the pharmaceutical composition; (ii) a diluent (e.g., mannitol) having a total mass that is about 58% of the total mass of the pharmaceutical composition; (iii) a lubricant (e.g., magnesium stearate) having a total mass that is about 1.5% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 98.5% of the total mass of the pharmaceutical composition; (ii) a diluent (e.g., mannitol) having a total mass that is about 0% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 25.1% of the total mass of the pharmaceutical composition; (ii) a diluent (e.g., microcrystalline cellulose) having a total mass that is about 73.4% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 10% of the total mass of the pharmaceutical composition; (ii) a diluent (e.g., mannitol) having a total mass that is about 87.5% of the total mass of the pharmaceutical composition; (iii) a lubricant (e.g., magnesium stearate) having a total mass that is about 1.5% of the total mass of the pharmaceutical composition; and (iv) a glidant (e.g., colloidal silicon dioxide) having a total mass that is about 1% of the total mass of the pharmaceutical composition.

In certain embodiments, the solid dosage forms provided herein comprise: (i) a pharmaceutical agent having a total pharmaceutical agent mass that is about 90% of the total mass of the pharmaceutical composition; (ii) a diluent (e.g., mannitol) having a total mass that is about 7.5% of the total mass of the pharmaceutical composition; (iii) a lubricant (e.g., magnesium stearate) having a total mass that is about 1.5% of the total mass of the pharmaceutical composition, and (iv) a glidant (e.g., colloidal silicon dioxide) having a total mass that is about 1% of the total mass of the pharmaceutical composition.

In certain embodiments, the solid dosage forms of a pharmaceutical agent as described herein comprise capsules. In some embodiments, the capsule is a size 00, size 0, size 1, size 2, size 3, size 4, or size 5 capsule. In some embodiments, the capsule is a size 0 capsule. In some embodiments, the capsule comprises HPMC (hydroxyl propyl methyl cellulose) or gelatin. In some embodiments, the capsule comprises HPMC (hydroxyl propyl methyl cellulose). In some embodiments, the capsule is banded. In some embodiments, the capsule is banded with an HPMC-based banding solution.

In some embodiments, the solid dosage form is enterically coated (e.g., comprises an enteric coating; e.g., is coated with an enteric coating).

In some embodiments, the solid dosage form is enteric coated to dissolve at pH 5.5.

In some embodiments, the enteric coating comprises a polymethacrylate-based copolymer. In some embodiments, the enteric coating comprises poly(methacrylic acid-co-ethyl acrylate).

In some embodiments, the enteric coating comprises a methacrylic acid ethyl acrylate (MAE) copolymer (1:1).

In some embodiments, the enteric coating comprises methacrylic acid ethyl acrylate (MAE) copolymer (1:1) (such as Kollicoat MAE 100P).

In some embodiments, the enteric coating comprises a Eudragit copolymer, e.g., a Eudragit L (e.g., Eudragit L 100-55; Eudragit L 30 D-55), a Eudragit S, a Eudragit RL, a Eudragit RS, a Eudragit E, or a Eudragit FS (e.g., Eudragit FS 30 D).

In some embodiments, the enteric coating comprises cellulose acetate phthalate (CAP), cellulose acetate trimellitate (CAT), poly(vinyl acetate phthalate) (PVAP), hydroxypropyl methylcellulose phthalate (HPMCP), a fatty acid, a wax, shellac (esters of aleurtic acid), a plastic, a plant fiber, zein, Aqua-Zein (an aqueous zein formulation containing no alcohol), amylose starch, a starch derivative, a dextrin, a methyl acrylate-methacrylic acid copolymer, cellulose acetate succinate, hydroxypropyl methyl cellulose acetate succinate (hypromellose acetate succinate), a methyl methacrylate-methacrylic acid copolymer, or sodium alginate.

In some embodiments, the enteric coating comprises an anionic polymeric material.

The pharmaceutical agent can be of bacterial origin (e.g., mixture of selected strains or agents (e.g., components) thereof, such as microbial extracellular vesicles (mEVs) of the mixture of selected strains). The pharmaceutical agent can be of bacterial origin (e.g., a single selected strain and/or agents (e.g., components) thereof, such as microbial extracellular vesicles (mEVs) of that single selected strain). The pharmaceutical agent can be a powder that comprises the bacteria and/or components thereof, and, can comprise additional agents such as, e.g., cryoprotectant. For example, in some embodiments, the pharmaceutical agent is a lyophilized powder of bacteria and/or components thereof (e.g., mEVs) that optionally, further comprise additional agents, such as a cryoprotectant. In some embodiments, the pharmaceutical agent has one or more beneficial immune effects outside the gastrointestinal tract, e.g., when the solid dosage form is orally administered.

In some embodiments, the pharmaceutical agent modulates immune effects outside the gastrointestinal tract in the subject, e.g., when the solid dosage form is orally administered.

In some embodiments, the pharmaceutical agent causes a systemic effect (e.g., an effect outside of the gastrointestinal tract), e.g., when the solid dosage form is orally administered.

In some embodiments, the pharmaceutical agent acts on immune cells and/or epithelial cells in the small intestine (e.g., causing a systemic effect (e.g., an effect outside of the gastrointestinal tract), e.g., when the solid dosage form is orally administered.

In some embodiments, the pharmaceutical agent comprises isolated bacteria (e.g., from one or more strains of bacteria (e.g., bacteria of interest) (e.g., a therapeutically effective amount thereof)). E.g., wherein at least 50%, at least 75%, at least 80%, at least 85%, at least 90%, at least 95%, or at least 99% of the content of the pharmaceutical agent is the isolated bacteria (e.g., bacteria of interest).

In some embodiments, the pharmaceutical agent comprises bacteria.

In some embodiments, the pharmaceutical agent comprises microbial extracellular vesicles (mEV).

In some embodiments, the pharmaceutical agent comprises bacteria and microbial extracellular vesicles (mEV).

In some embodiments, the pharmaceutical agent has one or more beneficial immune effects outside the gastrointestinal tract, e.g., when the solid dosage form is orally administered.

In some embodiments, the pharmaceutical agent modulates immune effects outside the gastrointestinal tract in the subject, e.g., when the solid dosage form is orally administered.

In some embodiments, the pharmaceutical agent causes a systemic effect (e.g., an effect outside of the gastrointestinal tract), e.g., when the solid dosage form is orally administered.

In some embodiments, the pharmaceutical agent acts on immune cells and/or epithelial cells in the small intestine (e.g., causing a systemic effect (e.g., an effect outside of the gastrointestinal tract), e.g., when the solid dosage form is orally administered.

In some embodiments, the pharmaceutical agent comprises isolated bacteria (e.g., from one or more strains of bacteria (e.g., bacteria of interest) (e.g., a therapeutically effective amount thereof)). E.g., wherein at least 50%, at least 75%, at least 80%, at least 85%, at least 90%, at least 95%, or at least 99% of the content of the pharmaceutical agent is the isolated bacteria (e.g., bacteria of interest).

In some embodiments, the pharmaceutical agent comprises bacteria that have been gamma irradiated, UV irradiated, heat inactivated, acid treated, or oxygen sparged.

In some embodiments, the pharmaceutical agent comprises live bacteria.

In some embodiments, the pharmaceutical agent comprises dead bacteria.

In some embodiments, the pharmaceutical agent comprises non-replicating bacteria.

In some embodiments, the pharmaceutical agent comprises bacteria from one strain of bacteria.

In some embodiments, the bacteria are lyophilized (e.g., the lyophilized product further comprises a pharmaceutically acceptable excipient) (e.g., a powder form).

In some embodiments, the bacteria are gamma irradiated.

In some embodiments, the bacteria are UV irradiated.

In some embodiments, the bacteria are heat inactivated (e.g., at 50° C. for two hours or at 90° C. for two hours).

In some embodiments, the bacteria are acid treated.

In some embodiments, the bacteria are oxygen sparged (e.g., at 0.1 vvm for two hours).

In some embodiments, the bacteria are Gram positive bacteria.

In some embodiments, the bacteria are Gram negative bacteria.

In some embodiments, the bacteria are aerobic bacteria.

In some embodiments, the bacteria are anaerobic bacteria. In some embodiments, the anaerobic bacteria comprise obligate anaerobes. In some embodiments, the anaerobic bacteria comprise facultative anaerobes.

In some embodiments, the bacteria are acidophile bacteria.

In some embodiments, the bacteria are alkaliphile bacteria.

In some embodiments, the bacteria are neutralophile bacteria.

In some embodiments, the bacteria are fastidious bacteria.

In some embodiments, the bacteria are nonfastidious bacteria.

In some embodiments, the bacteria are of a taxonomic group (e.g., class, order, family, genus, species or strain) listed in Table 1, Table 2, or Table 3.

In some embodiments, the bacteria are of a taxonomic group (e.g., class, order, family, genus, species or strain) listed in Table 4.

In some embodiments, the bacteria are a bacterial strain listed in Table 1, Table 2, or Table 3.

In some embodiments, the bacteria are a bacterial strain listed in Table 4.

In some embodiments, the bacteria are of a taxonomic group (e.g., class, order, family, genus, species or strain) listed in Table J.

In some embodiments, the bacteria are a bacterial strain listed in Table J.

In some embodiments, the Gram negative bacteria belong to class Negativicutes.

In some embodiments, the Gram negative bacteria belong to family Veillonellaceae, Selenomonadaceae, Acidaminococcaceae, or Sporomusaceae.

In some embodiments, the bacteria of the genus Megasphaera, Selenomonas, Propionospora, or Acidaminococcus.

In some embodiments, the bacteria are Megasphaera sp., Selenomonas felix, Acidaminococcus intestine, or Propionospora sp. bacteria.

In some embodiments, the bacteria are of the genus Lactococcus, Prevotella. Bifidobacterium, or Veillonella.

In some embodiments, the bacteria are Lactococcus lactis cremoris bacteria.

In some embodiments, the bacteria are Prevotella histicola bacteria.

In some embodiments, the bacteria are Bifidobacterium animalis bacteria.

In some embodiments, the bacteria are Veillonella parvula bacteria.

In some embodiments, the bacteria are Lactococcus lactis cremoris bacteria. In some embodiments, the Lactococcus lactis cremoris bacteria are a strain comprising at least 90% (or at least 97%) genomic, 16S and/or CRISPR sequence identity to the nucleotide sequence of the Lactococcus lactis cremoris Strain A (ATCC designation number PTA-125368). In some embodiments, the Lactococcus bacteria are a strain comprising at least 99% genomic, 16S and/or CRISPR sequence identity to the nucleotide sequence of the Lactococcus lactis cremoris Strain A (ATCC designation number PTA-125368). In some embodiments, the Lactococcus bacteria are Lactococcus lactis cremoris Strain A (ATCC designation number PTA-125368).

In some embodiments, the bacteria are Prevotella bacteria. In some embodiments, the Prevotella bacteria are a strain comprising at least 90% (or at least 97%) genomic, 16S and/or CRISPR sequence identity to the nucleotide sequence of the Prevotella Strain B 50329 (NRRL accession number B 50329). In some embodiments, the Prevotella bacteria are a strain comprising at least 99% genomic, 16S and/or CRISPR sequence identity to the nucleotide sequence of the Prevotella Strain B 50329 (NRRL accession number B 50329). In some embodiments, the Prevotella bacteria are Prevotella Strain B 50329 (NRRL accession number B 50329).

In some embodiments, the bacteria of the pharmaceutical agent or from which the mEVs of the pharmaceutical agent are obtained are Prevotella bacteria, e.g., a strain comprising at least 90% or at least 99% genomic, 16S and/or CRISPR sequence identity to the nucleotide sequence of the Prevotella Strain C (ATCC Accession Number PTA-126140). In some embodiments, the bacteria of the pharmaceutical agent or from which the mEVs of the pharmaceutical agent are obtained are Prevotella bacteria, e.g., Prevotella Strain C (ATCC Accession Number PTA-126140).

In some embodiments, the bacteria are Bifidobacterium bacteria. In some embodiments, the Bifidobacterium bacteria are from a strain comprising at least 90% (or at least 97%) genomic, 16S and/or CRISPR sequence identity to the nucleotide sequence of the Bifidobacterium bacteria deposited as ATCC designation number PTA-125097. In some embodiments, the Bifidobacterium bacteria are a strain comprising at least 99% genomic, 16S and/or CRISPR sequence identity to the nucleotide sequence of the Bifidobacterium bacteria deposited as ATCC designation number PTA-125097. In some embodiments, the Bifidobacterium bacteria are Bifidobacterium bacteria deposited as ATCC designation number PTA-125097.

In some embodiments, the bacteria are Veillonella bacteria. In some embodiments, the Veillonella bacteria are a strain comprising at least 90% (or at least 97%) genomic, 16S and/or CRISPR sequence identity to the nucleotide sequence of the Veillonella bacteria deposited as ATCC designation number PTA-125691. In some embodiments, the Veillonella bacteria are a strain comprising at least 99% genomic, 16S and/or CRISPR sequence identity to the nucleotide sequence of the Veillonella bacteria deposited as ATCC designation number PTA-125691. In some embodiments, the Veillonella bacteria are Veillonella bacteria deposited as ATCC designation number PTA-125691.

In some embodiments, the bacteria are from Ruminococcus gnavus bacteria. In some embodiments, the Ruminococcus gnavus bacteria are a strain comprising at least 90% (or at least 97%) genomic, 16S and/or CRISPR sequence identity to the nucleotide sequence of the Ruminococcus gnavus bacteria deposited as ATCC designation number PTA-126695. In some embodiments, the Ruminococcus gnavus bacteria are a strain comprising at least 99% genomic, 16S and/or CRISPR sequence identity to the nucleotide sequence of the Ruminococcus gnavus bacteria deposited as ATCC designation number PTA-126695. In some embodiments, the Ruminococcus gnavus bacteria are Ruminococcus gnavus bacteria deposited as ATCC designation number PTA-126695.

In some embodiments, the bacteria are Megasphaera sp. bacteria. In some embodiments, the Megasphaera sp. bacteria are a strain comprising at least 90% (or at least 97%) genomic, 16S and/or CRISPR sequence identity to the nucleotide sequence of the Megasphaera sp. bacteria deposited as ATCC designation number PTA-126770. In some embodiments, the Megasphaera sp. bacteria are a strain comprising at least 99% genomic, 16S and/or CRISPR sequence identity to the nucleotide sequence of the Megasphaera sp. bacteria deposited as ATCC designation number PTA-126770. In some embodiments, the Megasphaera sp. bacteria are Megasphaera sp. bacteria deposited as ATCC designation number PTA-126770.

In some embodiments, the bacteria are Fournierella massiliensis bacteria. In some embodiments, the Fournierella massiliensis bacteria are a strain comprising at least 90% (or at least 97%) genomic, 16S and/or CRISPR sequence identity to the nucleotide sequence of the Fournierella massiliensis bacteria deposited as ATCC designation number PTA-126696. In some embodiments, the Fournierella massiliensis bacteria are a strain comprising at least 99% genomic, 16S and/or CRISPR sequence identity to the nucleotide sequence of the Fournierella massiliensis bacteria deposited as ATCC designation number PTA-126696. In some embodiments, the Fournierella massiliensis bacteria are Fournierella massiliensis bacteria deposited as ATCC designation number PTA-126696.

In some embodiments, the bacteria are Harryflintia acetispora bacteria. In some embodiments, the Harryflintia acetispora bacteria are a strain comprising at least 90% (or at least 97%) genomic, 16S and/or CRISPR sequence identity to the nucleotide sequence of the Harryflintia acetispora bacteria deposited as ATCC designation number PTA-126694. In some embodiments, the Harryflintia acetispora bacteria are a strain comprising at least 99% genomic, 16S and/or CRISPR sequence identity to the nucleotide sequence of the Harryflintia acetispora bacteria deposited as ATCC designation number PTA-126694. In some embodiments, the Harryflintia acetispora bacteria are Harryflintia acetispora bacteria deposited as ATCC designation number PTA-126694.

In some embodiments, the bacteria are of the family Acidaminococcaceae, Alcaligenaceae, Akkermansiaceae, Bacteriodaceae, Bifidobacteriaceae, Burkholderiaceae, Catabacteriaceae, Clostridiaceae, Coriobacteriaceae, Enterobacteriaceae, Enterococcaceae, Fusobacteriaceae, Lachnospiraceae, Listeraceae, Mycobacteriaceae, Neisseriaceae, Odoribacteraceae, Oscillospiraceae, Peptococcaceae, Peptostreptococcaceae, Porphyromonadaceae, Prevotellaceac, Propionibacteraceae, Rikenellaceae, Ruminococcaceae, Selenomonadaceae, Sporomusaceae, Streptococcaceae, Streptomycetaceae, Sutterellaceae, Synergistaceae, or Veillonellaceae.

In some embodiments, the bacteria are of the genus Akkermansia, Christensenella, Blautia, Enterococcus, Eubacterium, Roseburia, Bacteroides, Parabacteroides, or Erysipelatoclostridium.

In some embodiments, the bacteria are Blautia hydrogenotrophica, Blautia stercoris, Blautia wexlerae, Eubacterium faccium, Eubacterium contortum, Eubacterium rectale, Enterococcus faecalis, Enterococcus durans, Enterococcus villorum, Enterococcus gallinarum; Bifidobacterium lactis, Bifidobacterium bifidium, Bifidobacterium longum, Bifidobacterium animalis, or Bifidobacterium breve bacteria.

In some embodiments, the bacteria are BCG (bacillus Calmette-Guerin), Parabacteroides, Blautia, Veillonella, Lactobacillus salivarius, Agathobaculum, Ruminococcus gnavus, Paraclostridium benzoelyticum, Turicibacter sanguinus, Burkholderia, Klebsiella quasipneumoniae ssp similpneumoniae, Klebsiella oxytoca, Tyzzerela nexilis, or Neisseria bacteria.

In some embodiments, the bacteria are Blautia hydrogenotrophica bacteria.

In some embodiments, the bacteria are Blautia stercoris bacteria.

In some embodiments, the bacteria are Blautia wexlerae bacteria.

In some embodiments, the bacteria are Enterococcus gallinarum bacteria.

In some embodiments, the bacteria are Enterococcus faecium bacteria.

In some embodiments, the bacteria are Bifidobacterium bifidium bacteria.

In some embodiments, the bacteria are Bifidobacterium breve bacteria.

In some embodiments, the bacteria are Bifidobacterium longum bacteria.

In some embodiments, the bacteria are Roseburia hominis bacteria.

In some embodiments, the bacteria are Bacteroides thetaiotaomicron bacteria.

In some embodiments, the bacteria are Bacteroides coprocola bacteria.

In some embodiments, the bacteria are Erysipelatoclostridium ramosum bacteria.

In some embodiments, the bacteria are Megasphera massiliensis bacteria.

In some embodiments, the bacteria are Eubacterium bacteria.

In some embodiments, the bacteria are Parabacteroides distasonis bacteria.

In some embodiments, the bacteria are Lactobacillus plantarum bacteria.

In some embodiments, the bacteria are bacteria of the Negativicutes class.

In some embodiments, the bacteria are of the Veillonellaceae family.

In some embodiments, the bacteria are of the Selenomonadaceae family.

In some embodiments, the bacteria are of the Acidaminococcaceae family.

In some embodiments, the bacteria are of the Sporomusaceae family.

In some embodiments, the bacteria are of the Megasphaera genus.

In some embodiments, the bacteria are of the Selenomonas genus.

In some embodiments, the bacteria are of the Propionospora genus.

In some embodiments, the bacteria are of the Acidaminococcus genus.

In some embodiments, the bacteria are Megasphaera sp. bacteria.

In some embodiments, the bacteria are Selenomonasfelix bacteria.

In some embodiments, the bacteria are Acidaminococcus intestini bacteria.

In some embodiments, the bacteria are Propionospora sp. bacteria.

In some embodiments, the bacteria are bacteria of the Clostridia class.

In some embodiments, the bacteria are of the Oscillospriraceae family.

In some embodiments, the bacteria are of the Faecalibacterium genus.

In some embodiments, the bacteria are of the Fournierella genus.

In some embodiments, the bacteria are of the Harryflintia genus.

In some embodiments, the bacteria are of the Agathobaculum genus.

In some embodiments, the bacteria are Faecalibacterium prausnitzii (e.g., Faecalibacterium prausnitzii Strain A) bacteria.

In some embodiments, the bacteria are Fournierella massiliensis (e.g., Fournierella massiliensis Strain A) bacteria.

In some embodiments, the bacteria are Harryflintia acetispora (e.g., Harryflintia acerispora Strain A) bacteria.

In some embodiments, the bacteria are Agathobaculum sp. (e.g., Agathobaculum sp. Strain A) bacteria.

In some embodiments, the bacteria are a strain of Agathobacrlum sp. In some embodiments, the Agathobaculum sp. strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Agathobaculum sp. Strain A (ATCC Deposit Number PTA-125892). In some embodiments, the Agathobaculum sp. strain is the Agathobaculum sp. Strain A (ATCC Deposit Number PTA-125892).

In some embodiments, the bacteria are of the class Bacteroidia [phylum Bacteroidota]. In some embodiments, the bacteria are of order Bacteroidales. In some embodiments, the bacteria are of the family Porphyromonoadaceae. In some embodiments, the bacteria are of the family Prevotellaceae. In some embodiments, the bacteria are of the class Bacterordia wherein the cell envelope structure of the bacteria is diderm. In some embodiments, the bacteria are of the class Bacteroidia that stain Gram negative. In some embodiments, the bacteria are of the class Bacteroidia wherein the bacteria is diderm and the bacteria stain Gram negative.

In some embodiments, the bacteria are of the class Clostridia [phylum Firmicutes]. In some embodiments, the bacteria are of the order Eubacteriales. In some embodiments, the bacteria are of the family Oscillispiraceae. In some embodiments, the bacteria are of the family Lachnospiraceae. In some embodiments, the bacteria are of the family Peptostreptococcaceae. In some embodiments, the bacteria are of the family Clostridiales family XIII/Incertae sedis 41. In some embodiments, the bacteria are of the class Clostridia wherein the cell envelope structure of the bacteria is monoderm. In some embodiments, the bacteria are of the class Clostridia that stain Gram negative. In some embodiments, the bacteria are of the class Clostridia that stain Gram positive. In some embodiments, the bacteria are of the class Clostridia wherein the cell envelope structure of the bacteria is monoderm and the bacteria stain Gram negative. In some embodiments, the bacteria are of the class Clostridia wherein the cell envelope structure of the bacteria is monoderm and the bacteria stain Gram positive.

In some embodiments, the bacteria are of the class Negativicutes [phylum Firmicutes]. In some embodiments, the bacteria are of the order Veillonellales. In some embodiments, the bacteria are of the family Veillonellaceae. In some embodiments, the bacteria are of the order Selenomonadales. In some embodiments, the bacteria are of the family Selenomonadaceae. In some embodiments, the bacteria are of the family Sporomusaceae. In some embodiments, the bacteria are of the class Negativicutes wherein the cell envelope structure of the bacteria is diderm. In some embodiments, the bacteria are of the class Negativicutes that stain Gram negative. In some embodiments, the bacteria are of the class Negativicutes wherein the cell envelope structure of the bacteria is diderm and the bacteria stain Gram negative.

In some embodiments, the bacteria are of the class Synergistia [phylum Synergistota]. In some embodiments, the bacteria are of the order Synergistales. In some embodiments, the bacteria are of the family Synergistaceae. In some embodiments, the bacteria are of the class Synergistia wherein the cell envelope structure of the bacteria is diderm. In some embodiments, the bacteria are of the class Synergistia that stain Gram negative. In some embodiments, the bacteria are of the class Synergistia wherein the cell envelope structure of the bacteria is diderm and the bacteria stain Gram negative.

In some embodiments, the bacteria are bacteria that produce metabolites, e.g., the bacteria produce butyrate, iosine, proprionate, or tryptophan metabolites.

In some embodiments, the bacteria produce butyrate. In some embodiments, the bacteria are from the genus Blautia; Christensella; Copracoccus; Eubacterium; Lachnosperacea; Megasphaera; or Roseburia.

In some embodiments, the bacteria produce iosine. In some embodiments, the bacteria are from the genus Bifidobacterium; Lactobacillus; or Olsenella.

In some embodiments, the bacteria produce proprionate. In some embodiments, the bacteria are from the genus Akkermansia; Bacteroides; Dialister; Eubacterium; Megasphaera; Parabacteriodes; Prevotella; Ruminococcus; or Veillonella.

In some embodiments, the bacteria produce tryptophan metabolites. In some embodiments, the bacteria are from the genus Lactobacillus or Peptostreptococcus.

In some embodiments, the bacteria are bacteria that produce inhibitors of histone deacetylase 3 (HDAC3). In some embodiments, the bacteria are from the species Bariatricus massiliensis, Faecalibacterium prausnitzii, Megasphaera massiliensis or Roseburia intestinalis.

In some embodiments, the bacteria are from the genus Alloiococcus; Bacillus; Catenibacterium; Corynebacterium; Cupriavidus; Enhwirobacter; Exiguobacterium; Faecalibacterium; Geobacillus; Methylobacterium; Micrococcus; Morganella; Proteus; Pseudomonas; Rhizobium; or Sphingomonas.

In some embodiments, the bacteria are from the genus Cutibacterium.

In some embodiments, the bacteria are from the species Cutibacterium avidum.

In some embodiments, the bacteria are from the genus Lactobacillus.

In some embodiments, the bacteria are from the species Lactobacillus gasseri.

In some embodiments, the bacteria are from the genus Dysosmobacter.

In some embodiments, the bacteria are from the species Dysosmobacter welbionis.

In some embodiments, the bacteria of the genus Leuconostoc.

In some embodiments, the bacteria of the genus Lactobacillus.

In some embodiments, the bacteria are of the genus Akkermansia muciniphila; Bacillus; Blautia; Cupriavidus; Enhydrobacter; Faecalibacterium; Lactobacillus; Lactococcus; Micrococcus; Morganella; Propionibacterium; Proteus; Rhizobiurm; or Streptococcus.

In some embodiments, the bacteria are Leuconostoc holzapfelii bacteria.

In some embodiments, the bacteria are Akkermansia muciniphila; Cupriavidus metallidurans; Faecalibacterium prausnitzii; Lactobacillus casei; Lactobacillus plantarum; Lactobacillus paracasei; Lactobacillus plantarum; Lactobacillus rhamnosus; Lactobacillus sakei; or Streptococcus pyogenes bacteria.

In some embodiments, the bacteria are Lactobacillus casei; Iactobacilhus plantarum; Lactobacillus paracasei; Lactobacillus plantarum; Lactobacillus rhamnosus; or Lactobacillus sakei bacteria.

In some embodiments, the bacteria are Megasphaera sp. bacteria (e.g., from the strain with accession number NCIMB 43385, NCIMB 43386 or NCIMB 43387).

In some embodiments, the bacteria are Megasphaera massiliensis bacteria (e.g., from the strain with accession number NCIMB 42787, NCIMB 43388 or NCIMB 43389).

In some embodiments, the bacteria are Megasphaera massiliensis bacteria (e.g., from the strain with accession number DSM 26228).

In some embodiments, the bacteria are Parabacteroides distasonis bacteria (e.g., from the strain with accession number NCIMB 42382).

In some embodiments, the bacteria are Megasphaera massiliensis bacteria (e.g., from the strain with accession number NCIMB 43388 or NCIMB 43389), or a derivative thereof. See, e.g., WO 2020/120714. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of Megasphaera massiliensis bacteria from the strain with accession number NCIMB 43388 or NCIMB 43389. In some embodiments, the Megasphaera massiliensis bacteria is the strain with accession number NCIMB 43388 or NCIMB 43389.

In some embodiments, the bacteria are Megasphaera massiliensis bacteria strain deposited under accession number NCIMB 42787, or a derivative thereof. See, e.g., WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Megasphaera massiliensis bacteria strain deposited under accession number NCIMB 42787. In some embodiments, the Megasphaera massiliensis bacteria is the strain deposited under accession number NCIMB 42787.

In some embodiments, the bacteria are Megasphaera spp. bacteria from the strain with accession number NCIMB 43385, NCIMB 43386 or NCIMB 43387, or a derivative thereof. See, e.g., WO 2020/120714. In some embodiments, the Megasphaera sp. bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Megasphaera sp. from a strain with accession number NCIMB 43385, NCIMB 43386 or NCIMB 43387. In some embodiments, the Megasphaera sp. bacteria is the strain with accession number NCIMB 43385, NCIMB 43386 or NCIMB 43387.

In some embodiments, the bacteria are Parabacteroides distasonis bacteria deposited under accession number NCIMB 42382, or a derivative thereof. See, e.g., WO 2018/229216. In some embodiments, the Parabacteroides distasonis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Parabacteroides distasonis bacteria deposited under accession number NCIMB 42382. In some embodiments, the Parabacteroides distasonis bacteria is the strain deposited under accession number NCIMB 42382.

In some embodiments, the bacteria are Megasphaera massiliensis bacteria deposited under accession number DSM 26228, or a derivative thereof. See, e.g., WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of Megasphaera massiliensis bacteria deposited under accession number DSM 26228. In some embodiments, the Megasphaera massiliensis bacteria is the strain deposited under accession number DSM 26228.

In some embodiments, the pharmaceutical agent comprises isolated mEVs (e.g., from one or more strains of bacteria (e.g., bacteria of interest)) (e.g., a therapeutically effective amount thereof). E.g., wherein at least 50%, at least 75%, at least 80%, at least 85%, at least 90%, at least 95%, or at least 99% of the content of the pharmaceutical agent is isolated mEV of bacteria (e.g., bacteria of interest).

In some embodiments, the pharmaceutical agent comprises mEVs and the mEVs comprise secreted mEVs (smEVs).

In some embodiments, the pharmaceutical agent comprises mEVs and the mEVs comprise processed mEVs (pmEVs).

In some embodiments, the pharmaceutical agent comprises pmEVs and the pmEVs are produced from bacteria that have been gamma irradiated, UV irradiated, heat inactivated, acid treated, or oxygen sparged.

In some embodiments, the pharmaceutical agent comprises pmEVs and the pmEVs are produced from live bacteria.

In some embodiments, the pharmaceutical agent comprises pmEVs and the pmEVs are produced from dead bacteria.

In some embodiments, the pharmaceutical agent comprises pmEVs and the pmEVs are produced from non-replicating bacteria.

In some embodiments, the pharmaceutical agent comprises mEVs and the mEVs are from one strain of bacteria.

In some embodiments, the mEVs are lyophilized (e.g., the lyophilized product further comprises a pharmaceutically acceptable excipient).

In some embodiments, the mEVs are gamma irradiated.

In some embodiments, the mEVs are UV irradiated.

In some embodiments, the mEVs are heat inactivated (e.g., at 50° C. for two hours or at 90° C. for two hours).

In some embodiments, the mEVs are acid treated.

In some embodiments, the mEVs are oxygen sparged (e.g., at 0.1 vvm for two hours).

In some embodiments, the mEVs are from Gram positive bacteria.

In some embodiments, the mEVs are from Gram negative bacteria.

In some embodiments, the mEVs are from aerobic bacteria.

In some embodiments, the mEVs are from anaerobic bacteria. In some embodiments, the anaerobic bacteria comprise obligate anaerobes. In some embodiments, the anaerobic bacteria comprise facultative anaerobes.

In some embodiments, the mEVs are from acidophile bacteria.

In some embodiments, the mEVs are from alkaliphile bacteria.

In some embodiments, the mEVs are from neutralophile bacteria.

In some embodiments, the mEVs are from fastidious bacteria.

In some embodiments, the mEVs are from nonfastidious bacteria.

In some embodiments, the mEVs are from bacteria of a taxonomic group (e.g., class, order, family, genus, species or strain) listed in Table 1, Table 2, or Table 3.

In some embodiments, the mEVs are from a bacterial strain listed in Table 1, Table 2, or Table 3.

In some embodiments, the mEVs are from bacteria of a taxonomic group (e.g., class, order, family, genus, species or strain) listed in Table J.

In some embodiments, the mEVs are from a bacterial strain listed in Table J.

In some embodiments, the Gram negative bacteria belong to class Negativicutes.

In some embodiments, the Gram negative bacteria belong to family Veillonellaceae, Selenomonadaceae, Acidaminococcaceae, or Sporomusaceae.

In some embodiments, the mEVs are from bacteria of the genus Megasphaera, Selenomonas, Propionospora, or Acidaminococcus.

In some embodiments, the mEVs are Megasphaera sp., Selenomonas felix, Acidaminococcus intestine, or Propionospora sp. bacteria.

In some embodiments, the mEVs are from bacteria of the genus Lactococcus, Prevotella, Bifidobacterium, or Veillonella.

In some embodiments, the mEVs are from Lactococcus lactis cremoris bacteria.

In some embodiments, the mEVs are from Prevotella histicola bacteria.

In some embodiments, the mEVs are from Bifidobacterium animalis bacteria.

In some embodiments, the mEVs are from Veillonella parvula bacteria.

In some embodiments, the mEVs are from Lactococcus lactis cremoris bacteria. In some embodiments, the Lactococcus lactis cremoris bacteria are from a strain comprising at least 90% (or at least 97%) genomic, 16S and/or CRISPR sequence identity to the nucleotide sequence of the Lactococcus lactis cremoris Strain A (ATCC designation number PTA-125368). In some embodiments, the Lactococcus bacteria are from a strain comprising at least 99% genomic, 16S and/or CRISPR sequence identity to the nucleotide sequence of the Lactococcus lactis cremoris Strain A (ATCC designation number PTA-125368). In some embodiments, the Lactococcus bacteria are from Lactococcus lactis cremoris Strain A (ATCC designation number PTA-125368).

In some embodiments, the mEVs are from Prevotella bacteria. In some embodiments, the Prevotella bacteria are from a strain comprising at least 90% (or at least 97%) genomic, 16S and/or CRISPR sequence identity to the nucleotide sequence of the Prevotella Strain B 50329 (NRRL accession number B 50329). In some embodiments, the Prevotella bacteria are from a strain comprising at least 99% genomic, 16S and/or CRISPR sequence identity to the nucleotide sequence of the Prevotella Strain B 50329 (NRRL accession number B 50329). In some embodiments, the Prevotella bacteria are from Prevotella Strain B 50329 (NRRL accession number B 50329).

In some embodiments, the mEVs are from Bifidobacterium bacteria. In some embodiments, the Bifidobacterium bacteria are from a strain comprising at least 90% (or at least 97%) genomic, 16S and/or CRISPR sequence identity to the nucleotide sequence of the Bifidobacterium bacteria deposited as ATCC designation number PTA-125097. In some embodiments, the Bifidobacterium bacteria are from a strain comprising at least 99% genomic, 16S and/or CRISPR sequence identity to the nucleotide sequence of the Bifidobacterium bacteria deposited as ATCC designation number PTA-125097. In some embodiments, the Bifidobacterium bacteria are from Bifidobacterium bacteria deposited as ATCC designation number PTA-125097.

In some embodiments, the mEVs are from Veillonella bacteria. In some embodiments, the Veillonella bacteria are from a strain comprising at least 90% (or at least 97%) genomic, 16S and/or CRISPR sequence identity to the nucleotide sequence of the Veillonella bacteria deposited as ATCC designation number PTA-125691. In some embodiments, the Veillonella bacteria are from a strain comprising at least 99% genomic, 16S and/or CRISPR sequence identity to the nucleotide sequence of the Veillonella bacteria deposited as ATCC designation number PTA-125691. In some embodiments, the Veillonella bacteria are from Veillonella bacteria deposited as ATCC designation number PTA-125691.

In some embodiments, the mEVs are from Ruminococcus gnavus bacteria. In some embodiments, the Ruminococcus gnavus bacteria are from a strain comprising at least 90% (or at least 97%) genomic, 16S and/or CRISPR sequence identity to the nucleotide sequence of the Ruminococcus gnavus bacteria deposited as ATCC designation number PTA-126695. In some embodiments, the Ruminococcus gnavus bacteria are from a strain comprising at least 99% genomic, 16S and/or CRISPR sequence identity to the nucleotide sequence of the Ruminococcus gnavus bacteria deposited as ATCC designation number PTA-126695. In some embodiments, the Ruminococcus gnavus bacteria are from Ruminococcus gnavus bacteria deposited as ATCC designation number PTA-126695.

In some embodiments, the mEVs are from Megasphaera sp. bacteria. In some embodiments, the Megasphaera sp. bacteria are from a strain comprising at least 90% (or at least 97%) genomic, 16S and/or CRISPR sequence identity to the nucleotide sequence of the Megasphaera sp. bacteria deposited as ATCC designation number PTA-126770. In some embodiments, the Megasphaera sp. bacteria are from a strain comprising at least 99% genomic, 16S and/or CRISPR sequence identity to the nucleotide sequence of the Megasphaera sp. bacteria deposited as ATCC designation number PTA-126770. In some embodiments, the Megasphaera sp. bacteria are from Megasphaera sp. bacteria deposited as ATCC designation number PTA-126770.

In some embodiments, the mEVs are from Fournierella massiliensis bacteria. In some embodiments, the Fournierella massiliensis bacteria are from a strain comprising at least 90% (or at least 97%) genomic, 16S and/or CRISPR sequence identity to the nucleotide sequence of the Fournierella massiliensis bacteria deposited as ATCC designation number PTA-126696. In some embodiments, the Fournierella massiliensis bacteria are from a strain comprising at least 99% genomic, 16S and/or CRISPR sequence identity to the nucleotide sequence of the Fournierella massiliensis bacteria deposited as ATCC designation number PTA-126696. In some embodiments, the Fournierella massiliensis bacteria are from Fournierella massiliensis bacteria deposited as ATCC designation number PTA-126696.

In some embodiments, the mEVs are from Harryflintia acetispora bacteria. In some embodiments, the Harryflintia acetispora bacteria are from a strain comprising at least 90% (or at least 97%) genomic, 16S and/or CRISPR sequence identity to the nucleotide sequence of the Harryflintia acetispora bacteria deposited as ATCC designation number PTA-126694. In some embodiments, the Harryflintia acetispora bacteria are from a strain comprising at least 99% genomic, 16S and/or CRISPR sequence identity to the nucleotide sequence of the Harryflintia acetispora bacteria deposited as ATCC designation number PTA-126694. In some embodiments, the Harryflintia acetispora bacteria are from Harryflintia acetispora bacteria deposited as ATCC designation number PTA-126694.

In some embodiments, the mEVs are from bacteria of the family Acidaminococcaceae, Alcaligenaceae, Akkermansiaceae, Bacteriodaceae, Bifidobacteriaceae, Burkholderiaceae, Catabacteriaceae, Clostridiaceae, Coriobacteriaceae, Enterobacteriaceae, Enterococcaceae, Fusobacteriaceae, Lachnospiraceae, Listeraceae, Mycobacteriaceae, Neisseriaceae, Odoribacteraceae, Oscillospiraceae, Peptococcaceae, Peptostreptococcaceac, Porphyromonadaceae, Prevotellaceae, Propionibacteraceae, Rikenellaceae, Ruminococcaceae, Selenomonadaceae, Sporomusaceae, Streptococcaceae, Streptomycetaceae, Sutterellaceae, Synergistaceae, or Veillonellaceae.

In some embodiments, the mEVs are from bacteria of the genus Akkermansia, Christensenella, Blautia, Enterococcus, Eubacterium, Roseburia, Bacteroides, Parabacteroides, or Erysipelatoclostridium.

In some embodiments, the mEVs are from Blautia hydrogenotrophica, Blautia stercoris, Blautia wexlerae, Eubacterium faecium, Eubacterium contortum, Eubacterium rectale, Enterococcus faecalis, Enterococcus durans, Enterococcus villorum, Enterococcus gallinarum; Bifidobacterium lactis, Bifidobacterium bifidium, Bifidobacterium longum, Bifidobacterium animalis, or Bifidobacterium breve bacteria.

In some embodiments, the mEVs are from BCG (bacillus Calmette-Guerin), Parabacteroides, Blautia, Veillonella, Lactobacillus salivarius, Agathobaculum, Ruminococcus gnavus, Paraclostridium benzoelyticum, Turicibacter sanguinus, Burkholderia, Klebsiella quasipneumoniae ssp similpneumoniae, Klebsiella oxytoca, Tyzzerela nexilis, or Neisseria bacteria.

In some embodiments, the mEVs are from Blautia hydrogenotrophica bacteria.

In some embodiments, the mEVs are from Blautia stercoris bacteria.

In some embodiments, the mEVs are from Blautia wexlerae bacteria.

In some embodiments, the mEVs are from Enterococcus gallinarum bacteria.

In some embodiments, the mEVs are from Enterococcus faecium bacteria.

In some embodiments, the mEVs are from Bifidobacterium bifidium bacteria.

In some embodiments, the mEVs are from Bifidobacterium breve bacteria.

In some embodiments, the mEVs are from Bifidobacterium longum bacteria.

In some embodiments, the mEVs are from Roseburia hominis bacteria.

In some embodiments, the mEVs are from Bacteroides thetaiotaomicron bacteria.

In some embodiments, the mEVs are from Bacteroides coprocola bacteria.

In some embodiments, the mEVs are from Erysipelatoclostridium ramosum bacteria.

In some embodiments, the mEVs are from Megasphera massiliensis bacteria.

In some embodiments, the mEVs are from Eubacterium bacteria.

In some embodiments, the mEVs are from Parabacteroides distasonis bacteria.

In some embodiments, the mEVs are from Lactobacillus plantarum bacteria.

In some embodiments, the mEVs are from bacteria of the Negativicutes class.

In some embodiments, the mEVs are from bacteria of the Veillonellaceae family.

In some embodiments, the mEVs are from bacteria of the Selenomonadaceae family.

In some embodiments, the mEVs are from bacteria of the Acidaminococcaceae family.

In some embodiments, the mEVs are from bacteria of the Sporomusaceae family.

In some embodiments, the mEVs are from bacteria of the Megasphaera genus.

In some embodiments, the mEVs are from bacteria of the Selenomonas genus.

In some embodiments, the mEVs are from bacteria of the Propionospora genus.

In some embodiments, the mEVs are from bacteria of the Acidaminococcus genus.

In some embodiments, the mEVs are from Megasphaera sp. bacteria.

In some embodiments, the mEVs are from Selenomonas felix bacteria.

In some embodiments, the mEVs are from Acidaminococcus intestini bacteria.

In some embodiments, the mEVs are from Propionospora sp. bacteria.

In some embodiments, the mEVs are from bacteria of the Clostridia class.

In some embodiments, the mEVs are from bacteria of the Oscillospriraceae family.

In some embodiments, the mEVs are from bacteria of the Faecalibacterium genus.

In some embodiments, the mEVs are from bacteria of the Fournierella genus.

In some embodiments, the mEVs are from bacteria of the Harryflintia genus.

In some embodiments, the mEVs are from bacteria of the Agathobaculum genus.

In some embodiments, the mEVs are from Faecalibacterium prausnitzii (e.g., Faecalibacterium prausnitzii Strain A) bacteria.

In some embodiments, the mEVs are from Fournierella massiliensis (e.g., Fournierella massiliensis Strain A) bacteria.

In some embodiments, the mEVs are from Harryflintia acetispora (e.g., Harryflintia acetispora Strain A) bacteria.

In some embodiments, the mEVs are from Agathobaculum sp. (e.g., Agathobaculum sp. Strain A) bacteria.

In some embodiments, the mEVs are from a strain of Agathobaculum sp. In some embodiments, the Agathobaculum sp. strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Agathobaculum sp. Strain A (ATCC Deposit Number PTA-125892). In some embodiments, the Agathobaculum sp. strain is the Agathobaculum sp. Strain A (ATCC Deposit Number PTA-125892).

In some embodiments, the mEVs are from bacteria of the class Bacteroidia [phylum Bacteroidota]. In some embodiments, the mEVs are from bacteria of order Bacteroidales. In some embodiments, the mEVs are from bacteria of the family Porphyromonoadaceae. In some embodiments, the mEVs are from bacteria of the family Prevotellaceae. In some embodiments, the mEVs are from bacteria of the class Bacteroidia wherein the cell envelope structure of the bacteria is diderm. In some embodiments, the mEVs are from bacteria of the class Bacteroidia that stain Gram negative. In some embodiments, the mEVs are from bacteria of the class Bacteroidia wherein the bacteria is diderm and the bacteria stain Gram negative.

In some embodiments, the mEVs are from bacteria of the class Clostridia [phylum Firmicutes]. In some embodiments, the mEVs are from bacteria of the order Eubacteriales. In some embodiments, the mEVs are from bacteria of the family Oscillispiraceae. In some embodiments, the mEVs are from bacteria of the family Lachnospiraceae. In some embodiments, the mEVs are from bacteria of the family Peptostreptococcaceae. In some embodiments, the mEVs are from bacteria of the family Clostridiales family XIII/Incertae sedis 41. In some embodiments, the mEVs are from bacteria of the class Clostridia wherein the cell envelope structure of the bacteria is monoderm. In some embodiments, the mEVs are from bacteria of the class Clostridia that stain Gram negative. In some embodiments, the mEVs are from bacteria of the class Clostridia that stain Gram positive. In some embodiments, the mEVs are from bacteria of the class Clostridia wherein the cell envelope structure of the bacteria is monoderm and the bacteria stain Gram negative. In some embodiments, the mEVs are from bacteria of the class Clostridia wherein the cell envelope structure of the bacteria is monoderm and the bacteria stain Gram positive.

In some embodiments, the mEVs are from bacteria of the class Negativicutes [phylum Firmicutes]. In some embodiments, the mEVs are from bacteria of the order Veillonellales. In some embodiments, the mEVs are from bacteria of the family Veillonelloceae. In some embodiments, the mEVs are from bacteria of the order Selenomonadales. In some embodiments, the mEVs are from bacteria of the family Selenomonadaceae. In some embodiments, the mEVs are from bacteria of the family Sporomusaceae. In some embodiments, the mEVs are from bacteria of the class Negativicutes wherein the cell envelope structure of the bacteria is diderm. In some embodiments, the mEVs are from bacteria of the class Negativicutes that stain Gram negative. In some embodiments, the mEVs are from bacteria of the class Negativicutes wherein the cell envelope structure of the bacteria is diderm and the bacteria stain Gram negative.

In some embodiments, the mEVs are from bacteria of the class Synergistia [phylum Synergistota]. In some embodiments, the mEVs are from bacteria of the order Synergistales. In some embodiments, the mEVs are from bacteria of the family Synergistaceae. In some embodiments, the mEVs are from bacteria of the class Synergistia wherein the cell envelope structure of the bacteria is diderm. In some embodiments, the mEVs are from bacteria of the class Synergistia that stain Gram negative. In some embodiments, the mEVs are from bacteria of the class Synergistia wherein the cell envelope structure of the bacteria is diderm and the bacteria stain Gram negative.

In some embodiments, the mEVs are from bacteria that produce metabolites, e.g., the bacteria produce butyrate, iosine, proprionate, or tryptophan metabolites.

In some embodiments, the mEVs are from bacteria that produce butyrate. In some embodiments, the bacteria are from the genus Blautia; Christensella; Copracoccus; Eubacterium; Lachnosperacea; Megasphaera; or Roseburia.

In some embodiments, the mEVs are from bacteria that produce iosine. In some embodiments, the bacteria are from the genus Bifidobacterium; Lactobacillus; or Olsenella.

In some embodiments, the mEVs are from bacteria that produce proprionate. In some embodiments, the mEVs are from bacteria from the genus Akkermansia; Bacteroides; Dialister; Eubacterium; Megasphaera; Parabacteriodes; Prevotella; Ruminococcus; or Veillonella.

In some embodiments, the mEVs are from bacteria that produce tryptophan metabolites. In some embodiments, the mEVs are from bacteria from the genus Lactobacillus or Peptostreptococcus.

In some embodiments, the mEVs are from bacteria that produce inhibitors of histone deacetylase 3 (HDAC3). In some embodiments, the mEVs are from bacteria from the species Bariatricus massiliensis, Faecalibacterium prausnitzii, Megasphaera massiliensis or Roseburia intestinalis.

In some embodiments, the mEVs are from bacteria of the genus Alloiococcus; Bacillus; Catenibacterium; Corynebacterium; Cupriavidus; Enhydrobacter; Exiguobacterium; Faecalibacterium; Geobacillus; Methylobacterium; Micrococcus; Morganella; Proteus; Pseudomonas; Rhizobium; or Sphingomonas.

In some embodiments, the mEVs are from bacteria of the genus Cutibacterium.

In some embodiments, the mEVs are from bacteria of the species Cutibacterium avidum.

In some embodiments, the mEVs are from bacteria of the genus Lactobacillus.

In some embodiments, the mEVs are from bacteria of the species Lactobacillus gasseri.

In some embodiments, the mEVs are from bacteria of the genus Dysosmobacter.

In some embodiments, the mEVs are from bacteria of the species Dysosmobacter welbionis.

In some embodiments, the mEVs are from bacteria of the genus Leuconostoc.

In some embodiments, the mEVs are from bacteria of the genus Lactobacillus.

In some embodiments, the mEVs are from bacteria of the genus Akkermansia muciniphila; Bacillus; Blautia; Cupriavidus; Enhydrobacter; Faecalibacterium; Lactobacillus; Lactococcus; Micrococcus; Morganella; Propionibacterium; Proteus; Rhizobium; or Streptococcus.

In some embodiments, the mEVs are from Leuconostoc holzapfelii bacteria.

In some embodiments, the mEVs are from Akkermansia muciniphila; Cupriavidus metallidurans; Faecalibacterium prausnitzii; Lactobacillus casei; Lactobacillus plantarum; Lactobacillus paracasei; Lactobacillus plantarum; Lactobacillus rhamnosus; Lactobacillus sakei; or Streptococcus pyogenes bacteria.

In some embodiments, the mEVs are from Lactobacillus casei; Lactobacillus plantarum; Lactobacillus paracasei; Lactobacillus plantarum; Lactobacillus rhamnosus; or Lactobacillus sakei bacteria.

In some embodiments, the mEVs are from Megasphaera sp. bacteria (e.g., from the strain with accession number NCIMB 43385, NCIMB 43386 or NCIMB 43387).

In some embodiments, the mEVs are from Megasphaera massiliensis bacteria (e.g., from the strain with accession number NCIMB 42787, NCIMB 43388 or NCIMB 43389).

In some embodiments, the mEVs are from Megasphaera massiliensis bacteria (e.g., from the strain with accession number DSM 26228).

In some embodiments, the mEVs are from Parabacteroides distasonis bacteria (e.g., from the strain with accession number NCIMB 42382).

In some embodiments, the mEVs are from Megasphaera massiliensis bacteria (e.g., from the strain with accession number NCIMB 43388 or NCIMB 43389), or a derivative thereof. See, e.g., WO 2020/120714. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of Megasphaera massiliensis bacteria from the strain with accession number NCIMB 43388 or NCIMB 43389. In some embodiments, the Megasphaera massiliensis bacteria is the strain with accession number NCIMB 43388 or NCIMB 43389.

In some embodiments, the mEVs are from Megasphaera massiliensis bacteria strain deposited under accession number NCIMB 42787, or a derivative thereof. See, e.g., WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Megasphaera massiliensis bacteria strain deposited under accession number NCIMB 42787. In some embodiments, the Megasphaera massiliensis bacteria is the strain deposited under accession number NCIMB 42787.

In some embodiments, the mEVs are from Megasphaera spp. bacteria from the strain with accession number NCIMB 43385, NCIMB 43386 or NCIMB 43387, or a derivative thereof. See, e.g., WO 2020/120714. In some embodiments, the Megasphaera sp. bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Megasphaera sp. from a strain with accession number NCIMB 43385, NCIMB 43386 or NCIMB 43387. In some embodiments, the Megasphaera sp. bacteria is the strain with accession number NCIMB 43385, NCIMB 43386 or NCIMB 43387.

In some embodiments, the mEVs are from Parabacteroides distasonis bacteria deposited under accession number NCIMB 42382, or a derivative thereof. See, e.g., WO 2018/229216. In some embodiments, the Parabacteroides distasonis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Parabacteroides distasonis bacteria deposited under accession number NCIMB 42382. In some embodiments, the Parabacteroides distasonis bacteria is the strain deposited under accession number NCIMB 42382.

In some embodiments, the mEVs are from Megasphaera massiliensis bacteria deposited under accession number DSM 26228, or a derivative thereof. See, e.g., WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of Megasphaera massiliensis bacteria deposited under accession number DSM 26228. In some embodiments, the Megasphaera massiliensis bacteria is the strain deposited under accession number DSM 26228.

In some embodiments, the pharmaceutical agent comprises Prevotella histicola bacteria and the dose of bacteria is about 1×10⁷ to about 2×10¹² (e.g., about 3×10¹⁰ or about 1.5×10¹¹) cells (e.g., wherein cell number is determined by total cell count, which is determined by Coulter counter), wherein the dose is per capsule. In some embodiments, the pharmaceutical agent comprises about 1×10⁷ to about 2×10¹¹ cells of Prevotella histicola bacteria. In some embodiments, the pharmaceutical agent comprises about 1.6×10¹⁰ cells of Prevotella histicola bacteria. In some embodiments, the pharmaceutical agent comprises about 8.0×10¹⁰ cells of Prevotella histicola bacteria. In some embodiments, the pharmaceutical agent comprises about 1.6×10¹¹ cells of Prevotella histicola bacteria. In some embodiments, the pharmaceutical agent comprises about 3.2×10¹¹ cells of Prevotella histicola bacteria.

In some embodiments, the pharmaceutical agent comprises Prevotella histicola bacteria and the dose of bacteria is about 1×10⁹, about 3×10⁹, about 5×10⁹, about 1.5×10¹⁰, or about 5×10¹⁰ cells (e.g., TCC (total cell count)), wherein the dose is per capsule. In some embodiments, the pharmaceutical agent comprises bacteria and the dose of bacteria is about 8×10¹⁰ cells, wherein the dose is per capsule. In some embodiments, the pharmaceutical agent comprises bacteria and the dose of bacteria is about 1.6×10¹¹ cells, wherein the dose is per capsule. In some embodiments, the pharmaceutical agent comprises bacteria and the dose of bacteria is about 3.2×10¹¹ cells, wherein the dose is per capsule.

In some embodiments, the pharmaceutical agent comprises a powder comprising bacteria and the dose of the pharmaceutical agent (e.g., a powder comprising bacteria) is about 10 mg to about 3500 mg, wherein the dose is per capsule.

In some embodiments, the pharmaceutical agent comprises a powder comprising bacteria and/and the dose of the pharmaceutical agent (e.g., a powder comprising bacteria) is about 30 mg to about 1300 mg (by weight of bacteria powder) (about 25, about 30, about 35, about 50, about 75, about 100, about 120, about 150, about 250, about 300, about 350, about 400, about 500, about 600, about 700, about 750, about 800, about 900, about 1000, about 1100, about 1200, about 1250, about 1300, about 2000, about 2500, about 3000, or about 3500 mg wherein the dose is per capsule.

In some embodiments, the pharmaceutical agent comprises bacteria and the dose of pharmaceutical agent (e.g., bacteria) is about 2×10⁶ to about 2×10¹⁶ particles (e.g., wherein particle count is determined by NTA (nanoparticle tracking analysis)), wherein the dose is per capsule.

In some embodiments, the pharmaceutical agent comprises bacteria and the dose of pharmaceutical agent (e.g., bacteria) is about 5 mg to about 900 mg total protein (e.g., wherein total protein is determined by Bradford assay or BCA), wherein the dose is per capsule.

In some embodiments, the solid dosage form further comprises one or more additional therapeutic agents.

In some aspects, the disclosure provides a method of treating a subject (e.g., human) (e.g., a subject in need of treatment), the method comprising administering to the subject a solid dosage form provided herein.

In some aspects, the disclosure provides use of a solid dosage form provided herein for the preparation of a medicament for treating a subject (e.g., human) (e.g., a subject in need of treatment).

In some embodiments, the solid dosage form is orally administered (e.g., is for oral administration).

In some embodiments, the solid dosage form is administered to a subject that is in a fed or fasting state. In some embodiments, the solid dosage form is administered to a subject on an empty stomach (e.g., one hour before eating or two hours after eating). In some embodiments, the solid dosage form is administered to a subject one hour before eating. In some embodiments, the solid dosage form is administered to a subject two hours after eating.

In some embodiments, the solid dosage form is administered (e.g., is for administration) 1, 2, 3, or 4 times a day. In some embodiments, 1, 2, 3, or 4 solid dosage forms (e.g., capsules) are administered (e.g., are for administration) 1, 2, 3, or 4 times a day. In some embodiments, 2, 4, 6, 8, or 10 solid dosage forms (e.g., capsules) are administered (e.g., are for administration) 1, 2, 3, or 4 times a day.

In some embodiments, the solid dosage form provides release of the pharmaceutical agent in the small intestine, e.g., in the upper small intestine, of the pharmaceutical agent contained in the solid dosage form.

In some embodiments, the solid dosage form delivers the pharmaceutical agent to the small intestine, wherein the pharmaceutical agent can act on immune cells and/or epithelial cells in the small intestine, e.g., in the upper small intestine, e.g., to cause effects throughout the body (e.g., systemic effect).

In some embodiments, the pharmaceutical agent provides one or more beneficial immune effects outside the gastrointestinal tract, e.g., when orally administered.

In some embodiments, the pharmaceutical agent modulates immune effects outside the gastrointestinal tract in the subject, e.g., when orally administered.

In some embodiments, the pharmaceutical agent causes a systemic effect (e.g., an effect outside of the gastrointestinal tract), e.g., when orally administered.

In some embodiments, the pharmaceutical agent acts on immune cells and/or epithelial cells in the small intestine (e.g., upper small intestine) (e.g., causing a systemic effect (e.g., an effect outside of the gastrointestinal tract), e.g., when orally administered.

In some embodiments, the solid dosage form is administered orally and has one or more beneficial immune effects outside the gastrointestinal tract (e.g., interaction between the agent and cells in the small intestine modulates a systemic immune response).

In some embodiments, the solid dosage form is administered orally and modulates immune effects outside the gastrointestinal tract (e.g., interaction between agent and cells in the small intestine (e.g., upper small intestine) modulates a systemic immune response).

In some embodiments, the solid dosage form is administered orally and activates innate antigen presenting cells (e.g., in the small intestine, e.g., upper small intestine).

In some embodiments, the subject is in need of treatment (and/or prevention) of a cancer.

In some embodiments, the subject is in need of treatment (and/or prevention) of an autoimmune disease.

In some embodiments, the subject is in need of treatment (and/or prevention) of an inflammatory disease.

In some embodiments, the subject is in need of treatment (and/or prevention) of a metabolic disease.

In some embodiments, the subject is in need of treatment (and/or prevention) of a dysbiosis.

In some embodiments, the subject is in need of decreased inflammatory cytokine expression (e.g., decreased IL-8, IL-6, IL-1β, and/or TNFα expression levels).

In some embodiments, the subject is in need of treatment (and/or prevention) of bacterial septic shock, cytokine storm and/or viral infection.

In some embodiments, the subject is in need of treatment (and/or prevention) of a viral infection.

In some embodiments, the viral infection is a coronavirus infection, an influenza infection, and/or a respiratory syncytial virus infection.

In some embodiments the viral infection is a SARS-CoV-2 infection.

In some embodiments, the solid dosage form is administered in combination with a therapeutic agent (e.g., additional therapeutic agent).

In certain aspects, provided herein are methods of preparing a solid dosage form of a pharmaceutical composition, the method comprising combining into a pharmaceutical composition a pharmaceutical agent (e.g., Prevotella histicola or Veillonella parvula bacteria disclosed herein or a powder comprising the bacteria) and a diluent.

In certain embodiments, the total pharmaceutical agent mass is at least 2.5%, 50%, 10%, 15%, 200%, 25%, 30%, 35%, 40%, 45%, 50%, 55%, 60%, 65%, 70%, 75%, 80%, 85%, 90%, or 95% of the total mass of the pharmaceutical composition. In some embodiments the total pharmaceutical agent mass is no more than 95%, 90%, 85%, 80%, 75%, 70%, 65%, 60%, 55%, 50%, 45%, 40%, 35%, 30%, 25%, 20%, 15%, 10%, 5%, or 2.5% of the total mass of the pharmaceutical composition. In some embodiments, the pharmaceutical agent has a total pharmaceutical agent mass that is at least 2.5% and no more than 95% of the total mass of the pharmaceutical composition.

In some embodiments, the total mass of the diluent is at least 1%, 5%, 10%, 15%, 20%, 25%, 30%, 35%, 40%, 45%, 50%, 55%, 60%, 65%, 700%, 75%, 80%, 85%, 90%, 95%, or 98% of the total mass of the pharmaceutical composition. In some embodiments, the total mass of the diluent is no more than 98%, 95%, 90%, 85%, 80%, 75%, 70%, 65%, 60%, 55%, 50%, 45%, 40%, 35%, 30%, 25%, 20%, 15%, 10%, 5%, or 1% of the total mass of the pharmaceutical composition. In some embodiments, the diluent has a total mass that is at least 1% and no more than 98% of the total mass of the pharmaceutical composition. In some embodiments, the diluent comprises mannitol.

In certain embodiments, the method further comprises combining a lubricant. In certain embodiments, the total lubricant mass is at least 0.1%, 0.5%, 1%, 2%, 3%, 4%, or 5% of the total mass of the pharmaceutical composition. In certain embodiments, the total lubricant mass is no more than 0.1%, 0.5%, 1%, 2%, 3%, 4%, or 5% of the total mass of the pharmaceutical composition. In certain embodiments, the total lubricant mass is about 0.1%, 0.5%, 1%, 2%, 3%, 4%, or 5% of the total mass of the pharmaceutical composition. In certain embodiments, the total lubricant mass is about 0.5% to about 1.5% of the total mass of the pharmaceutical composition. In certain embodiments, the total lubricant mass is about 1% of the total mass of the pharmaceutical composition. In some embodiments, the lubricant comprises magnesium stearate.

In certain embodiments, the method further comprises combining a glidant. In some embodiments, the glidant is colloidal silicon dioxide. In certain embodiments, the total glidant mass is at least 0.01%, 0.1%, 0.2%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is no more than 0.01%, 0.1%, 0.2%, 0.3%, 0.4%, 0.50, 0.6%, 0.7%, 0.8%, 0.9%, 1%, 1.5%, or 2% of the total mass of the pharmaceutical composition. In certain embodiments, the total glidant mass is about 0.01%, 0.1%, 0.2%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is about 0.25% to about 0.75% of the total mass of the pharmaceutical composition. In certain embodiments, the total glidant mass is about 0.5% to about 1.5% of the total mass of the pharmaceutical composition. In certain embodiments, the total glidant mass is about 0.5% of the total mass of the pharmaceutical composition. In certain embodiments, the total glidant mass is about 1% of the total mass of the pharmaceutical composition.

In certain embodiments, the method provided herein comprises combining: (i) a pharmaceutical agent having a total pharmaceutical agent mass that is at least 4% and no more than 65% of the total mass of the pharmaceutical composition; (ii) a diluent (e.g., mannitol) having a total mass that is at least 35% and no more than 95% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is at least 5% and no more than 60% of the total mass of the pharmaceutical composition; (ii) a diluent (e.g., mannitol) having a total mass that is at least 38% and no more than 93% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is at least 20% and no more than 55% of the total mass of the pharmaceutical composition; (ii) a diluent (e.g., mannitol) having a total mass that is at least 45% and no more than 80% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is at least 8% and no more than 92% of the total mass of the pharmaceutical composition; (ii) a diluent (e.g., mannitol) having a total mass that is at least 5% and no more than 90% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is about 20% to about 50% of the total mass of the pharmaceutical composition, (ii) a diluent (e.g., mannitol) having a total mass that is about 50% to 80% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is at least 30% and no more than 50% of the total mass of the pharmaceutical composition; (ii) a diluent (e.g., mannitol) having a total mass that is at least 45% and no more than 70% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is about 50% of the total mass of the pharmaceutical composition; (ii) a diluent (e.g., mannitol) having a total mass that is about 48.5% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the method provided herein comprises combining: (i) a pharmaceutical agent having a total pharmaceutical agent mass that is at least 8% and no more than 92% of the total mass of the pharmaceutical composition; (ii) a diluent (e.g., mannitol) having a total mass that is at least 5% and no more than 90% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is about 10% to about 90% of the total mass of the pharmaceutical composition; (ii) a diluent (e.g., mannitol) having a total mass that is about 7% to about 88% of the total mass of the pharmaceutical composition; (iii) a lubricant (e.g., magnesium stearate) having a total mass that is about 1.5% of the total mass of the pharmaceutical composition; and (iv) a glidant (e.g., colloidal silicon dioxide) having a total mass that is about 1% of the total mass of the pharmaceutical composition.

In certain embodiments, the method provided herein comprises combining: (i) a pharmaceutical agent having a total pharmaceutical agent mass that is at least 10% and no more than 90% of the total mass of the pharmaceutical composition; (ii) a diluent (e.g., mannitol) having a total mass that is at least 8.5% and no more than 88.5% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is about 13.51% of the total mass of the pharmaceutical composition; (ii) a diluent (e.g., mannitol) having a total mass that is about 84.99% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the method provided herein comprises combining: (i) a pharmaceutical agent having a total pharmaceutical agent mass that is about 90.22% of the total mass of the pharmaceutical composition; (ii) a diluent (e.g., mannitol) having a total mass that is about 8.28% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the method provided herein comprises combining: (i) a pharmaceutical agent having a total pharmaceutical agent mass that is at least 5% and no more than 50% of the total mass of the pharmaceutical composition; (ii) a diluent (e.g., mannitol) having a total mass that is at least 50% and no more than 95% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is at least 5% and no more than 50% of the total mass of the pharmaceutical composition; (ii) a diluent (e.g., mannitol) having a total mass that is at least 50% and no more than 95% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is at least 8% and no more than 45% of the total mass of the pharmaceutical composition; (ii) a diluent (e.g., mannitol) having a total mass that is at least 55% and no more than 90% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is about 40% of the total mass of the pharmaceutical composition; (ii) a diluent (e.g., mannitol) having a total mass that is about 58% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the method provided herein comprises combining: (i) a pharmaceutical agent having a total pharmaceutical agent mass that is about 10.6% of the total mass of the pharmaceutical composition; (ii) a diluent (e.g., mannitol) having a total mass that is about 87.4% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In some embodiments, the method further comprises loading the pharmaceutical composition into a capsule. In some embodiments, the capsule comprises HPMC.

In some embodiments, the method further comprises banding the capsule. In some embodiments, the capsule is banded with an HPMC-based banding solution.

In some embodiments, the method further comprises enterically coating the capsule, thereby preparing an enterically coated capsule.

In certain embodiments, the method comprises performing wet granulation on a pharmaceutical agent prior to combining the pharmaceutical agent (e.g., bacteria (e.g., bacteria disclosed herein) and/or an agent of bacterial origin, such as mEVs (e.g., mEVs disclosed herein)) and one or more (e.g., one, two or three) excipients into a pharmaceutical composition. In some embodiments, the wet granulation comprises (i) mixing the pharmaceutical agent with a granulating fluid (e.g., water, ethanol, or isopropanol, alone or in combination). In some embodiments, the wet granulation comprises mixing the pharmaceutical agent with water. In some embodiments, the wet granulation comprises (ii) drying mixed pharmaceutical agent and granulating fluid (e.g., drying on a fluid bed dryer). In some embodiments, the wet granulation comprises (iii) milling the dried pharmaceutical agent and granulating fluid. The milled pharmaceutical agent and granulating fluid are then combined with the one or more (e.g., one, two or three) excipients to prepare a pharmaceutical composition.

As used herein, the percent of mass of a solid dosage form is on a percent weight:weight basis (% w:w).

### BRIEF DESCRIPTION OF THE DRAWINGS

FIG. 1 is a graph showing a Total Cells/Capsule Stability Profile over time long-term (2-8° C.) and accelerated (25° C./60% RH) storage conditions for Batch A. The trace ending at 6 months (diamonds) in the graph provides values for accelerated (25° C./60% RH) storage conditions. The trace ending at 18 months (circles) in the graph provides values for long-term (2-8° C.) storage conditions. Total Cell Count (TCC) was determined by Coulter counter.

FIG. 2 is a graph showing a Water Content Stability Profile over time long-term (2-8° C.) and accelerated (25° C./60% RH) storage conditions for Batch A. The trace ending at 6 months (diamonds) in the graph provides values for accelerated (25° C./60% RH) storage conditions. The trace ending at 18 months (circles) in the graph provides values for long-term (2-8° C.) storage conditions. Water content was determined by the Karl Fisher method.

FIG. 3 is a graph showing Total Cells/Capsule Stability Profile over time long-term (2-8° C.) and accelerated (25° C./60% RH) storage conditions for Batch B. The lower trace (diamonds) in the graph provides values for accelerated (25° C./60% RH) storage conditions. The upper trace (circles) in the graph provides values for long-term (2-8° C.) storage conditions. Total Cell Count (TCC) was determined by Coulter counter.

FIG. 4 is a graph showing a Water Content Stability Profile over time long-term (2-8° C.) and accelerated (25° C./60% RH) storage conditions for Batch B. The upper trace (diamonds) in the graph provides values for accelerated (25° C./60% RH) storage conditions. The lower trace (circles) in the graph provides values for long-term (2-8° C.) storage conditions. Water content was determined by the Karl Fisher method.

FIG. 5 is a graph showing a Total Cells/Capsule Stability Profile over time long-term (2-8° C.) and accelerated (25° C./60% RH) storage conditions for Batch C. The trace ending at 6 months (diamonds) in the graph provides values for accelerated (25° C./60% RH) storage conditions. The trace ending at 18 months (circles) in the graph provides values for long-term (2-8° C.) storage conditions. Total Cell Count (TCC) was determined by Coulter counter.

FIG. 6 is a graph showing a Water Content Stability Profile over time long-term (2-8° C.) and accelerated (25° C./60% RH) storage conditions for Batch C. The trace ending at 6 months (diamonds) in the graph provides values for accelerated (25° C./60% RH) storage conditions. The trace ending at 18 months (circles) in the graph provides values for long-term (2-8° C.) storage conditions. Water content was determined by the Karl Fisher method.

FIG. 7 is a graph showing a Total Cells/Capsule Stability Profile over time long-term (2-8° C.) and accelerated (25° C./60% RH) storage conditions for Batch F. The lower trace (diamonds) in the graph provides values for accelerated (25° C./60% RH) storage conditions. The upper trace (circles) in the graph provides values for long-term (2-8° C.) storage conditions. Total Cell Count (TCC) was determined by Coulter counter.

FIG. 8 is a graph showing a Water Content Stability Profile over time long-term (2-8° C.) and accelerated (25° C./60% RH) storage conditions for Batch F. The trace that ends in the lower position (diamonds) in the graph provides values for accelerated (25° C./60% RH) storage conditions. The trace that ends in the upper position (circles) in the graph provides values for long-term (2-8° C.) storage conditions. Water content was determined by the Karl Fisher method.

FIG. 9 is a graph showing Total Cells/Capsule Stability Profile over time long-term (2-8° C. (abbreviation: 5° C.)) storage conditions for the low dose batch. Total Cell Count (TCC) was determined by Coulter counter.

FIG. 10 is a graph showing Total Cells/Capsule Stability Profile overtime long-term (2-8° C. (abbreviation: 5° C.)) storage conditions for the high dose batch. Total Cell Count (TCC) was determined by Coulter counter.

FIG. 11 is a graph showing Water Content Stability Profile over time long-term (2-8° C. (abbreviation: 5° C.)) and accelerated (25° C./60% RH (abbreviation: 25° C.)) storage conditions for the low dose batch. The traces for the two conditions overlap. Water content was determined by the Karl Fisher method.

FIG. 12 is a graph showing Water Content Stability Profile over time long-term (2-8° C. (abbreviation: 5° C.)) and accelerated (25° C./60% RH (abbreviation: 25° C.)) storage conditions for the high dose batch. The traces for the two conditions largely overlap until the 3-month time point. The upper trace at the 3-month time point in the graph provides values for long-term (2-8° C.) storage conditions. The lower trace at the 3-month time point in the graph provides values for accelerated (25° C./60% RH) storage conditions. Water content was determined by the Karl Fisher method.

FIGS. 13A and B are graphs showing 6-month Stability Profiles for the high dose batch. FIG. 13A is a graph showing Total Cells/Capsule Stability Profile overtime long-term (2-8° C. (abbreviation: 5° C.)) and accelerated (25° C./60% RH (abbreviation: 25° C.)) storage conditions for the high dose batch. Total Cell Count (TCC) was determined by Coulter counter. FIG. 13B is a graph showing Moisture Content Stability Profile over time long-term (2-8° C.) and accelerated (25° C./60% RH (abbreviation: 25° C.)) storage conditions for the high dose batch. Water content was determined by the Karl Fisher method.

FIGS. 14A and B are graphs showing 6-month Stability Profiles for the low dose batch. FIG. 14A is a graph showing Total Cells/Capsule Stability Profile over time long-term (2-8° C. (abbreviation: 5° C.)) and accelerated (25° C./60% RH (abbreviation: 25° C.)) storage conditions for the low dose batch. Total Cell Count (TCC) was determined by Coulter counter. FIG. 14B is a graph showing Moisture Content Stability Profile over time long-term (2-8° C.) and accelerated (25° C./60% RH (abbreviation: 25° C.)) storage conditions for the low dose batch. Water content was determined by the Karl Fisher method.

FIGS. 15A and B are graphs showing 6-month Stability Profiles for a second high dose batch. FIG. 15A is a graph showing Total Cells/Capsule Stability Profile over time long-term (2-8° C. (abbreviation: 5° C.)) and accelerated (25° C./60% RH (abbreviation: 25° C.)) storage conditions for the second high dose batch. Total Cell Count (TCC) was determined by Coulter counter. FIG. 15B is a graph showing Moisture Content Stability Profile over time long-term (2-8° C.) and accelerated (25° C./60% RH (abbreviation: 25° C.)) storage conditions for the second high dose batch. Water content was determined by the Karl Fisher method.

FIGS. 16A and B are graphs showing 6-month Stability Profiles for a second low dose batch. FIG. 8A is a graph showing Total Cells/Capsule Stability Profile over time long-term (2-8° C. (abbreviation: 5° C.)) and accelerated (25° C./60% RH (abbreviation: 25° C.)) storage conditions for the second dose batch. Total Cell Count (TCC) was determined by Coulter counter. FIG. 8B is a graph showing Moisture Content Stability Profile over time long-term (2-8° C.) and accelerated (25° C./60% RH (abbreviation: 25° C.)) storage conditions for the second low dose batch. Water content was determined by the Karl Fisher method.

### DETAILED DESCRIPTION

This disclosure is based, in part, on the discovery that a solid dosage form is prepared with or without a diluent (e.g., mannitol or microcrystalline cellulose). For example, for a solid dosage form to contain a given amount (e.g., dose) of active ingredient (e.g., bacteria and agents (e.g., components) of bacterial origin (e.g., microbial extracellular vesicles, or mEVs)), the amount of pharmaceutical agent (that contains the active ingredient) incorporated into a solid dosage form may be adjusted depending on the amount of active ingredient contained in a given preparation (e.g., batch) of pharmaceutical agent. The amount of diluent (such as mannitol or microcrystalline cellulose) is then adjusted accordingly. For example, if the amount of pharmaceutical agent is increased, the amount of diluent is decreased; and vice versa. As described herein, adjustments can be made to the amounts of pharmaceutical agent and diluent, yet the amount of one or more excipients (e.g., one, two or three excipients) remains constant, e.g., batch to batch for a given solid dosage form recipe. Similarly, the amounts of magnesium stearate and colloidal silica can also remain constant, e.g., batch to batch for a given solid dosage form recipe.

The disclosure provides solid dosage forms that comprise bacteria (e.g., powder comprising bacteria) that maintain their stability, e.g., for three, six, twelve, eighteen and/or twenty-four months under long-term (2-8° C.) and/or accelerated (25° C./60% RH) storage conditions, e.g., as determined by total cell count (TCC), e.g., as determined by Coulter counter and described herein. For example, as described herein, stability is maintained wherein the TCC range is set at 50% to 150% of the target amount. e.g., at a given time point (e.g., at a three, six, twelve, eighteen and/or twenty-four month time point under long-term (2-8° C.) and/or accelerated (25° C./60% RH) storage conditions), and the solid dosage form comprises a TCC within the set TCC range.

The disclosure provides solid dosage forms that comprise Prevotella histicola (e.g., Prevotella histicola powder) that maintain their stability, e.g., for three, six, twelve, eighteen and/or twenty-four months under long-term (2-8° C.) and/or accelerated (25° C./60% RH) storage conditions, e.g., as determined by total cell count (TCC), e.g., as determined by Coulter counter and described herein. For example, as described herein, stability is maintained wherein the TCC range is set at 50% to 150% of the target amount, e.g., at a given time point (e.g., at a three, six, twelve, eighteen and/or twenty-four month time point under long-term (2-8° C.) and/or accelerated (25° C./60% RH) storage conditions), and the solid dosage form comprises a TCC within the set TCC range. For example, for a target amount of 1.6×10¹⁰TCC/capsule, the acceptable TCC range is set at 0.8×10¹⁰ to 2.4×10¹⁰ and stability is maintained wherein the solid dosage form comprises a TCC within the set TCC range; for a target amount of 8×10¹⁰ TCC/capsule, the acceptable TCC range is set at 4×10¹⁰ to 1.2×10¹¹ and stability is maintained wherein the solid dosage form comprises a TCC within the set TCC range, for a target amount of 3.2×10¹¹ TCC/capsule, the acceptable TCC range is set at 1.6×10¹¹ to 4.8×10¹¹ and stability is maintained wherein the solid dosage form comprises a TCC within the set TCC range.

The disclosure provides solid dosage forms that comprise Prevotella histicola (e.g., Prevotella histicola powder) that have the water content between about 0.5% and about 8.3% (e.g., about 1% to about 6%, e.g., about 3.5%, e.g., about 5.2%, e.g., about 3.4%, e.g., about 4.0%. e.g., about 1.9%. e.g., about 5.9%, or e.g., about 0.99%). e.g., as determined by the Karl-Fischer method for water content analysis provided in Ph. Eur. method 2.5.32, and as described herein. In some embodiments, the solid dosage forms maintain their water content, e.g., for three, six, twelve, eighteen and/or twenty-four months under long-term (2-8° C.) and/or accelerated (25° C./60% RH) storage conditions.

The disclosure provides solid dosage forms that comprise Veillonella parvula (e.g., Veillonella parvula powder) that maintain their stability. e.g., for three, six, twelve, eighteen and/or twenty-four months under long-term (2-8° C.) and/or accelerated (25° C./60% RH) storage conditions, e.g., as determined by total cell count (TCC), e.g., as determined by Coulter counter and described herein. For example, as described herein, stability is maintained wherein the TCC range is set at 50% to 150% of the target amount, e.g., at a given time point (e.g., at a three, six, twelve, eighteen and/or twenty-four month time point under long-term (2-8° C.) and/or accelerated (25° C./60% RH) storage conditions), and the solid dosage form comprises a TCC within the set TCC range. For example, for a target amount of 4.5×10¹⁰ TCC/capsule, the acceptable TCC range is set at 2.25×10¹⁰ to 6.75×10¹⁰ and stability is maintained wherein the solid dosage form comprises a TCC within the set TCC range; for a target amount of 1.5×10¹¹ TCC/capsule, the acceptable TCC range is set at 7.5×10¹⁰ to 2.25×10¹¹ and stability is maintained wherein the solid dosage form comprises a TCC within the set TCC range.

The disclosure provides solid dosage forms that comprise Veillonella parvula (e.g., Veillonella parvula powder) that have the water content between about 0.5% and about %, between about 0.5% and about 6%, between about 1.5% and about 12%, (e.g., about 0.5% to about 5%, e.g., about 0.7%, e.g., about 4.3%, e.g., about 1.1%, or e.g., about 3.6%), e.g., as determined by the Karl-Fischer method for water content analysis provided in Ph. Eur. method 2.5.32, and as described herein. In some embodiments, the solid dosage forms maintain their water content, e.g., for three, five, six, twelve, eighteen and/or twenty-four months under long-term (2-8° C.) and/or accelerated (25° C./60% RH) storage conditions.

As described herein, for a solid dosage form to contain a given amount (e.g., dose) of active ingredient (e.g., pharmaceutical agent, e.g, Veillonella parvula bacteria (e.g., bacteria and/or a powder comprising bacteria), the amount of pharmaceutical agent (that contains the active ingredient) incorporated into a solid dosage form may be adjusted depending on the amount of active ingredient contained in a given preparation (e.g., batch) of pharmaceutical agent. The amount of diluent (such as mannitol) is then adjusted accordingly. For example, if the amount of pharmaceutical agent is increased, the amount of diluent is decreased; and vice versa. As described herein, adjustments can be made to the amounts of pharmaceutical agent and diluent, yet the amount of one or more excipients (e.g., one, two or three excipients) remains constant, e.g., batch to batch for a given solid dosage form recipe. Similarly, the amounts of magnesium stearate and colloidal silica can also remain constant, e.g., batch to batch for a given solid dosage form recipe.

For example, in the working examples provided herein, pharmaceutical agent containing Veillonella parvula powder was used to prepare two capsule solid dosage forms. Both preparations contained 1.5% magnesium stearate and 0.5% colloidal silica. Yet in one preparation, the pharmaceutical agent was used at 60%. In the other, it was used at 5%. To adjust for the differing amounts of pharmaceutical agent, the amount of mannitol was differed: 38% mannitol when 60% pharmaceutical agent was used; 93% mannitol when 5% pharmaceutical agent was used.

For example, in the working examples provided herein, pharmaceutical agent containing Lactococcus lactis ssp, cremoris powder was used to prepare a capsule solid dosage form. In the preparation, the active ingredient totaled at 98.5% (w:w) and the magnesium stearate and colloidal silica were each 1% and 0.5%, respectively. No diluent was used.

For example, in the working examples provided herein, pharmaceutical agent containing Lactococcus lactis ssp, cremoris powder was used to prepare a capsule solid dosage form. In the preparation, the active ingredient totaled at 25.1% (w:w), microcrystalline cellulose was used as the diluent and totaled at 73.4%, and the magnesium stearate and colloidal silica were each 1% and 0.5%, respectively.

For example, in the working examples provided herein, pharmaceutical agent containing Prevotella histicola powder was used to prepare two capsule solid dosage forms. Both preparations contained 1.0% magnesium stearate and 0.5% colloidal silica. Yet in one preparation, the pharmaceutical agent was used at 90.22%. In the other, it was used at 50%. To adjust for the differing amounts of pharmaceutical agent, the amount of mannitol was differed: 8.28% mannitol when 90.22% pharmaceutical agent was used; 48.5% mannitol when 50% pharmaceutical agent was used.

For example, in the working examples provided herein, pharmaceutical agent containing Veillonella parvula powder (e.g., in which the Veillonella parvula bacteria were gamma irradiated) was used to prepare two capsule solid dosage forms. Both preparations contained 1.5% magnesium stearate and 0.5% colloidal silica. Yet in one preparation, the pharmaceutical agent was used at 60%. In the other, it was used at 5%. To adjust for the differing amounts of pharmaceutical agent, the amount of mannitol was differed: 38% mannitol when 60% pharmaceutical agent was used; 93% mannitol when 5% pharmaceutical agent was used.

### Definitions

The term “about” when used before a numerical value indicates that the value may vary within a reasonable range, such as within +10%, 5% or 1% of the stated value.

“Adjuvant” or “Adjuvant therapy” broadly refers to an agent that affects an immunological or physiological response in a subject (e.g., human). For example, an adjuvant might help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition (e.g., a pharmaceutical composition such as a solid dosage form of a pharmaceutical agent as described herein) to a subject. Examples of routes of administration include oral administration, rectal administration, topical administration, inhalation (nasal) or injection. Administration by injection includes intravenous (IV), intramuscular (IM), and subcutaneous (SC) administration. A pharmaceutical composition described herein can be administered in any form by any effective route, including but not limited to oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In preferred embodiments, a pharmaceutical composition described herein is administered orally, rectally, topically, intravesically, by injection into or adjacent to a draining lymph node, intravenously, by inhalation or aerosol, or subcutaneously. In another preferred embodiment, a pharmaceutical composition described herein is administered orally, or intravenously. In another embodiment, a pharmaceutical composition described herein is administered orally.

“Cancer” broadly refers to an uncontrolled, abnormal growth of a host's own cells leading to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood forming tissue (e.g., bone marrow tissue); lymphomas and myelomas which are cancers of immune cells; and central nervous system cancers which include cancers from brain and spinal tissue. “Cancer(s) and” “neoplasm(s)” are used herein interchangeably. As used herein, “cancer” refers to all types of cancer or neoplasm or malignant tumors including leukemias, carcinomas and sarcomas, whether new or recurring. Specific examples of cancers are; carcinomas, sarcomas, myelomas, leukemias, lymphomas and mixed type tumors. Non-limiting examples of cancers are new or recurring cancers of the brain, melanoma, bladder, breast, cervix, colon, head and neck, kidney, lung, non-small cell lung, mesothelioma, ovary, prostate, sarcoma, stomach, uterus and medulloblastoma. In some embodiments, the cancer comprises a solid tumor. In some embodiments, the cancer comprises a metastasis.

A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CnH2nOn. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffnose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

“Cellular augmentation” broadly refers to the influx of cells or expansion of cells in an environment that are not substantially present in the environment prior to administration of a composition and not present in the composition itself. Cells that augment the environment include immune cells, stromal cells, bacterial and fungal cells.

“Clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity.

A “combination” of bacteria from two or more strains includes the physical co-existence of the bacteria, either in the same material or product or in physically connected products, as well as the temporal co-administration or co-localization of the bacteria from the two or more strains.

A “combination” of mEVs (such as smEVs and/or pmEVs) from two or more microbial (such as bacteria) strains includes the physical co-existence of the microbes from which the mEVs (such as smEVs and/or pmEVs) are obtained, either in the same material or product or in physically connected products, as well as the temporal co-administration or co-localization of the mEVs (such as smEVs and/or pmEVs) from the two or more strains.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state. Properties that may be decreased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size).

“Dysbiosis” refers to a state of the microbiota or microbiome of the gut or other body area, including, e.g., mucosal or skin surfaces (or any other microbiome niche) in which the normal diversity and/or function of the host gut microbiome ecological networks “microbiome”) are disrupted. A state of dysbiosis may result in a diseased state, or it may be unhealthy under only certain conditions or only if present for a prolonged period. Dysbiosis may be due to a variety of factors, including, environmental factors, infectious agents, host genotype, host diet and/or stress. A dysbiosis may result in: a change (e.g., increase or decrease) in the prevalence of one or more bacteria types (e.g., anaerobic), species and/or strains, change (e.g., increase or decrease) in diversity of the host microbiome population composition; a change (e.g., increase or reduction) of one or more populations of symbiont organisms resulting in a reduction or loss of one or more beneficial effects; overgrowth of one or more populations of pathogens (e.g., pathogenic bacteria); and/or the presence of, and/or overgrowth of, symbiotic organisms that cause disease only when certain conditions are present.

The term “ecological consortium” is a group of bacteria which trades metabolites and positively co-regulates one another, in contrast to two bacteria which induce host synergy through activating complementary host pathways for improved efficacy.

As used herein, “engineered bacteria” are any bacteria that have been genetically altered from their natural state by human activities, and the progeny of any such bacteria. Engineered bacteria include, for example, the products of targeted genetic modification, the products of random mutagenesis screens and the products of directed evolution.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Mrtin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

As used herein, the term “immune disorder” refers to any disease, disorder or disease symptom caused by an activity of the immune system, including autoimmune diseases, inflammatory diseases and allergies. Immune disorders include, but are not limited to, autoimmune diseases (e.g., psoriasis, atopic dermatitis, lupus, scleroderma, hemolytic anemia, vasculitis, type one diabetes, Grave's disease, rheumatoid arthritis, multiple sclerosis, Goodpasture's syndrome, pernicious anemia and/or myopathy), inflammatory diseases (e.g., acne vulgaris, asthma, celiac disease, chronic prostatitis, glomerulonephritis, inflammatory bowel disease, pelvic inflammatory disease, reperfusion injury, rheumatoid arthritis, sarcoidosis, transplant rejection, vasculitis and/or interstitial cystitis), and/or an allergies (e.g., food allergies, drug allergies and/or environmental allergies).

“Immunotherapy” is treatment that uses a subject's immune system to treat disease (e.g., immune disease, inflammatory disease, metabolic disease) and includes, for example, cytokines, cell therapy, CAR-T cells, and dendritic cell therapy.

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10\hat{ }3 fold, 10\hat{ }4 fold, 10\hat{ }5 fold, 10\hat{ }6 fold, and/or 10\hat{ }7 fold greater after treatment when compared to a pre-treatment state. Properties that may be increased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size).

“Innate immune agonists” or “immuno-adjuvants” are small molecules, proteins, or other agents that specifically target innate immune receptors including Toll-Like Receptors (TLR), NOD receptors, RLRs, C-type lectin receptors, STING-cGAS Pathway components, inflammasome complexes. For example, LPS is a TLR-4 agonist that is bacterially derived or synthesized and aluminum can be used as an immune stimulating adjuvant. immuno-adjuvants are a specific class of broader adjuvant or adjuvant therapy. Examples of STING agonists include, but are not limited to, 2′3′-cGAMP, 3′3′-cGAMP, c-di-AMP, c-di-GMP, 2′2′-cGAMP, and 2′3′-cGAM(PS)2 (Rp/Sp) (Rp, Sp-isomers of the bis-phosphorothioate analog of 2′3′-cGAMP). Examples of TLR agonists include, but are not limited to, TLR1, TLR2, TLR3, TLR4, TLR5, TLR6, TLR7, TLR8, TLR9, TLR10 and TLR1 1. Examples of NOD agonists include, but are not limited to, N-acetylmuramyl-L-alanyl-D-isoglutamine (muramyldipeptide (MDP)), gamma-D-glutamyl-meso-diaminopimelic acid (iE-DAP), and desmuramylpeptides (DMP).

The “internal transcribed spacer” or “ITS” is a piece of non-functional RNA located between structural ribosomal RNAs (rRNA) on a common precursor transcript often used for identification of eukaryotic species in particular fungi. The rRNA of fungi that forms the core of the ribosome is transcribed as a signal gene and consists of the 8S, 5.8S and 28S regions with ITS4 and 5 between the 8S and 5.85 and 5.8S and 28S regions, respectively. These two intercistronic segments between the 18S and 5.85 and 5.8S and 28S regions are removed by splicing and contain significant variation between species for barcoding purposes as previously described (Schoch et al Nuclear ribosomal internal transcribed spacer (ITS) region as a universal DNA barcode marker for Fungi. PNAS 109:6241-6246. 2012). 18S rDNA is traditionally used for phylogenetic reconstruction however the ITS can serve this function as it is generally highly conserved but contains hypervariable regions that harbor sufficient nucleotide diversity to differentiate genera and species of most fungus.

The term “isolated” or “enriched” encompasses a microbe (such as a bacterium), an mEV (such as an smEV and/or pmEV) or other entity or substance that has been (1) separated from at least some of the components with which it was associated when initially produced (whether in nature or in an experimental setting), and/or (2) produced, prepared, purified, and/or manufactured by the hand of man. Isolated microbes or mEVs may be separated from at least about 10%, about 20%, about 30%, about 40%, about 50%, about 60%, about 70%, about 80%, about 90%, or more of the other components with which they were initially associated. In some embodiments, isolated microbes or mEVs are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a microbe or mEV or other material that has been separated from at least some of the components with which it was associated either when initially produced or generated (e.g., whether in nature or in an experimental setting), or during any time after its initial production. A microbe or a microbial population or mEVs may be considered purified if it is isolated at or after production, such as from a material or environment containing the microbe or microbial population, and a purified microbe or microbial population or mEVs may contain other materials up to about 10%, about 20%, about 30%, about 40%, about 50%, about 60%, about 70%, about 80%, about 90%, or above about 90% and still be considered “isolated.” In some embodiments, purified microbes or microbial population or mEVs are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components (such as mEVs) thereof are generally purified from residual habitat products.

As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans).

The term “LPS mutant or lipopolysaccharide mutant” broadly refers to selected bacteria that comprises loss of LPS. Loss of LPS might be due to mutations or disruption to genes involved in lipid A biosynthesis, such as lpxA, lpxC, and lpxD. Bacteria comprising LPS mutants can be resistant to aminoglycosides and polymyxins (polymyxin B and colistin).

“Metabolite” as used herein refers to any and all molecular compounds, compositions, molecules, ions, co-factors, catalysts or nutrients used as substrates in any cellular or microbial metabolic reaction or resulting as product compounds, compositions, molecules, ions, co-factors, catalysts or nutrients from any cellular or microbial metabolic reaction.

“Microbial extracellular vesicles” (mEVs) can be obtained from microbes such as bacteria, archaea, fungi, microscopic algae, protozoans, and parasites. In some embodiments, the mEVs are obtained from bacteria. mEVs include secreted microbial extracellular vesicles (smEVs) and processed microbial extracellular vesicles (pmEVs). “Secreted microbial extracellular vesicles” (smEVs) are naturally-produced vesicles derived from microbes. smEVs are comprised of microbial lipids and/or microbial proteins and/or microbial nucleic acids and/or microbial carbohydrate moieties, and are isolated from culture supernatant. The natural production of these vesicles can be artificially enhanced (e.g., increased) or decreased through manipulation of the environment in which the bacterial cells are being cultured (e.g., by media or temperature alterations). Further, smEV compositions may be modified to reduce, increase, add, or remove microbial components or foreign substances to alter efficacy, immune stimulation, stability, immune stimulatory capacity, stability, organ targeting (e.g., lymph node), absorption (e.g., gastrointestinal), and/or yield (e.g., thereby altering the efficacy). As used herein, the term “purified smEV composition” or “smEV composition” refers to a preparation of smEVs that have been separated from at least one associated substance found in a source material (e.g., separated from at least one other microbial component) or any material associated with the smEVs in any process used to produce the preparation. It can also refer to a composition that has been significantly enriched for specific components. “Processed microbial extracellular vesicles” (pmEVs) are a non-naturally-occurring collection of microbial membrane components that have been purified from artificially lysed microbes (e.g., bacteria) (e.g., microbial membrane components that have been separated from other, intracellular microbial cell components), and which may comprise particles of a varied or a selected size range, depending on the method of purification. A pool of pmEVs is obtained by chemically disrupting (e.g., by lysozyme and/or lysostaphin) and/or physically disrupting (e.g., by mechanical force) microbial cells and separating the microbial membrane components from the intracellular components through centrifugation and/or ultracentrifugation, or other methods. The resulting pmEV mixture contains an enrichment of the microbial membranes and the components thereof (e.g., peripherally associated or integral membrane proteins, lipids, glycans, polysaccharides, carbohydrates, other polymers), such that there is an increased concentration of microbial membrane components, and a decreased concentration (e.g., dilution) of intracellular contents, relative to whole microbes. For gram-positive bacteria, pmEVs may include cell or cytoplasmic membranes. For gram-negative bacteria, a pmEV may include inner and outer membranes, pmEVs may be modified to increase purity, to adjust the size of particles in the composition, and/or modified to reduce, increase, add or remove, microbial components or foreign substances to alter efficacy, immune stimulation, stability, immune stimulatory capacity, stability, organ targeting (e.g., lymph node), absorption (e.g., gastrointestinal), and/or yield (e.g., thereby altering the efficacy), pmEVs can be modified by adding, removing, enriching for, or diluting specific components, including intracellular components from the same or other microbes. As used herein, the term “purified pmEV composition” or “pmEV composition” refers to a preparation of pmEVs that have been separated from at least one associated substance found in a source material (e.g., separated from at least one other microbial component) or any material associated with the pmEVs in any process used to produce the preparation. It can also refer to a composition that has been significantly enriched for specific components.

“Microbe” refers to any natural or engineered organism characterized as a archaeaon, parasite, bacterium, fungus, microscopic alga, protozoan, and the stages of development or life cycle stages (e.g., vegetative, spore (including sporulation, dormancy, and germination), latent, biofilm) associated with the organism. Examples of gut microbes include: Actinomyces graevenitzii, Actinomyces odontolyticus, Akkermansia muciniphila, Bacteroides caccae, Bacteroides fragilis, Bacteroides putredinis, Bacteroides thetaiotaomicron, Bacteroides vultagus, Bifidobacterium adolescentis, Bifidobacterium bifidum, Bilophila wadsworthia, Blautia, Butyrivibrio, Campylobacter gracilis, Clostridia cluster III, Clostridia cluster, IV Clostridia cluster IX (Acidaminococcaceae group), Clostridia cluster XI, Clostridia cluster XIII (Peptostreptococcus group). Clostridia cluster XIV, Clostridia cluster XV Collinsella aerofaciens, Coprococcus, Corynebacterium sunsvallense, Desulfomonas pigra, Dorea formicigenerans, Dorea longicatena, Eschernchia coli, Eubacterium hadrum, Eubacterium rectale, Faecalibacteria prausnitzii, Gemella, Lactococcus, Lanchnospira, Mollicutes cluster XVI, Mollicutes cluster XVIII, Prevotella, Rothia mucilaginosa, Ruminococcus callidus, Ruminococcus gnavus, Ruminococcus torques, and Streptococcus.

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state microbiome. The microbiome may be native to the subject or patient, or components of the microbiome may be modulated, introduced, or depleted due to changes in health state or treatment conditions (e.g., antibiotic treatment, exposure to different microbes). In some aspects, the microbiome occurs at a mucosal surface. In some aspects, the microbiome is a gut microbiome.

A “microbiome profile” or a “microbiome signature” of a tissue or sample refers to an at least partial characterization of the bacterial makeup of a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present or absent in a microbiome.

“Modified” in reference to a bacteria broadly refers to a bacteria that has undergone a change from its wild-type form. Bacterial modification can result from engineering bacteria. Examples of bacterial modifications include genetic modification, gene expression modification, phenotype modification, formulation modification, chemical modification, and dose or concentration. Examples of improved properties are described throughout this specification and include, e.g., attenuation, auxotrophy, homing, or antigenicity. Phenotype modification might include, by way of example, bacteria growth in media that modify the phenotype of a bacterium such that it increases or decreases virulence.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 16S, OTUs that share ≥97% average nucleotide identity across the entire 16S or some variable region of the 16S are considered the same OTU. See e.g., Claesson M J, Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38; e200. Konstantinidis K T, Ramette A, and Tiedje J M. 2006. The bacterial species definition in the genomic era. Philos Trans R Soc Lond B Biol Sci 361: 1929-1940. For complete genomes, MLSTs, specific genes, other than 16S, or sets of genes OTUs that share ≥95% average nucleotide identity are considered the same OTU. See e.g., Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940. OTUs are frequently defined by comparing sequences between organisms. Generally, sequences with no more than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic clade are provided herein.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

The terms “polynucleotide,” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides; coding or non-coding regions of a gene or gene fragment, loci (locus) defined from linkage analysis, exons, introns, messenger RNA (mRNA), micro RNA (miRNA), silencing RNA (siRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, the term “preventing” a disease or condition in a subject refers to administering to the subject to a pharmaceutical treatment. e.g., the administration of one or more agents (e.g., pharmaceutical agent), such that onset of at least one symptom of the disease or condition is delayed or prevented.

As used herein, a substance is “pure” if it is substantially free of other components. The terms “purify,” “purifying” and “purified” refer to an mEV (such as an smEV and/or a pmEV) preparation or other material that has been separated from at least some of the components with which it was associated either when initially produced or generated (e.g., whether in nature or in an experimental setting), or during any time after its initial production. An mEV (such as an smEV and/or a pmEV) preparation or compositions may be considered purified if it is isolated at or after production, such as from one or more other bacterial components, and a purified microbe or microbial population may contain other materials up to about 10%, about 20%, about 30%, about 40%, about 50%, about 60%, about 70%, about 80%, about 90%, or above about 90% and still be considered “purified.” In some embodiments, purified mEVs (such as smEVs and/or pmEVs) are more than about 80%, about 85%, about 90%, about 91%, about 92%, about 93%, about 94%, about 95%, about 96%, about 97%, about 98%, about 99%, or more than about 99% pure. mEV (such as a smEV and/or a pmEV) compositions (or preparations) are, e.g., purified from residual habitat products.

As used herein, the term “purified mEV composition” or “mEV composition” refers to a preparation that includes mEVs (such as smEVs and/or pmEVs) that have been separated from at least one associated substance found in a source material (e.g., separated from at least one other bacterial component) or any material associated with the mEVs (such as smEVs and/or pmEVs) in any process used to produce the preparation. It also refers to a composition that has been significantly enriched or concentrated. In some embodiments, the mEVs (such as smEVs and/or pmEVs) are concentrated by 2 fold, 3-fold, 4-fold, 5-fold, 10-fold, 100-fold, 1000-fold, 10,000-fold or more than 10,000 fold.

“Residual habitat products” refers to material derived from the habitat for microbiota within or on a subject. For example, fermentation cultures of microbes can contain contaminants, e.g., other microbe strains or forms (e.g., bacteria, virus, mycoplasm, and/or fungus). For example, microbes live in feces in the gastrointestinal tract, on the skin itself, in saliva, mucus of the respiratory tract, or secretions of the genitourinary tract (i.e., biological matter associated with the microbial community). Substantially free of residual habitat products means that the microbial composition no longer contains the biological matter associated with the microbial environment on or in the culture or human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms. Substantially free of residual habitat products may also mean that the microbial composition contains no detectable cells from a culture contaminant or a human or animal and that only microbial cells are detectable. In one embodiment, substantially free of residual habitat products may also mean that the microbial composition contains no detectable viral (including bacteria, viruses (e.g., phage)), fungal, mycoplasmal contaminants. In another embodiment, it means that fewer than 1×10⁻²%, 1×10⁻³%, 1×10⁻⁴%, 1×10⁻⁵%, 1×10⁻⁶%, 1×10⁻⁷%, 1×10⁻⁸% of the viable cells in the microbial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate purity include genetic analysis (e.g., PCR, DNA sequencing), serology and antigen analysis, enzymatic and metabolic analysis, and methods using instrumentation such as flow cytometry with reagents that distinguish desired constituents from contaminants.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

The terms “subject” or “patient” refers to any mammal. A subject or a patient described as “in need thereof” refers to one in need of a treatment (or prevention) for a disease. Mammals (i.e., mammalian animals) include humans, laboratory animals (e.g., primates, rats, mice), livestock (e.g., cows, sheep, goats, pigs), and household pets (e.g., dogs, cats, rodents). The subject may be a human. The subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. The subject may be healthy, or may be suffering from a cancer at any developmental stage, wherein any of the stages are either caused by or opportunistically supported of a cancer associated or causative pathogen, or may be at risk of developing a cancer, or transmitting to others a cancer associated or cancer causative pathogen. In some embodiments, a subject has lung cancer, bladder cancer, prostate cancer, plasmacytoma, colorectal cancer, rectal cancer, Merkel Cell carcinoma, salivary gland carcinoma, ovarian cancer, and/or melanoma. The subject may have a tumor. The subject may have a tumor that shows enhanced macropinocytosis with the underlying genomics of this process including Ras activation. In other embodiments, the subject has another cancer. In some embodiments, the subject has undergone a cancer therapy.

As used herein, a “systemic effect” in a subject treated with a pharmaceutical composition containing bacteria or mEVs (e.g., a pharmaceutical agent comprising bacteria or mEVs) of the instant invention means a physiological effect occurring at one or more sites outside the gastrointestinal tract. Systemic effect(s) can result from immune modulation (e.g., via an increase and/or a reduction of one or more immune cell types or subtypes (e.g., CD8+ T cells) and/or one or more cytokines). Such systemic effect(s) may be the result of the modulation by bacteria or mEVs of the instant invention on immune or other cells (such as epithelial cells) in the gastrointestinal tract which then, directly or indirectly, result in the alteration of activity (activation and/or deactivation) of one or more biochemical pathways outside the gastrointestinal tract. The systemic effect may include treating or preventing a disease or condition in a subject.

As used herein, the term “treating” a disease in a subject or “treating” a subject having or suspected of having a disease refers to administering to the subject to a pharmaceutical treatment, e.g., the administration of one or more agents, such that at least one symptom of the disease is decreased or prevented from worsening. Thus, in one embodiment, “treating” refers inter alia to delaying progression, expediting remission, inducing remission, augmenting remission, speeding recovery, increasing efficacy of or decreasing resistance to alternative therapeutics, or a combination thereof.

As used herein, a value is “greater than” another value if it is higher by any amount (e.g., each of 100, 50, 20, 12, 11, 10.6, 10.1, 10.01, and 10.001 is at least 10). Similarly, as used herein, a value is “less than” another value if it is lower by any amount (e.g., each of 1, 2, 4, 6, 8, 9, 9.2, 9.4, 9.6, 9.8, 9.9, 9.99, 9.999 is no more than 10). In contrast, as used herein, a test value “is” an anchor value when the test value rounds to the anchor value (e.g., if “an ingredient mass is 10% of a total mass,” in which case 10% is the anchor value, the test values of 9.5, 9.6, 9.7, 9.8, 9.9, 10, 10.1, 10.2, 10.3, and 10.4 would also meet the “ingredient mass is 10% of the total mass” feature).

### Bacteria

The pharmaceutical agent of the pharmaceutical compositions disclosed herein can comprise bacteria and/or microbial extracellular vesicles (mEVs) (such as smEVs and/or pmEVs). For example, the pharmaceutical agent of the pharmaceutical compositions disclosed herein can comprise a powder comprising bacteria and/or microbial extracellular vesicles (mEVs) (such as smEVs and/or pmEVs). Within a pharmaceutical agent that contains bacteria and mEVs, the mEVs can be from the same bacterial origin (e.g., same strain) as the bacteria of the pharmaceutical agent. The pharmaceutical agent can contain bacteria and/or mEVs from one or more strains.

In some embodiments, the bacteria of the pharmaceutical agent or from which the mEVs of the pharmaceutical agent are obtained are modified to reduce toxicity or other adverse effects, to enhance delivery) (e.g., oral delivery) (e.g., by improving acid resistance, muco-adherence and/or penetration and/or resistance to bile acids, digestive enzymes, resistance to anti-microbial peptides and/or antibody neutralization), to target desired cell types (e.g., M-cells, goblet cells, enterocytes, dendritic cells, macrophages), to enhance their immunomodulatory and/or therapeutic effect of the bacteria and/or mEVs (e.g., either alone or in combination with another therapeutic agent), and/or to enhance immune activation or suppression by the bacteria and/or mEVs (such as smEVs and/or pmEVs) (e.g., through modified production of polysaccharides, pili, fimbriae, adhesins). In some embodiments, the engineered bacteria described herein are modified to improve bacteria and/or mEV (such as smEV and/or pmEV) manufacturing (e.g., higher oxygen tolerance, stability, improved freeze-thaw tolerance, shorter generation times). For example, in some embodiments, the engineered bacteria described include bacteria harboring one or more genetic changes, such change being an insertion, deletion, translocation, or substitution, or any combination thereof, of one or more nucleotides contained on the bacterial chromosome or endogenous plasmid and/or one or more foreign plasmids, wherein the genetic change may result in the overexpression and/or underexpression of one or more genes. The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, or any combination thereof.

Examples of taxonomic groups (e.g., class, order, family, genus, species or strain) of bacteria that can be used as a source of bacteria and/or mEVs (such as smEVs and/or pmEVs) for a pharmaceutical agent described herein are provided herein (e.g., listed in Table 1, Table 2, Table 3, and/or Table 4 and/or elsewhere in the specification (e.g., Table J)). In some embodiments, the bacterial strain is a bacterial strain having a genome that has at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to a strain listed herein. In some embodiments, the bacteria of the pharmaceutical agent or from which the mEVs of the pharmaceutical agent are obtained are oncotrophic bacteria. In some embodiments, the bacteria of the pharmaceutical agent or from which the mEVs of the pharmaceutical agent are obtained are immunomodulatory bacteria. In some embodiments, the bacteria of the pharmaceutical agent or from which the mEVs of the pharmaceutical agent are obtained are immunostimulatory bacteria. In some embodiments, the bacteria of the pharmaceutical agent or from which the mEVs of the pharmaceutical agent are obtained are immunosuppressive bacteria. In some embodiments, the bacteria of the pharmaceutical agent or from which the mEVs of the pharmaceutical agent are obtained are immunomodulatory bacteria. In certain embodiments, the bacteria of the pharmaceutical agent or from which the mEVs of the pharmaceutical agent are obtained are generated from a combination of bacterial strains provided herein. In some embodiments, the combination is a combination of at least 2, 3, 4, 5, 6, 7, 8, 9, 10, 11, 12, 13, 14, 15, 20, 25, 30, 35, 40, 45 or 50 bacterial strains. In some embodiments, the combination includes the bacteria of the pharmaceutical agent or from which the mEVs of the pharmaceutical agent are obtained are from bacterial strains listed herein and/or bacterial strains having a genome that has at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to a strain listed herein (e.g., listed in Table 1, Table 2, and/or Table 3 and/or elsewhere in the specification (e.g., Table J)). In certain embodiments, the bacteria of the pharmaceutical agent or from which the mEVs of the pharmaceutical agent are obtained are generated from a bacterial strain provided herein. In some embodiments, the bacteria of the pharmaceutical agent or from which the mEVs of the pharmaceutical agent are obtained are from a bacterial strain listed herein (e.g., listed in Table 1, Table 2, and/or Table 3 and/or elsewhere in the specification (e.g., Table J)) and/or a bacterial strain having a genome that has at least 80%, 85%, 90%, 91%, 92%, 93%, 94%, 95%, 96%, 97%, 98%, 99%, 99.1%, 99.2%, 99.3/%, 99.4%, 99.5%, 99.6%, 99.7%, 99.8% or 99.9% sequence identity to a strain listed herein (e.g., listed in Table 1, Table 2, Table 3, and/or Table 4 and/or elsewhere in the specification (e.g., Table J)).

In some embodiments, the bacteria of the pharmaceutical agent or from which the mEVs of the pharmaceutical agent are obtained are Gram negative bacteria.

In some embodiments, the Gram negative bacteria belong to the class Negativicutes. The Negativicutes represent a unique class of microorganisms as they are the only diderm members of the Firmicutes phylum. These anaerobic organisms can be found in the environment and are normal commensals of the oral cavity and GI tract of humans. Because these organisms have an outer membrane, the yields of EVs from this class were investigated. It was found that on a per cell basis these bacteria produce a high number of vesicles (10-150 EVs/cell). The EVs from these organisms are broadly stimulatory and highly potent in in vitro assays. Investigations into their therapeutic applications in several oncology and inflammation in vivo models have shown their therapeutic potential. The Negativicutes class includes the families Veillonellaceae, Selenomonadaceae, Acidaminococcaceae, and Sporomusaceae. The Negativicutes class includes the genera Megasphaera, Selenomonas, Propionospora, and Acidaminococcus. Exemplary Negativicutes species include, but are not limited to, Megasphaera sp. Selenomonas felix, Acidaminococcus intestine, and Propionospora sp.

In some embodiments, the bacteria of the pharmaceutical agent or from which the mEVs of the pharmaceutical agent are obtained are Gram positive bacteria.

In some embodiments, the bacteria of the pharmaceutical agent or from which the mEVs of the pharmaceutical agent are obtained are aerobic bacteria.

In some embodiments, the bacteria of the pharmaceutical agent or from which the mEVs of the pharmaceutical agent are obtained are anaerobic bacteria. In some embodiments, the anaerobic bacteria comprise obligate anaerobes. In some embodiments, the anaerobic bacteria comprise facultative anaerobes.

In some embodiments, the bacteria of the pharmaceutical agent or from which the mEVs of the pharmaceutical agent are obtained are acidophile bacteria.

In some embodiments, the bacteria of the pharmaceutical agent or from which the mEVs of the pharmaceutical agent are obtained are alkaliphile bacteria.

In some embodiments, the bacteria of the pharmaceutical agent or from which the mEVs of the pharmaceutical agent are obtained are neutralophile bacteria.

In some embodiments, the bacteria of the pharmaceutical agent or from which the mEVs of the pharmaceutical agent are obtained are fastidious bacteria.

In some embodiments, the bacteria of the pharmaceutical agent or from which the mEVs of the pharmaceutical agent are obtained are nonfastidious bacteria.

In some embodiments, the bacteria of the pharmaceutical agent or from which the mEVs of the pharmaceutical agent are obtained or the mEVs themselves are lyophilized.

In some embodiments, the bacteria of the pharmaceutical agent or from which the mEVs of the pharmaceutical agent are obtained or the mEVs themselves are gamma irradiated (e.g., at 17.5 or 25 kGy).

In some embodiments, the bacteria of the pharmaceutical agent or from which the mEVs of the pharmaceutical agent are obtained or the mEVs themselves are UV irradiated.

In some embodiments, the bacteria of the pharmaceutical agent or from which the mEVs of the pharmaceutical agent are obtained or the mEVs themselves are heat inactivated (e.g., at 50° C. for two hours or at 90° C. for two hours).

In some embodiments, the bacteria of the pharmaceutical agent or from which the mEVs of the pharmaceutical agent are obtained or the mEVs themselves are acid treated.

In some embodiments, the bacteria of the pharmaceutical agent or from which the mEVs of the pharmaceutical agent are obtained or the mEVs themselves are oxygen sparged (e.g., at 0.1 vvm for two hours).

The phase of growth can affect the amount or properties of bacteria and/or mEVs produced by bacteria. For example, in the methods of mEVs preparation provided herein, mEVs can be isolated, e.g., from a culture, at the start of the log phase of growth, midway through the log phase, and/or once stationary phase growth has been reached.

In certain embodiments, the bacteria of the pharmaceutical agent or from which the mEVs of the pharmaceutical agent are obtained from obligate anaerobic bacteria. Examples of obligate anaerobic bacteria include gram-negative rods (including the genera of Bacteroides, Prevotella, Porphyromonas, Fusobacterium, Bilophila and Sutterella spp.), gram-positive cocci (primarily Peptostreptococcus spp.), gram-positive spore-forming (Clostridum spp.), non-spore-forming bacilli (Actinomyces, Propionibacterium, Eubacterium, Lactobacillus and Bifidobacterium spp.), and gram-negative cocci (mainly Veillonella spp.). In some embodiments, the obligate anaerobic bacteria are of a genus selected from the group consisting of Agathobacidum, Atopobium, Blautia, Burkholderia, Dielma, Longicatena, Paraclostridium, Turicibacter, and Tyzzerella.

The Negativicutes class includes the families Veillonellaceae, Selenomonadaceae, Acidaminococcaceae, and Sporomusaceae. The Negativicules class includes the genera Megasphaera, Selenomonas, Propionospora, and Acidaminococcus. Exemplary Negativicutes species include, but are not limited to, Megasphaera sp. Selenomonas felix, Acidaminococcus intestini, and Propionospora sp.

In some embodiments, the bacteria of the pharmaceutical agent or from which the mEVs of the pharmaceutical agent are obtained are of the Negativicutes class.

In some embodiments, the bacteria of the pharmaceutical agent or from which the mEVs of the pharmaceutical agent are obtained are of the Veillonellaceae family.

In some embodiments, the bacteria of the pharmaceutical agent or from which the mEVs of the pharmaceutical agent are obtained are of the Selenomonadaceae family.

In some embodiments, the bacteria of the pharmaceutical agent or from which the mEVs of the pharmaceutical agent are obtained are of the Acidaminococcaceae family.

In some embodiments, the bacteria of the pharmaceutical agent or from which the mEVs of the pharmaceutical agent are obtained are of the Sporomusaceae family.

In some embodiments, the bacteria of the pharmaceutical agent or from which the mEVs of the pharmaceutical agent are obtained are of the Megasphaera genus.

In some embodiments, the bacteria of the pharmaceutical agent or from which the mEVs of the pharmaceutical agent are obtained are of the Selenomonas genus.

In some embodiments, the bacteria of the pharmaceutical agent or from which the mEVs of the pharmaceutical agent are obtained are of the Propionospora genus.

In some embodiments, the bacteria of the pharmaceutical agent or from which the mEVs of the pharmaceutical agent are obtained are of the Acidaminococcus genus.

In some embodiments, the bacteria of the pharmaceutical agent or from which the mEVs of the pharmaceutical agent are obtained are Megasphaera sp. bacteria.

In some embodiments, the bacteria of the pharmaceutical agent or from which the mEVs of the pharmaceutical agent are obtained are Selenomonas felix bacteria.

In some embodiments, the bacteria of the pharmaceutical agent or from which the mEVs of the pharmaceutical agent are obtained are Acidaminococcus intestini bacteria.

In some embodiments, the bacteria of the pharmaceutical agent or from which the mEVs of the pharmaceutical agent are obtained are Propionospora sp. bacteria.

The Oscillospriraceae family within the Clostridia class of microorganisms are common commensal organisms of vertebrates.

In some embodiments, the bacteria of the pharmaceutical agent or from which the mEVs of the pharmaceutical agent are obtained are of the Clostridia class.

In some embodiments, the bacteria of the pharmaceutical agent or from which the mEVs of the pharmaceutical agent are obtained are of the Oscillospriraceae family.

In some embodiments, the bacteria of the pharmaceutical agent or from which the mEVs of the pharmaceutical agent are obtained are of the Faecalibacterium genus.

In some embodiments, the bacteria of the pharmaceutical agent or from which the mEVs of the pharmaceutical agent are obtained are of the Fournierella genus.

In some embodiments, the bacteria of the pharmaceutical agent or from which the mEVs of the pharmaceutical agent are obtained are of the Harrylintia genus.

In some embodiments, the bacteria of the pharmaceutical agent or from which the mEVs of the pharmaceutical agent are obtained are of the Agathobaculum genus.

In some embodiments, the bacteria of the pharmaceutical agent or from which the mEVs of the pharmaceutical agent are obtained are Faecalibacterium prausnitzii (e.g., Faecalibacterium prausnitzii Strain A) bacteria.

In some embodiments, the bacteria of the pharmaceutical agent or from which the mEVs of the pharmaceutical agent are obtained are Fournierella massiliensis (e.g., Fournierella massiliensis Strain A) bacteria.

In some embodiments, the bacteria of the pharmaceutical agent or from which the mEVs of the pharmaceutical agent are obtained are Harryflintia acetispora (e.g., Harryflintia acetispora Strain A) bacteria.

In some embodiments, the bacteria of the pharmaceutical agent or from which the mEVs of the pharmaceutical agent are obtained are Agathobaculum sp. (e.g., Agathobaculum sp. Strain A) bacteria.

In some embodiments, the bacteria of the pharmaceutical agent or from which the mEVs of the pharmaceutical agent are obtained are bacteria of a genus selected from the group consisting of Echerichia, Klebsiella, Lactobacillus, Shigella, and Staphylococcus.

In some embodiments, the bacteria of the pharmaceutical agent or from which the mEVs of the pharmaceutical agent are obtained are a species selected from the group consisting of Blautia massiliensis, Paraclostridium benzoelyticum, Dielma fastidiosa, Longicatena caecimuris, Lactococcus lactis cremoris, Tyzzerella nexilis, Hungatella effluvia, Klebsiella quasipneumoniae subsp. Similipneumoniae, Klebsiella oxytoca, and Veillonella tobetsuensis.

In some embodiments, the bacteria of the pharmaceutical agent or from which the mEVs of the pharmaceutical agent are obtained are a Prevotella bacteria selected from the group consisting of Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, and Prevotella veroralis.

In some embodiments, the bacteria of the pharmaceutical agent or from which the mEVs of the pharmaceutical agent are obtained are a strain of bacteria comprising a genomic sequence that is at least 80%, at least 85%, at least 90%, at least 91%, at least 92%, at least 93%, at least 94%, at least 95%, at least 96%, at least 97%, at least 98%, or at least 99% sequence identity (e.g., at least 99.1%, 99.2%, 99.3%, 99.4%, 99.5% sequence identity, at least 99.6% sequence identity, at least 99.7% sequence identity, at least 99.8% sequence identity, at least 99.9% sequence identity) to the genomic sequence of the strain of bacteria deposited with the ATCC Deposit number as provided in Table 3. In some embodiments, the bacteria of the pharmaceutical agent or from which the mEVs of the pharmaceutical agent are obtained are a strain of bacteria comprising a 16S sequence that is at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the 16S sequence of the strain of bacteria deposited with the ATCC Deposit number as provided in Table 3.

The Negativicutes class includes the families Veillonellaceae. Selenomonadaceae, Acidaminococcaceae, and Sporomusaceae. The Negativicutes class includes the genera Megasphaera. Selenomonas, Propionospora, and Acidaminococcus. Exemplary Negativicutes species include, but are not limited to, Megasphaera sp. Selenomonas felix, Acidaminococcus intestini, and Propionospora sp.

In some embodiments, the bacteria of the pharmaceutical agent or from which the mEVs of the pharmaceutical agent are obtained are of the Negativicutes class.

In some embodiments, the bacteria of the pharmaceutical agent or from which the mEVs of the pharmaceutical agent are obtained are of the Veillonellaceae family.

In some embodiments, the bacteria of the pharmaceutical agent or from which the mEVs of the pharmaceutical agent are obtained are of the Selenomonadaceae family.

In some embodiments, the bacteria of the pharmaceutical agent or from which the mEVs of the pharmaceutical agent are obtained are of the Acidaminococcaceae family.

In some embodiments, the bacteria of the pharmaceutical agent or from which the mEVs of the pharmaceutical agent are obtained are of the Sporomusaceae family.

In some embodiments, the bacteria of the pharmaceutical agent or from which the mEVs of the pharmaceutical agent are obtained are of the Megasphaera genus.

In some embodiments, the bacteria of the pharmaceutical agent or from which the mEVs of the pharmaceutical agent are obtained are of the Selenomonas genus.

In some embodiments, the bacteria of the pharmaceutical agent or from which the mEVs of the pharmaceutical agent are obtained are of the Propionospora genus.

In some embodiments, the bacteria of the pharmaceutical agent or from which the mEVs of the pharmaceutical agent are obtained are of the Acidaminococcus genus.

In some embodiments, the bacteria of the pharmaceutical agent or from which the mEVs of the pharmaceutical agent are obtained are Megasphaera sp. bacteria.

In some embodiments, the bacteria of the pharmaceutical agent or from which the mEVs of the pharmaceutical agent are obtained are Selenomonas felix bacteria.

In some embodiments, the bacteria of the pharmaceutical agent or from which the mEVs of the pharmaceutical agent are obtained are Acidaminococcus intestini bacteria.

In some embodiments, the bacteria of the pharmaceutical agent or from which the mEVs of the pharmaceutical agent are obtained are Propionospora sp. bacteria.

The Oscillospriraceae family within the Clostndia class of microorganisms are common commensal organisms of vertebrates.

In some embodiments, the bacteria of the pharmaceutical agent or from which the mEVs of the pharmaceutical agent are obtained are of the Clostridia class.

In some embodiments, the bacteria of the pharmaceutical agent or from which the mEVs of the pharmaceutical agent are obtained are of the Oscillospriraceae family.

In some embodiments, the bacteria of the pharmaceutical agent or from which the mEVs of the pharmaceutical agent are obtained are of the Faecalibacterium genus.

In some embodiments, the bacteria of the pharmaceutical agent or from which the mEVs of the pharmaceutical agent are obtained are of the Fournierella genus.

In some embodiments, the bacteria of the pharmaceutical agent or from which the mEVs of the pharmaceutical agent are obtained are of the Harryflintia genus.

In some embodiments, the bacteria of the pharmaceutical agent or from which the mEVs of the pharmaceutical agent are obtained are of the Agathobaculum genus.

In some embodiments, the bacteria of the pharmaceutical agent or from which the mEVs of the pharmaceutical agent are obtained are Faecalibacterium prausnitzii (e.g., Faecalibacterium prausnitzii Strain A) bacteria.

In some embodiments, the bacteria of the pharmaceutical agent or from which the mEVs of the pharmaceutical agent are obtained are Fournierella massiliensis (e.g., Fournierella massiliensis Strain A) bacteria.

In some embodiments, the bacteria of the pharmaceutical agent or from which the mEVs of the pharmaceutical agent are obtained are Harryflintia acetispora (e.g., Harryflintia acetispora Strain A) bacteria.

In some embodiments, the bacteria of the pharmaceutical agent or from which the mEVs of the pharmaceutical agent are obtained are Agathobaculum sp. (e.g., Agathobaculum sp. Strain A) bacteria.

In some embodiments, the bacteria of the pharmaceutical agent or from which the mEVs of the pharmaceutical agent are obtained are a strain of Agathobaculum sp. In some embodiments, the Agathobaculum sp. strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Agathobaculum sp. Strain A (ATCC Deposit Number PTA-125892). In some embodiments, the Agathobaculum sp. strain is the Agathobaculum sp. Strain A (ATCC Deposit Number PTA-125892).

In some embodiments, the bacteria of the pharmaceutical agent or from which the mEVs of the pharmaceutical agent are obtained are of the class Bacteroidia [phylum Bacteroidota]. In some embodiments, the bacteria of the pharmaceutical agent or from which the mEVs of the pharmaceutical agent are obtained are bacteria of order Bacteroidales. In some embodiments, the bacteria of the pharmaceutical agent or from which the mEVs of the pharmaceutical agent are obtained are of the family Porphyromonoadaceae. In some embodiments, the bacteria of the pharmaceutical agent or from which the mEVs of the pharmaceutical agent are obtained are of the family Prevotellaceae. In some embodiments, the bacteria of the pharmaceutical agent or from which the mEVs of the pharmaceutical agent are obtained are bacteria of the class Bacteroidia wherein the cell envelope structure of the bacteria is diderm. In some embodiments, the bacteria of the pharmaceutical agent or from which the mEVs of the pharmaceutical agent are obtained are bacteria of the class Bacteroidia that stain Gram negative. In some embodiments, the bacteria of the pharmaceutical agent or from which the mEVs of the pharmaceutical agent are obtained are bacteria of the class Bacteroidia wherein the bacteria is diderm and the bacteria stain Gram negative.

In some embodiments, the bacteria of the pharmaceutical agent or from which the mEVs of the pharmaceutical agent are obtained are bacteria of the class Clostridia [phylum Firmicutes]. In some embodiments, the bacteria of the pharmaceutical agent or from which the mEVs of the pharmaceutical agent are obtained are of the order Eubacteriales. In some embodiments, the bacteria of the pharmaceutical agent or from which the mEVs of the pharmaceutical agent are obtained are of the family Oscillispiraceae. In some embodiments, the bacteria of the pharmaceutical agent or from which the mEVs of the pharmaceutical agent are obtained are of the family Lachnospiraceae. In some embodiments, the bacteria of the pharmaceutical agent or from which the mEVs of the pharmaceutical agent are obtained are of the family Peptostreptococcaceae. In some embodiments, the bacteria of the pharmaceutical agent or from which the mEVs of the pharmaceutical agent are obtained are of the family Clostridiales family XIII/Incertae sedis 41. In some embodiments, the bacteria of the pharmaceutical agent or from which the mEVs of the pharmaceutical agent are obtained are of the class Clostridia wherein the cell envelope structure of the bacteria is monoderm. In some embodiments, the bacteria of the pharmaceutical agent or from which the mEVs of the pharmaceutical agent are obtained are of the class Clostridia that stain Gram negative. In some embodiments, the bacteria of the pharmaceutical agent or from which the mEVs of the pharmaceutical agent are obtained are of the class Clostridia that stain Gram positive. In some embodiments, the bacteria of the pharmaceutical agent or from which the mEVs of the pharmaceutical agent are obtained are of the class Clostridia wherein the cell envelope structure of the bacteria is monoderm and the bacteria stain Gram negative. In some embodiments, the bacteria of the pharmaceutical agent or from which the mEVs of the pharmaceutical agent are obtained are of the class Clostridia wherein the cell envelope structure of the bacteria is monoderm and the bacteria stain Gram positive.

In some embodiments, the bacteria of the pharmaceutical agent or from which the mEVs of the pharmaceutical agent are obtained are of the class Negativicutes [phylum Firmicutes]. In some embodiments, the bacteria of the pharmaceutical agent or from which the mEVs of the pharmaceutical agent are obtained are of the order Veillonellales. In some embodiments, the bacteria of the pharmaceutical agent or from which the mEVs of the pharmaceutical agent are obtained are of the family Veillonelloceae. In some embodiments, the bacteria of the pharmaceutical agent or from which the mEVs of the pharmaceutical agent are obtained are of the order Selenomonadales. In some embodiments, the bacteria of the pharmaceutical agent or from which the mEVs of the pharmaceutical agent are obtained are bacteria of the family Selenomonadaceae. In some embodiments, the bacteria of the pharmaceutical agent or from which the mEVs of the pharmaceutical agent are obtained are of the family Sporomusaceae. In some embodiments, t the bacteria of the pharmaceutical agent or from which the mEVs of the pharmaceutical agent are obtained are of the class Negativicutes wherein the cell envelope structure of the bacteria is diderm. In some embodiments, the bacteria of the pharmaceutical agent or from which the mEVs of the pharmaceutical agent are obtained are of the bacteria of the pharmaceutical agent or from which the mEVs of the pharmaceutical agent are obtained are the EVs are from bacteria of the class Negativicutes wherein the cell envelope structure of the bacteria is diderm and the bacteria stain Gram negative.

In some embodiments, the bacteria of the pharmaceutical agent or from which the mEVs of the pharmaceutical agent are obtained are of the class Synergistia [phylum Synergistota]. In some embodiments, the bacteria of the pharmaceutical agent or from which the mEVs of the pharmaceutical agent are obtained are of the order Synergistales. In some embodiments, the bacteria of the pharmaceutical agent or from which the mEVs of the pharmaceutical agent are obtained are of the family Synergistaceae. In some embodiments, the bacteria of the pharmaceutical agent or from which the mEVs of the pharmaceutical agent are obtained are of the class Synergistia wherein the cell envelope structure of the bacteria is diderm. In some embodiments, the bacteria of the pharmaceutical agent or from which the mEVs of the pharmaceutical agent are obtained are of the class Synergistia that stain Gram negative. In some embodiments, the bacteria of the pharmaceutical agent or from which the mEVs of the pharmaceutical agent are obtained are of the class Synergistia wherein the cell envelope structure of the bacteria is diderm and the bacteria stain Gram negative.

In some embodiments, the bacteria of the pharmaceutical agent or from which the mEVs of the pharmaceutical agent are obtained are from one strain of bacteria, e.g., a strain provided herein.

In some embodiments, the bacteria of the pharmaceutical agent or from which the mEVs of the pharmaceutical agent are obtained are from one strain of bacteria (e.g., a strain provided herein) or from more than one strain provided herein.

In some embodiments, the bacteria of the pharmaceutical agent or from which the mEVs of the pharmaceutical agent are obtained are Lactococcus lactis cremoris bacteria. e.g., a strain comprising at least 90% or at least 99% genomic, 16S and/or CRISPR sequence identity to the nucleotide sequence of the Lactococcus lactis cremoris Strain A (ATCC designation number PTA-125368). In some embodiments, the bacteria of the pharmaceutical agent or from which the mEVs of the pharmaceutical agent are obtained are Lactococcus bacteria. e.g., Lactococcus lactis cremoris Strain A (ATCC designation number PTA-125368).

In some embodiments, the bacteria of the pharmaceutical agent or from which the mEVs of the pharmaceutical agent are obtained are Prevotella bacteria, e.g., a strain comprising at least 90% or at least 99% genomic, 16S and/or CRISPR sequence identity to the nucleotide sequence of the Prevotella Strain B 50329 (NRRL accession number B 50329). In some embodiments, the bacteria of the pharmaceutical agent or from which the mEVs of the pharmaceutical agent are obtained are Prevotella bacteria, e.g., Prevotella Strain B 50329 (NRRL accession number B 50329).

In some embodiments, the bacteria of the pharmaceutical agent or from which the mEVs of the pharmaceutical agent are obtained are Prevotella bacteria, e.g., a strain comprising at least 90% or at least 99% genomic, 16S and/or CRISPR sequence identity to the nucleotide sequence of the Prevotella Strain C (ATCC Accession Number PTA-126140). In some embodiments, the bacteria of the pharmaceutical agent or from which the mEVs of the pharmaceutical agent are obtained are Prevotella bacteria, e.g., Prevotella Strain C (ATCC Accession Number PTA-126140).

In some embodiments, the bacteria of the pharmaceutical agent or from which the mEVs of the pharmaceutical agent are obtained are Bifidobacterium bacteria, e.g., a strain comprising at least 90% or at least 99% genomic, 16S and/or CRISPR sequence identity to the nucleotide sequence of the Bifidobacterium bacteria deposited as ATCC designation number PTA-125097. In some embodiments, the bacteria of the pharmaceutical agent or from which the mEVs of the pharmaceutical agent are obtained are Bifidobacterium bacteria, e.g., Bifidobacterium bacteria deposited as ATCC designation number PTA-125097.

In some embodiments, the bacteria of the pharmaceutical agent or from which the mEVs of the pharmaceutical agent are obtained are Veillonella bacteria. e.g., a strain comprising at least 90% or at least 99% genomic, 16S and/or CRISPR sequence identity to the nucleotide sequence of the Veillonella bacteria deposited as ATCC designation number PTA-125691. In some embodiments, the bacteria of the pharmaceutical agent or from which the mEVs of the pharmaceutical agent are obtained are Veillonella bacteria, e.g., Veillonella bacteria deposited as ATCC designation number PTA-125691.

In some embodiments, the bacteria of the pharmaceutical agent or from which the mEVs of the pharmaceutical agent are obtained are Ruminococcus gnavus bacteria. In some embodiments, the Ruminococcus gnavus bacteria are a strain comprising at least 90% (or at least 97%) genomic, 16S and/or CRISPR sequence identity to the nucleotide sequence of the Ruminococcus gnavus bacteria deposited as ATCC designation number PTA-126695. In some embodiments, the Ruminococcus gnavus bacteria are a strain comprising at least 99% genomic, 16S and/or CRISPR sequence identity to the nucleotide sequence of the Ruminococcus gnavus bacteria deposited as ATCC designation number PTA-126695. In some embodiments, the Ruminococcus gnavus bacteria are Ruminococcus gnavus bacteria deposited as ATCC designation number PTA-126695.

In some embodiments, the bacteria of the pharmaceutical agent or from which the mEVs of the pharmaceutical agent are obtained are Megasphaera sp. bacteria. In some embodiments, the Megasphaera sp. bacteria are a strain comprising at least 90% (or at least 97%) genomic, 16S and/or CRISPR sequence identity to the nucleotide sequence of the Megasphaera sp. bacteria deposited as ATCC designation number PTA-126770. In some embodiments, the Megasphaera sp. bacteria are a strain comprising at least 99% genomic, 16S and/or CRISPR sequence identity to the nucleotide sequence of the Megasphaera sp. bacteria deposited as ATCC designation number PTA-126770. In some embodiments, the Megasphaera sp. bacteria are Megasphaera sp. bacteria deposited as ATCC designation number PTA-126770.

In some embodiments, the bacteria of the pharmaceutical agent or from which the mEVs of the pharmaceutical agent are obtained are Fournierella massihensis bacteria. In some embodiments, the Fournierella massiliensis bacteria are a strain comprising at least 90% (or at least 97%) genomic, 16S and/or CRISPR sequence identity to the nucleotide sequence of the Fournierella massiliensis bacteria deposited as ATCC designation number PTA-126696. In some embodiments, the Fournierella massihensis bacteria are a strain comprising at least 99% genomic, 16S and/or CRISPR sequence identity to the nucleotide sequence of the Fournierella massiliensis bacteria deposited as ATCC designation number PTA-126696. In some embodiments, the Fournierella massiliensis bacteria are Fournierella massiliensis bacteria deposited as ATCC designation number PTA-126696.

In some embodiments, the bacteria of the pharmaceutical agent or from which the mEVs of the pharmaceutical agent are obtained are Harryflintia acetispora bacteria. In some embodiments, the Harryflintia acetispora bacteria are a strain comprising at least 90% (or at least 97%) genomic, 16S and/or CRISPR sequence identity to the nucleotide sequence of the Harryflintia acetispora bacteria deposited as ATCC designation number PTA-126694. In some embodiments, the Harryflintia acetispora bacteria are a strain comprising at least 99% genomic, 16S and/or CRISPR sequence identity to the nucleotide sequence of the Harryflintia acetispora bacteria deposited as ATCC designation number PTA-126694. In some embodiments, the Harryflintia acetispora bacteria are Harryflintia acetispora bacteria deposited as ATCC designation number PTA-126694.

In some embodiments, the bacteria of the pharmaceutical agent or from which the mEVs of the pharmaceutical agent are obtained are bacteria that produce metabolites, e.g., the bacteria produce butyrate, iosine, proprionate, or tryptophan metabolites.

In some embodiments, the bacteria of the pharmaceutical agent or from which the mEVs of the pharmaceutical agent are obtained produce butyrate. In some embodiments, the bacteria are from the genus Blautia; Christensella; Copracoccus; Eubacterium; Lachnosperacea; Megasphaera; or Roseburia.

In some embodiments, the bacteria of the pharmaceutical agent or from which the mEVs of the pharmaceutical agent are obtained produce iosine. In some embodiments, the bacteria are from the genus Bifidobacterium; Lactobacillus; or Olsenella.

In some embodiments, the bacteria of the pharmaceutical agent or from which the mEVs of the pharmaceutical agent are obtained produce proprionate. In some embodiments, the bacteria are from the genus Akkermansia; Bacteroides; Dialister; Eubacterium; Megasphaera; Parabacteriodes; Prevotella; Ruminococcus; or Veillonella.

In some embodiments, the bacteria of the pharmaceutical agent or from which the mEVs of the pharmaceutical agent are obtained produce tryptophan metabolites. In some embodiments, the bacteria are from the genus Lactobacillus or Peptostreptococcus.

In some embodiments, the bacteria of the pharmaceutical agent or from which the mEVs of the pharmaceutical agent are obtained are bacteria that produce inhibitors of histone deacetylase 3 (HDAC3). In some embodiments, the bacteria are from the species Bariatricus massiliensis, Faecalibacterium prausnitzii, Megasphaera massiliensis or Roseburia intestinalis. In some embodiments, the bacteria are from the genus Alloiococcus; Bacillus; Catenibacterium; Corynebacterium; Cupriavidus; Enhydrobacter; Exiguobacterium; Faecalibacterium; Geobacillus; Methylobacterium; Micrococcus; Morganella; Proteus; Pseudomonas; Rhizobium; or Sphingomonas. In some embodiments, the bacteria are from the genus Cutibacterium. In some embodiments, the bacteria are from the species Cutibacterium avidum. In some embodiments, the bacteria are from the genus Lactobacillus. In some embodiments, the bacteria are from the species Lactobacillus gasseri. In some embodiments, the bacteria are from the genus Dysosmobacter. In some embodiments, the bacteria are from the species Dysosmobacter welbionis.

Applicant represents that the ATCC is a depository affording permanence of the deposit and ready accessibility thereto by the public if a patent is granted. All restrictions on the availability to the public of the material so deposited will be irrevocably removed upon the granting of a patent. The material will be available during the pendency of the patent application to one determined by the Commissioner to be entitled thereto under 37 CFR 1.14 and 35 U.S.C. 122. The deposited material will be maintained with all the care necessary to keep it viable and uncontaminated for a period of at least five years after the most recent request for the furnishing of a sample of the deposited plasmid, and in any case, for a period of at least thirty (30) years after the date of deposit or for the enforceable life of the patent, whichever period is longer. Applicant acknowledges its duty to replace the deposit should the depository be unable to furnish a sample when requested due to the condition of the deposit.

In some embodiments, the bacteria of the pharmaceutical agent or from which the mEVs of the pharmaceutical agent are obtained are of the genus Alloiococcus; Bacillus; Catenibacterium; Corynebacterium; Cupriavidus; Enhydrobacter; Exiguobacterium; Faecalibacterium; Geobacillus; Methylobacterium; Micrococcus; Morganella; Proteus; Pseudomonas; Rhizobium; or Sphingomonas.

In some embodiments, the bacteria of the pharmaceutical agent or from which the mEVs of the pharmaceutical agent are obtained are of the Cutibacterium genus. In some embodiments, the bacteria of the pharmaceutical agent or from which the mEVs of the pharmaceutical agent are obtained are Cutibacterium avidum bacteria.

In some embodiments, the bacteria of the pharmaceutical agent or from which the mEVs of the pharmaceutical agent are obtained are of the genus Leuconostoc.

In some embodiments, the bacteria of the pharmaceutical agent or from which the mEVs of the pharmaceutical agent are obtained are of the genus Lactobacillus.

In some embodiments, the bacteria of the pharmaceutical agent or from which the mEVs of the pharmaceutical agent are obtained are of the genus Akkermansia muciniphila; Bacillus; Blautia; Cupriavidus; Enhydrobacter; Faecalibacterium; Lactobacillus; Lactococcus; Micrococcus; Morganella; Propionibacterium; Proteus; Rhizobium; or Streptococcus.

In some embodiments, the bacteria of the pharmaceutical agent or from which the mEVs of the pharmaceutical agent are obtained are Leuconostoc holzapfelii bacteria.

In some embodiments, the bacteria of the pharmaceutical agent or from which the mEVs of the pharmaceutical agent are obtained are Akkermansia muciniphila; Cupriavidus metallidurans; Faecalibacterium prausnitzii; Lactobacillus casei; Lactobacillus plantarum; Lactobacillus paracasei; Lactobacillus plantarum; Lactobacillus rhamnosus; Lactobacillus sakei; or Streptococcus pyogenes bacteria.

In some embodiments, the bacteria of the pharmaceutical agent or from which the mEVs of the pharmaceutical agent are obtained are Lactobacillus casei; Lactobacillus plantarum; Lactobacillus paracasei; Lactobacillus plantarum; Lactobacillus rhamnosus; or Lactobacillus sakei bacteria.

In some embodiments, the bacteria of the pharmaceutical agent or from which the mEVs of the pharmaceutical agent are obtained are Megasphaera sp. bacteria (e.g., from the strain with accession number NCIMB 43385, NCIMB 43386 or NCIMB 43387).

In some embodiments, the bacteria of the pharmaceutical agent or from which the mEVs of the pharmaceutical agent are obtained are Megasphaera massiliensis bacteria (e.g., from the strain with accession number NCIMB 42787, NCIMB 43388 or NCIMB 43389).

In some embodiments, the bacteria of the pharmaceutical agent or from which the mEVs of the pharmaceutical agent are obtained are Megasphaera massiliensis bacteria (e.g., from the strain with accession number DSM 26228).

In some embodiments, the bacteria of the pharmaceutical agent or from which the mEVs of the pharmaceutical agent are obtained are Parabacteriodes distasonis bacteria (e.g., from the strain with accession number NCIMB 42382).

In some embodiments, the bacteria of the pharmaceutical agent or from which the mEVs of the pharmaceutical agent are obtained are Megasphaera massiliensis bacteria (e.g., from the strain with accession number NCIMB 43388 or NCIMB 43389), or a derivative thereof. See, e.g., WO 2020/120714. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of Megasphaera massiliensis bacteria from the strain with accession number NCIMB 43388 or NCIMB 43389. In some embodiments, the Megasphaera massiliensis bacteria is the strain with accession number NCIMB 43388 or NCIMB 43389.

In some embodiments, the bacteria of the pharmaceutical agent or from which the mEVs of the pharmaceutical agent are obtained are Megasphaera massiliensis bacteria strain deposited under accession number NCIMB 42787, or a derivative thereof. See, e.g., WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Megasphaera massiliensis bacteria strain deposited under accession number NCIMB 42787. In some embodiments, the Megasphaera massiliensis bacteria is the strain deposited under accession number NCIMB 42787.

In some embodiments, the bacteria of the pharmaceutical agent or from which the mEVs of the pharmaceutical agent are obtained are Megasphaera spp. bacteria from the strain with accession number NCIMB 43385, NCIMB 43386 or NCIMB 43387, or a derivative thereof. See, e.g., WO 2020/120714. In some embodiments, the Megasphaera sp. bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Megasphaera sp. from a strain with accession number NCIMB 43385, NCIMB 43386 or NCIMB 43387. In some embodiments, the Megasphaera sp. bacteria is the strain with accession number NCIMB 43385, NCIMB 43386 or NCIMB 43387.

In some embodiments, the bacteria of the pharmaceutical agent or from which the mEVs of the pharmaceutical agent are obtained are Parabacteroides distasonis bacteria deposited under accession number NCIMB 42382, or a derivative thereof. See, e.g., WO 2018/229216. In some embodiments, the Parabacteroides distasonis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Parabacteroides distasonis bacteria deposited under accession number NCIMB 42382. In some embodiments, the Parabacteroides distasonis bacteria is the strain deposited under accession number NCIMB 42382.

In some embodiments, the bacteria of the pharmaceutical agent or from which the mEVs of the pharmaceutical agent are obtained are Megasphaera massiliensis bacteria deposited under accession number DSM 26228, or a derivative thereof. See, e.g., WO 2018/229216. In some embodiments, the Megasphaera massiliensis bacteria is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of Megasphaera massiliensis bacteria deposited under accession number DSM 26228. In some embodiments, the Megasphaera massiliensis bacteria is the strain deposited under accession number DSM 26228.

TABLE 1
Bacteria by Class

| Class | Order | Family | Genus | Species |
|---|---|---|---|---|
| Actinobacter | Actinomycetales | Mycobacteriaceae | Mycobacterium | |
| | | Streptomycetaceae | Streptomyces | Streptomyces |
| | | | | lividans, |
| | | | | Streptomyces |
| | | | | coelicolor, |
| | | | | Streptomyces |
| | | | | sudanesis, |
| | | | | Streptomyces |
| | | | | somaliensis |
| | Bifidobacteriales | Bifidobacteriaceae | Bifidobacterium | Bifidobacterium |
| | | | | adolescentis, |
| | | | | Bifidobacterium |
| | | | | animalis, |
| | | | | Bifidobacterium |
| | | | | bifidum, |
| | | | | Bifidobacterium |
| | | | | breve, |
| | | | | Bifidobacterium |
| | | | | lactis, |
| | | | | Bifidobacterium |
| | | | | longum, |
| | | | | Bifidobacterium |
| | | | | pseudocatenulatum |
| | Coriobacteriales | Coriobacteriaceae | Collinsella | Collinsella |
| | | | | aerofaciens |
| | | | Olsenella | Olsenellafaecalis |
| | Propionibacteriales | Propionibacteraceae | Propionibacterium | |
| Bacilli | Bacillales | Bacillales | Gemella | Gemella |
| | | incertaesedis | | haemolysans, |
| | | family XI | | Gemella |
| | | | | morbillorum |
| | | Listeraceae | Listeria | Listeria |
| | | | | monocytogenes, |
| | | | | Listeria welshimeri |
| | Lactobacilluses | Enterococcaceae | Enterococcus | Enterococcus |
| | | | | durans, |
| | | | | Enterococcus |
| | | | | faecium, |
| | | | | Enterococcus |
| | | | | faecalis, |
| | | | | Enterococcus |
| | | | | gallinarum, |
| | | | | Enterococcus |
| | | | | villorum |
| | | | Lactobacillus | Lactobacillus casei, |
| | | | | Lactobacillus |
| | | | | fermentum, |
| | | | | Lactococcus lactis |
| | | | | cremoris, |
| | | | | Lactobacillus |
| | | | | mucosae, |
| | | | | Lactobacillus |
| | | | | plantarum, |
| | | | | Lactobacillus |
| | | | | reuteri, |
| | | | | Lactobacillus |
| | | | | rhamnosus, L. |
| | | | | salvarius |
| | | Streptococcaceae | Lactococcus | |
| | | | Staphylococcus | Staphylococcus |
| | | | | aureus |
| | | | Streptococcus | Streptococcus |
| | | | | agalactiae, |
| | | | | Streptococcus |
| | | | | aureus, |
| | | | | Streptococcus |
| | | | | australi, |
| | | | | Streptococcus |
| | | | | mutans, |
| | | | | Streptococcus |
| | | | | parasanguinis, |
| | | | | Streptococcus |
| | | | | pneumoniae, |
| | | | | Streptococcus |
| | | | | pyogenes, |
| | | | | Streptococcus |
| | | | | salivraius |
| Bacteriodes | Bacteroidales | Bacteriodaceae | Bacteriodes | Bacteroides caccae, |
| | | | | Bacteroides |
| | | | | cellulosilyticus, |
| | | | | Bacteroides |
| | | | | coprocola, |
| | | | | Bacteroides dorei, |
| | | | | Bacteroides fragilis, |
| | | | | Bacteroides ovatus, |
| | | | | Bacteroides |
| | | | | putredinis, |
| | | | | Bacteroides |
| | | | | salanitronis, |
| | | | | Bacteroides |
| | | | | thetaiotaomicron, |
| | | | | Bacteroides |
| | | | | vulgatus |
| | | Odoribacteraceae | Odoribacter | Odoribacter |
| | | | | splanchnicus |
| | | Porphyromonadaceae | Parabacteriodes | Parabacteriodes |
| | | | | distasonis, |
| | | | | Parabacteroides |
| | | | | goldsteinii, P |
| | | | | Parabacteriodes |
| | | | | merdae |
| | | | Porphyromonas | Porphyromonas |
| | | | | gingivalis |
| | | Prevotellaceae | Prevotella | Prevotella |
| | | | | albensis, |
| | | | | Prevotella amnii, |
| | | | | Prevotella |
| | | | | aurantiaca, |
| | | | | Prevotella |
| | | | | baroniae, |
| | | | | Prevotella |
| | | | | bergensis, |
| | | | | Prevotella bivia, |
| | | | | Prevotella brevis, |
| | | | | Prevotella bryantii, |
| | | | | Prevotella buccae, |
| | | | | Prevotella |
| | | | | buccalis, |
| | | | | Prevotella |
| | | | | colorans, |
| | | | | Prevotella |
| | | | | corporis, |
| | | | | Prevotella copri, |
| | | | | Prevotella dentalis, |
| | | | | Prevotella |
| | | | | dentasini, |
| | | | | Prevotella |
| | | | | denticola, |
| | | | | Prevotella disiens,, |
| | | | | Prevotella enoeca, |
| | | | | Prevotella falsenii, |
| | | | | Prevotella fusca, |
| | | | | Prevotella |
| | | | | heparinolytica, |
| | | | | Prevotella |
| | | | | histicola, |
| | | | | Prevotella |
| | | | | intermedia, |
| | | | | Prevotella jejuni, , |
| | | | | Prevotella |
| | | | | loescheii, |
| | | | | Prevotella |
| | | | | maculosa, |
| | | | | Prevotella marshii, |
| | | | | Prevotella |
| | | | | melaninogenica, |
| | | | | Prevotella micans, |
| | | | | Prevotella |
| | | | | multiformis, |
| | | | | Prevotella |
| | | | | multisaccharivorax, |
| | | | | Prevotella |
| | | | | nanceiensis, |
| | | | | Prevotella |
| | | | | nigrescens, |
| | | | | Prevotella oralis, |
| | | | | Prevotella oris, , |
| | | | | Prevotella oryzae, |
| | | | | Prevotella |
| | | | | oulorum, |
| | | | | Prevotella pallens, |
| | | | | Prevotella |
| | | | | paludivivens, |
| | | | | Prevotella |
| | | | | pleuritidis |
| | | | | Prevotella |
| | | | | ruminicola, |
| | | | | Prevotella |
| | | | | saccharolytica, |
| | | | | Prevotella salivae, |
| | | | | Prevotella scopos, |
| | | | | Prevotella shahii, |
| | | | | Prevotella |
| | | | | stercorea, |
| | | | | Prevotella |
| | | | | tannerae, |
| | | | | Prevotella |
| | | | | timonensis, |
| | | | | Prevotella |
| | | | | veroralis, |
| | | | | Prevotella |
| | | | | zoogleoformans |
| | | Rikenellaceae | Alstipes | Alistipes communis, |
| | | | | Alistipes dispar, A. |
| | | | | finegoldii, Alistipes |
| | | | | indistinctus, |
| | | | | Alistipes ihumii, |
| | | | | Alistipes inops,, |
| | | | | Alistipes |
| | | | | massiliensis,, |
| | | | | Alistipes megaguti, |
| | | | | Alistipes obesi, |
| | | | | Alistipes |
| | | | | onderdonkii, |
| | | | | Alistipes |
| | | | | provencensis, |
| | | | | Alistipes putredinis, |
| | | | | Alistipes |
| | | | | senegalensis, , |
| | | | | Alistipes shahii, |
| | | | | Alistipes timonensis |
| Betaproteoba | Burkholderiales | Alcaligenaceae | Paenalcaligenes | Paenalcaligenes |
| cteria | | | | hominis |
| | | | Bordella | Bordella pertussis |
| | | Burkholderiaceae | Burkholderia | Burkholderia mallei, |
| | | | | Burkholderia |
| | | | | pseudomallei |
| | | | Ralstonia | Ralstonia |
| | | | | solanacearum |
| | | Neisseriaceae | Neisseria | Neisseria |
| | | | | meningitidis |
| | | Sutterellaceae | Sutterella | Sutterella |
| | | | | parvirubra, |
| | | | | Sutterella |
| | | | | stercoricanis, |
| | | | | Sutterella |
| | | | | wadsworthensis |
| Clostridia | Clostridiales | Catabacteriaceae | Catabacter | Catabacter |
| | | | | hongkongensis |
| | | Clostridiaceae | Aminiphila | Anaerosphaera |
| | | | | aminiphila |
| | | | Christensenellaceae | C. massiliensis, C. |
| | | | | minuta, |
| | | | | C. timonensis |
| | | | Hungatella | Hungatella effluvia |
| | | Eubacteriaceae | Eubacterium | Eubacterium |
| | | | | contortum, |
| | | | | Enterococcus |
| | | | | durans, |
| | | | | Eubacterium |
| | | | | eligens, |
| | | | | Eubacterium |
| | | | | faecium |
| | | | | Enterococcus |
| | | | | faecalis, |
| | | | | Enterococcus |
| | | | | gallinarum, |
| | | | | Eubacterium |
| | | | | hadrum, |
| | | | | Eubacterium hallii, |
| | | | | Eubacterium |
| | | | | limosum, |
| | | | | Eubacterium |
| | | | | ramulus, |
| | | | | Eubacterium |
| | | | | rectale, |
| | | | | Enterococcus |
| | | | | villorum |
| | | Lachnospiraceae | Anaerostipes | Anaerostipes |
| | | | | caccae, |
| | | | | Anaerostipes hadrus |
| | | | Blautia | Blautia |
| | | | | hydrogenotrophica, |
| | | | | Blautia massiliensis, |
| | | | | Blautia stercoris, |
| | | | | Blautia wexlerae |
| | | | Catonella | Catonella morbi |
| | | | Coprococcus | Coprococcus catus, |
| | | | | Coprococcus comes, |
| | | | | Coprococcus |
| | | | | eutactus |
| | | | Dialister | Dialister invisus, |
| | | | | Dialister |
| | | | | micraeophilus, |
| | | | | Dialister |
| | | | | succinatiphilus |
| | | | Dorea | Dorea |
| | | | | formicigenerans, |
| | | | | Dorea longicatena, |
| | | | Johnsonella | Johnsonella ignava |
| | | | Oribacterium | Oribacterium |
| | | | | parvum, |
| | | | | Oribacterium sinus |
| | | | Lachnobacterium | |
| | | | Lachnoclostridium | |
| | | | Lacrimispora | Lacrimispora |
| | | | | sacchaarolytica |
| | | | Roseburia | Roseburia hominis, |
| | | | | seburia intestinalis |
| | | | Tyzzerella | Tyzzerella nexilis |
| | | Oscillospiraceae | Oscillibacter | Oscillibacter |
| | | | | valericigenes |
| | | | Harryflintia | Harryflinta |
| | | | | acetispora |
| | | Peptococcaceae | | |
| | | Peptostreptococcaceae | Paraclostridium | Paraclostridium |
| | | | | benzoelyticum |
| | | | Peptostreptococcus | Peptostreptococcus |
| | | | | russellii |
| | | Ruminococcaceae | | Agathobaculum sp. |
| | | | Fournierella | Fournierella |
| | | | | masssiliensis |
| | | | Ruminococcus | Ruminococcus |
| | | | | albus, |
| | | | | Ruminococcus |
| | | | | bromii, Ruminococcus |
| | | | | callidus, |
| | | | | Ruminococcus |
| | | | | gnavus, |
| | | | | Ruminococcus |
| | | | | inulinivorans, |
| | | | | Ruminococcus |
| | | | | obeum, |
| | | | | Ruminococcus |
| | | | | torques |
| | | | Faecalibacterium | Faecalibacterium |
| | | | | prasusnitzii |
| | | Clostridiales family | | Intestimonas |
| | | XIII/Incertae sedis | | butyriciproducens |
| Fusobacteria | Fusobacteriales | Fusobacteriaceae | Fusobacterium | Fusobacterium |
| | | | | nucleatum, |
| | | | | Fusobacterium |
| | | | | naviforme |
| | | Leptotrichiaceae | Leptotrichia | |
| | | | Sneathia | |
| Gammaprote | Enterobacterales | Enterobacteriaceae | Klebsiella | Klebsiella oxytoca,, |
| obacteria | | | | Klebsiella |
| | | | | pneumoniae, |
| | | | | Klebsiella |
| | | | | quasipneumoniae |
| | | | | subsp. |
| | | | | Similipneumoniae, |
| | | | Escherichia | Escherichia coli |
| | | | | strain Nissle 1917 |
| | | | | (EcN) |
| | | | | Escherichia coli |
| | | | | strain ECOR12 |
| | | | | Escherichia coli |
| | | | | strain ECOR63 |
| | | | Shigella | |
| Negativicutes | | Acidaminococcaceae | Acidaminococcus | Acidaminococcus |
| | | | | fermentans, |
| | | | | Acidaminococcus |
| | | | | intestine |
| | | | Phascolarctobacterium | Phascolarctobacterium |
| | | | | faecium, |
| | | | | Phascolarctobacterium |
| | | | | succinatutens |
| | | Selenomonadaceae | Selenomonas | Selenomonas felix, |
| | | | | Selemonadales |
| | | | | incertae sedis, |
| | | | | Selenomonas |
| | | | | sputigena |
| | | Sporomusaceae | Selenomonadales | |
| | | Veillonellaceae | Allisonella | |
| | | | Anaeroglobus | Anaeroglobus |
| | | | | germinatus |
| | | | Caecibacter | |
| | | | Colibacter | |
| | | | Veillonella | Veillonella parvula |
| | | | Megasphaera | Megasphera |
| | | | | elsedenii, |
| | | | | Megasphaera |
| | | | | massiliensis, |
| | | | | Megasphera |
| | | | | micronuciformis |
| | | | | Megasphaera sp |
| | | | Massilibacillus | Massilibacillus |
| | | | | massiliensis |
| | | | Propionispira | |
| | | | Negativicoccus | Negativicoccus |
| | | | | succinicivornas |
| | | | Veillonella | Veillonella dispar, |
| | | | | Veillonella parvula, |
| | | | | Veillonella ratti, |
| | | | | Veillonella |
| | | | | tobetsuensis |
| | Synergistales | Synergistaceae | Aminobacterium | Aminobacterium |
| | | | | mobile |
| | | | Cloacibacillus | Cloacibacillus |
| | | | | evryensis |
| | | | Rarimicrobium | Rarimicrobium |
| | | | | hominis |
| Verrucomicro | Verrucomicrobiales | Akkermansiaceae | Akkermansia | Akkermansia |
| bia | | | | mucinophila |

TABLE 2
Exemplary Bacterial Strains

| | Public DB |
|---|---|
| OTU | Accession |
| Actinobacillus actinomycetemcomitans | AY362885 |
| Actinobacillus minor | ACFT01000025 |
| Actinobacillus pleuropneumoniae | NR_074857 |
| Actinobacillus succinogenes | CP000746 |
| Actinobacillus ureae | AEVG01000167 |
| Actinobaculum massiliae | AF487679 |
| Actinobaculum schaalii | AY957507 |
| Actinobaculum sp. BM#101342 | AY282578 |
| Actinobaculum sp. P2P_19 P1 | AY207066 |
| Akkermansia muciniphila | CP001071 |
| Alistipes finegoldii | NR_043064 |
| Alistipes indistinctus | AB490804 |
| Alistipes onderdonkii | NR_043318 |
| Alistipes putredinis | ABFK02000017 |
| Alistipes shahii | FP929032 |
| Alistipes sp. HGB5 | AENZ01000082 |
| Alistipes sp. JC50 | JF824804 |
| Alistipes sp. RMA 9912 | GQ140629 |
| Anaerostipes caccae | ABAX03000023 |
| Anaerostipes sp. 3_2_56FAA | ACWB01000002 |
| Bacillus aeolius | NR_025557 |
| Bacillus aerophilus | NR_042339 |
| Bacillus aestuarii | GQ980243 |
| Bacillus alcalophilus | X76436 |
| Bacillus amyloliquefaciens | NR_075005 |
| Bacillus anthracis | AAEN01000020 |
| Bacillus atrophaeus | NR_075016 |
| Bacillus badius | NR_036893 |
| Bacillus cereus | ABDJ01000015 |
| Bacillus circulans | AB271747 |
| Bacillus clausii | FN397477 |
| Bacillus coagulans | DQ297928 |
| Bacillus firmus | NR_025842 |
| Bacillus flexus | NR_024691 |
| Bacillus fordii | NR_025786 |
| Bacillus gelatini | NR_025595 |
| Bacillus halmapalus | NR_026144 |
| Bacillus halodurans | AY144582 |
| Bacillus herbersteinensis | NR_042286 |
| Bacillus horti | NR_036860 |
| Bacillus idriensis | NR_043268 |
| Bacillus lentus | NR_040792 |
| Bacillus licheniformis | NC_006270 |
| Bacillus megaterium | GU252124 |
| Bacillus nealsonii | NR_044546 |
| Bacillus niabensis | NR_043334 |
| Bacillus niacini | NR_024695 |
| Bacillus pocheonensis | NR_041377 |
| Bacillus pumilus | NR_074977 |
| Bacillus safensis | JQ624766 |
| Bacillus simplex | NR_042136 |
| Bacillus sonorensis | NR_025130 |
| Bacillus sp. 10403023 MM10403188 | CAET01000089 |
| Bacillus sp. 2_A_57_CT2 | ACWD01000095 |
| Bacillus sp. 2008724126 | GU252108 |
| Bacillus sp. 2008724139 | GU252111 |
| Bacillus sp. 7_16AIA | FN397518 |
| Bacillus sp. 9_3AIA | FN397519 |
| Bacillus sp. AP8 | JX101689 |
| Bacillus sp. B27(2008) | EU362173 |
| Bacillus sp. BT1B_CT2 | ACWC01000034 |
| Bacillus sp. GB1.1 | FJ897765 |
| Bacillus sp. GB9 | FJ897766 |
| Bacillus sp. HU19.1 | FJ897769 |
| Bacillus sp. HU29 | FJ897771 |
| Bacillus sp. HU33.1 | FJ897772 |
| Bacillus sp. JC6 | JF824800 |
| Bacillus sp. oral taxon F26 | HM099642 |
| Bacillus sp. oral taxon F28 | HM099650 |
| Bacillus sp. oral taxon F79 | HM099654 |
| Bacillus sp. SRC_DSF1 | GU797283 |
| Bacillus sp. SRC_DSF10 | GU797292 |
| Bacillus sp. SRC_DSF2 | GU797284 |
| Bacillus sp. SRC_DSF6 | GU797288 |
| Bacillus sp. tc09 | HQ844242 |
| Bacillus sp. zh168 | FJ851424 |
| Bacillus sphaericus | DQ286318 |
| Bacillus sporothermodurans | NR_026010 |
| Bacillus subtilis | EU627588 |
| Bacillus thermoamylovorans | NR_029151 |
| Bacillus weihenstephanensis | NR_074926 |
| Bacteroidales bacterium ph8 | JN837494 |
| Bacteroidales genomosp. P1 | AY341819 |
| Bacteroidales genomosp. P2 oral clone MB1_G13 | DQ003613 |
| Bacteroidales genomosp. P3 oral clone MB1_G34 | DQ003615 |
| Bacteroidales genomosp. P4 oral clone MB2_G17 | DQ003617 |
| Bacteroidales genomosp. P5 oral clone MB2_P04 | DQ003619 |
| Bacteroidales genomosp. P6 oral clone MB3_C19 | DQ003634 |
| Bacteroidales genomosp. P7 oral clone MB3_P19 | DQ003623 |
| Bacteroidales genomosp. P8 oral clone MB4_G15 | DQ003626 |
| Bacteroides acidifaciens | NR_028607 |
| Bacteroides barnesiae | NR_041446 |
| Bacteroides caccae | EU136686 |
| Bacteroides cellulosilyticus | ACCH01000108 |
| Bacteroides clarus | AFBM01000011 |
| Bacteroides coagulans | AB547639 |
| Bacteroides coprocola | ABIY02000050 |
| Bacteroides coprophilus | ACBW01000012 |
| Bacteroides dorei | ABWZ01000093 |
| Bacteroides eggerthii | ACWG01000065 |
| Bacteroides faecis | GQ496624 |
| Bacteroides finegoldii | AB222699 |
| Bacteroides fluxus | AFBN01000029 |
| Bacteroides fragilis | AP006841 |
| Bacteroides galacturonicus | DQ497994 |
| Bacteroides helcogenes | CP002352 |
| Bacteroides heparinolyticus | JN867284 |
| Bacteroides intestinalis | ABJL02000006 |
| Bacteroides massiliensis | AB200226 |
| Bacteroides nordii | NR_043017 |
| Bacteroides oleiciplenus | AB547644 |
| Bacteroides ovatus | ACWH01000036 |
| Bacteroides pectinophilus | ABVQ01000036 |
| Bacteroides plebeius | AB200218 |
| Bacteroides pyogenes | NR_041280 |
| Bacteroides salanitronis | CP002530 |
| Bacteroides salyersiae | EU136690 |
| Bacteroides sp. 1_1_14 | ACRP01000155 |
| Bacteroides sp. 1_1_30 | ADCL01000128 |
| Bacteroides sp. 1_1_6 | ACIC01000215 |
| Bacteroides sp. 2_1_22 | ACPQ01000117 |
| Bacteroides sp. 2_1_56FAA | ACWI01000065 |
| Bacteroides sp. 2_2_4 | ABZZ01000168 |
| Bacteroides sp. 20_3 | ACRQ01000064 |
| Bacteroides sp. 3_1_19 | ADCJ01000062 |
| Bacteroides sp. 3_1_23 | ACRS01000081 |
| Bacteroides sp. 3_1_33FAA | ACPS01000085 |
| Bacteroides sp. 3_1_40A | ACRT01000136 |
| Bacteroides sp. 3_2_5 | ACIB01000079 |
| Bacteroides sp. 315_5 | FJ848547 |
| Bacteroides sp. 31SF15 | AJ583248 |
| Bacteroides sp. 31SF18 | AJ583249 |
| Bacteroides sp. 35AE31 | AJ583244 |
| Bacteroides sp. 35AE37 | AJ583245 |
| Bacteroides sp. 35BE34 | AJ583246 |
| Bacteroides sp. 35BE35 | AJ583247 |
| Bacteroides sp. 4_1_36 | ACTC01000133 |
| Bacteroides sp. 4_3_47FAA | ACDR02000029 |
| Bacteroides sp. 9_1_42FAA | ACAA01000096 |
| Bacteroides sp. AR20 | AF139524 |
| Bacteroides sp. AR29 | AF139525 |
| Bacteroides sp. B2 | EU722733 |
| Bacteroides sp. D1 | ACAB02000030 |
| Bacteroides sp. D2 | ACGA01000077 |
| Bacteroides sp. D20 | ACPT01000052 |
| Bacteroides sp. D22 | ADCK01000151 |
| Bacteroides sp. F_4 | AB470322 |
| Bacteroides sp. NB_8 | AB117565 |
| Bacteroides sp. WH2 | AY895180 |
| Bacteroides sp. XB12B | AM230648 |
| Bacteroides sp. XB44A | AM230649 |
| Bacteroides stercoris | ABFZ02000022 |
| Bacteroides thetaiotaomicron | NR_074277 |
| Bacteroides uniformis | AB050110 |
| Bacteroides ureolyticus | GQ167666 |
| Bacteroides vulgatus | CP000139 |
| Bacteroides xylanisolvens | ADKP01000087 |
| Bacteroidetes bacterium oral taxon D27 | HM099638 |
| Bacteroidetes bacterium oral taxon F31 | HM099643 |
| Bacteroidetes bacterium oral taxon F44 | HM099649 |
| Barnesiella intestinihominis | AB370251 |
| Bifidobacteriaceae genomosp. C1 | AY278612 |
| Bifidobacterium adolescentis | AAXD02000018 |
| Bifidobacterium angulatum | ABYS02000004 |
| Bifidobacterium animalis | CP001606 |
| Bifidobacterium bifidum | ABQP01000027 |
| Bifidobacterium breve | CP002743 |
| Bifidobacterium catenulatum | ABXY01000019 |
| Bifidobacterium dentium | CP001750 |
| Bifidobacterium gallicum | ABXB03000004 |
| Bifidobacterium infantis | AY151398 |
| Bifidobacterium kashiwanohense | AB491757 |
| Bifidobacterium longum | ABQQ01000041 |
| Bifidobacterium pseudocatenulatum | ABXX02000002 |
| Bifidobacterium pseudolongum | NR_043442 |
| Bifidobacterium scardovii | AJ307005 |
| Bifidobacterium sp. HM2 | AB425276 |
| Bifidobacterium sp. HMLN12 | JF519685 |
| Bifidobacterium sp. M45 | HM626176 |
| Bifidobacterium sp. MSX5B | HQ616382 |
| Bifidobacterium sp. TM_7 | AB218972 |
| Bifidobacterium thermophilum | DQ340557 |
| Bifidobacterium urinalis | AJ278695 |
| Blautia coccoides | AB571656 |
| Blautia glucerasea | AB588023 |
| Blautia glucerasei | AB439724 |
| Blautia hansenii | ABYU02000037 |
| Blautia hydrogenotrophica | ACBZ01000217 |
| Blautia luti | AB691576 |
| Blautia producta | AB600998 |
| Blautia schinkii | NR_026312 |
| Blautia sp. M25 | HM626178 |
| Blautia stercoris | HM626177 |
| Blautia wexlerae | EF036467 |
| Bordetella bronchiseptica | NR_025949 |
| Bordetella holmesii | AB683187 |
| Bordetella parapertussis | NR_025950 |
| Bordetella pertussis | BX640418 |
| Borrelia afzelii | ABCU01000001 |
| Borrelia burgdorferi | ABGI01000001 |
| Borrelia crocidurae | DQ057990 |
| Borrelia duttonii | NC_011229 |
| Borrelia garinii | ABJV01000001 |
| Borrelia hermsii | AY597657 |
| Borrelia hispanica | DQ057988 |
| Borrelia persica | HM161645 |
| Borrelia recurrentis | AF107367 |
| Borrelia sp. NE49 | AJ224142 |
| Borrelia spielmanii | ABKB01000002 |
| Borrelia turicatae | NC_008710 |
| Borrelia valaisiana | ABCY01000002 |
| Brucella ovis | NC_009504 |
| Brucella sp. 83_13 | ACBQ01000040 |
| Brucella sp. BO1 | EU053207 |
| Brucella suis | ACBK01000034 |
| Burkholderia ambifaria | AAUZ01000009 |
| Burkholderia cenocepacia | AAHI01000060 |
| Burkholderia cepacia | NR_041719 |
| Burkholderia mallei | CP000547 |
| Burkholderia multivorans | NC_010086 |
| Burkholderia oklahomensis | DQ108388 |
| Burkholderia pseudomallei | CP001408 |
| Burkholderia rhizoxinica | HQ005410 |
| Burkholderia sp. 383 | CP000151 |
| Burkholderia xenovorans | U86373 |
| Burkholderiales bacterium 1_1_47 | ADCQ01000066 |
| Butyrivibrio crossotus | ABWN01000012 |
| Butyrivibrio fibrisolvens | U41172 |
| Chlamydia muridarum | AE002160 |
| Chlamydia psittaci | NR_036864 |
| Chlamydia trachomatis | U68443 |
| Chlamydiales bacterium NS11 | JN606074 |
| Citrobacter amalonaticus | FR870441 |
| Citrobacter braakii | NR_028687 |
| Citrobacter farmeri | AF025371 |
| Citrobacter freundii | NR_028894 |
| Citrobacter gillenii | AF025367 |
| Citrobacter koseri | NC_009792 |
| Citrobacter murliniae | AF025369 |
| Citrobacter rodentium | NR_074903 |
| Citrobacter sedlakii | AF025364 |
| Citrobacter sp. 30_2 | ACDJ01000053 |
| Citrobacter sp. KMSI_3 | GQ468398 |
| Citrobacter werkmanii | AF025373 |
| Citrobacter youngae | ABWL02000011 |
| Cloacibacillus evryensis | GQ258966 |
| Clostridiaceae bacterium END_2 | EF451053 |
| Clostridiaceae bacterium JC13 | JF824807 |
| Clostridiales bacterium 1_7_47FAA | ABQR01000074 |
| Clostridiales bacterium 9400853 | HM587320 |
| Clostridiales bacterium 9403326 | HM587324 |
| Clostridiales bacterium oral clone P4PA_66 P1 | AY207065 |
| Clostridiales bacterium oral taxon 093 | GQ422712 |
| Clostridiales bacterium oral taxon F32 | HM099644 |
| Clostridiales bacterium ph2 | JN837487 |
| Clostridiales bacterium SY8519 | AB477431 |
| Clostridiales genomosp. BVAB3 | CP001850 |
| Clostridiales sp. SM4_1 | FP929060 |
| Clostridiales sp. SS3_4 | AY305316 |
| Clostridiales sp. SSC_2 | FP929061 |
| Clostridium acetobutylicum | NR_074511 |
| Clostridium aerotolerans | X76163 |
| Clostridium aldenense | NR_043680 |
| Clostridium aldrichii | NR_026099 |
| Clostridium algidicarnis | NR_041746 |
| Clostridium algidixylanolyticum | NR_028726 |
| Clostridium aminovalericum | NR_029245 |
| Clostridium amygdalinum | AY353957 |
| Clostridium argentinense | NR_029232 |
| Clostridium asparagiforme | ACCJ01000522 |
| Clostridium baratii | NR_029229 |
| Clostridium bartlettii | ABEZ02000012 |
| Clostridium beijerinckii | NR_074434 |
| Clostridium bifermentans | X73437 |
| Clostridium bolteae | ABCC02000039 |
| Clostridium botulinum | NC_010723 |
| Clostridium butyricum | ABDT01000017 |
| Clostridium cadaveris | AB542932 |
| Clostridium carboxidivorans | FR733710 |
| Clostridium carnis | NR_044716 |
| Clostridium celatum | X77844 |
| Clostridium celerecrescens | JQ246092 |
| Clostridium cellulosi | NR_044624 |
| Clostridium chauvoei | EU106372 |
| Clostridium citroniae | ADLJ01000059 |
| Clostridium clariflavum | NR_041235 |
| Clostridium clostridiiformes | M59089 |
| Clostridium clostridioforme | NR_044715 |
| Clostridium coccoides | EF025906 |
| Clostridium cochlearium | NR_044717 |
| Clostridium cocleatum | NR_026495 |
| Clostridium colicanis | FJ957863 |
| Clostridium colinum | NR_026151 |
| Clostridium difficile | NC_013315 |
| Clostridium disporicum | NR_026491 |
| Clostridium estertheticum | NR_042153 |
| Clostridium fallax | NR_044714 |
| Clostridium favososporum | X76749 |
| Clostridium felsineum | AF270502 |
| Clostridium frigidicarnis | NR_024919 |
| Clostridium gasigenes | NR_024945 |
| Clostridium ghonii | AB542933 |
| Clostridium glycolicum | FJ384385 |
| Clostridium glycyrrhizinilyticum | AB233029 |
| Clostridium haemolyticum | NR_024749 |
| Clostridium hathewayi | AY552788 |
| Clostridium hiranonis | AB023970 |
| Clostridium histolyticum | HF558362 |
| Clostridium hylemonae | AB023973 |
| Clostridium indolis | AF028351 |
| Clostridium innocuum | M23732 |
| Clostridium irregulare | NR_029249 |
| Clostridium isatidis | NR_026347 |
| Clostridium kluyveri | NR_074165 |
| Clostridium lactatifermentans | NR_025651 |
| Clostridium lavalense | EF564277 |
| Clostridium leptum | AJ305238 |
| Clostridium limosum | FR870444 |
| Clostridium magnum | X77835 |
| Clostridium malenominatum | FR749893 |
| Clostridium mayombei | FR733682 |
| Clostridium methylpentosum | ACEC01000059 |
| Clostridium nexile | X73443 |
| Clostridium novyi | NR_074343 |
| Clostridium orbiscindens | Y18187 |
| Clostridium oroticum | FR749922 |
| Clostridium paraputrificum | AB536771 |
| Clostridium perfringens | ABDW01000023 |
| Clostridium phytofermentans | NR_074652 |
| Clostridium piliforme | D14639 |
| Clostridium putrefaciens | NR_024995 |
| Clostridium quinii | NR_026149 |
| Clostridium ramosum | M23731 |
| Clostridium rectum | NR_029271 |
| Clostridium saccharogumia | DQ100445 |
| Clostridium saccharolyticum | CP002109 |
| Clostridium sardiniense | NR_041006 |
| Clostridium sartagoforme | NR_026490 |
| Clostridium scindens | AF262238 |
| Clostridium septicum | NR_026020 |
| Clostridium sordellii | AB448946 |
| Clostridium sp. 7_2_43FAA | ACDK01000101 |
| Clostridium sp. D5 | ADBG01000142 |
| Clostridium sp. HGF2 | AENW01000022 |
| Clostridium sp. HPB_46 | AY862516 |
| Clostridium sp. JC122 | CAEV01000127 |
| Clostridium sp. L2_50 | AAYW02000018 |
| Clostridium sp. LMG 16094 | X95274 |
| Clostridium sp. M62_1 | ACFX02000046 |
| Clostridium sp. MLG055 | AF304435 |
| Clostridium sp. MT4 E | FJ159523 |
| Clostridium sp. NMBHI_1 | JN093130 |
| Clostridium sp. NML 04A032 | EU815224 |
| Clostridium sp. SS2_1 | ABGC03000041 |
| Clostridium sp. SY8519 | AP012212 |
| Clostridium sp. TM_40 | AB249652 |
| Clostridium sp. YIT 12069 | AB491207 |
| Clostridium sp. YIT 12070 | AB491208 |
| Clostridium sphenoides | X73449 |
| Clostridium spiroforme | X73441 |
| Clostridium sporogenes | ABKW02000003 |
| Clostridium sporosphaeroides | NR_044835 |
| Clostridium stercorarium | NR_025100 |
| Clostridium sticklandii | L04167 |
| Clostridium straminisolvens | NR_024829 |
| Clostridium subterminale | NR_041795 |
| Clostridium sulfidigenes | NR_044161 |
| Clostridium symbiosum | ADLQ01000114 |
| Clostridium tertium | Y18174 |
| Clostridium tetani | NC_004557 |
| Clostridium thermocellum | NR_074629 |
| Clostridium tyrobutyricum | NR_044718 |
| Clostridium viride | NR_026204 |
| Clostridium xylanolyticum | NR_037068 |
| Collinsella aerofaciens | AAVN02000007 |
| Collinsella intestinalis | ABXH02000037 |
| Collinsella stercoris | ABXJ01000150 |
| Collinsella tanakaei | AB490807 |
| Coprobacillus cateniformis | AB030218 |
| Coprobacillus sp. 29_1 | ADKX01000057 |
| Coprobacillus sp. D7 | ACDT01000199 |
| Coprococcus catus | EU266552 |
| Coprococcus comes | ABVR01000038 |
| Coprococcus eutactus | EF031543 |
| Coprococcus sp. ART55_1 | AY350746 |
| Dialister invisus | ACIM02000001 |
| Dialister micraerophilus | AFBB01000028 |
| Dialister microaerophilus | AENT01000008 |
| Dialister pneumosintes | HM596297 |
| Dialister propionicifaciens | NR_043231 |
| Dialister sp. oral taxon 502 | GQ422739 |
| Dialister succinatiphilus | AB370249 |
| Dorea formicigenerans | AAXA02000006 |
| Dorea longicatena | AJ132842 |
| Enhydrobacter aerosaccus | ACYI01000081 |
| Enterobacter aerogenes | AJ251468 |
| Enterobacter asburiae | NR_024640 |
| Enterobacter cancerogenus | Z96078 |
| Enterobacter cloacae | FP929040 |
| Enterobacter cowanii | NR_025566 |
| Enterobacter hormaechei | AFHR01000079 |
| Enterobacter sp. 247BMC | HQ122932 |
| Enterobacter sp. 638 | NR_074777 |
| Enterobacter sp. JC163 | JN657217 |
| Enterobacter sp. SCSS | HM007811 |
| Enterobacter sp. TSE38 | HM156134 |
| Enterobacteriaceae bacterium 9_2_54FAA | ADCU01000033 |
| Enterobacteriaceae bacterium CF01Ent_1 | AJ489826 |
| Enterobacteriaceae bacterium Smarlab 3302238 | AY538694 |
| Enterococcus avium | AF133535 |
| Enterococcus caccae | AY943820 |
| Enterococcus casseliflavus | AEWT01000047 |
| Enterococcus durans | AJ276354 |
| Enterococcus faecalis | AE016830 |
| Enterococcus faecium | AM157434 |
| Enterococcus gallinarum | AB269767 |
| Enterococcus gilvus | AY033814 |
| Enterococcus hawaiiensis | AY321377 |
| Enterococcus hirae | AF061011 |
| Enterococcus italicus | AEPV01000109 |
| Enterococcus mundtii | NR_024906 |
| Enterococcus raffinosus | FN600541 |
| Enterococcus sp. BV2CASA2 | JN809766 |
| Enterococcus sp. CCRI_16620 | GU457263 |
| Enterococcus sp. F95 | FJ463817 |
| Enterococcus sp. RfL6 | AJ133478 |
| Enterococcus thailandicus | AY321376 |
| Erysipelotrichaceae bacterium 3_1_53 | ACTJ01000113 |
| Erysipelotrichaceae bacterium 5_2_54FAA | |
| Escherichia albertii | ABKX01000012 |
| Escherichia coli | NC_008563 |
| Escherichia fergusonii | CU928158 |
| Escherichia hermannii | HQ407266 |
| Escherichia sp. 1_1_43 | ACID01000033 |
| Escherichia sp. 4_1_40B | ACDM02000056 |
| Escherichia sp. B4 | EU722735 |
| Escherichia vulneris | NR_041927 |
| Eubacteriaceae bacterium P4P_50 P4 | AY207060 |
| Eubacterium barkeri | NR_044661 |
| Eubacterium biforme | ABYT01000002 |
| Eubacterium brachy | U13038 |
| Eubacterium budayi | NR_024682 |
| Eubacterium callanderi | NR_026330 |
| Eubacterium cellulosolvens | AY178842 |
| Eubacterium contortum | FR749946 |
| Eubacterium coprostanoligenes | HM037995 |
| Eubacterium cylindroides | FP929041 |
| Eubacterium desmolans | NR_044644 |
| Eubacterium dolichum | L34682 |
| Eubacterium eligens | CP001104 |
| Eubacterium fissicatena | FR749935 |
| Eubacterium hadrum | FR749933 |
| Eubacterium hallii | L34621 |
| Eubacterium infirmum | U13039 |
| Eubacterium limosum | CP002273 |
| Eubacterium moniliforme | HF558373 |
| Eubacterium multiforme | NR_024683 |
| Eubacterium nitritogenes | NR_024684 |
| Eubacterium nodatum | U13041 |
| Eubacterium ramulus | AJ011522 |
| Eubacterium rectale | FP929042 |
| Eubacterium ruminantium | NR_024661 |
| Eubacterium saburreum | AB525414 |
| Eubacterium saphenum | NR_026031 |
| Eubacterium siraeum | ABCA03000054 |
| Eubacterium sp. 3_1_31 | ACTL01000045 |
| Eubacterium sp. AS15b | HQ616364 |
| Eubacterium sp. OBRC9 | HQ616354 |
| Eubacterium sp. oral clone GI038 | AY349374 |
| Eubacterium sp. oral clone IR009 | AY349376 |
| Eubacterium sp. oral clone JH012 | AY349373 |
| Eubacterium sp. oral clone JI012 | AY349379 |
| Eubacterium sp. oral clone JN088 | AY349377 |
| Eubacterium sp. oral clone JS001 | AY349378 |
| Eubacterium sp. oral clone OH3A | AY947497 |
| Eubacterium sp. WAL 14571 | FJ687606 |
| Eubacterium tenue | M59118 |
| Eubacterium tortuosum | NR_044648 |
| Eubacterium ventriosum | L34421 |
| Eubacterium xylanophilum | L34628 |
| Eubacterium yurii | AEES01000073 |
| Fusobacterium canifelinum | AY162222 |
| Fusobacterium genomosp. C1 | AY278616 |
| Fusobacterium genomosp. C2 | AY278617 |
| Fusobacterium gonidiaformans | ACET01000043 |
| Fusobacterium mortiferum | ACDB02000034 |
| Fusobacterium naviforme | HQ223106 |
| Fusobacterium necrogenes | X55408 |
| Fusobacterium necrophorum | AM905356 |
| Fusobacterium nucleatum | ADVK01000034 |
| Fusobacterium periodonticum | ACJY01000002 |
| Fusobacterium russii | NR_044687 |
| Fusobacterium sp. 1_1_41FAA | ADGG01000053 |
| Fusobacterium sp. 11_3_2 | ACUO01000052 |
| Fusobacterium sp. 12_1B | AGWJ01000070 |
| Fusobacterium sp. 2_1_31 | ACDC02000018 |
| Fusobacterium sp. 3_1_27 | ADGF01000045 |
| Fusobacterium sp. 3_1_33 | ACQE01000178 |
| Fusobacterium sp. 3_1_36A2 | ACPU01000044 |
| Fusobacterium sp. 3_1_5R | ACDD01000078 |
| Fusobacterium sp. AC18 | HQ616357 |
| Fusobacterium sp. ACB2 | HQ616358 |
| Fusobacterium sp. AS2 | HQ616361 |
| Fusobacterium sp. CM1 | HQ616371 |
| Fusobacterium sp. CM21 | HQ616375 |
| Fusobacterium sp. CM22 | HQ616376 |
| Fusobacterium sp. D12 | ACDG02000036 |
| Fusobacterium sp. oral clone ASCF06 | AY923141 |
| Fusobacterium sp. oral clone ASCF11 | AY953256 |
| Fusobacterium ulcerans | ACDH01000090 |
| Fusobacterium varium | ACIE01000009 |
| Gemella haemolysans | ACDZ02000012 |
| Gemella morbillorum | NR_025904 |
| Gemella morbillorum | ACRX01000010 |
| Gemella sanguinis | ACRY01000057 |
| Gemella sp. oral clone ASCE02 | AY923133 |
| Gemella sp. oral clone ASCF04 | AY923139 |
| Gemella sp. oral clone ASCF12 | AY923143 |
| Gemella sp. WAL 1945J | EU427463 |
| Klebsiella oxytoca | AY292871 |
| Klebsiella pneumoniae | CP000647 |
| Klebsiella sp. AS10 | HQ616362 |
| Klebsiella sp. Co9935 | DQ068764 |
| Klebsiella sp. enrichment culture | HM195210 |
| clone SRC_DSD25 | |
| Klebsiella sp. OBRC7 | HQ616353 |
| Klebsiella sp. SP_BA | FJ999767 |
| Klebsiella sp. SRC_DSD1 | GU797254 |
| Klebsiella sp. SRC_DSD11 | GU797263 |
| Klebsiella sp. SRC_DSD12 | GU797264 |
| Klebsiella sp. SRC_DSD15 | GU797267 |
| Klebsiella sp. SRC_DSD2 | GU797253 |
| Klebsiella sp. SRC_DSD6 | GU797258 |
| Klebsiella variicola | CP001891 |
| Lachnobacterium bovis | GU324407 |
| Lachnospira multipara | FR733699 |
| Lachnospira pectinoschiza | L14675 |
| Lachnospiraceae bacterium 1_1_57FAA | ACTM01000065 |
| Lachnospiraceae bacterium 1_4_56FAA | ACTN01000028 |
| Lachnospiraceae bacterium 2_1_46FAA | ADLB01000035 |
| Lachnospiraceae bacterium 2_1_58FAA | ACTO01000052 |
| Lachnospiraceae bacterium 3_1_57FAA_CT1 | ACTP01000124 |
| Lachnospiraceae bacterium 4_1_37FAA | ADCR01000030 |
| Lachnospiraceae bacterium 5_1_57FAA | ACTR01000020 |
| Lachnospiraceae bacterium 5_1_63FAA | ACTS01000081 |
| Lachnospiraceae bacterium 6_1_63FAA | ACTV01000014 |
| Lachnospiraceae bacterium 8_1_57FAA | ACWQ01000079 |
| Lachnospiraceae bacterium 9_1_43BFAA | ACTX01000023 |
| Lachnospiraceae bacterium A4 | DQ789118 |
| Lachnospiraceae bacterium DJF VP30 | EU728771 |
| Lachnospiraceae bacterium ICM62 | HQ616401 |
| Lachnospiraceae bacterium MSX33 | HQ616384 |
| Lachnospiraceae bacterium oral taxon 107 | ADDS01000069 |
| Lachnospiraceae bacterium oral taxon F15 | HM099641 |
| Lachnospiraceae genomosp. C1 | AY278618 |
| Lactobacillus acidipiscis | NR_024718 |
| Lactobacillus acidophilus | CP000033 |
| Lactobacillus alimentarius | NR_044701 |
| Lactobacillus amylolyticus | ADNY01000006 |
| Lactobacillus amylovorus | CP002338 |
| Lactobacillus antri | ACLL01000037 |
| Lactobacillus brevis | EU194349 |
| Lactobacillus buchneri | ACGH01000101 |
| Lactobacillus casei | CP000423 |
| Lactobacillus catenaformis | M23729 |
| Lactobacillus coleohominis | ACOH01000030 |
| Lactobacillus coryniformis | NR_044705 |
| Lactobacillus crispatus | ACOG01000151 |
| Lactobacillus curvatus | NR_042437 |
| Lactobacillus delbrueckii | CP002341 |
| Lactobacillus dextrinicus | NR_036861 |
| Lactobacillus farciminis | NR_044707 |
| Lactobacillus fermentum | CP002033 |
| Lactobacillus gasseri | ACOZ01000018 |
| Lactobacillus gastricus | AICN01000060 |
| Lactobacillus genomosp. C1 | AY278619 |
| Lactobacillus genomosp. C2 | AY278620 |
| Lactobacillus helveticus | ACLM01000202 |
| Lactobacillus hilgardii | ACGP01000200 |
| Lactobacillus hominis | FR681902 |
| Lactobacillus iners | AEKJ01000002 |
| Lactobacillus jensenii | ACQD01000066 |
| Lactobacillus johnsonii | AE017198 |
| Lactobacillus kalixensis | NR_029083 |
| Lactobacillus kefiranofaciens | NR_042440 |
| Lactobacillus kefiri | NR_042230 |
| Lactobacillus kimchii | NR_025045 |
| Lactobacillus leichmannii | JX986966 |
| Lactobacillus mucosae | FR693800 |
| Lactobacillus murinus | NR_042231 |
| Lactobacillus nodensis | NR_041629 |
| Lactobacillus oeni | NR_043095 |
| Lactobacillus oris | AEKL01000077 |
| Lactobacillus parabrevis | NR_042456 |
| Lactobacillus parabuchneri | NR_041294 |
| Lactobacillus paracasei | ABQV01000067 |
| Lactobacillus parakefiri | NR_029039 |
| Lactobacillus pentosus | JN813103 |
| Lactobacillus perolens | NR_029360 |
| Lactobacillus plantarum | ACGZ02000033 |
| Lactobacillus pontis | HM218420 |
| Lactobacillus reuteri | ACGW02000012 |
| Lactobacillus rhamnosus | ABWJ01000068 |
| Lactobacillus rogosae | GU269544 |
| Lactobacillus ruminis | ACGS02000043 |
| Lactobacillus sakei | DQ989236 |
| Lactobacillus salivarius | AEBA01000145 |
| Lactobacillus saniviri | AB602569 |
| Lactobacillus senioris | AB602570 |
| Lactobacillus sp. 66c | FR681900 |
| Lactobacillus sp. BT6 | HQ616370 |
| Lactobacillus sp. KLDS 1.0701 | EU600905 |
| Lactobacillus sp. KLDS 1.0702 | EU600906 |
| Lactobacillus sp. KLDS 1.0703 | EU600907 |
| Lactobacillus sp. KLDS 1.0704 | EU600908 |
| Lactobacillus sp. KLDS 1.0705 | EU600909 |
| Lactobacillus sp. KLDS 1.0707 | EU600911 |
| Lactobacillus sp. KLDS 1.0709 | EU600913 |
| Lactobacillus sp. KLDS 1.0711 | EU600915 |
| Lactobacillus sp. KLDS 1.0712 | EU600916 |
| Lactobacillus sp. KLDS 1.0713 | EU600917 |
| Lactobacillus sp. KLDS 1.0716 | EU600921 |
| Lactobacillus sp. KLDS 1.0718 | EU600922 |
| Lactobacillus sp. KLDS 1.0719 | EU600923 |
| Lactobacillus sp. oral clone HT002 | AY349382 |
| Lactobacillus sp. oral clone HT070 | AY349383 |
| Lactobacillus sp. oral taxon 052 | GQ422710 |
| Lactobacillus tucceti | NR_042194 |
| Lactobacillus ultunensis | ACGU01000081 |
| Lactobacillus vaginalis | ACGV01000168 |
| Lactobacillus vini | NR_042196 |
| Lactobacillus vitulinus | NR_041305 |
| Lactobacillus zeae | NR_037122 |
| Lactococcus garvieae | AF061005 |
| Lactococcus lactis | CP002365 |
| Lactococcus raffinolactis | NR_044359 |
| Listeria grayi | ACCR02000003 |
| Listeria innocua | JF967625 |
| Listeria ivanovii | X56151 |
| Listeria monocytogenes | CP002003 |
| Listeria welshimeri | AM263198 |
| Megasphaera elsdenii | AY038996 |
| Megasphaera genomosp. C1 | AY278622 |
| Megasphaera genomosp. type_1 | ADGP01000010 |
| Megasphaera micronuciformis | AECS01000020 |
| Megasphaera sp. BLPYG_07 | HM990964 |
| Megasphaera sp. UPII 199_6 | AFIJ01000040 |
| Microbacterium gubbeenense | NR_025098 |
| Microbacterium lacticum | EU714351 |
| Mitsuokella jalaludinii | NR_028840 |
| Mitsuokella multacida | ABWK02000005 |
| Mitsuokella sp. oral taxon 521 | GU413658 |
| Mitsuokella sp. oral taxon G68 | GU432166 |
| Mycobacterium abscessus | AGQU01000002 |
| Mycobacterium africanum | AF480605 |
| Mycobacterium alsiensis | AJ938169 |
| Mycobacterium avium | CP000479 |
| Mycobacterium chelonae | AB548610 |
| Mycobacterium colombiense | AM062764 |
| Mycobacterium elephantis | AF385898 |
| Mycobacterium gordonae | GU142930 |
| Mycobacterium intracellulare | GQ153276 |
| Mycobacterium kansasii | AF480601 |
| Mycobacterium lacus | NR_025175 |
| Mycobacterium leprae | FM211192 |
| Mycobacterium lepromatosis | EU203590 |
| Mycobacterium mageritense | FR798914 |
| Mycobacterium mantenii | FJ042897 |
| Mycobacterium marinum | NC_010612 |
| Mycobacterium microti | NR_025234 |
| Mycobacterium neoaurum | AF268445 |
| Mycobacterium parascrofulaceum | ADNV01000350 |
| Mycobacterium paraterrae | EU919229 |
| Mycobacterium phlei | GU142920 |
| Mycobacterium seoulense | DQ536403 |
| Mycobacterium smegmatis | CP000480 |
| Mycobacterium sp. 1761 | EU703150 |
| Mycobacterium sp. 1776 | EU703152 |
| Mycobacterium sp. 1781 | EU703147 |
| Mycobacterium sp. 1791 | EU703148 |
| Mycobacterium sp. 1797 | EU703149 |
| Mycobacterium sp. AQIGA4 | HM210417 |
| Mycobacterium sp. B10_07.09.0206 | HQ174245 |
| Mycobacterium sp. GN_10546 | FJ497243 |
| Mycobacterium sp. GN_10827 | FJ497247 |
| Mycobacterium sp. GN_11124 | FJ652846 |
| Mycobacterium sp. GN_9188 | FJ497240 |
| Mycobacterium sp. GR_2007_210 | FJ555538 |
| Mycobacterium sp. HE5 | AJ012738 |
| Mycobacterium sp. NLA001000736 | HM627011 |
| Mycobacterium sp. W | DQ437715 |
| Mycobacterium tuberculosis | CP001658 |
| Mycobacterium ulcerans | AB548725 |
| Mycobacterium vulneris | EU834055 |
| Mycoplasma agalactiae | AF010477 |
| Mycoplasma amphoriforme | AY531656 |
| Mycoplasma arthritidis | NC_011025 |
| Mycoplasma bovoculi | NR_025987 |
| Mycoplasma faucium | NR_024983 |
| Mycoplasma fermentans | CP002458 |
| Mycoplasma flocculare | X62699 |
| Mycoplasma genitalium | L43967 |
| Mycoplasma hominis | AF443616 |
| Mycoplasma orale | AY796060 |
| Mycoplasma ovipneumoniae | NR_025989 |
| Mycoplasma penetrans | NC_004432 |
| Mycoplasma pneumoniae | NC_000912 |
| Mycoplasma putrefaciens | U26055 |
| Mycoplasma salivarium | M24661 |
| Mycoplasmataceae genomosp. P1 oral | DQ003614 |
| clone MB1_G23 | |
| Neisseria bacilliformis | AFAY01000058 |
| Neisseria cinerea | ACDY01000037 |
| Neisseria elongata | ADBF01000003 |
| Neisseria flavescens | ACQV01000025 |
| Neisseria genomosp. P2 oral clone MB5_P15 | DQ003630 |
| Neisseria gonorrhoeae | CP002440 |
| Neisseria lactamica | ACEQ01000095 |
| Neisseria macacae | AFQE01000146 |
| Neisseria meningitidis | NC_003112 |
| Neisseria mucosa | ACDX01000110 |
| Neisseria pharyngis | AJ239281 |
| Neisseria polysaccharea | ADBE01000137 |
| Neisseria sicca | ACKO02000016 |
| Neisseria sp. KEM232 | GQ203291 |
| Neisseria sp. oral clone AP132 | AY005027 |
| Neisseria sp. oral clone JC012 | AY349388 |
| Neisseria sp. oral strain B33KA | AY005028 |
| Neisseria sp. oral taxon 014 | ADEA01000039 |
| Neisseria sp. SMC_A9199 | FJ763637 |
| Neisseria sp. TM10_1 | DQ279352 |
| Neisseria subflava | ACEO01000067 |
| Odoribacter laneus | AB490805 |
| Odoribacter splanchnicus | CP002544 |
| Oscillibacter sp. G2 | HM626173 |
| Oscillibacter valericigenes | NR_074793 |
| Oscillospira guilliermondii | AB040495 |
| Paenibacillus barcinonensis | NR_042272 |
| Paenibacillus barengoltzii | NR_042756 |
| Paenibacillus chibensis | NR_040885 |
| Paenibacillus cookii | NR_025372 |
| Paenibacillus durus | NR_037017 |
| Paenibacillus glucanolyticus | D78470 |
| Paenibacillus lactis | NR_025739 |
| Paenibacillus lautus | NR_040882 |
| Paenibacillus pabuli | NR_040853 |
| Paenibacillus polymyxa | NR_037006 |
| Paenibacillus popilliae | NR_040888 |
| Paenibacillus sp. CIP 101062 | HM212646 |
| Parabacteroides distasonis | CP000140 |
| Parabacteroides goldsteinii | AY974070 |
| Parabacteroides gordonii | AB470344 |
| Parabacteroides johnsonii | ABYH01000014 |
| Parabacteroides merdae | EU136685 |
| Parabacteroidessp. D13 | ACPW01000017 |
| Parabacteroidessp. NS31_3 | JN029805 |
| Peptococcus niger | NR_029221 |
| Peptococcus sp. oral clone JM048 | AY349389 |
| Peptococcus sp. oral taxon 167 | GQ422727 |
| Peptoniphilus asaccharolyticus | D14145 |
| Peptoniphilus duerdenii | EU526290 |
| Peptoniphilus harei | NR_026358 |
| Peptoniphilus indolicus | AY153431 |
| Peptoniphilus ivorii | Y07840 |
| Peptoniphilus lacrimalis | ADDO01000050 |
| Peptoniphilus sp. gpac007 | AM176517 |
| Peptoniphilus sp. gpac018A | AM176519 |
| Peptoniphilus sp. gpac077 | AM176527 |
| Peptoniphilus sp. gpac148 | AM176535 |
| Peptoniphilus sp. JC140 | JF824803 |
| Peptoniphilus sp. oral taxon 386 | ADCS01000031 |
| Peptoniphilus sp. oral taxon 836 | AEAA01000090 |
| Peptostreptococcaceae bacterium ph1 | JN837495 |
| Peptostreptococcus anaerobius | AY326462 |
| Peptostreptococcus micros | AM176538 |
| Peptostreptococcus sp. 9succ1 | X90471 |
| Peptostreptococcus sp. oral clone AP24 | AB175072 |
| Peptostreptococcus sp. oral clone FJ023 | AY349390 |
| Peptostreptococcus sp. P4P_31 P3 | AY207059 |
| Peptostreptococcus stomatis | ADGQ01000048 |
| Porphyromonadaceae bacterium NML 060648 | EF184292 |
| Porphyromonas asaccharolytica | AENO01000048 |
| Porphyromonas endodontalis | ACNN01000021 |
| Porphyromonas gingivalis | AE015924 |
| Porphyromonas levii | NR_025907 |
| Porphyromonas macacae | NR_025908 |
| Porphyromonas somerae | AB547667 |
| Porphyromonas sp. oral clone BB134 | AY005068 |
| Porphyromonas sp. oral clone F016 | AY005069 |
| Porphyromonas sp. oral clone P2PB_52 P1 | AY207054 |
| Porphyromonas sp. oral clone P4GB_100 P2 | AY207057 |
| Porphyromonas sp. UQD 301 | EU012301 |
| Porphyromonas uenonis | ACLR01000152 |
| Prevotella albensis | NR_025300 |
| Prevotella amnii | AB547670 |
| Prevotella bergensis | ACKS01000100 |
| Prevotella bivia | ADFO01000096 |
| Prevotella brevis | NR_041954 |
| Prevotella buccae | ACRB01000001 |
| Prevotella buccalis | JN867261 |
| Prevotella copri | ACBX02000014 |
| Prevotella corporis | L16465 |
| Prevotella dentalis | AB547678 |
| Prevotella denticola | CP002589 |
| Prevotella disiens | AEDO01000026 |
| Prevotella genomosp. C1 | AY278624 |
| Prevotella genomosp. C2 | AY278625 |
| Prevotella genomosp. P7 oral clone MB2_P31 | DQ003620 |
| Prevotella genomosp. P8 oral clone MB3_P13 | DQ003622 |
| Prevotella genomosp. P9 oral clone MB7_G16 | DQ003633 |
| Prevotella heparinolytica | GQ422742 |
| Prevotella histicola | JN867315 |
| Prevotella intermedia | AF414829 |
| Prevotella loescheii | JN867231 |
| Prevotella maculosa | AGEK01000035 |
| Prevotella marshii | AEEI01000070 |
| Prevotella melaninogenica | CP002122 |
| Prevotella micans | AGWK01000061 |
| Prevotella multiformis | AEWX01000054 |
| Prevotella multisaccharivorax | AFJE01000016 |
| Prevotella nanceiensis | JN867228 |
| Prevotella nigrescens | AFPX01000069 |
| Prevotella oralis | AEPE01000021 |
| Prevotella oris | ADDV01000091 |
| Prevotella oulorum | L16472 |
| Prevotella pallens | AFPY01000135 |
| Prevotella ruminicola | CP002006 |
| Prevotella salivae | AB108826 |
| Prevotella sp. BI_42 | AJ581354 |
| Prevotella sp. CM38 | HQ610181 |
| Prevotella sp. ICM1 | HQ616385 |
| Prevotella sp. ICM55 | HQ616399 |
| Prevotella sp. JCM 6330 | AB547699 |
| Prevotella sp. oral clone AA020 | AY005057 |
| Prevotella sp. oral clone ASCG10 | AY923148 |
| Prevotella sp. oral clone ASCG12 | DQ272511 |
| Prevotella sp. oral clone AU069 | AY005062 |
| Prevotella sp. oral clone CY006 | AY005063 |
| Prevotella sp. oral clone DA058 | AY005065 |
| Prevotella sp. oral clone FL019 | AY349392 |
| Prevotella sp. oral clone FU048 | AY349393 |
| Prevotella sp. oral clone FW035 | AY349394 |
| Prevotella sp. oral clone GI030 | AY349395 |
| Prevotella sp. oral clone GI032 | AY349396 |
| Prevotella sp. oral clone GI059 | AY349397 |
| Prevotella sp. oral clone GU027 | AY349398 |
| Prevotella sp. oral clone HF050 | AY349399 |
| Prevotella sp. oral clone ID019 | AY349400 |
| Prevotella sp. oral clone IDR_CEC_0055 | AY550997 |
| Prevotella sp. oral clone IK053 | AY349401 |
| Prevotella sp. oral clone IK062 | AY349402 |
| Prevotella sp. oral clone P4PB_83 P2 | AY207050 |
| Prevotella sp. oral taxon 292 | GQ422735 |
| Prevotella sp. oral taxon 299 | ACWZ01000026 |
| Prevotella sp. oral taxon 300 | GU409549 |
| Prevotella sp. oral taxon 302 | ACZK01000043 |
| Prevotella sp. oral taxon 310 | GQ422737 |
| Prevotella sp. oral taxon 317 | ACQH01000158 |
| Prevotella sp. oral taxon 472 | ACZS01000106 |
| Prevotella sp. oral taxon 781 | GQ422744 |
| Prevotella sp. oral taxon 782 | GQ422745 |
| Prevotella sp. oral taxon F68 | HM099652 |
| Prevotella sp. oral taxon G60 | GU432133 |
| Prevotella sp. oral taxon G70 | GU432179 |
| Prevotella sp. oral taxon G71 | GU432180 |
| Prevotella sp. SEQ053 | JN867222 |
| Prevotella sp. SEQ065 | JN867234 |
| Prevotella sp. SEQ072 | JN867238 |
| Prevotella sp. SEQ116 | JN867246 |
| Prevotella sp. SG12 | GU561343 |
| Prevotella sp. sp24 | AB003384 |
| Prevotella sp. sp34 | AB003385 |
| Prevotella stercorea | AB244774 |
| Prevotella tannerae | ACIJ02000018 |
| Prevotella timonensis | ADEF01000012 |
| Prevotella veroralis | ACVA01000027 |
| Prevotellaceae bacterium P4P_62 P1 | AY207061 |
| Propionibacteriaceae bacterium NML 02_0265 | EF599122 |
| Propionibacterium acidipropionici | NC_019395 |
| Propionibacterium acnes | ADJM01000010 |
| Propionibacterium avidum | AJ003055 |
| Propionibacterium freudenreichii | NR_036972 |
| Propionibacterium granulosum | FJ785716 |
| Propionibacterium jensenii | NR_042269 |
| Propionibacterium propionicum | NR_025277 |
| Propionibacterium sp. 434_HC2 | AFIL01000035 |
| Propionibacterium sp. H456 | AB177643 |
| Propionibacterium sp. LG | AY354921 |
| Propionibacterium sp. oral taxon 192 | GQ422728 |
| Propionibacterium sp. S555a | AB264622 |
| Propionibacterium thoenii | NR_042270 |
| Pseudomonas aeruginosa | AABQ07000001 |
| Pseudomonas fluorescens | AY622220 |
| Pseudomonas gessardii | FJ943496 |
| Pseudomonas mendocina | AAUL01000021 |
| Pseudomonas monteilii | NR_024910 |
| Pseudomonas poae | GU188951 |
| Pseudomonas pseudoalcaligenes | NR_037000 |
| Pseudomonas putida | AF094741 |
| Pseudomonas sp. 2_1_26 | ACWU01000257 |
| Pseudomonas sp. G1229 | DQ910482 |
| Pseudomonas sp. NP522b | EU723211 |
| Pseudomonas stutzeri | AM905854 |
| Pseudomonas tolaasii | AF320988 |
| Pseudomonas viridiflava | NR_042764 |
| Ralstonia pickettii | NC_010682 |
| Ralstonia sp. 5_7_47FAA | ACUF01000076 |
| Roseburia cecicola | GU233441 |
| Roseburia faecalis | AY804149 |
| Roseburia faecis | AY305310 |
| Roseburia hominis | AJ270482 |
| Roseburia intestinalis | FP929050 |
| Roseburia inulinivorans | AJ270473 |
| Roseburia sp. 11SE37 | FM954975 |
| Roseburia sp. 11SE38 | FM954976 |
| Rothia aeria | DQ673320 |
| Rothia dentocariosa | ADDW01000024 |
| Rothia mucilaginosa | ACVO01000020 |
| Rothia nasimurium | NR_025310 |
| Rothia sp. oral taxon 188 | GU470892 |
| Ruminobacter amylophilus | NR_026450 |
| Ruminococcaceae bacterium D16 | ADDX01000083 |
| Ruminococcus albus | AY445600 |
| Ruminococcus bromii | EU266549 |
| Ruminococcus callidus | NR_029160 |
| Ruminococcus champanellensis | FP929052 |
| Ruminococcus flavefaciens | NR_025931 |
| Ruminococcus gnavus | X94967 |
| Ruminococcus hansenii | M59114 |
| Ruminococcus lactaris | ABOU02000049 |
| Ruminococcus obeum | AY169419 |
| Ruminococcus sp. 18P13 | AJ515913 |
| Ruminococcus sp. 5_1_39BFAA | ACII01000172 |
| Ruminococcus sp. 9SE51 | FM954974 |
| Ruminococcus sp. ID8 | AY960564 |
| Ruminococcus sp. K_1 | AB222208 |
| Ruminococcus torques | AAVP02000002 |
| Salmonella bongori | NR_041699 |
| Salmonella enterica | NC_011149 |
| Salmonella enterica | NC_011205 |
| Salmonella enterica | DQ344532 |
| Salmonella enterica | ABEH02000004 |
| Salmonella enterica | ABAK02000001 |
| Salmonella enterica | NC_011080 |
| Salmonella enterica | EU118094 |
| Salmonella enterica | NC_011094 |
| Salmonella enterica | AE014613 |
| Salmonella enterica | ABFH02000001 |
| Salmonella enterica | ABEM01000001 |
| Salmonella enterica | ABAM02000001 |
| Salmonella typhimurium | DQ344533 |
| Salmonella typhimurium | AF170176 |
| Selenomonas artemidis | HM596274 |
| Selenomonas dianae | GQ422719 |
| Selenomonas flueggei | AF287803 |
| Selenomonas genomosp. C1 | AY278627 |
| Selenomonas genomosp. C2 | AY278628 |
| Selenomonas genomosp. P5 | AY341820 |
| Selenomonas genomosp. P6 oral clone MB3_C41 | DQ003636 |
| Selenomonas genomosp. P7 oral clone MB5_C08 | DQ003627 |
| Selenomonas genomosp. P8 oral clone MB5_P06 | DQ003628 |
| Selenomonas infelix | AF287802 |
| Selenomonas noxia | GU470909 |
| Selenomonas ruminantium | NR_075026 |
| Selenomonas sp. FOBRC9 | HQ616378 |
| Selenomonas sp. oral clone FT050 | AY349403 |
| Selenomonas sp. oral clone GI064 | AY349404 |
| Selenomonas sp. oral clone GT010 | AY349405 |
| Selenomonas sp. oral clone HU051 | AY349406 |
| Selenomonas sp. oral clone IK004 | AY349407 |
| Selenomonas sp. oral clone IQ048 | AY349408 |
| Selenomonas sp. oral clone JI021 | AY349409 |
| Selenomonas sp. oral clone JS031 | AY349410 |
| Selenomonas sp. oral clone OH4A | AY947498 |
| Selenomonas sp. oral clone P2PA_80 P4 | AY207052 |
| Selenomonas sp. oral taxon 137 | AENV01000007 |
| Selenomonas sp. oral taxon 149 | AEEJ01000007 |
| Selenomonas sputigena | ACKP02000033 |
| Serratia fonticola | NR_025339 |
| Serratia liquefaciens | NR_042062 |
| Serratia marcescens | GU826157 |
| Serratia odorifera | ADBY01000001 |
| Serratia proteamaculans | AAUN01000015 |
| Shigella boydii | AAKA01000007 |
| Shigella dysenteriae | NC_007606 |
| Shigella flexneri | AE005674 |
| Shigella sonnei | NC_007384 |
| Sphingobacterium faecium | NR_025537 |
| Sphingobacterium mizutaii | JF708889 |
| Sphingobacterium multivorum | NR_040953 |
| Sphingobacterium spiritivorum | ACHA02000013 |
| Sphingomonas echinoides | NR_024700 |
| Sphingomonas sp. oral clone FI012 | AY349411 |
| Sphingomonas sp. oral clone FZ016 | AY349412 |
| Sphingomonas sp. oral taxon A09 | HM099639 |
| Sphingomonas sp. oral taxon F71 | HM099645 |
| Staphylococcaceae bacterium NML 92_0017 | AY841362 |
| Staphylococcus aureus | CP002643 |
| Staphylococcus auricularis | JQ624774 |
| Staphylococcus capitis | ACFR01000029 |
| Staphylococcus caprae | ACRH01000033 |
| Staphylococcus carnosus | NR_075003 |
| Staphylococcus cohnii | JN175375 |
| Staphylococcus condimenti | NR_029345 |
| Staphylococcus epidermidis | ACHE01000056 |
| Staphylococcus equorum | NR_027520 |
| Staphylococcus fleurettii | NR_041326 |
| Staphylococcus haemolyticus | NC_007168 |
| Staphylococcus hominis | AM157418 |
| Staphylococcus lugdunensis | AEQA01000024 |
| Staphylococcus pasteuri | FJ189773 |
| Staphylococcus pseudintermedius | CP002439 |
| Staphylococcus saccharolyticus | NR_029158 |
| Staphylococcus saprophyticus | NC_007350 |
| Staphylococcus sciuri | NR_025520 |
| Staphylococcus sp. clone bottae7 | AF467424 |
| Staphylococcus sp. H292 | AB177642 |
| Staphylococcus sp. H780 | AB177644 |
| Staphylococcus succinus | NR_028667 |
| Staphylococcus vitulinus | NR_024670 |
| Staphylococcus warneri | ACPZ01000009 |
| Staphylococcus xylosus | AY395016 |
| Streptobacillus moniliformis | NR_027615 |
| Streptococcus agalactiae | AAJO01000130 |
| Streptococcus alactolyticus | NR_041781 |
| Streptococcus anginosus | AECT01000011 |
| Streptococcus australis | AEQR01000024 |
| Streptococcus bovis | AEEL01000030 |
| Streptococcus canis | AJ413203 |
| Streptococcus constellatus | AY277942 |
| Streptococcus cristatus | AEVC01000028 |
| Streptococcus downei | AEKN01000002 |
| Streptococcus dysgalactiae | AP010935 |
| Streptococcus equi | CP001129 |
| Streptococcus equinus | AEVB01000043 |
| Streptococcus gallolyticus | FR824043 |
| Streptococcus genomosp. C1 | AY278629 |
| Streptococcus genomosp. C2 | AY278630 |
| Streptococcus genomosp. C3 | AY278631 |
| Streptococcus genomosp. C4 | AY278632 |
| Streptococcus genomosp. C5 | AY278633 |
| Streptococcus genomosp. C6 | AY278634 |
| Streptococcus genomosp. C7 | AY278635 |
| Streptococcus genomosp. C8 | AY278609 |
| Streptococcus gordonii | NC_009785 |
| Streptococcus infantarius | ABJK02000017 |
| Streptococcus infantis | AFNN01000024 |
| Streptococcus intermedius | NR_028736 |
| Streptococcus lutetiensis | NR_037096 |
| Streptococcus massiliensis | AY769997 |
| Streptococcus milleri | X81023 |
| Streptococcus mitis | AM157420 |
| Streptococcus mutans | AP010655 |
| Streptococcus oligofermentans | AY099095 |
| Streptococcus oralis | ADMV01000001 |
| Streptococcus parasanguinis | AEKM01000012 |
| Streptococcus pasteurianus | AP012054 |
| Streptococcus peroris | AEVF01000016 |
| Streptococcus pneumoniae | AE008537 |
| Streptococcus porcinus | EF121439 |
| Streptococcus pseudopneumoniae | FJ827123 |
| Streptococcus pseudoporcinus | AENS01000003 |
| Streptococcus pyogenes | AE006496 |
| Streptococcus ratti | X58304 |
| Streptococcus salivarius | AGBV01000001 |
| Streptococcus sanguinis | NR_074974 |
| Streptococcus sinensis | AF432857 |
| Streptococcus sp. 16362 | JN590019 |
| Streptococcus sp. 2_1_36FAA | ACOI01000028 |
| Streptococcus sp. 2285_97 | AJ131965 |
| Streptococcus sp. 69130 | X78825 |
| Streptococcus sp. AC15 | HQ616356 |
| Streptococcus sp. ACS2 | HQ616360 |
| Streptococcus sp. AS20 | HQ616366 |
| Streptococcus sp. BS35a | HQ616369 |
| Streptococcus sp. C150 | ACRI01000045 |
| Streptococcus sp. CM6 | HQ616372 |
| Streptococcus sp. CM7 | HQ616373 |
| Streptococcus sp. ICM10 | HQ616389 |
| Streptococcus sp. ICM12 | HQ616390 |
| Streptococcus sp. ICM2 | HQ616386 |
| Streptococcus sp. ICM4 | HQ616387 |
| Streptococcus sp. ICM45 | HQ616394 |
| Streptococcus sp. M143 | ACRK01000025 |
| Streptococcus sp. M334 | ACRL01000052 |
| Streptococcus sp. OBRC6 | HQ616352 |
| Streptococcus sp. oral clone ASB02 | AY923121 |
| Streptococcus sp. oral clone ASCA03 | DQ272504 |
| Streptococcus sp. oral clone ASCA04 | AY923116 |
| Streptococcus sp. oral clone ASCA09 | AY923119 |
| Streptococcus sp. oral clone ASCB04 | AY923123 |
| Streptococcus sp. oral clone ASCB06 | AY923124 |
| Streptococcus sp. oral clone ASCC04 | AY923127 |
| Streptococcus sp. oral clone ASCC05 | AY923128 |
| Streptococcus sp. oral clone ASCC12 | DQ272507 |
| Streptococcus sp. oral clone ASCD01 | AY923129 |
| Streptococcus sp. oral clone ASCD09 | AY923130 |
| Streptococcus sp. oral clone ASCD10 | DQ272509 |
| Streptococcus sp. oral clone ASCE03 | AY923134 |
| Streptococcus sp. oral clone ASCE04 | AY953253 |
| Streptococcus sp. oral clone ASCE05 | DQ272510 |
| Streptococcus sp. oral clone ASCE06 | AY923135 |
| Streptococcus sp. oral clone ASCE09 | AY923136 |
| Streptococcus sp. oral clone ASCE10 | AY923137 |
| Streptococcus sp. oral clone ASCE12 | AY923138 |
| Streptococcus sp. oral clone ASCF05 | AY923140 |
| Streptococcus sp. oral clone ASCF07 | AY953255 |
| Streptococcus sp. oral clone ASCF09 | AY923142 |
| Streptococcus sp. oral clone ASCG04 | AY923145 |
| Streptococcus sp. oral clone BW009 | AY005042 |
| Streptococcus sp. oral clone CH016 | AY005044 |
| Streptococcus sp. oral clone GK051 | AY349413 |
| Streptococcus sp. oral clone GM006 | AY349414 |
| Streptococcus sp. oral clone P2PA_41 P2 | AY207051 |
| Streptococcus sp. oral clone P4PA_30 P4 | AY207064 |
| Streptococcus sp. oral taxon 071 | AEEP01000019 |
| Streptococcus sp. oral taxon G59 | GU432132 |
| Streptococcus sp. oral taxon G62 | GU432146 |
| Streptococcus sp. oral taxon G63 | GU432150 |
| Streptococcus sp. SHV515 | Y07601 |
| Streptococcus suis | FM252032 |
| Streptococcus thermophilus | CP000419 |
| Streptococcus uberis | HQ391900 |
| Streptococcus urinalis | DQ303194 |
| Streptococcus vestibularis | AEKO01000008 |
| Streptococcus viridans | AF076036 |
| Sutterella morbirenis | AJ832129 |
| Sutterella parvirubra | AB300989 |
| Sutterella sanguinus | AJ748647 |
| Sutterella sp. YIT 12072 | AB491210 |
| Sutterella stercoricanis | NR_025600 |
| Sutterella wadsworthensis | ADMF01000048 |
| Synergistes genomosp. C1 | AY278615 |
| Synergistes sp. RMA 14551 | DQ412722 |
| Synergistetes bacterium ADV897 | GQ258968 |
| Synergistetes bacterium LBVCM1157 | GQ258969 |
| Synergistetes bacterium oral taxon 362 | GU410752 |
| Synergistetes bacterium oral taxon D48 | GU430992 |
| Turicibacter sanguinis | AF349724 |
| Veillonella atypica | AEDS01000059 |
| Veillonella dispar | ACIK02000021 |
| Veillonella genomosp. P1 oral clone MB5_P17 | DQ003631 |
| Veillonella montpellierensis | AF473836 |
| Veillonella parvula | ADFU01000009 |
| Veillonella sp. 3_1_44 | ADCV01000019 |
| Veillonella sp. 6_1_27 | ADCW01000016 |
| Veillonella sp. ACP1 | HQ616359 |
| Veillonella sp. AS16 | HQ616365 |
| Veillonella sp. BS32b | HQ616368 |
| Veillonella sp. ICM51a | HQ616396 |
| Veillonella sp. MSA12 | HQ616381 |
| Veillonella sp. NVG 100cf | EF108443 |
| Veillonella sp. OK11 | JN695650 |
| Veillonella sp. oral clone ASCA08 | AY923118 |
| Veillonella sp. oral clone ASCB03 | AY923122 |
| Veillonella sp. oral clone ASCG01 | AY923144 |
| Veillonella sp. oral clone ASCG02 | AY953257 |
| Veillonella sp. oral clone OH1A | AY947495 |
| Veillonella sp. oral taxon 158 | AENU01000007 |
| Veillonellaceae bacterium oral taxon 131 | GU402916 |
| Veillonellaceae bacterium oral taxon 155 | GU470897 |
| Vibrio cholerae | AAUR01000095 |
| Vibrio fluvialis | X76335 |
| Vibrio furnissii | CP002377 |
| Vibrio mimicus | ADAF01000001 |
| Vibrio parahaemolyticus | AAWQ01000116 |
| Vibrio sp. RC341 | ACZT01000024 |
| Vibrio vulnificus | AE016796 |
| Yersinia aldovae | AJ871363 |
| Yersinia aleksiciae | AJ627597 |
| Yersinia bercovieri | AF366377 |
| Yersinia enterocolitica | FR729477 |
| Yersinia frederiksenii | AF366379 |
| Yersinia intermedia | AF366380 |
| Yersinia kristensenii | ACCA01000078 |
| Yersinia mollaretii | NR_027546 |
| Yersinia pestis | AE013632 |
| Yersinia pseudotuberculosis | NC_009708 |
| Yersinia rohdei | ACCD01000071 |

TABLE 3
Exemplary Bacterial Strains

| Strain | Deposit Number |
|---|---|
| Parabacteroides goldsteinii | PTA-126574 |
| Bifidobacterium animalis ssp. lactis | PTA-125097 |
| Strain A | |
| Blautia Massiliensis Strain A | PTA-125134 |
| Prevotella Strain B | NRRL accession Number B 50329 |
| Prevotella Histicola | PTA-126140 |
| Blautia Strain A | PTA-125346 |
| Lactococcus lactis cremoris Strain A | PTA-125368 |
| Lactobacillus salivarius | PTA-125893 |
| Ruminococcus gnavus strain | PTA-125706 |
| Tyzzerella nexilis strain | PTA-125707 |
| Paraclostridium benzoelyticum | PTA-125894 |
| Ruminococcus gnavus (also referred | PTA-126695 |
| to as Mediterraneibacter gnavus) | |
| Veillonella parvula | PTA-125710 |
| Veillonella atypica Strain A | PTA-125709 |
| Veillonella atypica Strain B | PTA-125711 |
| Veillonella parvula Strain A | PTA-125691 |
| Veillonella parvula Strain B | PTA-125711 |
| Veillonella tobetsuensis Strain A | PTA-125708 |
| Agathobaculum sp. | PTA-125892 |
| Turicibacter sanguinis | PTA-125889 |
| Klebsiella quasipneumoniae subsp. | PTA-125891 |
| similipneumoniae | |
| Klebsiella oxytoca | PTA-125890 |
| Megasphaera Sp. Strain A | PTA-126770 |
| Megasphaera Sp. | PTA-126837 |
| Harryflintia acetispora | PTA-126694 |
| Fournierella massiliensis | PTA-126696 |

TABLE 4
Exemplary Bacterial Strains

| | |
|---|---|
| Escherichia coli | NCIMB 12210 |
| Enterococcus faecalis | NCIMB 13280 |
| Bacteroides fragilis | DSM 2151 |
| Bacteroides vulgatus | DSM 1447 |
| Bacteroides ovatus | DSM 1896 |
| Megasphaera massiliensis | DSM 26228 |
| Megasphaera elsdenii | NCIMB 8927 |
| Megasphaera massiliensis | NCIMB 42787 |
| Bifidobacterium breve | DSM 20213 |
| Bifidobacterium longum subsp. longum | DSM 20219 |
| Faecalibacterium prausnitzii | DSM17677 |
| Anaerostipes hadrus | DSM 3319 |
| Blautia coccoides | DSM 935 |
| Dorea longicatena | DSM 13814 |
| Parabacteroides distasonis | DSM 20701 |
| Faecalicatena contorta | DSM3982 |
| Ruminococcus gnavus | ATCC29149 |
| Megasphaera massiliensis | NCIMB 43388 |
| Megasphaera massinensis | NCIMB 43389 |
| Megasphaera spp | NCIMB 43385 |
| Megasphaera spp. | NCIMB 43386 |
| Megasphaera spp | NCIMB 43387 |
| Parabacteroides distasonis (also referred | NCIMB 42382 |
| to as “Parabacteroides sp 755”) | |

Modified Bacteria and mEVs

In some aspects, the bacteria and/or mEVs (such as smEVs and/or pmEVs) described herein are modified such that they comprise, are linked to, and/or are bound by a therapeutic moiety.

In some embodiments, the therapeutic moiety is a cancer-specific moiety. In some embodiments, the cancer-specific moiety has binding specificity for a cancer cell (e.g., has binding specificity for a cancer-specific antigen). In some embodiments, the cancer-specific moiety comprises an antibody or antigen binding fragment thereof. In some embodiments, the cancer-specific moiety comprises a T cell receptor or a chimeric antigen receptor (CAR). In some embodiments, the cancer-specific moiety comprises a ligand for a receptor expressed on the surface of a cancer cell or a receptor-binding fragment thereof. In some embodiments, the cancer-specific moiety is a bipartite fusion protein that has two parts: a first part that binds to and/or is linked to the bacterium and a second part that is capable of binding to a cancer cell (e.g., by having binding specificity for a cancer-specific antigen). In some embodiments, the first part is a fragment of or a full-length peptidoglycan recognition protein, such as PGRP. In some embodiments the first part has binding specificity for the mEV (e.g., by having binding specificity for a bacterial antigen). In some embodiments, the first and/or second part comprises an antibody or antigen binding fragment thereof. In some embodiments, the first and/or second part comprises a T cell receptor or a chimeric antigen receptor (CAR). In some embodiments, the first and/or second part comprises a ligand for a receptor expressed on the surface of a cancer cell or a receptor-binding fragment thereof. In certain embodiments, co-administration of the cancer-specific moiety with the pharmaceutical agent (either in combination or in separate administrations) increases the targeting of the pharmaceutical agent to the cancer cells.

In some embodiments, the bacteria and/or mEVs described herein can be modified such that they comprise, are linked to, and/or are bound by a magnetic and/or paramagnetic moiety (e.g., a magnetic bead). In some embodiments, the magnetic and/or paramagnetic moiety is comprised by and/or directly linked to the bacteria. In some embodiments, the magnetic and/or paramagnetic moiety is linked to and/or a part of a bacteria- or a mEV-binding moiety that binds to the bacteria or mEV. In some embodiments, the bacteria- or mEV-binding moiety is a fragment of or a full-length peptidoglycan recognition protein, such as PGRP. In some embodiments the bacteria- or mEV-binding moiety has binding specificity for the bacteria or mEV (e.g., by having binding specificity for a bacterial antigen). In some embodiments, the bacteria- or mEV-binding moiety comprises an antibody or antigen binding fragment thereof. In some embodiments, the bacteria- or mEV-binding moiety comprises a T cell receptor or a chimeric antigen receptor (CAR). In some embodiments, the bacteria- or mEV-binding moiety comprises a ligand for a receptor expressed on the surface of a cancer cell or a receptor-binding fragment thereof. In certain embodiments, co-administration of the magnetic and/or paramagnetic moiety with the bacteria or mEVs (either together or in separate administrations) can be used to increase the targeting of the mEVs (e.g., to cancer cells and/or a part of a subject where cancer cells are present.

Production of Processed Microbial Extracellular Vesicles (pmEVs)

In certain aspects, the pmEVs described herein can be prepared using any method known in the art.

In some embodiments, the pmEVs are prepared without a pmEV purification step. For example, in some embodiments, bacteria from which the pmEVs described herein are released are killed using a method that leaves the bacterial pmEVs intact, and the resulting bacterial components, including the pmEVs, are used in the methods and compositions described herein. In some embodiments, the bacteria are killed using an antibiotic (e.g., using an antibiotic described herein). In some embodiments, the bacteria are killed using UV irradiation.

In some embodiments, the pmEVs described herein are purified from one or more other bacterial components. Methods for purifying pmEVs from bacteria (and optionally, other bacterial components) are known in the art. In some embodiments, pmEVs are prepared from bacterial cultures using methods described in Thein, et al. (J. Proteome Res. 9(12):6135-6147 (2010)) or Sandrini, et al. (Bio-protocol 4(21); e1287 (2014)), each of which is hereby incorporated by reference in its entirety. In some embodiments, the bacteria are cultured to high optical density and then centrifuged to pellet bacteria (e.g., at 10,000-15,000×g for 10-15 min at room temperature or 4° C.). In some embodiments, the supernatants are discarded and cell pellets are frozen at −80° C. In some embodiments, cell pellets are thawed on ice and resuspended in 100 mM Tris-HCl, pH 7.5 supplemented with 1 mg/mL DNase I. In some embodiments, cells are lysed using an Emulsiflex C-3 (Avestin, Inc.) under conditions recommended by the manufacturer. In some embodiments, debris and unlysed cells are pelleted by centrifugation at 10,000×g for 15 min at 4° C. In some embodiments, supernatants are then centrifuged at 120,000×g for 1 hour at 4° C. In some embodiments, pellets are resuspended in ice-cold 100 mM sodium carbonate, pH 11, incubated with agitation for 1 hr at 4° C., and then centrifuged at 120,000×g for 1 hour at 4° C. In some embodiments, pellets are resuspended in 100 mM Tris-HCl, pH 7.5, re-centrifuged at 120,000×g for 20 min at 4° C., and then resuspended in 0.1 M Tris-HCl, pH 7.5 or in PBS. In some embodiments, samples are stored at −20° C.

In certain aspects, pmEVs are obtained by methods adapted from Sandrini et al, 2014. In some embodiments, bacterial cultures are centrifuged at 10,000-15,500×g for 10-15 min at room temp or at 4° C. In some embodiments, cell pellets are frozen at −80° C., and supernatants are discarded. In some embodiments, cell pellets are thawed on ice and resuspended in 10 mM Tris-HCl, pH 8.0, 1 mM EDTA supplemented with 0.1 mg/mL lysozyme. In some embodiments, samples are incubated with mixing at room temp or at 37° C. for 30 min. In some embodiments, samples are re-frozen at −80° C., and thawed again on ice. In some embodiments, DNase I is added to a final concentration of 1.6 mg/mL and MgCl2 to a final concentration of 100 mM. In some embodiments, samples are sonicated using a QSonica Q500 sonicator with 7 cycles of 30 sec on and 30 sec off. In some embodiments, debris and unlysed cells are pelleted by centrifugation at 10,000×g for 15 min, at 4° C. In some embodiments, supernatants are then centrifuged at 110,000×g for 15 min at 4° C. In some embodiments, pellets are resuspended in 10 mM Tris-HCl, pH 8.0, 2% Triton X-100 and incubated 30-60 min with mixing at room temperature. In some embodiments, samples are centrifuged at 110,000×g for 15 min at 4° C. In some embodiments, pellets are resuspended in PBS and stored at −20° C.

In certain aspects, a method of forming (e.g., preparing) isolated bacterial pmEVs, described herein, comprises the steps of: (a) centrifuging a bacterial culture, thereby forming a first pellet and a first supernatant, wherein the first pellet comprises cells; (b) discarding the first supernatant; (c) resuspending the first pellet in a solution; (d) lysing the cells; (e) centrifuging the lysed cells, thereby forming a second pellet and a second supernatant; (f) discarding the second pellet and centrifuging the second supernatant, thereby forming a third pellet and a third supernatant; (g) discarding the third supernatant and resuspending the third pellet in a second solution, thereby forming the isolated bacterial pmEVs.

In some embodiments, the method further comprises the steps of: (h) centrifuging the solution of step (g), thereby forming a fourth pellet and a fourth supernatant, (i) discarding the fourth supernatant and resuspending the fourth pellet in a third solution. In some embodiments, the method further comprises the steps of: (j) centrifuging the solution of step (i), thereby forming a fifth pellet and a fifth supernatant; and (k) discarding the fifth supernatant and resuspending the fifth pellet in a fourth solution.

In some embodiments, the centrifugation of step (a) is at 10,000×g. In some embodiments the centrifugation of step (a) is for 10-15 minutes. In some embodiments, the centrifugation of step (a) is at 4° C., or room temperature. In some embodiments, step (b) further comprises freezing the first pellet at −80° C. In some embodiments, the solution in step (c) is 100 mM Tris-HCl, pH 7.5 supplemented with 1 mg/ml DNaseI. In some embodiments, the solution in step (c) is 10 mM Tris-HCl, pH 8.0, 1 mM EDTA, supplemented with 0.1 mg/ml lysozyme. In some embodiments, step (c) further comprises incubating for 30 minutes at 37° C., or room temperature. In some embodiments, step (c) further comprises freezing the first pellet at −80° C. In some embodiments, step (c) further comprises adding DNase I to a final concentration of 1.6 mg/ml. In some embodiments, step (c) further comprises adding MgCl₂ to a final concentration of 100 mM. In some embodiments, the cells are lysed in step (d) via homogenization. In some embodiments, the cells are lysed in step (d) via emulsiflex C3. In some embodiments, the cells are lysed in step (d) via sonication. In some embodiments, the cells are sonicated in 7 cycles, wherein each cycle comprises 30 seconds of sonication and 30 seconds without sonication. In some embodiments, the centrifugation of step (e) is at 10,000×g. In some embodiments, the centrifugation of step (e) is for 15 minutes. In some embodiments, the centrifugation of step (e) is at 4° C., or room temperature.

In some embodiments, the centrifugation of step (f) is at 120,000×g. In some embodiments, the centrifugation of step (f) is at 110,000×g. In some embodiments, the centrifugation of step (f) is for 1 hour. In some embodiments, the centrifugation of step (f) is for 15 minutes. In some embodiments, the centrifugation of step (f) is at 4° C., or room temperature. In some embodiments, the second solution in step (g) is 100 mM sodium carbonate, pH 11. In some embodiments, the second solution in step (g) is 10 mM Tris-HCl pH 8.0, 2% triton X-100. In some embodiments, step (g) further comprises incubating the solution for 1 hour at 4° C. In some embodiments, step (g) further comprises incubating the solution for 30-60 minutes at room temperature. In some embodiments, the centrifugation of step (h) is at 120,000×g. In some embodiments, the centrifugation of step (h) is at 110,000×g. In some embodiments, the centrifugation of step (h) is for 1 hour. In some embodiments, the centrifugation of step (h) is for 15 minutes. In some embodiments, the centrifugation of step (h) is at 4° C., or room temperature. In some embodiments, the third solution in step (i) is 100 mM Tris-HCl, pH 7.5. In some embodiments, the third solution in step (i) is PBS. In some embodiments, the centrifugation of step (j) is at 120,000×g. In some embodiments, the centrifugation of step (j) is for 20 minutes. In some embodiments, the centrifugation of step (j) is at 4° C., or room temperature. In some embodiments, the fourth solution in step (k) is 100 mM Tris-HCl, pH 7.5 or PBS.

pmEVs obtained by methods provided herein may be further purified by size based column chromatography, by affinity chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in 35% Optiprep in PBS. In some embodiments, if filtration was used to concentrate the filtered supernatant, the concentrate is diluted using 60% Optiprep to a final concentration of 35% Optiprep. Samples are applied to a 35-60% discontinuous sucrose gradient and centrifuged at 200,000×g for 3-24 hours at 4° C.

In some embodiments, to confirm sterility and isolation of the pmEV preparations, pmEVs are serially diluted onto agar medium used for routine culture of the bacteria being tested, and incubated using routine conditions. Non-sterile preparations are passed through a 0.22 um filter to exclude intact cells. To further increase purity, isolated pmEVs may be DNase or proteinase K treated.

In some embodiments, the sterility of the pmEV preparations can be confirmed by plating a portion of the pmEVs onto agar medium used for standard culture of the bacteria used in the generation of the pmEVs and incubating using standard conditions.

In some embodiments select pmEVs are isolated and enriched by chromatography and binding surface moieties on pmEVs. In other embodiments, select pmEVs are isolated and/or enriched by fluorescent cell sorting by methods using affinity reagents, chemical dyes, recombinant proteins or other methods known to one skilled in the art.

The pmEVs can be analyzed, e.g., as described in Jeppesen, et al. Cell 177:428 (2019).

In some embodiments, pmEVs are lyophilized.

In some embodiments, pmEVs are gamma irradiated (e.g., at 17.5 or 25 kGy).

In some embodiments, pmEVs are UV irradiated.

In some embodiments, pmEVs are heat inactivated (e.g., at 50° C. for two hours or at 90° C. for two hours).

In some embodiments, pmEVs are acid treated.

In some embodiments, pmEVs are oxygen sparged (e.g., at 0.1 vvm for two hours).

The phase of growth can affect the amount or properties of bacteria. In the methods of pmEV preparation provided herein, pmEVs can be isolated, e.g., from a culture, at the start of the log phase of growth, midway through the log phase, and/or once stationary phase growth has been reached.

Production of Secreted Microbial Extracellular Vesicles (smEVs)

In certain aspects, the smEVs described herein can be prepared using any method known in the art.

In some embodiments, the smEVs are prepared without a smEV purification step. For example, in some embodiments, bacteria described herein are killed using a method that leaves the smEVs intact and the resulting bacterial components, including the smEVs, are used in the methods and compositions described herein. In some embodiments, the bacteria are killed using an antibiotic (e.g., using an antibiotic described herein). In some embodiments, the bacteria are killed using UV irradiation. In some embodiments, the bacteria are heat-killed.

In some embodiments, the smEVs described herein are purified from one or more other bacterial components. Methods for purifying smEVs from bacteria are known in the art. In some embodiments, smEVs are prepared from bacterial cultures using methods described in S. Bin Park, et al. PLoS ONE. 6(3):e17629 (2011) or G. Nodheim, et al. PLoS ONE. 10(9); e0134353 (2015) or Jeppesen, et al. Cell 177:428 (2019), each of which is hereby incorporated by reference in its entirety. In some embodiments, the bacteria are cultured to high optical density and then centrifuged to pellet bacteria (e.g., at 10,000×g for 30 min at 4° C., at 15,500×g for 15 min at 4° C.). In some embodiments, the culture supernatants are then passed through filters to exclude intact bacterial cells (e.g., a 0.22 μm filter). In some embodiments, the supernatants are then subjected to tangential flow filtration, during which the supernatant is concentrated, species smaller than 100 kDa are removed, and the media is partially exchanged with PBS. In some embodiments, filtered supernatants are centrifuged to pellet bacterial smEVs (e.g., at 100,000-150,000×g for 1-3 hours at 4° C., at 200,000×g for 1-3 hours at 4° C.). In some embodiments, the smEVs are further purified by resuspending the resulting smEV pellets (e.g., in PBS), and applying the resuspended smEVs to an Optiprep (iodixanol) gradient or gradient (e.g., a 30-60% discontinuous gradient, a 0-45% discontinuous gradient), followed by centrifugation (e.g., at 200,000×g for 4-20 hours at 4° C.). smEV bands can be collected, diluted with PBS, and centrifuged to pellet the smEVs (e.g., at 150,000×g for 3 hours at 4° C., at 200,000×g for 1 hour at 4° C.). The purified smEVs can be stored, for example, at −80° C., or −20° C. until use. In some embodiments, the smEVs are further purified by treatment with DNase and/or proteinase K.

For example, in some embodiments, cultures of bacteria can be centrifuged at 11,000×g for 20-40 min at 4° C. to pellet bacteria. Culture supernatants may be passed through a 0.22 μm filter to exclude intact bacterial cells. Filtered supernatants may then be concentrated using methods that may include, but are not limited to, ammonium sulfate precipitation, ultracentrifugation, or filtration. For example, for ammonium sulfate precipitation, 1.5-3 M ammonium sulfate can be added to filtered supernatant slowly, while stirring at 4° C. Precipitations can be incubated at 4° C. for 8-48 hours and then centrifuged at 11,000×g for 20-40 min at 4° C. The resulting pellets contain bacteria smEVs and other debris. Using ultracentrifugation, filtered supernatants can be centrifuged at 100,000-200,000×g for 1-16 hours at 4° C. The pellet of this centrifugation contains bacteria smEVs and other debris such as large protein complexes. In some embodiments, using a filtration technique, such as through the use of an Amicon Ultra spin filter or by tangential flow filtration, supernatants can be filtered so as to retain species of molecular weight >50 or 100 kDa.

Alternatively, smEVs can be obtained from bacteria cultures continuously during growth, or at selected time points during growth, for example, by connecting a bioreactor to an alternating tangential flow (ATF) system (e.g., XCell ATF from Repligen). The ATF system retains intact cells (>0.22 um) in the bioreactor, and allows smaller components (e.g., smEVs, free proteins) to pass through a filter for collection. For example, the system may be configured so that the <0.22 um filtrate is then passed through a second filter of 100 kDa, allowing species such as smEVs between 0.22 um and 100 kDa to be collected, and species smaller than 100 kDa to be pumped back into the bioreactor. Alternatively, the system may be configured to allow for medium in the bioreactor to be replenished and/or modified during growth of the culture. smEVs collected by this method may be further purified and/or concentrated by ultracentrifugation or filtration as described above for filtered supernatants.

smEVs obtained by methods provided herein may be further purified by size-based column chromatography, by affinity chromatography, by ion-exchange chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in PBS and 3 volumes of 60% Optiprep are added to the sample. In some embodiments, if filtration was used to concentrate the filtered supernatant, the concentrate is diluted using 60% Optiprep to a final concentration of 35% Optiprep. Samples are applied to a 0-45% discontinuous Optiprep gradient and centrifuged at 200,000×g for 3-24 hours at 4° C., e.g., 4-24 hours at 4° C.

In some embodiments, to confirm sterility and isolation of the smEV preparations, smEVs are serially diluted onto agar medium used for routine culture of the bacteria being tested, and incubated using routine conditions. Non-sterile preparations are passed through a 0.22 um filter to exclude intact cells. To further increase purity, isolated smEVs may be DNase or proteinase K treated.

In some embodiments, for preparation of smEVs used for in vivo injections, purified smEVs are processed as described previously (G. Norheim, et al. PLoS ONE. 10(9): e0134353 (2015)). Briefly, after sucrose gradient centrifugation, bands containing smEVs are resuspended to a final concentration of 50 μg/mL in a solution containing 3% sucrose or other solution suitable for in vivo injection known to one skilled in the art. This solution may also contain adjuvant, for example aluminum hydroxide at a concentration of 0-0.5% (w/v). In some embodiments, for preparation of smEVs used for in vivo injections, smEVs in PBS are sterile-filtered to <0.22 um.

In certain embodiments, to make samples compatible with further testing (e.g., to remove sucrose prior to TEM imaging or in vitro assays), samples are buffer exchanged into PBS or 30 mM Tris, pH 8.0 using filtration (e.g., Amicon Ultra columns), dialysis, or ultracentrifugation (200,000×g, ≥3 hours, 4° C.) and resuspension.

In some embodiments, the sterility of the smEV preparations can be confirmed by plating a portion of the smEVs onto agar medium used for standard culture of the bacteria used in the generation of the smEVs and incubating using standard conditions.

In some embodiments, select smEVs are isolated and enriched by chromatography and binding surface moieties on smEVs. In other embodiments, select smEVs are isolated and/or enriched by fluorescent cell sorting by methods using affinity reagents, chemical dyes, recombinant proteins or other methods known to one skilled in the art.

The smEVs can be analyzed, e.g., as described in Jeppesen, et al. Cell 177:428 (2019).

In some embodiments, smEVs are lyophilized.

In some embodiments, smEVs are gamma irradiated (e.g., at 17.5 or 25 kGy).

In some embodiments, smEVs are UV irradiated.

In some embodiments, smEVs are heat inactivated (e.g., at 50° C. for two hours or at 90° C. for two hours).

In some embodiments, smEVs s are acid treated.

In some embodiments, smEVs are oxygen sparged (e.g., at 0.1% vvm for two hours).

The phase of growth can affect the amount or properties of bacteria and/or smEVs produced by bacteria. For example, in the methods of smEV preparation provided herein, smEVs can be isolated, e.g., from a culture, at the start of the log phase of growth, midway through the log phase, and/or once stationary phase growth has been reached.

The growth environment (e.g., culture conditions) can affect the amount of smEVs produced by bacteria. For example, the yield of smEVs can be increased by an smEV inducer, as provided in Table 5.

TABLE 5
Culture Techniques to Increase smEV Production

| smEV | | |
|---|---|---|
| inducement | smEV inducer | Acts on |
| Temperature | Heat | stress response |
| | RT to 37° C. temp change | simulates infection |
| | 37 to 40° C. temp change | febrile infection |
| ROS | Plumbagin | oxidative stress response |
| | Cumene hydroperoxide | oxidative stress response |
| | Hydrogen Peroxide | oxidative stress response |
| Antibiotics | Ciprofloxacin | bacterial SOS response |
| | Gentamycin | protein synthesis |
| | Polymyxin B | outer membrane |
| | D-cylcloserine | cell wall |
| Osmolyte | NaCl | osmotic stress |
| Metal Ion | Iron Chelation | iron levels |
| Stress | EDTA | removes divalent cations |
| | Low Hemin | iron levels |
| Media | Lactate | growth |
| additives or | Amino acid deprivation | stress |
| removal | Hexadecane | stress |
| | Glucose | growth |
| | Sodium bicarbonate | ToxT induction |
| | PQS | vesiculator (from bacteria) |
| | Diamines + DFMO | membrane anchoring |
| | | (negativicutes only) |
| | High nutrients | enhanced growth |
| | Low nutrients | |
| Other | Oxygen | oxygen stress in anaerobe |
| mechanisms | No Cysteine | oxygen stress in anaerobe |
| | Inducing biofilm or | |
| | floculation | |
| | Diauxic Growth | |
| | Phage | |
| | Urea | |

In the methods of smEVs preparation provided herein, the method can optionally include exposing a culture of bacteria to a smEV inducer prior to isolating smEVs from the bacterial culture. The culture of bacteria can be exposed to a smEV inducer at the start of the log phase of growth, midway through the log phase, and/or once stationary phase growth has been reached.

Solid Dosage Form Compositions

In certain embodiments, provided herein are solid dosage forms (e.g., pharmaceutical products having a solid dosage form) comprising a pharmaceutical agent that contains bacteria and/or mEVs (such as smEVs and/or pmEVs). In some embodiments, the pharmaceutical agent can optionally contain one or more additional components, such as a cryoprotectant. The pharmaceutical agent can be lyophilized (e.g., resulting in a powder). The pharmaceutical agent can be combined with one or more excipients (e.g., pharmaceutically acceptable excipients) in the solid dose form. The pharmaceutical agent is also referred to as drug substance. The solid dosage form is also referred to as solid dose form.

In certain aspects provided herein are solid dosage forms of pharmaceutical compositions. The pharmaceutical composition is also referred to as drug product. In certain embodiments, the solid dosage form comprises a pharmaceutical agent (e.g., bacteria and/or an agent (e.g., component) of bacterial origin, such as mEVs, a powder comprising bacteria and/or an agent (e.g., component) of bacterial origin, such as mEVs) and a diluent. In certain embodiments, the total pharmaceutical agent mass is at least 2.5%, 5%, 10%, 15%, 20%, 25%, 30%, 35%, 40%, 45%, 50%, 55%, 60%, 65%, 70%, 75%, 80%, 85%, 90%, or 95% of the total mass of the pharmaceutical composition. In some embodiments, the total pharmaceutical agent mass is no more than 95%, 90%, 85%, 80%, 75%, 70%, 65%, 60%, 55%, 50%, 45%, 40%, 35%, 30%, 25%, 20%, 15%, 10%, 5%, or 2.5% of the total mass of the pharmaceutical composition.

In some embodiments, the total mass of the diluent is at least 1%, 5%, 10%, 15%, 20%, 25%, 30%, 35%, 40%, 45%, 50%, 55%, 60%, 65%, 70%, 75%, 80%, 85%, 90%, 95%, or 98% of the total mass of the pharmaceutical composition. In some embodiments, the total mass of the diluent is no more than 98%, 95%, 90%, 85%, 80%, 75%, 70%, 65%, 60%, 55%, 50%, 45%, 40%, 35%, 30%, 25%, 20%, 15%, 10%, 5%, or 1% of the total mass of the pharmaceutical composition. In some embodiments, the diluent comprises mannitol.

In certain embodiments, the solid dosage form provided herein comprises a lubricant. In certain embodiments, the total lubricant mass is at least 0.1%, 0.5%, 1%, 2%, 3%, 4%, or 5% of the total mass of the pharmaceutical composition. In certain embodiments, the total lubricant mass is no more than 0.1%, 0.5%, 1%, 2%, 3%, 4%, or 5% of the total mass of the pharmaceutical composition. In certain embodiments, the total lubricant mass is about 0.1%, 0.5%, 1%, 2%, 3%, 4%, or 5% of the total mass of the pharmaceutical composition. In certain embodiments, the total lubricant mass is about 0.5% to about 1.5% of the total mass of the pharmaceutical composition. In certain embodiments, the total lubricant mass is about 1% of the total mass of the pharmaceutical composition. In some embodiments, the lubricant comprises magnesium stearate.

In certain embodiments, the solid dosage forms provided herein comprise a glidant. In some embodiments, the glidant is colloidal silicon dioxide. In certain embodiments, the total glidant mass is at least 0.01%, 0.1%, 0.2%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is no more than 0.01%, 0.1%, 0.2%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is about 0.01%, 0.1%, 0.2%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is about 0.25% to about 0.75% of the total mass of the pharmaceutical composition. In certain embodiments, the total glidant mass is about 0.5% to about 1.5% of the total mass of the pharmaceutical composition. In certain embodiments, the total glidant mass is about 0.5% of the total mass of the pharmaceutical composition. In certain embodiments, the total glidant mass is about 1% of the total mass of the pharmaceutical composition.

In some embodiments, the diluent is selected from the group consisting of lactose, sucrose, dextrose, dextrates, maltodextrin, mannitol, xylitol, sorbitol, cyclodextrins, calcium phosphate, calcium sulfate, starches, modified starches, microcrystalline cellulose, microcellulose, and talc. In some embodiments the diluent is microcrystalline cellulose. In some embodiments, the disintegrating agent is selected from the group consisting of natural starch, a pregelatinized starch, a sodium starch, methylcrystalline cellulose, methylcellulose, croscarmellose, croscarmellose sodium, cross-linked sodium carboxymethylcellulose, crosslinked carboxymethylcellulose, cross-linked croscarmellose, cross-linked starch such as sodium starch glycolate, cross-linked polymer such as crospovidone, cross-linked polyvinylpyrrolidone, sodium alginate, a clay, or a gum. In some embodiments, the disintegrating agent is croscarmellose sodium. In some embodiments, the surfactant is selected from the group consisting of sodium lauryl sulfate, sorbitan monooleate, polyoxyethylene sorbitan monooleate, polysorbates, polaxomers, bile salts, glyceryl monostearate, copolymers of ethylene oxide and propylene oxide. In some embodiments, the surfactant is sodium lauryl sulfate. In some embodiments, the lubricant is selected from the group consisting of stearic acid, calcium hydroxide, talc, corn starch, sodium stearyl fumerate, stearic acid, sodium stearates, magnesium stearate, zinc stearate, and waxes. In some embodiments, the lubricant is magnesium stearate.

In certain embodiments, the solid dosage forms provided herein comprise: (i) a pharmaceutical agent having a total pharmaceutical agent mass that is at least 4% and no more than 65% of the total mass of the pharmaceutical composition; (ii) a diluent (e.g., mannitol) having a total mass that is at least 35% and no more than 95% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is at least 5% and no more than 60% of the total mass of the pharmaceutical composition; (ii) a diluent (e.g., mannitol) having a total mass that is at least 38% and no more than 93% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is at least 20% and no more than 55% of the total mass of the pharmaceutical composition; (ii) a diluent (e.g., mannitol) having a total mass that is at least 45% and no more than 80% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is about 20% to about 50% of the total mass of the pharmaceutical composition; (ii) a diluent (e.g., mannitol) having a total mass that is about 50% to 80% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition. In certain embodiments, the solid dosage forms provided herein comprise: (i) a pharmaceutical agent having a total pharmaceutical agent mass that is at least 30% and no more than 50% of the total mass of the pharmaceutical composition; (ii) a diluent (e.g., mannitol) having a total mass that is at least 45% and no more than 70% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is about 50% of the total mass of the pharmaceutical composition; (ii) a diluent (e.g., mannitol) having a total mass that is about 48.5% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is at least 8% and no more than 92% of the total mass of the pharmaceutical composition; (ii) a diluent (e.g., mannitol) having a total mass that is at least 5% and no more than 90% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is about 10% to about 90% of the total mass of the pharmaceutical composition; (ii) a diluent (e.g., mannitol) having a total mass that is about 7% to about 88% of the total mass of the pharmaceutical composition; (iii) a lubricant (e.g., magnesium stearate) having a total mass that is about 1.5% of the total mass of the pharmaceutical composition; and (iv) a glidant (e.g., colloidal silicon dioxide) having a total mass that is about 1% of the total mass of the pharmaceutical composition.

In certain embodiments, the solid dosage forms provided herein comprise: (i) a pharmaceutical agent having a total pharmaceutical agent mass that is at least 10% and no more than 90% of the total mass of the pharmaceutical composition; (ii) a diluent (e.g., mannitol) having a total mass that is at least 8.5% and no more than 88.5% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is about 13.51% of the total mass of the pharmaceutical composition; (ii) a diluent (e.g., mannitol) having a total mass that is about 84.99% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 90.22% of the total mass of the pharmaceutical composition; (ii) a diluent (e.g., mannitol) having a total mass that is about 8.28% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is at least 5% and no more than 50% of the total mass of the pharmaceutical composition; (ii) a diluent (e.g., mannitol) having a total mass that is at least 50% and no more than 95% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is at least 8% and no more than 45% of the total mass of the pharmaceutical composition; (ii) a diluent (e.g., mannitol) having a total mass that is at least 55% and no more than 90% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is about 40% of the total mass of the pharmaceutical composition; (ii) a diluent (e.g., mannitol) having a total mass that is about 58% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 10.6% of the total mass of the pharmaceutical composition; (ii) a diluent (e.g., mannitol) having a total mass that is about 87.4% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 13.51% of the total mass of the pharmaceutical composition; (ii) a diluent (e.g., mannitol) having a total mass that is about 84.99% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 90.22% of the total mass of the pharmaceutical composition; (ii) a diluent (e.g., mannitol) having a total mass that is about 8.28% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 50% of the total mass of the pharmaceutical composition; (ii) a diluent (e.g., mannitol) having a total mass that is about 48.5% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 5% of the total mass of the pharmaceutical composition; (ii) a diluent (e.g., mannitol) having a total mass that is about 93% of the total mass of the pharmaceutical composition; (iii) a lubricant (e.g., magnesium stearate) having a total mass that is about 1.5% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 60% of the total mass of the pharmaceutical composition; (ii) a diluent (e.g., mannitol) having a total mass that is about 38% of the total mass of the pharmaceutical composition; (iii) a lubricant (e.g., magnesium stearate) having a total mass that is about 1.5% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 30% to about 50% of the total mass of the pharmaceutical composition; (ii) a diluent (e.g., In certain embodiments, the solid dosage forms provided herein comprise: (i) a pharmaceutical agent having a total pharmaceutical agent mass that is about 10% to about 90% of the total mass of the pharmaceutical composition; (ii) a diluent (e.g., mannitol) having a total mass that is about 45% to 70% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the solid dosage forms provided herein comprise: (i) a pharmaceutical agent having a total pharmaceutical agent mass that is about 2.5% to about 70% of the total mass of the pharmaceutical composition; (ii) a diluent (e.g., mannitol) having a total mass that is about 30% to 98% of the total mass of the pharmaceutical composition; (iii) a lubricant (e.g., magnesium stearate) having a total mass that is at least 0.5% and no more than 2.5% of the total mass of the pharmaceutical composition; and (iv) a glidant (e.g., colloidal silicon dioxide) having a total mass that is at least 0.1% and no more than 1% of the total mass of the pharmaceutical composition.

Thus, in certain embodiments, provided herein are solid dosage forms comprising a pharmaceutical agent that contains bacteria. The bacteria can be live bacteria (e.g., powder or biomass thereof); non-live (dead) bacteria (e.g., powder or biomass thereof); non replicating bacteria (e.g., powder or biomass thereof); gamma irradiated bacteria (e.g., powder or biomass thereof); and/or lyophilized bacteria (e.g., powder or biomass thereof).

In certain embodiments, provided herein are solid dosage forms comprising a pharmaceutical agent that contains mEVs. The mEVs can be from culture media (e.g., culture supernatant). The mEVs can be from live bacteria (e.g., powder or biomass thereof); the mEVs can be from non-live (dead) bacteria (e.g., powder or biomass thereof); the mEVs can be from non-replicating bacteria (e.g., powder or biomass thereof); the mEVs can be from gamma irradiated bacteria (e.g., powder or biomass thereof); and/or the mEVs can be from lyophilized bacteria (e.g., powder or biomass thereof).

In some embodiments, the pharmaceutical agent comprises mEVs substantially or entirely free of bacteria (e.g., whole bacteria), bacteria (e.g., live bacteria, dead (e.g., killed), non-replicating bacteria, attenuated bacteria. In some embodiments, the pharmaceutical compositions comprise both mEVs and bacteria (e.g., whole bacteria) (e.g., live bacteria, killed bacteria, attenuated bacteria). In some embodiments, the pharmaceutical agents comprise bacteria and/or mEVs from one or more (e.g., 1, 2, 3, 4, 5, 6, 7, 8, 9, 10, or more) of the bacteria strains or species or taxonomic groups listed herein. In some embodiments, the pharmaceutical agents comprise bacteria and/or mEVs from one of the bacteria strains or species or taxonomic groups listed herein. In some embodiments, the pharmaceutical agents comprise bacteria and/or mEVs from one of the bacteria strains or species described herein, e.g., Lactococcus, Prevotella, Bifrdobacterium, Veillonella, Fournierella, Harryflintia, Megasphaera; e.g., Lactococcus lactis cremoris; Prevotella histicola; Bifidobacterium animalis lactis; Veillonella parvula; Fournierella massiliensis; Harryflintia acetispora; or Megasphaera sp.

In some embodiments, the pharmaceutical agents comprise lyophilized Prevotella histicola bacteria.

In some embodiments, the pharmaceutical agents comprise lyophilized bacteria and/or mEVs. In some embodiments, the pharmaceutical agent comprises gamma irradiated bacteria and/or mEVs. The mEVs (such as smEVs and/or pmEVs) can be gamma irradiated after the mEVs are isolated (e.g., prepared).

In some embodiments, to quantify the numbers of mEVs (such as smEVs and/or pmEVs) and/or bacteria present in a sample, electron microscopy (e.g., EM of ultrathin frozen sections) can be used to visualize the mEVs (such as smEVs and/or pmEVs) and/or bacteria and count their relative numbers. Alternatively, nanoparticle tracking analysis (NTA), Coulter counting, or dynamic light scattering (DLS) or a combination of these techniques can be used. NTA and the Coulter counter count particles and show their sizes. DLS gives the size distribution of particles, but not the concentration. Bacteria frequently have diameters of 1-2 um (microns). The full range is 0.2-20 um. Combined results from Coulter counting and NTA can reveal the numbers of bacteria and/or mEVs (such as smEVs and/or pmEVs) in a given sample. Coulter counting reveals the numbers of particles with diameters of 0.7-10 um. For most bacterial and/or mEV (such as smEV and/or pmEV) samples, the Coulter counter alone can reveal the number of bacteria and/or mEVs (such as smEVs and/or pmEVs) in a sample, pmEVs are 20-600 nm in diameter. For NTA, a Nanosight instrument can be obtained from Malvern Pananlytical. For example, the NS300 can visualize and measure particles in suspension in the size range 10-2000 nm. NTA allows for counting of the numbers of particles that are, for example, 50-1000 nm in diameter. DLS reveals the distribution of particles of different diameters within an approximate range of 1 nm-3 um.

mEVs can be characterized by analytical methods known in the art (e.g., Jeppesen, et al. Cell 177:428 (2019)).

In some embodiments, the bacteria and/or mEVs may be quantified based on particle count. For example, total particle count of a bacteria and/or mEV preparation can be measured using NTA.

In some embodiments, the bacteria and/or mEVs may be quantified based on the amount of protein, lipid, or carbohydrate. For example, total protein content of a bacteria and/or preparation can be measured using the Bradford assay or BCA.

In some embodiments, mEVs are isolated away from one or more other bacterial components of the source bacteria or bacterial culture. In some embodiments, bacteria are isolated away from one or more other bacterial components of the source bacterial culture. In some embodiments, the pharmaceutical agent further comprises other bacterial components.

In certain embodiments, the mEV preparation obtained from the source bacteria may be fractionated into subpopulations based on the physical properties (e.g., sized, density, protein content, binding affinity) of the subpopulations. One or more of the mEV subpopulations can then be incorporated into the pharmaceutical agents of the invention.

In certain aspects, provided herein are solid dosage forms comprising pharmaceutical agents that comprise bacteria and/or mEVs (such as smEVs and/or pmEVs) useful for the treatment and/or prevention of disease (e.g., a cancer, an autoimmune disease, an inflammatory disease, a metabolic disease, or a dysbiosis); or treatment and/or prevention of bacterial septic shock, cytokine storm and/or viral infection (such as a coronavirus infection, an influenza infection, and/or a respiratory syncytial virus infection); or to decrease inflammatory cytokine expression (e.g., decreased IL-8, IL-6, IL-1β, and/or TNFα expression levels), as well as methods of making and/or identifying such bacteria and/or mEVs, and methods of using pharmaceutical agents and solid dosage forms thereof (e.g., for the treatment of a cancer, an autoimmune disease, an inflammatory disease, or a metabolic disease dysbiosis, bacterial septic shock, cytokine storm and/or viral infection, or to decrease inflammatory cytokine expression), either alone or in combination with other therapeutics. In some embodiments, the pharmaceutical agents comprise both mEVs (such as smEVs and/or pmEVs) and bacteria (e.g., whole bacteria) (e.g., live bacteria, dead (e.g., killed) bacteria, non-replicating bacteria, attenuated bacteria). In some embodiments, the pharmaceutical agents comprise bacteria in the absence of mEVs (such as smEVs and/or pmEVs). In some embodiments, the pharmaceutical agents comprise mEVs (such as smEVs and/or pmEVs) in the absence of bacteria. In some embodiments, the pharmaceutical agents comprise mEVs (such as smEVs and/or pmEVs) and/or bacteria from one or more of the bacteria strains or species listed in Table 1, Table 2, and/or Table 3. In some embodiments, the pharmaceutical compositions comprise mEVs (such as smEVs and/or pmEVs) and/or bacteria from one of the bacteria strains or species or taxonomic groups listed herein. In some embodiments, the pharmaceutical agents comprise bacteria and/or mEVs from one of the bacteria strains or species described herein, e.g., Lactococcus, Prevotella, Bifidobacterium, Veillonella, Fournierella, Harryflintia, Megasphaera; e.g., Lactococcus lactis cremoris; Prevotella histicola; Bifidobacterium animalis lactis; Veillonella parvula; Fournierella massiliensis; Harryflintia acetispora; or Megasphaera sp.

In certain aspects, provided are pharmaceutical agents for administration to a subject (e.g., human subject). In some embodiments, the pharmaceutical agents are combined with additional active and/or inactive materials in order to produce a final product, which may be in single dosage unit or in a multi-dose format. In some embodiments, the pharmaceutical agent is combined with an adjuvant such as an immuno-adjuvant (e.g., a STING agonist, a TLR agonist, or a NOD agonist).

In some embodiments, the solid dosage form comprises at least one carbohydrate.

In some embodiments, the solid dosage form comprises at least one lipid. In some embodiments, the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0).

In some embodiments, the solid dosage form comprises at least one mineral or mineral source. Examples of minerals include, without limitation; chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, the solid dosage form comprises at least one vitamin. The at least one vitamin can be fat-soluble or water-soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, the solid dosage form comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

Suitable excipients that can be included in the solid dosage form can be one or more pharmaceutically acceptable excipients known in the art. For example, see Rowe, Sheskey, and Quinn, eds., Handbook of Pharmaceutical Excipients, sixth ed.: 2009; Pharmaceutical Press and American Pharmacists Association.

### Solid Dosage Forms

The solid dosage form described herein can be a capsule.

The solid dosage forms of a pharmaceutical agent as described herein can comprise capsules. In some embodiments, the capsule is a size 00, size 0, size 1, size 2, size 3, size 4, or size 5 capsule. In some embodiments, the capsule comprises HPMC (hydroxyl propyl methyl cellulose) or gelatin. In some embodiments, the capsule comprises HPMC (hydroxyl propyl methyl cellulose). In some embodiments, the capsule is banded.

In some embodiments, the solid dosage form is enterically coated (e.g., comprises an enteric coating; e.g., is coated with an enteric coating).

### Coating

The solid dosage form (e.g., capsule) described herein can be enterically coated. The enteric coating allows for release of the pharmaceutical agent, e.g., in the small intestine, e.g., upper small intestine, e.g., duodenum and/or jejunum. In some embodiments, the solid dosage form is enteric coated to dissolve at pH 5.5.

Release of the pharmaceutical agent in the small intestine, e.g., in the upper small intestine, e.g., in the duodenum, or in the jejunum, allows the pharmaceutical agent to target and affect cells (e.g., epithelial cells and/or immune cells) located at these specific locations, e.g., which can cause a local effect in the small intestine and/or cause a systemic effect (e.g., an effect outside of the gastrointestinal tract).

EUDRAGIT is the brand name for a diverse range of polymethacrylate-based copolymers. It includes anionic, cationic, and neutral copolymers based on methacrylic acid and methacrylic/acrylic esters or their derivatives.

Examples of other materials that can be used in the enteric coating (e.g., the one enteric coating or the inner enteric coating and/or the outer enteric coating) include cellulose acetate phthalate (CAP), cellulose acetate trimellitate (CAT), poly(vinyl acetate phthalate) (PVAP), hydroxypropyl methylcellulose phthalate (HPMCP), fatty acids, waxes, shellac (esters of aleurtic acid), plastics, plant fibers, zein, AQUA-ZEIN® (an aqueous zein formulation containing no alcohol), amylose starch, starch derivatives, dextrins, methyl acrylate-methacrylic acid copolymers, cellulose acetate succinate, hydroxypropyl methyl cellulose acetate succinate (hypromellose acetate succinate), methyl methacrylate-methacrylic acid copolymers, and/or sodium alginate.

The enteric coating can include a polymethacrylate-based copolymer.

The enteric coating can include poly(methacrylic acid-co-ethyl acrylate).

The enteric coating can include a methacrylic acid ethyl acrylate (MAE) copolymer (1:1).

The enteric coating can include methacrylic acid ethyl acrylate (MAE) copolymer (1:1) (such as Kollicoat MAE 100P).

The enteric coating can include a Eudragit copolymer. e.g., a Eudragit L (e.g., Eudragit L 100-55; Eudragit L 30 D-55), a Eudragit S, a Eudragit RL, a Eudragit RS, a Eudragit E, or a Eudragit FS (e.g., Eudragit FS 30 D).

Other examples of materials that can be used in the enteric coating (e.g., the one enteric coating or the inner enteric coating and/or the outer enteric coating) include those described in, e.g., U.S. Pat. Nos. 6,312,728, 6,623,759; 4,775,536; 5,047,258; 5,292,522; 6,555,124; 6,638,534; U.S. 2006/0210631: U.S. 2008/200482; U.S. 2005/0271778; U.S. 2004/0028737; WO 2005/044240.

See also, e.g., U.S. Pat. No. 9,233,074, which provides pH dependent, enteric polymers that can be used with the solid dosage forms provided herein, including methacrylic acid copolymers, polyvinylacetate phthalate, hydroxypropylmethyl cellulose acetate succinate, hydroxypropylmethyl cellulose phthalate and cellulose acetate phthalate; suitable methacrylic acid copolymers include; poly(methacrylic acid, methyl methacrylate) 1:1 sold, for example, under the Eudragit L100 trade name; poly(methacrylic acid, ethyl acrylate) 1:1 sold, for example, under the Eudragit L100-55 trade name; partially-neutralized poly(methacrylic acid, ethyl acrylate) 1:1 sold, for example, under the Kollicoat MAE-100P trade name; and poly(methacrylic acid, methyl methacrylate) 1:2 sold, for example, under the Eudragit S100 trade name.

### Dose

The dose of the pharmaceutical agent (e.g., for human subjects) is the dose per capsule.

In embodiments where dose is determined by total cell count (TCC), total cell count can be determined by Coulter counter.

In some embodiments, the pharmaceutical agent comprises bacteria and the dose of bacteria is about 1×10⁷ to about 2×10¹² (e.g., about 3×10¹⁰ or about 1.5×10¹¹ or about 1.5×10¹²) cells (e.g., wherein cell number is determined by total cell count, which is determined by Coulter counter), wherein the dose is per capsule. In some embodiments, the pharmaceutical agent comprises bacteria and the dose of bacteria is about 1×10¹⁰ to about 2×10¹² (e.g., about 1.6×10¹¹ or about 8×10¹¹ or about 9.6×10¹¹ about 12.8×10¹¹ or about 1.6×10¹¹) cells (e.g., wherein cell number is determined by total cell count, which is determined by Coulter counter), wherein the dose is per capsule.

In some embodiments, the pharmaceutical agent comprises bacteria and the dose of bacteria is about 1×10⁹, about 3×10⁹, about 5×10⁹ about 1.5×10¹⁰, about 3×10¹⁰, about 5×10¹⁰, about 1.5×10¹¹, about 1.5×10¹², or about 2×10¹² cells, wherein the dose is per capsule.

In some embodiments, the pharmaceutical agent comprises mEVs and the dose of mEVs is about 1×10⁵, to about 7×10¹³ particles (e.g., wherein particle count is determined by NTA (nanoparticle tracking analysis)), wherein the dose is per capsule. In some embodiments, the pharmaceutical agent comprises mEVs and the dose of mEVs is about 1×10¹⁰ to about 7×10¹³ particles (e.g., wherein particle count is determined by NTA (nanoparticle tracking analysis)), wherein the dose is per capsule.

In some embodiments, wherein the pharmaceutical agent comprises mEVs, the dose of mEVs is about 2×106 to about 2×1016 particles (e.g., wherein particle count is determined by NTA (nanoparticle tracking analysis)), wherein the dose is per capsule.

In some embodiments wherein the pharmaceutical agent comprises Prevotella histicola bacteria, the dose is total cell count of about 1×10⁷ to about 1×10¹² cells (e.g., wherein cell number is determined by total cell count, which is determined by Coulter counter) per capsule.

In some embodiments, wherein the pharmaceutical agent comprises Prevotella histicola bacteria, the dose is about 3×10¹⁰ or about 1.5×10¹¹ cells (e.g., wherein cell number is determined by total cell count, which is determined by Coulter counter) per capsule. In some embodiments, wherein the pharmaceutical agent comprises Prevotella histicola bacteria, the dose is about 8×10¹⁰ or about 1.6×10¹¹ cells (e.g., wherein cell number is determined by total cell count, which is determined by Coulter counter) per capsule.

In some aspects, the disclosure provides a method of treating a subject (e.g., human) (e.g., a subject in need of treatment), the method comprising administering to the subject a solid dosage form provided herein.

In some aspects, the disclosure provides use of a solid dosage form provided herein for the preparation of a medicament for treating a subject (e.g., human) (e.g., a subject in need of treatment).

In some embodiments, the solid dosage form is orally administered (e.g., is for oral administration).

In some embodiments, the solid dosage form is administered (e.g., is for administration) 1, 2, 3, or 4 times a day. In some embodiments, 1, 2, 3, 4 or 5 solid dosage forms (e.g., capsules) are administered (e.g., are for administration) 1, 2, 3, or 4 times a day. In some embodiments, 2, 4, 6, 8, or 10 solid dosage forms (e.g., capsules) are administered (e.g., are for administration) 1, 2, 3, or 4 times a day. In some embodiments, 1 solid dosage form (e.g., capsule) is administered (e.g., is for administration) 1 or 2 times a day. In some embodiments, 2 solid dosage forms (e.g., capsules) are administered (e.g., are for administration) 1 or 2 times a day. In some embodiments, 3 solid dosage forms (e.g., capsules) are administered (e.g., are for administration) 1 or 2 times a day. In some embodiments, 4 solid dosage forms (e.g., capsules) are administered (e.g., are for administration) 1 or 2 times a day. In some embodiments, 5 solid dosage forms (e.g., capsules) are administered (e.g., are for administration) 1 or 2 times a day.

In some embodiments, 1 solid dosage form (e.g., capsule) is administered (e.g., is for administration) 1 or 2 times a day, wherein the solid dosage form comprises a dose of bacteria of about 3.2×10¹¹ cells. In some embodiments. 2 solid dosage forms (e.g., capsules) are administered (e.g., are for administration) 1 or 2 times a day, wherein the solid dosage form comprises a dose of bacteria of about 3.2×10¹¹ cells. In some embodiments, 3 solid dosage forms (e.g., capsules) are administered (e.g., are for administration) 1 or 2 times a day, wherein the solid dosage form comprises a dose of bacteria of about 3.2×10¹¹ cells. In some embodiments, 4 solid dosage forms (e.g., capsules) are administered (e.g., are for administration) 1 or 2 times a day, wherein the solid dosage form comprises a dose of bacteria of about 3.2×10¹¹ cells. In some embodiments, 5 solid dosage forms (e.g., capsules) are administered (e.g., are for administration) 1 or 2 times a day, wherein the solid dosage form comprises a dose of bacteria of about 3.2×10¹¹ cells.

In some embodiments, 1 solid dosage form (e.g., capsule) is administered (e.g., is for administration) per day, wherein the solid dosage form comprises a dose of bacteria of about 3.2×10¹¹ cells (e.g., resulting in a total of about 3.2×10¹¹ cells being administered). In some embodiments, 2 solid dosage forms (e.g., capsules) are administered (e.g., are for administration) per day, wherein the solid dosage form comprises a dose of bacteria of about 3.2×10¹¹ cells (e.g., resulting in a total of about 6.4×10¹¹ cells being administered with the 2 tablets). In some embodiments, 3 solid dosage forms (e.g., capsules) are administered (e.g., are for administration) per day, wherein the solid dosage form comprises a dose of bacteria of about 3.2×10¹¹ cells (e.g., resulting in a total of about 9.6×10¹¹ cells being administered with the 3 tablets). In some embodiments, 4 solid dosage forms (e.g., capsules) are administered (e.g., are for administration) per a day, wherein the solid dosage form comprises a dose of bacteria of about 3.2×10¹¹ cells (e.g., resulting in a total of about 12.8×10¹¹ cells being administered with the 4 tablets). In some embodiments, 5 solid dosage forms (e.g., capsules) are administered (e.g., are for administration) per a day, wherein the solid dosage form comprises a dose of bacteria of about 3.2×10¹¹ cells (e.g., resulting in a total of about 16×10¹¹ cells being administered with the 5 capsules).

In some embodiments, the pharmaceutical agent dose can be a milligram (mg) dose determined by weight the pharmaceutical agent (e.g., a powder comprising bacteria and/or an agent of bacterial origin, such as mEVs). The dose of the pharmaceutical agent is per capsule.

For example, to administer a 1×dose of the pharmaceutical agent of about 400 mg, about 200 mg of the pharmaceutical agent is present per capsule and two capsules are administered, resulting in a dose of about 400 mg. The two capsules can be administered, for example, 1× or 2× daily.

In some embodiments, the dose can be about 3 mg to about 125 mg of the pharmaceutical agent, per capsule.

In some embodiments, the dose can be about 35 mg to about 1200 mg (e.g., about 35 mg, about 125 mg, about 350 mg, or about 1200 mg) of the pharmaceutical agent.

In some embodiments, the pharmaceutical agent comprises a powder comprising bacteria and/or mEVs and the dose of the pharmaceutical agent (e.g., a powder comprising bacteria and/or mEVs) is about 10 mg to about 1500 mg, wherein the dose is per capsule.

In some embodiments, the dose of the pharmaceutical agent can be about 30 mg to about 3500 mg (about 25, about 50, about 75, about 100, about 150, about 250, about 300, about 350, about 400, about 500, about 600, about 750, about 1000, about 1250, about 1300, about 2000, about 2500, about 3000, or about 3500 mg).

A human dose can be calculated appropriately based on allometric scaling of a dose administered to a model organism (e.g., mouse).

In some embodiments, one or two capsules can be administered one or two times a day.

In some embodiments, one or two capsules can be administered daily.

In some embodiments, 3, 4, or 5 capsules can be administered one or two times a day.

In some embodiments, 3, 4, or 5 capsules can be administered daily.

In some embodiments, 4 capsules can be administered one or two times a day.

In some embodiments, 4 capsules can be administered daily.

The pharmaceutical agent contains the bacteria and/or an agent of bacterial origin, such as mEVs, or contains a powder comprising bacteria and/or an agent of bacterial origin, such as mEVs, and can also contain one or more additional components, such as a cryoprotectant, etc.

In some embodiments, the mg (by weight) dose of the pharmaceutical agent is, e.g., about 1 mg to about 500 mg per capsule, or per tablet, or per total number of minitablets, e.g., used in a capsule.

The dose of the pharmaceutical agent (e.g., for human subjects) is the dose per capsule.

In embodiments where dose is determined by total cell count (TCC), total cell count can be determined by Coulter counter.

In some embodiments, the pharmaceutical agent comprises isolated Veillonella parvula bacteria (e.g., from one or more strains of bacteria (e.g., bacteria of interest) (e.g., a therapeutically effective amount thereof)). E.g., wherein at least 50%, at least 75%, at least 80%, at least 85%, at least 90%, at least 95%, or at least 99% of the content of the pharmaceutical agent is the isolated Veillonella parvula bacteria (e.g., bacteria of interest).

In some embodiments, the pharmaceutical agent comprises isolated Veillonella parvula bacteria (e.g., from one or more strains of bacteria (e.g., bacteria of interest) (e.g., a therapeutically effective amount thereof)). E.g., wherein at least 50%, at least 75%, at least 80%, at least 85%, at least 90%, at least 95%, or at least 99% of the content of the pharmaceutical agent is the isolated Veillonella parvula bacteria (e.g., bacteria of interest).

In some embodiments, the Veillonella parvula bacteria are from a strain comprising at least 90% (or at least 97%) genomic, 16S and/or CRISPR sequence identity to the nucleotide sequence of the Veillonella parvula Strain A (ATCC Deposit Number PTA-125691). In some embodiments, the Veillonella parvula bacteria are from a strain comprising at least 99% genomic, 16S and/or CRISPR sequence identity to the nucleotide sequence of the Veillonella parvula Strain A (ATCC Deposit Number PTA-125691). In some embodiments, the Prevotella bacteria are from Veillonella parvula Strain A (ATCC Deposit Number PTA-125691).

In some embodiments, at least 50%, 60%, 70%, 80%, or 90% of the bacteria in the pharmaceutical composition are Veillonella parvila Strain A.

In some embodiments, the pharmaceutical agent comprises at least 1×10⁵, 5×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸ or 1×10⁹ colony forming units of a Veillonella bacteria described herein (e.g., Veillonella parvula bacteria strain A (ATCC Deposit Number PTA-125691)).

In some embodiments, the Prevotella histicola bacteria are from a strain comprising at least 90% (or at least 97%) genomic, 16S and/or CRISPR sequence identity to the nucleotide sequence of the Prevotella Strain B 50329 (NRRL accession number B 50329). In some embodiments, the Prevotella bacteria are from a strain comprising at least 99% genomic, 16S and/or CRISPR sequence identity to the nucleotide sequence of the Prevotella Strain B 50329 (NRRL accession number B 50329). In some embodiments, the Prevotella bacteria are from Prevotella Strain B 50329 (NRRL accession number B 50329).

In some embodiments, the bacteria of the pharmaceutical agent or from which the mEVs of the pharmaceutical agent are obtained are Prevotella bacteria, e.g., a strain comprising at least 90% or at least 99% genomic, 16S and/or CRISPR sequence identity to the nucleotide sequence of the Prevotella Strain C (ATCC Accession Number PTA-126140). In some embodiments, the bacteria of the pharmaceutical agent or from which the mEVs of the pharmaceutical agent are obtained are Prevotella bacteria, e.g., Prevotella Strain C (ATCC Accession Number PTA-126140).

In some embodiments, the pharmaceutical agent comprises Prevotella histicola bacteria and the dose of bacteria is about 1×10¹¹ to about 2×10¹¹ (e.g., about 3×10¹⁰ or about 1.5×10¹¹) cells (e.g., wherein cell number is determined by total cell count, which is determined by Coulter counter), wherein the dose is per capsule. In some embodiments, the pharmaceutical agent comprises about 1×10⁷ to about 2×10¹² cells of Prevotella histicola bacteria. In some embodiments, the pharmaceutical agent comprises about 1.6×10¹⁰ cells of Prevotella histicola bacteria. In some embodiments, the pharmaceutical agent comprises about 8.0×10¹¹ cells of Prevotella histicola bacteria. In some embodiments, the pharmaceutical agent comprises about 1.6×10¹¹ cells of Prevotella histicola bacteria.

In some embodiments, the pharmaceutical agent comprises Prevotella histicola bacteria and the dose of bacteria is about 1×10⁹, about 3×10⁹, about 5×10⁹, about 1.5×10¹⁰, or about 5×10¹⁰ cells (e.g., TCC (total cell count)), wherein the dose is per capsule. In some embodiments, the pharmaceutical agent comprises bacteria and the dose of bacteria is about 8×10¹⁰ cells, wherein the dose is per capsule. In some embodiments, the pharmaceutical agent comprises bacteria and the dose of bacteria is about 1.6×10¹¹ cells, wherein the dose is per capsule.

In some embodiments, the pharmaceutical agent dose can be a milligram (mg) dose determined by weight the pharmaceutical agent (e.g., a powder comprising bacteria). The dose of the pharmaceutical agent is per capsule.

For example, to administer a 1×dose of the pharmaceutical agent of about 400 mg, about 200 mg of the pharmaceutical agent is present per capsule and two capsules are administered, resulting in a dose of about 400 mg. The two capsules can be administered, for example, 1× or 2× daily.

In some embodiments, the dose can be about 3 mg to about 125 mg of the pharmaceutical agent per capsule.

In some embodiments, the dose can be about 35 mg to about 1200 mg (e.g., about 35 mg, about 125 mg, about 350 mg, or about 1200 mg) of the pharmaceutical agent.

In some embodiments, the dose of the pharmaceutical agent can be about 30 mg to about 3500 mg (about 25, about 50, about 75, about 100, about 150, about 250, about 300, about 350, about 400, about 500, about 600, about 750, about 1000, about 1250, about 1300, about 2000, about 2500, about 3000, or about 3500 mg).

A human dose can be calculated appropriately based on allometric scaling of a dose administered to a model organism (e.g., mouse).

In some embodiments, one or two capsules can be administered one or two times a day.

In some embodiments, five or ten capsules can be administered daily.

The pharmaceutical agent contains the bacteria or contains a powder comprising bacteria, and can also contain one or more additional components, such as a cryoprotectant.

In some embodiments, the mg (by weight) dose of the pharmaceutical agent is, e.g., about 1 mg to about 500 mg per capsule.

### Methods of Use

The solid dosage forms described herein allow, e.g., for oral administration of a pharmaceutical agent contained therein.

The solid dosage forms described herein can be used in the treatment and/or prevention of a cancer, inflammation, autoimmunity, a metabolic condition, or a dysbiosis.

The solid dosage forms described herein can be used in the treatment and/or prevention of bacterial septic shock, cytokine storm and/or viral infection (such as a coronavirus infection, an influenza infection, and/or a respiratory syncytial virus infection).

The solid dosage forms described herein can be used to decrease inflammatory cytokine expression (e.g., decreased IL-8, IL-6, IL-1β, and/or TNFα expression levels).

Methods of using a solid dosage form (e.g., for oral administration) (e.g., for pharmaceutical use) comprising a pharmaceutical agent (e.g., a therapeutically effective amount thereof), wherein the pharmaceutical agent comprises bacteria and/or microbial extracellular vesicles (mEVs), and wherein the solid dosage form further comprises the disclosed components are described herein.

The methods and administered solid dosage forms described herein allow, e.g., for oral administration of a pharmaceutical agent contained therein. The solid dosage form can be administered to a subject is a fed or fasting state. The solid dosage form can be administered, e.g., on an empty stomach (e.g., one hour before eating or two hours after eating). The solid dosage form can be administered one hour before eating. The solid dosage form can be administered two hours after eating.

A solid dosage form for use in the treatment and/or prevention of a cancer, inflammation, autoimmunity, a metabolic condition, or a dysbiosis is provided herein.

A solid dosage form for use in the treatment and/or prevention of bacterial septic shock, cytokine storm and/or viral infection (such as a coronavirus infection, an influenza infection, and/or a respiratory syncytial virus infection) is provided herein.

A solid dosage form for use in decrease inflammatory cytokine expression (e.g., decreased IL-8, IL-6, IL-1β, and/or TNFα expression levels) is provided herein.

Use of a solid dosage form for the preparation of a medicament for the treatment and/or prevention of a cancer, inflammation, autoimmunity, a metabolic condition, or a dysbiosis is provided herein.

Use of a solid dosage form for the preparation of a medicament for the treatment and/or prevention of bacterial septic shock, cytokine storm and/or viral infection (such as a coronavirus infection, an influenza infection, and/or a respiratory syncytial virus infection) is provided herein.

Use of a solid dosage form for the preparation of a medicament for decreasing inflammatory cytokine expression (e.g., decreased IL-8, IL-6, IL-1, and/or TNFα expression levels) is provided herein.

### Method of Making Solid Dosage Forms

The methods of preparing a solid dosage form of a pharmaceutical composition can comprise blending, encapsulation, banding, and coating of capsules.

In certain aspects, provided herein are methods of preparing a solid dosage form of a pharmaceutical composition, the method comprising combining (e.g., blending) into a pharmaceutical composition a pharmaceutical agent (e.g., bacteria disclosed herein and/or an agent of bacterial origin, such as mEVs (e.g., mEVs disclosed herein) or a powder comprising bacteria disclosed herein and/or an agent (e.g., component)) of bacterial origin, such as mEVs disclosed herein) and one or more additional components described herein. In certain aspects, provided herein are methods of preparing a solid dosage form of a pharmaceutical composition, the method comprising combining into a pharmaceutical composition a pharmaceutical agent (e.g., bacteria disclosed herein or a powder comprising the bacteria) and a diluent. In certain embodiments, the total pharmaceutical agent mass is at least 2.5%, 5%, 10%, 15%, 20%, 25%, 30%, 35%, 40%, 45%, 50%, 55%, 60%, 65%, 70%, or 75%, 80%, 85%, 90%, or 95% of the total mass of the pharmaceutical composition. In some embodiments the total pharmaceutical agent mass is no more than 95%, 90%, 85%, 80%, 75%, 70%, 65%, 60%, 55%, 50%, 45%, 40%, 35%, 30%, 25%, 20%, 15%, 10%, 5%, or 2.5% of the total mass of the pharmaceutical composition. In some embodiments, the pharmaceutical agent has a total pharmaceutical agent mass that is at least 2.5% and no more than 95% of the total mass of the pharmaceutical composition.

In some embodiments, the total mass of the diluent is at least 1%, 5%, 10%, 15%, 20%, 25%, 30%, 35%, 40%, 45%, 50%, 55%, 60%, 65%, 70%, 75%, 80%, 85%, 90%, 95%, or 98% of the total mass of the pharmaceutical composition. In some embodiments, the total mass of the diluent is no more than 98%, 95%, 90%, 85%, 80%, 75%, 70%, 65%, 60%, 55%, 50%, 45%, 40%, 35%, 30%, 25%, 20%, 15%, 10%, 5%, or 1% of the total mass of the pharmaceutical composition. In some embodiments, the diluent has a total mass that is at least 1% and no more than 98% of the total mass of the pharmaceutical composition. In some embodiments, the diluent comprises mannitol.

In certain embodiments, the method further comprises combining a lubricant. In certain embodiments, the total lubricant mass is at least 0.1%, 0.5%, 1%, 2%, 3%, 4%, or 5% of the total mass of the pharmaceutical composition. In certain embodiments, the total lubricant mass is no more than 0.1%, 0.5/a, 1%, 2%, 3%, 4%, or 5% of the total mass of the pharmaceutical composition. In certain embodiments, the total lubricant mass is about 0.1%, 0.5%, 1%, 2%, 3%, 4%, or 5% of the total mass of the pharmaceutical composition. In certain embodiments, the total lubricant mass is about 0.5% to about 1.5% of the total mass of the pharmaceutical composition. In certain embodiments, the total lubricant mass is about 1% of the total mass of the pharmaceutical composition. In some embodiments, the lubricant comprises magnesium stearate.

In certain embodiments, the method further comprises combining a glidant. In some embodiments, the glidant is colloidal silicon dioxide. In certain embodiments, the total glidant mass is at least 0.01%, 0.1%, 0.2%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is no more than 0.01%, 0.1%, 0.2%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is about 0.01%, 0.1%, 0.2%, 0.3%, 0.4%, 0.5%, 0.6%, 0.7%, 0.8%, 0.9%, 1%, 1.5%, or 2% of the total mass of the pharmaceutical composition. In certain embodiments, the total glidant mass is about 0.25% to about 0.75% of the total mass of the pharmaceutical composition. In certain embodiments, the total glidant mass is about 0.5% to about 1.5% of the total mass of the pharmaceutical composition. In certain embodiments, the total glidant mass is about 0.5% of the total mass of the pharmaceutical composition. In certain embodiments, the total glidant mass is about 1% of the total mass of the pharmaceutical composition.

In certain embodiments, the method provided herein comprises combining: (i) a pharmaceutical agent having a total pharmaceutical agent mass that is at least 4% and no more than 65% of the total mass of the pharmaceutical composition; (ii) a diluent (e.g., mannitol) having a total mass that is at least 35% and no more than 95% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is at least 5% and no more than 60% of the total mass of the pharmaceutical composition; (ii) a diluent (e.g., mannitol) having a total mass that is at least 38% and no more than 93% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is at least 20% and no more than 55% of the total mass of the pharmaceutical composition; (ii) a diluent (e.g., mannitol) having a total mass that is at least 45% and no more than 80% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is at least 8% and no more than 92% of the total mass of the pharmaceutical composition; (ii) a diluent (e.g., mannitol) having a total mass that is at least 5% and no more than 90% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the solid dosage forms provided herein comprise: (i) a pharmaceutical agent having a total pharmaceutical agent mass that is about 10% to about 90% of the total mass of the pharmaceutical composition; (ii) a diluent (e.g., mannitol) having a total mass that is about 7% to about 88% of the total mass of the pharmaceutical composition; (iii) a lubricant (e.g., magnesium stearate) having a total mass that is about 1.5% of the total mass of the pharmaceutical composition; and (iv) a glidant (e.g., colloidal silicon dioxide) having a total mass that is about 1% of the total mass of the pharmaceutical composition.

In certain embodiments, the method provided herein comprises combining: (i) a pharmaceutical agent having a total pharmaceutical agent mass that is about 20% to about 50% of the total mass of the pharmaceutical composition; (ii) a diluent (e.g., mannitol) having a total mass that is about 50% to 80% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is at least 30% and no more than 50% of the total mass of the pharmaceutical composition; (ii) a diluent (e.g., mannitol) having a total mass that is at least 45% and no more than 70% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is about 50% of the total mass of the pharmaceutical composition; (ii) a diluent (e.g., mannitol) having a total mass that is about 48.5% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the method provided herein comprises combining: (i) a pharmaceutical agent having a total pharmaceutical agent mass that is at least 8% and no more than 92% of the total mass of the pharmaceutical composition; (ii) a diluent (e.g., mannitol) having a total mass that is at least 5% and no more than 90% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is at least 10% and no more than 90% of the total mass of the pharmaceutical composition; (ii) a diluent (e.g., mannitol) having a total mass that is at least 8.5% and no more than 88.5% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is about 13.51% of the total mass of the pharmaceutical composition; (ii) a diluent (e.g., mannitol) having a total mass that is about 84.99% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the method provided herein comprises combining: (i) a pharmaceutical agent having a total pharmaceutical agent mass that is about 90.22% of the total mass of the pharmaceutical composition; (ii) a diluent (e.g., mannitol) having a total mass that is about 8.28% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the method provided herein comprises combining: (i) a pharmaceutical agent having a total pharmaceutical agent mass that is at least 5% and no more than 50% of the total mass of the pharmaceutical composition; (ii) a diluent (e.g., mannitol) having a total mass that is at least 50% and no more than 95% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is at least 8% and no more than 45% of the total mass of the pharmaceutical composition; (ii) a diluent (e.g., mannitol) having a total mass that is at least 55% and no more than 90% of the total mass of the pharmaceutical composition; (iii) a lubricant (e.g., magnesium stearate) having a total mass that is at least 0.1% and no more than 5% of the total mass of the pharmaceutical composition; and (iv) a glidant (e.g., colloidal silicon dioxide) having a total mass that is at least 0.01% and no more than 2% of the total mass of the pharmaceutical composition.

In certain embodiments, the method provided herein comprises combining: (i) a pharmaceutical agent having a total pharmaceutical agent mass that is about 40% of the total mass of the pharmaceutical composition; (ii) a diluent (e.g., mannitol) having a total mass that is about 58% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In certain embodiments, the method provided herein comprises combining: (i) a pharmaceutical agent having a total pharmaceutical agent mass that is about 10.6% of the total mass of the pharmaceutical composition; (ii) a diluent (e.g., mannitol) having a total mass that is about 87.4% of the total mass of the pharmaceutical composition; (iii) a lubricant (e.g., magnesium stearate) having a total mass that is about 1% of the total mass of the pharmaceutical composition; and (iv) a glidant (e.g., colloidal silicon dioxide) having a total mass that is about 0.5% of the total mass of the pharmaceutical composition.

In some embodiments, the method further comprises loading the pharmaceutical composition into a capsule (e.g., encapsulation).

In some embodiments, the method further comprises banding the capsule after loading.

In some embodiments, the method further comprises enterically coating the capsule.

In some embodiments, the method further comprises loading the pharmaceutical composition into a capsule. In some embodiments, the capsule comprises HPMC.

In some embodiments, the method further comprises banding the capsule. In some embodiments, the capsule is banded with an HPMC-based banding solution.

In some embodiments, the method further comprises enterically coating the capsule, thereby preparing an enterically coated capsule.

As used herein, the percent of mass of a solid dosage form is on a percent weight:weight basis (% w:w).

In certain embodiments, the method comprises performing wet granulation on a pharmaceutical agent prior to combining the pharmaceutical agent (e.g., bacteria (e.g., bacteria disclosed herein) and/or an agent of bacterial origin, such as mEVs (e.g., mEVs disclosed herein)) (e.g., the pharmaceutical agent can be a powder comprising bacteria and/or an agent of bacterial origin, such as mEVs) and one or more (e.g., one, two or three) excipients into a pharmaceutical composition. In some embodiments, the wet granulation comprises mixing the pharmaceutical agent with a granulating fluid (e.g., water, ethanol, or isopropanol, alone or in combination) to prepare a mixed composition. In some embodiments, the granulating fluid comprises water. In some embodiments, the granulating fluid consists of water. In some embodiments, the wet granulation further comprises drying the mixed composition (e.g., drying on a fluid bed dryer) to prepare a dried composition. In some embodiments, the wet granulation further comprises milling the dried composition to prepare a milled composition. The milled composition can optionally be combined with the one or more (e.g., one, two or three) excipients to prepare a pharmaceutical composition.

### Granules and Wet Granulation

In some aspects, provided herein are granules that comprise a pharmaceutical agent, e.g., wherein the pharmaceutical agent comprises bacteria (e.g., bacteria disclosed herein) and/or an agent of bacterial origin, such as mEVs (e.g., mEVs disclosed herein) (e.g., the pharmaceutical agent can be a powder comprising bacteria and/or an agent of bacterial origin, such as mEVs). The granules comprise agglomerations of pharmaceutical agent (e.g., larger particles than the pharmaceutical agent particles (e.g., than of a powder)). The diameter of the granules is greater (e.g., about 1.5-fold to over 4-fold greater) than the diameter (e.g., average diameter) of the pharmaceutical agent (e.g., powder, e.g., powder particles). The granules can be produced by wet granulation.

For example, for Prevotella histicola Strain B smEVs, the diameter of granules after wet granulation is about 1.5-fold to over 4-fold larger than the diameter of the DS powder:

| Prevotella histicola smEVs | D₁₀ (μm) | D₅₀ (μm) | D₉₀ (μm) |
|---|---|---|---|
| HS DS granules | 8.22 | 110 | 386 |
| HS DS powder | 5.61 | 25.4 | 95.3 |

Granulation is the process of particle enlargement by agglomeration. Granulation can transform fine powders into free-flowing, dust-free granules that are easier to compress. During the granulation process, small fine or coarse particles are converted into larger agglomerates called granules. See, e.g., Shanmugam, Bioimpacts 5:55-63 (2015). Wet granulation involves the production of a granule by the addition of a liquid binder (e.g., granulating fluid) to a powder (e.g., that comprises a pharmaceutical agent, e.g., comprises bacteria (e.g., bacteria disclosed herein) and/or an agent of bacterial origin, such as mEVs (e.g., mEVs disclosed herein)).

Granulation, e.g., wet granulation, can allow for higher doses of bacteria (e.g., bacteria disclosed herein) and/or an agent of bacterial origin, such as mEVs (e.g., mEVs disclosed herein) to be formulated into a solid dose form (e.g., tablet, mini-tablet or capsule). For example, by performing wet granulation on a powder comprising mEVs, the dose of mEVs the dose of mEVs in a size 0 capsule is increased by 3 fold.

In certain aspects, provided herein are methods of wet granulation of a pharmaceutical agent, e.g., wherein the pharmaceutical agent comprises bacteria (e.g., bacteria disclosed herein) and/or an agent of bacterial origin, such as mEVs (e.g., mEVs disclosed herein) (e.g., the pharmaceutical agent can be a powder comprising bacteria and/or an agent of bacterial origin, such as mEVs).

In some embodiments, the wet granulation comprises mixing the pharmaceutical agent with a granulating fluid (e.g., water, ethanol, or isopropanol, alone or in combination) to prepare a mixed composition. In some embodiments, the granulating fluid comprises water. In some embodiments, the granulating fluid consists of water. In some embodiments, the wet granulation comprises drying the mixed composition (e.g., drying on a fluid bed dryer) to prepare a dried composition. In some embodiments, the wet granulation comprises milling the dried composition to prepare a milled composition. The milled composition can then optionally be combined with the one or more (e.g., one, two or three) excipients into a pharmaceutical composition. The wet granulation process can produce granules.

In some embodiments, the wet granulation comprises (i) mixing the pharmaceutical agent with a granulating fluid (e.g., water, ethanol, or isopropanol, alone or in combination) to prepare a mixed composition and (ii) drying the mixed composition (e.g., drying on a fluid bed dryer) to prepare a dried composition.

In some embodiments, the wet granulation comprises (i) mixing the pharmaceutical agent with a granulating fluid (e.g., water, ethanol, or isopropanol, alone or in combination) to prepare a mixed composition; (ii) drying the mixed composition (e.g., drying on a fluid bed dryer) to prepare a dried composition; and (iii) milling the dried composition to prepare a milled composition.

In some embodiments, the wet granulation comprises (i) mixing the pharmaceutical agent with a granulating fluid (e.g., water, ethanol, or isopropanol, alone or in combination) to prepare a mixed composition; (ii) drying the mixed composition (e.g., drying on a fluid bed dryer) to prepare a dried composition; (iii) milling the dried composition to prepare a milled composition; and (iv) combining the milled composition with the one or more (e.g., one, two or three) excipients into a pharmaceutical composition.

In some embodiments, provided herein are granules produced by wet granulation.

In some aspects, provided herein is a mixed composition, e.g., that comprises a pharmaceutical agent and a granulating fluid (e.g., water, ethanol, or isopropanol, alone or in combination).

In certain aspects, provided herein is a dried composition, e.g., that comprises a mixed composition that has been dried.

In certain aspects, provided herein is a milled composition. e.g., that comprises a dried composition that has been milled.

### Additional Aspects of the Solid Dosage Forms

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs), and wherein the solid dosage form further comprises the described components, can provide a therapeutically effective amount of the pharmaceutical agent to a subject, e.g., a human.

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs), and wherein the solid dosage form further comprises the described components, can provide a non-natural amount of the therapeutically effective components (e.g., present in the pharmaceutical agent) to a subject, e.g., a human.

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs), and wherein the solid dosage form further comprises the described components, can provide an unnatural quantity of the therapeutically effective components (e.g., present in the pharmaceutical agent) to a subject, e.g., a human.

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs), and wherein the solid dosage form further comprises the described components, can bring about one or more changes to a subject, e.g., human, e.g., to treat or prevent a disease or a health disorder.

The solid dosage forms, e.g., as described herein, comprising a pharmaceutical agent (e.g., a therapeutically effective amount thereof), wherein the pharmaceutical agent comprises bacteria and/or microbial extracellular vesicles (mEVs), and wherein the solid dosage form further comprises the described components, has potential for significant utility, e.g., to affect a subject, e.g., a human, e.g., to treat or prevent a disease or a health disorder.

### Additional Therapeutic Agents

In certain aspects, the methods provided herein include the administration to a subject of a solid dosage form described herein either alone or in combination with an additional therapeutic agent. In some embodiments, the additional therapeutic agent is an immunosuppressant, an anti-inflammatory agent, a steroid, and/or a cancer therapeutic.

In some embodiments, the solid dosage form is administered to the subject before the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, the solid dosage form is administered to the subject after the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours after or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the solid dosage form and the additional therapeutic agent are administered to the subject simultaneously or nearly simultaneously (e.g., administrations occur within an hour of each other).

In some embodiments, the additional therapeutic agent is a cancer therapeutic. In some embodiments, the cancer therapeutic is a chemotherapeutic agent. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate, hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In some embodiments, the cancer therapeutic is a cancer immunotherapy agent. Immunotherapy refers to a treatment that uses a subject's immune system to treat cancer, e.g., checkpoint inhibitors, cancer vaccines, cytokines, cell therapy, CAR-T cells, and dendritic cell therapy. Non-limiting examples of immunotherapies are checkpoint inhibitors include Nivolumab (BMS, anti-PD-1), Pembrolizumab (Merck, anti-PD-1), Ipilimumab (BMS, anti-CTLA-4). MED14736 (AstraZeneca, anti-PD-L1), and MPDL3280A (Roche, anti-PD-L1). Other immunotherapies may be tumor vaccines, such as Gardail, Cervarix, BCG, sipulencel-T, Gp100:209-217, AGS-003, DCVax-L, Algenpantucel-L, Tergenpantucel-L, TG4010, ProstAtak, Prostvac-V/R-TRICOM, Rindopepimul, E75 peptide acetate, IMA901, POL-103A, Belagenpumatucel-L, GSK1572932A, MDX-1279, GV1001, and Tecemotide. The immunotherapy agent may be administered via injection (e.g., intravenously, intratumorally, subcutaneously, or into lymph nodes), but may also be administered orally, topically, or via aerosol. Immunotherapies may comprise adjuvants such as cytokines.

In some embodiments, the immunotherapy agent is an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0020718C, AUR-012 and STI-A1010.

In some embodiments, the methods provided herein include the administration of a pharmaceutical composition described herein in combination with one or more additional therapeutic agents. In some embodiments, the methods disclosed herein include the administration of two immunotherapy agents (e.g., immune checkpoint inhibitor). For example, the methods provided herein include the administration of a pharmaceutical composition described herein in combination with a PD-1 inhibitor (such as pemrolizumab or nivolumab or pidilizumab) or a CLTA-4 inhibitor (such as ipilimumab) or a PD-L1 inhibitor.

In some embodiments, the immunotherapy agent is an antibody or antigen binding fragment thereof that, for example, binds to a cancer-associated antigen. Examples of cancer-associated antigens include, but are not limited to, adipophilin, AIM-2, ALDHIA1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF. CSNKlAI. CTAG1, CTAG2, cyclin D1, Cyclin-A1, dek-can fusion protein. DKK1, EFTUD2, Elongation factor 2, ENAH (hMena), Ep-CAM, EpCAM, EphA3, epithelial tumor antigen (“ETA”), ETV6-AML 1 fusion protein, EZH2, FGF5, FLT3-ITD, FN1, G250/MN/CAIX, GAGE-1,2,8, GAGE-3,4,5,6,7, GAS7, glypican-3, GnTV, gpI00/Pmell7. GPNMB. HAUS3, Hepsin, HER-2/neu, HERV-K-MEL, HLA-A11. HLA-A2, HLA-DOB, hsp70-2, IDOl, IGF2B3, IL13Ralpha2, Intestinal carboxyl esterase, K-ras, Kallikrein 4, KIF20A, KK-LC-1, KKLC1, KM-HN-1, KMHN1 also known as CCDCl 10, LAGE-1. LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MCI R. MCSP, mdm-2, ME1. Melan-A/MART-1, Meloe, Midkine. MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPPI R3B, PRAME, PRDX5, PSA, PSMA, P1PRK, RAB38/NY-MEL-1, RAGE-1, RBAF600, RGS5, RhoC, RNF43, RU2AS, SAGE, secemin 1, SIRT2, SNRPD1, SOX10, Sp17, SPA7, SSX-2, SSX-4, STEAP1, survivin, SYT-SSX1 or -SSX2 fusion protein, TAG-1, TAG-2. Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-1b/GAGED2a. In some embodiments, the antigen is a neo-antigen.

In some embodiments, the immunotherapy agent is a cancer vaccine and/or a component of a cancer vaccine (e.g., an antigenic peptide and/or protein). The cancer vaccine can be a protein vaccine, a nucleic acid vaccine or a combination thereof. For example, in some embodiments, the cancer vaccine comprises a polypeptide comprising an epitope of a cancer-associated antigen. In some embodiments, the cancer vaccine comprises a nucleic acid (e.g., DNA or RNA, such as mRNA) that encodes an epitope of a cancer-associated antigen. Examples of cancer-associated antigens include, but are not limited to, adipophilin, AIM-2, ALDH1A1, alpha-actinin-4, alpha-fetoprotein (“AFP”), ARTC1. B-RAF, BAGE-1. BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNKIA1, CTAG1, CTAG2, cyclin D1, Cyclin-A1, dek-can fusion protein, DKK1. EFTUD2, Elongation factor 2, ENAH (hMena). Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gpI00/Pmell7. GPNMB, HAUS3, Hepsin, HER-2/neu, HERV-K-MEL, HLA-A11, HLA-A2, HLA-DOB, hsp70-2, IDOl, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MCIR, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I. N-raw, NA88-A, neo-PAP, NFYC, NY-BR-1, NY-ESO-1/LAGE-2, OAI, OGT, OS-9, P polypeptide, p53, PAP, PAX5, PBF, pml-RARalpha fusion protein, polymorphic epithclial mucin (“PEM”), PPPIR3B, PRAME, PRDX5, PSA, PSMA, PTPRK, RAB38/NY-MEL-1, RAGE-1, RBAF600, RGS5. RhoC, RNF43, RU2AS, SAGE, secemin 1, SIRT2, SNRPD1. SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase. TRP-1/gp75, TRP-2, TRP2-INT2, tyrosinase, tyrosinase (“TYR”), VEGF, WT1, XAGE-1b/GAGED2a. In some embodiments, the antigen is a neo-antigen. In some embodiments, the cancer vaccine is administered with an adjuvant. Examples of adjuvants include, but are not limited to, an immune modulatory protein, Adjuvant 65, α-GalCer, aluminum phosphate, aluminum hydroxide, calcium phosphate, β-Glucan Peptide, CpG ODN DNA, GPI-0100, lipid A, lipopolysaccharide, Lipovant, Montanide, N-acetyl-muramyl-L-alanyl-D-isoglutamine, Pam3CSK4, quil A, cholera toxin (CT) and heat-labile toxin from enterotoxigenic Escherichia coli (LT) including derivatives of these (CTB, mmCT, CTA1-DD, LTB, LTK63, LTR72, dmLT) and trehalose dimycolate.

In some embodiments, the immunotherapy agent is an immune modulating protein to the subject. In some embodiments, the immune modulatory protein is a cytokine or chemokine. Examples of immune modulating proteins include, but are not limited to, B lymphocyte chemoattractant (“BLC”). C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon alpha (“IFN-alpha”), Interferon beta (“FN-beta”) Interferon gamma (“IFN-gamma”), Interleukin-1 alpha (“IL-1 alpha”), Interleukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”). Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17A-F (“IL-17A-F”), Interleukin-18 (“IL-18”), Interleukin-21 (“IL-21”), Interleukin-22 (“IL-22”), Interleukin-23 (“IL-23”), Interleukin-33 (“IL-33”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophage inflammatory protein-1-delta (“MIP-1 delta”), Platelet-derived growth factor subunit B (“PDGF-BB”). Chemokine (C-C motif) ligand 5. Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”). Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Ncurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing comples (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”). VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C—X—C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C—X—C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”). Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C-C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembrvonic antigen-related cell adhesion molecule 1 (biliary glycoprotein) (“CEACAM-1”), Death Receptor 6 (“DR6”), Deoxythymidine kinase (“Dtk”). Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta. IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRGl-beta1, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDARActivin A, Agouti-related protein (“AgRP”), Ribonuclease 5 (“Angiogenin”). Angiopoietin 1, Angiostatin, Catheprin S. CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 RI, IL-13R2, IL-17B, IL-2 Ra, IL-2 Rb, IL-23, LAP, Neuronal cell adhesion molecule (“NrCAM”), Plasminogen activator inhibitor-1 (“PAl-1”), Platelet derived growth factor receptors (“PDGF-AB”), Resistin, stromal cell-derived factor 1 (“SDF-1 beta”), sgpl30, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFRlAdiponectin. Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”). Galectin-3. Hepatocyte growth factor receptor (“HGF R”), IFN-gammalpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”). N-acetylmuramoyl-L-alanine amidase (“PGRP-5”), Serpin A4, Secreted frizzled related protein 3 (“sFRP-3”), Thrombomodulin, Tolllike receptor 2 (“TLR2”), Tumor necrosis factor receptor superfamily member 10A (“TRAIL R1”), Transferrin (“TRF”), WIF-IACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A. Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen 1, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNTI-inducible-signaling pathway protein 1 (“WISP-1”), and Receptor Activator of Nuclear Factor κ B (“RANK”).

In some embodiments, the cancer therapeutic is an anti-cancer compound. Exemplary anti-cancer compounds include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 131I-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®).

Exemplary anti-cancer compounds that modify the function of proteins that regulate gene expression and other cellular functions (e.g., HDAC inhibitors, retinoid receptor ligants) are Vorinostat (Zolinza®), Bexarotene (Targretin®) and Romidepsin (Istodax®). Alitretinoin (Panretin®), and Tretinoin (Vesanoid®).

Exemplary anti-cancer compounds that induce apoptosis (e.g., proteasome inhibitors, antifolates) are Bortezomib (Velcade®), Carfilzomib (Kyprolis™), and Pralatrexate (Folotyn®).

Exemplary anti-cancer compounds that increase anti-tumor immune response (e.g., anti CD20, anti CD52; anti-cytotoxic T-lymphocyte-associated antigen-4) are Rituximab (Rituxan®), Alemtuzumab (Campath®), Ofatumumab (Arzerra®), and Ipilimumab (Yervoy™).

Exemplary anti-cancer compounds that deliver toxic agents to cancer cells (e.g., anti-CD20-radionuclide fusions; IL-2-diphtheria toxin fusions; anti-CD30-monomethylauristatin E (MMAE)-fusions) are Tositumomab and 131I-tositumomab (Bexxar®) and Ibritumomab tiuxetan (Zevalin®), Denileukin diftitox (Ontak®), and Brentuximab vedotin (Adcetris®).

Other exemplary anti-cancer compounds are small molecule inhibitors and conjugates thereof of, e.g., Janus kinase, ALK, Bcl-2, PARP, PI3K, VEGF receptor, Braf, MEK, CDK, and HSP90.

Exemplary platinum-based anti-cancer compounds include, for example, cisplatin, carboplatin, oxaliplatin, satraplatin, picoplatin, Nedaplatin, Triplatin, and Lipoplatin. Other metal-based drugs suitable for treatment include, but are not limited to ruthenium-based compounds, ferrocene derivatives, titanium-based compounds, and gallium-based compounds.

In some embodiments, the cancer therapeutic is a radioactive moiety that comprises a radionuclide. Exemplary radionuclides include, but are not limited to Cr-51, Cs-131, Ce-134, Se-75, Ru-97, I-125, Eu-149, Os-189m, Sb-119, I-123, Ho-161, Sb-117, Ce-139, In-111, Rh-103m, Ga-67, T1-201, Pd-103, Au-195, Hg-197, Sr-87m, Pt-191, P-33, Er-169, Ru-103, Yb-169, Au-199, Sn-121, Tm-167, Yb-175, In-113m, Sn-113, Lu-177, Rh-105, Sn-117m, Cu-67, Sc-47, Pt-195m, Ce-141, I-131, Tb-161, As-77, Pt-197, Sm-153, Gd-159, Tm-173, Pr-143, Au-198, Tm-170, Re-186, Ag-111, Pd-109, Ga-73, Dy-165, Pm-149, Sn-123, Sr-89, Ho-166, P-32, Re-188, Pr-142, Ir-194, In-114m/In-114, and Y-90.

In some embodiments, an antibiotic is administered to the subject before the solid dosage form is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, an antibiotic is administered to the subject after the solid dosage form is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the solid dosage form and the antibiotic are administered to the subject simultaneously or nearly simultaneously (e.g., administrations occur within an hour of each other).

In some embodiments, the additional therapeutic is an antibiotic. For example, if the presence of a disease-associated bacteria and/or a disease-associated microbiome profile is detected, antibiotics can be administered, e.g., to eliminate the disease-associated bacteria from the subject. “Antibiotics” broadly refers to compounds capable of inhibiting or preventing a bacterial infection. Antibiotics can be classified in a number of ways, including their use for specific infections, their mechanism of action, their bioavailability, or their spectrum of target microbe (e.g., Gram-negative vs. Gram-positive bacteria, aerobic vs. anaerobic bacteria, etc.) and these may be used to kill specific bacteria in specific areas of the host (“niches”) (Leekha, et al 2011. General Principles of Antimicrobial Therapy. Mayo Clin Proc. 86(2): 156-167). In certain embodiments, antibiotics can be used to selectively target bacteria of a specific niche. In some embodiments, antibiotics known to treat a particular infection that includes a disease niche may be used to target disease-associated microbes, including disease-associated bacteria in that niche. In other embodiments, antibiotics are administered after the solid dosage form. In some embodiments, antibiotics are administered before the solid dosage form.

In some aspects, antibiotics can be selected based on their bactericidal or bacteriostatic properties. Bactericidal antibiotics include mechanisms of action that disrupt the cell wall (e.g., β-lactams), the cell membrane (e.g., daptomycin), or bacterial DNA (e.g., fluoroquinolones). Bacteriostatic agents inhibit bacterial replication and include sulfonamides, tetracyclines, and macrolides, and act by inhibiting protein synthesis. Furthermore, while some drugs can be bactericidal in certain organisms and bacteriostatic in others, knowing the target organism allows one skilled in the art to select an antibiotic with the appropriate properties. In certain treatment conditions, bacteriostatic antibiotics inhibit the activity of bactericidal antibiotics. Thus, in certain embodiments, bactericidal and bacteriostatic antibiotics are not combined.

Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

Aminoglycosides include, but are not limited to Amikacin, Gentamicin, Kanamycin, Neomycin, Netilmicin, Tobramycin, Paromomycin, and Spectinomycin. Aminoglycosides are effective, e.g., against Gram-negative bacteria, such as Escherichia coli, Klebsiella, Pseudomonas aeruginosa, and Francisella tularensis, and against certain aerobic bacteria but less effective against obligate/facultative anaerobes. Aminoglycosides are believed to bind to the bacterial 30S or 50S ribosomal subunit thereby inhibiting bacterial protein synthesis.

Ansamycins include, but are not limited to, Geldanamycin, Herbimycin, Rifamycin, and Streptovaricin. Geldanamycin and Herbimycin are believed to inhibit or alter the function of Heat Shock Protein 90.

Carbacephems include, but are not limited to, Loracarbef. Carbacephems are believed to inhibit bacterial cell wall synthesis.

Carbapenems include, but are not limited to. Ertapenem, Doripenem, Imipenem/Cilastatin, and Meropenem. Carbapenems are bactericidal for both Gram-positive and Gram-negative bacteria as broad-spectrum antibiotics. Carbapenems are believed to inhibit bacterial cell wall synthesis.

Cephalosporins include, but are not limited to. Cefadroxil, Cefazolin, Cefalotin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Cefibuten, Ceftizoxime, Ceftriaxone, Cefepime, Ceftaroline fosamil, and Ceftobiprole. Selected Cephalosporins are effective, e.g., against Gram-negative bacteria and against Gram-positive bacteria, including Pseudomonas, certain Cephalosporins are effective against methicillin-resistant Staphylococcus aureus (MRSA). Cephalosporins are believed to inhibit bacterial cell wall synthesis by disrupting synthesis of the peptidoglycan layer of bacterial cell walls.

Glycopeptides include, but are not limited to, Teicoplanin, Vancomycin, and Telavancin. Glycopeptides are effective, e.g., against aerobic and anaerobic Gram-positive bacteria including MRSA and Clostridium difficile. Glycopeptides are believed to inhibit bacterial cell wall synthesis by disrupting synthesis of the peptidoglycan layer of bacterial cell walls.

Lincosamides include, but are not limited to, Clindamycin and Lincomycin. Lincosamides are effective, e.g., against anaerobic bacteria, as well as Staphylococcus, and Streptococcus. Lincosamides are believed to bind to the bacterial 50S ribosomal subunit thereby inhibiting bacterial protein synthesis.

Lipopeptides include, but are not limited to, Daptomycin. Lipopeptides are effective, e.g., against Gram-positive bacteria. Lipopeptides are believed to bind to the bacterial membrane and cause rapid depolarization.

Macrolides include, but are not limited to, Azithromycin, Clarithromycin, Dirithromycin, Erythromycin, Roxithromycin, Troleandomycin, Telithromycin, and Spiramycin. Macrolides are effective, e.g., against Streptococcus and Mycoplasma. Macrolides are believed to bind to the bacterial or 50S ribosomal subunit, thereby inhibiting bacterial protein synthesis.

Monobactams include, but are not limited to, Aztreonam. Monobactams are effective. e.g., against Gram-negative bacteria. Monobactams are believed to inhibit bacterial cell wall synthesis by disrupting synthesis of the peptidoglycan layer of bacterial cell walls.

Nitrofurans include, but are not limited to, Furazolidone and Nitrofurantoin.

Oxazolidonones include, but are not limited to. Linezolid, Posizolid, Radezolid, and Torezolid. Oxazolidonones are believed to be protein synthesis inhibitors.

Penicillins include, but are not limited to, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V. Piperacillin, Temocillin and Ticarcillin. Penicillins are effective, e.g., against Gram-positive bacteria, facultative anaerobes, e.g., Streptococcus, Borrelia, and Trcponema. Penicillins are believed to inhibit bacterial cell wall synthesis by disrupting synthesis of the peptidoglycan layer of bacterial cell walls.

Penicillin combinations include, but are not limited to, Amoxicillin/clavulanate, Ampicillin/sulbactam, Piperacillin/tazobactam, and Ticarcillin/clavulanate.

Polypeptide antibiotics include, but are not limited to. Bacitracin, Colistin, and Polymyxin B and E. Polypeptide Antibiotics are effective, e.g., against Gram-negative bacteria. Certain polypeptide antibiotics are believed to inhibit isoprenyl pyrophosphate involved in synthesis of the peptidoglycan layer of bacterial cell walls, while others destabilize the bacterial outer membrane by displacing bacterial counter-ions.

Quinolones and Fluoroquinolone include, but are not limited to, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. Quinolones/Fluoroquinolone are effective, e.g., against Streptococcus and Neisseria. Quinolones/Fluoroquinolone are believed to inhibit the bacterial DNA gyrase or topoisomerase IV, thereby inhibiting DNA replication and transcription.

Sulfonamides include, but are not limited to, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole), and Sulfonamidochrysoidine. Sulfonamides are believed to inhibit folate synthesis by competitive inhibition of dihydropteroate synthetase, thereby inhibiting nucleic acid synthesis.

Tetracyclines include, but are not limited to, Demeclocycline, Doxycycline, Minocycline, Oxytetracycline, and Tetracycline. Tetracyclines are effective, e.g., against Gram-negative bacteria. Tetracyclines are believed to bind to the bacterial 30S ribosomal subunit thereby inhibiting bacterial protein synthesis.

Anti-mycobacterial compounds include, but are not limited to, Clofazimine, Dapsone, Capreomycin, Cycloserine, Ethambutol, Ethionamide. Isoniazid, Pyrazinamide. Rifampicin, Rifabutin, Rifapentine, and Streptomycin.

Suitable antibiotics also include arsphenamine, chloramphenicol, fosfomycin, fusidic acid, metronidazole, mupirocin, platensimycin, quinupristin/dalfopristin, tigecycline, tinidazole, trimethoprim amoxicillin/clavulanate, ampicillin/sulbactam, amphomycin ristocetin, azithromycin, bacitracin, buforin II, carbomycin, cecropin PI, clarithromycin, erythromycins, furazolidone, fusidic acid, Na fusidate, gramicidin, imipenem, indolicidin, josamycin, magainan II, metronidazole, nitroimidazoles, mikamycin, mutacin B-Ny266, mutacin B-JHI 140, mutacin J-T8, nisin, nisin A, novobiocin, oleandomycin, ostreogrycin, piperacillin/tazobactam, pristinamycin, ramoplanin, ranalexin, reuterin, rifaximin, rosamicin, rosaramicin, spectinomycin, spiramycin, staphylomycin, streptogramin, streptogramin A, synergistin, taurolidine, teicoplanin, telithromycin, ticarcillin/clavulanic acid, triacetyloleandomycin, tylosin, tyrocidin, tyrothricin, vancomycin, vemamycin, and virginiamycin.

In some embodiments, the additional therapeutic agent is an immunosuppressive agent, a DMARD, a pain-control drug, a steroid, a non-steroidal anti-inflammatory drug (NSAID), or a cytokine antagonist, and combinations thereof. Representative agents include, but are not limited to, cyclosporin, retinoids, corticosteroids, propionic acid derivative, acetic acid derivative, enolic acid derivatives, fenamic acid derivatives, Cox-2 inhibitors, lumiracoxib, ibuprophen, cholin magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966; rofecoxib, acetominophen, Celecoxib, Diclofenac, tramadol, piroxicam, meloxicam, tenoxicam, droxicam, lornoxicam, isoxicam, mefanamic acid, meclofenamic acid, flufenamic acid, tolfenamic, valdecoxib, parecoxib, etodolac, indomethacin, aspirin, ibuprophen, firocoxib, methotrexate (MTX), antimalarial drugs (e.g., hydroxychloroquine and chloroquine), sulfasalazine, Leflunomide, azathioprine, cyclosporin, gold salts, minocycline, cyclophosphamide, D-penicillamine, minocycline, auranofin, tacrolimus, myocrisin, chlorambucil, TNF alpha antagonists (e.g., TNF alpha antagonists or TNF alpha receptor antagonists), e.g., ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MabThera®), ABATACEPT (Orencia®), TOCILIZUMAB (RoActemra/Actemr®), integrin antagonists (TYSABRI® (natalizumab)), IL-1 antagonists (ACZ885 (Ilans)), Anakinra (Kincret®)), CD4 antagonists, IL-23 antagonists, IL-20 antagonists, IL-6 antagonists, BLyS antagonists (e.g., Atacicept, Benlysta®/LymphoStat-B® (belimumab)), p38 Inhibitors, CD20 antagonists (Ocrelizumab, Ofatumumab (Arzerra®)), interferon gamma antagonists (Fontolizumab), prednisolone, Prednisone, dexamethasone. Cortisol, cortisone, hydrocortisone, methylprednisolone, betamethasone, triamcinolone, beclometasome, fludrocortisone, deoxycorticosterone, aldosterone, Doxycycline, vancomycin, pioglitazone, SBI-087, SCIO-469, Cura-100, Oncoxin+Viusid, TwHF, Methoxsalen, Vitamin D—ergocalciferol, Milnacipran, Paclitaxel, rosig tazone, Tacrolimus (Prograf t), RADOOL, rapamune, rapamycin, fostamatinib. Fentanyl. XOMA 052, Fostamatinib disodium, rosightazone, Curcumin (Longvida™), Rosuvastatin, Maraviroc, ramipnl, Milnacipran, Cobiprostone, somatropin, tgAAC94 gene therapy vector, MK0359. GW856553, esomeprazole, everolimus, trastuzumab, JAK1 and JAK2 inhibitors, pan JAK inhibitors, e.g., tetracyclic pyridone 6 (P6), 325, PF-956980, denosumab, IL-6 antagonists, CD20 antagonistis, CTLA4 antagonists, IL-8 antagonists, IL-21 antagonists, IL-22 antagonist, integrin antagonists (Tysarbri® (natalizumab)), VGEF antagnosits, CXCL antagonists, MMP antagonists, defensin antagonists, IL-1 antagonists (including IL-1 beta antagonsits), and IL-23 antagonists (e.g., receptor decoys, antagonistic antibodies, etc.).

In some embodiments, the additional therapy can comprise a JAK inhibitor such as baricitinib, ruxolitinib, tofacitinib, and/or pacritinib.

In some embodiments, the additional therapeutic agent is an immunosuppressive agent. Examples of immunosuppressive agents include, but are not limited to, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines (e.g., vaccines used for vaccination where the amount of an allergen is gradually increased), cytokine inhibitors, such as anti-IL-6 antibodies, TNF inhibitors such as infliximab, adalimumab, certolizumab pegol, golimumab, or etanercept, and combinations thereof.

In some embodiments, the additional therapeutic agent is an RNA molecule, such as a double stranded RNA.

In some embodiments, the additional therapeutic agent is an anti-sense oligonucleotide.

### Administration

In certain aspects, provided herein is a method of delivering a solid dosage form described herein to a subject. In some embodiments of the methods provided herein, the solid dosage form is administered in conjunction with the administration of an additional therapeutic agent. In some embodiments, the solid dosage form comprises a pharmaceutical agent co-formulated with the additional therapeutic agent. In some embodiments, the solid dosage form is co-administered with the additional therapeutic agent. In some embodiments, the additional therapeutic agent is administered to the subject before administration of the solid dosage form (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the additional therapeutic agent is administered to the subject after administration of the solid dosage form (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments, the same mode of delivery is used to deliver both the solid dosage form and the additional therapeutic agent. In some embodiments, different modes of delivery are used to administer the solid dosage form and the additional therapeutic agent. For example, in some embodiments the solid dosage form is administered orally while the additional therapeutic agent is administered via injection (e.g., an intravenous and/or intramuscular).

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, and other compounds such as drugs being administered concurrently or near-concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate. The dose of a pharmaceutical agent (e.g., in a solid dosage form) described herein may be appropriately set or adjusted in accordance with the dosage form, the route of administration, the degree or stage of a target disease, and the like. For example, the general effective dose of the agents may range between 0.01 mg/kg body weight/day and 1000 mg/kg body weight/day, between 0.1 mg/kg body weight/day and 1000 mg/kg body weight/day, 0.5 mg/kg body weight/day and 500 mg/kg body weight/day, 1 mg/kg body weight/day and 100 mg/kg body weight/day, or between 5 mg/kg body weight/day and 50 mg/kg body weight/day. The effective dose may be 0.01, 0.05, 0.1, 0.5, 1, 2, 3, 5, 10, 20, 30, 40, 50, 60, 70, 80, 90, 100, 200, 500, or 1000 mg/kg body weight/day or more, but the dose is not limited thereto.

In some embodiments, the dose administered to a subject is sufficient to prevent disease (e.g., autoimmune disease, inflammatory disease, or metabolic disease), delay its onset, or slow or stop its progression, or relieve one or more symptoms of the disease. One skilled in the art will recognize that dosage will depend upon a variety of factors including the strength of the particular agent (e.g., pharmaceutical agent) employed, as well as the age, species, condition, and body weight of the subject. The size of the dose will also be determined by the route, timing, and frequency of administration as well as the existence, nature, and extent of any adverse side-effects that might accompany the administration of a particular pharmaceutical agent and the desired physiological effect.

Suitable doses and dosage regimens can be determined by conventional range-finding techniques known to those of ordinary skill in the art. Generally, treatment is initiated with smaller dosages, which are no more than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the pharmaceutical agents used in accordance with the invention vary depending on the active agent, the age, weight, and clinical condition of the recipient patient, and the experience and judgment of the clinician or practitioner administering the therapy, among other factors affecting the selected dosage. As another example, the dose should be sufficient to result in slowing of progression of the disease for which the subject is being treated, and preferably amelioration of one or more symptoms of the disease for which the subject is being treated.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a solid dosage form, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response. In one example, the time period can be a function of the time period for a subject to mount an immune response; for example, the time period can be more than the time period for a subject to mount an immune response, such as more than about one week, more than about ten days, more than about two weeks, or more than about a month, in another example, the time period can be no more than the time period for a subject to mount an immune response, such as no more than about one week, no more than about ten days, no more than about two weeks, or no more than about a month.

In some embodiments, the delivery of an additional therapeutic agent in combination with the solid dosage form described herein reduces the adverse effects and/or improves the efficacy of the additional therapeutic agent.

The effective dose of an additional therapeutic agent described herein is the amount of the additional therapeutic agent that is effective to achieve the desired therapeutic response for a particular subject, composition, and mode of administration, with the least toxicity to the subject. The effective dosage level can be identified using the methods described herein and will depend upon a variety of pharmacokinetic factors including the activity of the particular compositions or agents administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well known in the medical arts. In general, an effective dose of an additional therapeutic agent will be the amount of the additional therapeutic agent which is the lowest dose effective to produce a therapeutic effect. Such an effective dose will generally depend upon the factors described above.

The toxicity of an additional therapeutic agent is the level of adverse effects experienced by the subject during and following treatment. Adverse events associated with additional therapy toxicity can include, but are not limited to, abdominal pain, acid indigestion, acid reflux, allergic reactions, alopecia, anaphylaxis, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypennagnesemia, hypematremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heart beat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

### Immune Disorders

In some embodiments, the methods and solid dosage forms described herein relate to the treatment or prevention of a disease or disorder associated a pathological immune response, such as an autoimmune disease, an allergic reaction and/or an inflammatory disease. In some embodiments, the disease or disorder is an inflammatory bowel disease (e.g., Crohn's disease or ulcerative colitis). In some embodiments, the disease or disorder is psoriasis. In some embodiments, the disease or disorder is atopic dermatitis. In some embodiments, the disease or disorder is asthma.

The methods and solid dosage forms described herein can be used to treat any subject in need thereof. As used herein, a “subject in need thereof” includes any subject that has a disease or disorder associated with a pathological immune response (e.g., an inflammatory bowel disease), as well as any subject with an increased likelihood of acquiring a such a disease or disorder.

The solid dosage forms described herein can be used, for example, as a pharmaceutical composition for preventing or treating (reducing, partially or completely, the adverse effects of) an autoimmune disease, such as chronic inflammatory bowel disease, systemic lupus erythematosus, psoriasis, muckle-wells syndrome, rheumatoid arthritis, multiple sclerosis, or Hashimoto's disease, an allergic disease, such as a food allergy, pollenosis, or asthma; an infectious disease, such as an infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a pharmaceutical composition for suppressing rejection in organ transplantation or other situations in which tissue rejection might occur, a supplement, food, or beverage for improving immune functions; or a reagent for suppressing the proliferation or function of immune cells.

In some embodiments, the methods and solid dosage forms provided herein are useful for the treatment of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below.

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular immune disorders refers to a immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis.

Examples of nervous system immune disorders which may be treated with the methods and solid dosage forms described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system immune disorders which may be treated with the methods and solid dosage forms described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system immune disorders which may be treated with the methods and solid dosage forms described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The methods and solid dosage forms described herein may be used to treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease. Crohn's disease, diabetes mellitus type 1, giant cell arteritis, Goodpasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, Ord's thyroiditis, pemphigus, polyarteritis nodosa, polymyalgia, rheumatoid arthritis, Reiter's syndrome, Sjogren's syndrome, temporal arteritis. Wegener's granulomatosis, warm autoimmune haemolytic anemia, interstitial cystitis, Lyme disease, morphea, psoriasis, sarcoidosis, scleroderma, ulcerative colitis, and vitiligo.

The methods and solid dosage forms described herein may be used to treat T-cell mediated hypersensitivity diseases having an inflammatory component. Such conditions include, but are not limited to, contact hypersensitivity, contact dermatitis (including that due to poison ivy), uticaria, skin allergies, respiratory allergies (hay fever, allergic rhinitis, house dustmite allergy) and gluten-sensitive enteropathy (Celiac disease).

Other immune disorders which may be treated with the methods and solid dosage forms include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (e.g., sepsis).

### Metabolic Disorders

In some embodiments, the methods and solid dosage forms described herein relate to the treatment or prevention of a metabolic disease or disorder a, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), Nonalcoholic Steatohepatitis (NASH) or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema. In some embodiments, the methods and pharmaceutical compositions described herein relate to the treatment of Nonalcoholic Fatty Liver Disease (NAFLD) and Nonalcoholic Steatohepatitis (NASH).

The methods and solid dosage forms described herein can be used to treat any subject in need thereof. As used herein, a “subject in need thereof” includes any subject that has a metabolic disease or disorder, as well as any subject with an increased likelihood of acquiring a such a disease or disorder.

The solid dosage forms described herein can be used, for example, for preventing or treating (reducing, partially or completely, the adverse effects of) a metabolic disease, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), Nonalcoholic Steatohepatitis (NASH), or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema.

### Cancer

In some embodiments, the methods and solid dosage forms described herein relate to the treatment of cancer. In some embodiments, any cancer can be treated using the methods described herein. Examples of cancers that may treated by methods and solid dosage forms described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; lcydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer comprises breast cancer (e.g., triple negative breast cancer).

In some embodiments, the cancer comprises colorectal cancer (e.g., microsatellite stable (MSS) colorectal cancer).

In some embodiments, the cancer comprises renal cell carcinoma.

In some embodiments, the cancer comprises lung cancer (e.g., non small cell lung cancer).

In some embodiments, the cancer comprises bladder cancer.

In some embodiments, the cancer comprises gastroesophageal cancer.

In some embodiments, the methods and solid dosage forms provided herein relate to the treatment of a leukemia. The term “leukemia” includes broadly progressive, malignant diseases of the hematopoietic organs/systems and is generally characterized by a distorted proliferation and development of leukocytes and their precursors in the blood and bone marrow. Non-limiting examples of leukemia diseases include, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia. Rieder cell leukemia. Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

In some embodiments, the methods and solid dosage forms provided herein relate to the treatment of a carcinoma. The term “carcinoma” refers to a malignant growth made up of epithelial cells tending to infiltrate the surrounding tissues, and/or resist physiological and non-physiological cell death signals and gives rise to metastases. Non-limiting exemplary types of carcinomas include, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma. Kulchitzky-cell carcinoma, large-cell carcinoma, lenticular carcinoma, carcinoma lenticularc, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti.

In some embodiments, the methods and solid dosage forms provided herein relate to the treatment of a sarcoma. The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma. Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias that can be treated using the methods and solid dosage forms described herein include Hodgkin's Disease. Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, plasmacytoma, colorectal cancer, rectal cancer, and adrenal cortical cancer.

In some embodiments, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma. Cloudman's melanoma. S91 melanoma, nodular melanoma subungal melanoma, and superficial spreading melanoma.

Particular categories of tumors that can be treated using methods and solid dosage forms described herein include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pincaloma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma, plasmacytoma, colorectal cancer, and rectal cancer.

Cancers treated in certain embodiments also include precancerous lesions, e.g., actinic keratosis (solar keratosis), moles (dysplastic nevi), acitinic chelitis (farmer's lip), cutaneous homs, Barrett's esophagus, atrophic gastritis, dyskeratosis congenita, sideropenic dysphagia, lichen planus, oral submucous fibrosis, actinic (solar) elastosis and cervical dysplasia.

Cancers treated in some embodiments include non-cancerous or benign tumors, e.g., of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

### Other Diseases and Disorders

In some embodiments, the methods and solid dosage forms described herein relate to the treatment of liver diseases. Such diseases include, but are not limited to, Alagille Syndrome, Alcohol-Related Liver Disease, Alpha-1 Antitrypsin Deficiency, Autoimmune Hepatitis, Benign Liver Tumors. Biliary Atresia, Cirrhosis, Galactosemia, Gilbert Syndrome, Hemochromatosis, Hepatitis A, Hepatitis B, Hepatitis C, Hepatic Encephalopathy, Intrahepatic Cholestasis of Pregnancy (ICP), Lysosomal Acid Lipase Deficiency (LAL-D), Liver Cysts, Liver Cancer, Newborn Jaundice, Primary Biliary Cholangitis (PBC), Primary Sclerosing Cholangitis (PSC), Reye Syndrome, Type I Glycogen Storage Disease, and Wilson Disease.

The methods and solid dosage forms described herein may be used to treat neurodegenerative and neurological diseases. In certain embodiments, the neurodegenerative and/or neurological disease is Parkinson's disease, Alzheimer's disease, prion disease, Huntington's disease, motor neuron diseases (MND), spinocerebellar ataxia, spinal muscular atrophy, dystonia, idiopathicintracranial hypertension, epilepsy, nervous system disease, central nervous system disease, movement disorders, multiple sclerosis, encephalopathy, peripheral neuropathy or post-operative cognitive dysfunction.

### Dysbiosis

In recent years, it has become increasingly clear that the gut microbiome (also called the “gut microbiota”) can have a significant impact on an individual's health through microbial activity and influence (local and/or distal) on immune and other cells of the host (Walker, W. A., Dysbiosis. The Microbiota in Gastrointestinal Pathophysiology. Chapter 25, 2017; Weiss and Thierry, Mechanisms and consequences of intestinal dysbiosis. Cellular and Molecular Life Sciences. (2017) 74(16):2959-2977. Zurich Open Repository and Archive, doi: https://doi.org/10.1007/s00018-017-2509-x)).

A healthy host-gut microbiome homeostasis is sometimes referred to as a “eubiosis” or “normobiosis,” whereas a detrimental change in the host microbiome composition and/or its diversity can lead to an unhealthy imbalance in the microbiome, or a “dysbiosis” (Hooks and O'Malley. Dysbiosis and its discontents. American Society for Microbiology. October 2017. Vol. 8. Issue 5, mBio 8:e01492-17, https://doi.org/10.1128/mBio.01492-17). Dysbiosis, and associated local or distal host inflammatory or immune effects, may occur where microbiome homeostasis is lost or diminished, resulting in: increased susceptibility to pathogens; altered host bacterial metabolic activity; induction of host proinflammatory activity and/or reduction of host anti-inflammatory activity. Such effects are mediated in part by interactions between host immune cells (e.g., T cells, dendritic cells, mast cells, NK cells, intestinal epithelial lymphocytes (TEC), macrophages and phagocytes) and cytokines, and other substances released by such cells and other host cells.

A dysbiosis may occur within the gastrointestinal tract (a “gastrointestinal dysbiosis” or “gut dysbiosis”) or may occur outside the lumen of the gastrointestinal tract (a “distal dysbiosis”). Gastrointestinal dysbiosis is often associated with a reduction in integrity of the intestinal epithelial barrier, reduced tight junction integrity and increased intestinal permeability. Citi, S. Intestinal Barriers protect against disease, Science 359:1098-99 (2018); Srinivasan et al., TEER measurement techniques for in vitro barrier model systems. J Lab. Autom. 20:107-126 (2015). A gastrointestinal dysbiosis can have physiological and immune effects within and outside the gastrointestinal tract.

The presence of a dysbiosis has been associated with a wide variety of diseases and conditions including: infection, cancer, autoimmune disorders (e.g., systemic lupus erythematosus (SLE)) or inflammatory disorders (e.g., functional gastrointestinal disorders such as inflammatory bowel disease (IBD), ulcerative colitis, and Crohn's disease), neuroinflammatory diseases (e.g., multiple sclerosis), transplant disorders (e.g., graft-versus-host disease), fatty liver disease, type I diabetes, rheumatoid arthritis, Sjögren's syndrome, celiac disease, cystic fibrosis, chronic obstructive pulmonary disorder (COPD), and other diseases and conditions associated with immune dysfunction. Lynch et al., The Human Microbiome in Health and Disease, N. Engl. J. Med 0.375:2369-79 (2016), Carding et al., Dysbiosis of the gut microbiota in disease. Microb. Ecol. Health Dis. (2015): 26: 10: 3402/mehd.v26.2619; Levy et al, Dysbiosis and the Immune System, Nature Reviews Immunology 17:219 (April 2017)

Exemplary pharmaceutical compositions disclosed herein can treat a dysbiosis and its effects by modifying the immune activity present at the site of dysbiosis. As described herein, such compositions can modify a dysbiosis via effects on host immune cells, resulting in, e.g., an increase in secretion of anti-inflammatory cytokines and/or a decrease in secretion of pro-inflammatory cytokines, reducing inflammation in the subject recipient or via changes in metabolite production.

Exemplary pharmaceutical compositions and/or solid dosage forms disclosed herein that are useful for treatment of disorders associated with a dysbiosis contain one or more types of immunomodulatory bacteria (e.g., anti-inflammatory bacteria) and/or mEVs (microbial extracellular vesicles) derived from such bacteria. Such compositions are capable of affecting the recipient host's immune function, in the gastrointestinal tract, and/or a systemic effect at distal sites outside the subject's gastrointestinal tract.

Exemplary pharmaceutical compositions and/or solid dosage forms disclosed herein that are useful for treatment of disorders associated with a dysbiosis contain a population of immunomodulatory bacteria of a single bacterial species (e.g., a single strain) (e.g., anti-inflammatory bacteria) and/or mEVs derived from such bacteria. Such compositions are capable of affecting the recipient host's immune function, in the gastrointestinal tract, and/or a systemic effect at distal sites outside the subject's gastrointestinal tract.

In one embodiment, pharmaceutical compositions and/or solid dosage forms containing an isolated population of immunomodulatory bacteria (e.g., anti-inflammatory bacterial cells) and/or mEVs derived from such bacteria are administered (e.g., orally) to a mammalian recipient in an amount effective to treat a dysbiosis and one or more of its effects in the recipient. The dysbiosis may be a gastrointestinal tract dysbiosis or a distal dysbiosis.

In another embodiment, pharmaceutical compositions and/or solid dosage forms of the instant invention can treat a gastrointestinal dysbiosis and one or more of its effects on host immune cells, resulting in an increase in secretion of anti-inflammatory cytokines and/or a decrease in secretion of pro-inflammatory cytokines, reducing inflammation in the subject recipient.

In another embodiment, the pharmaceutical compositions and/or solid dosage forms can treat a gastrointestinal dysbiosis and one or more of its effects by modulating the recipient immune response via cellular and cytokine modulation to reduce gut permeability by increasing the integrity of the intestinal epithelial barrier.

In another embodiment, the pharmaceutical compositions and/or solid dosage forms can treat a distal dysbiosis and one or more of its effects by modulating the recipient immune response at the site of dysbiosis via modulation of host immune cells.

Other exemplary pharmaceutical compositions and/or solid dosage forms are useful for treatment of disorders associated with a dysbiosis, which compositions contain one or more types of bacteria and/or mEVs capable of altering the relative proportions of host immune cell subpopulations, e.g., subpopulations of T cells, immune lymphoid cells, dendritic cells, NK cells and other immune cells, or the function thereof, in the recipient.

Other exemplary pharmaceutical compositions and/or solid dosage forms are useful for treatment of disorders associated with a dysbiosis, which compositions contain a population of immunomodulatory bacteria and/or mEVs of a single bacterial species e.g., a single strain) capable of altering the relative proportions of immune cell subpopulations, e.g., T cell subpopulations, immune lymphoid cells, NK cells and other immune cells, or the function thereof, in the recipient subject.

In one embodiment, the invention provides methods of treating a gastrointestinal dysbiosis and one or more of its effects by orally administering to a subject in need thereof a pharmaceutical composition and/or solid dosage form which alters the microbiome population existing at the site of the dysbiosis. The pharmaceutical composition and/or solid dosage form can contain one or more types of immunomodulatory bacteria or mEVs or a population of immunomodulatory bacteria and/or mEVs of a single bacterial species (e.g., a single strain).

In one embodiment, the invention provides methods of treating a distal dysbiosis and one or more of its effects by orally administering to a subject in need thereof a pharmaceutical composition and/or solid dosage form which alters the subject's immune response outside the gastrointestinal tract. The pharmaceutical composition and/or solid dosage form can contain one or more types of immunomodulatory bacteria or mEVs or a population of immunomodulatory bacteria and/or mEVs of a single bacterial species (e.g., a single strain).

In exemplary embodiments, pharmaceutical compositions and/or solid dosage forms useful for treatment of disorders associated with a dysbiosis stimulate secretion of one or more anti-inflammatory cytokines by host immune cells. Anti-inflammatory cytokines include, but are not limited to, IL-10, IL-13, IL-9, IL-4, IL-5, TGFβ, and combinations thereof. In other exemplary embodiments, pharmaceutical compositions and/or solid dosage forms useful for treatment of disorders associated with a dysbiosis that decrease (e.g., inhibit) secretion of one or more pro-inflammatory cytokines by host immune cells. Pro-inflammatory cytokines include, but are not limited to, IFNγ, IL-12p70, IL-1α, IL-6, IL-8, MCP1, MIP1α, MIP1β, TNFα, and combinations thereof. Other exemplary cytokines are known in the art and are described herein.

In another aspect, the invention provides a method of treating or preventing a disorder associated with a dysbiosis in a subject in need thereof, comprising administering (e.g., orally administering) to the subject a therapeutic composition in the form of a probiotic or medical food comprising bacteria and/or mEVs in an amount sufficient to alter the microbiome at a site of the dysbiosis, such that the disorder associated with the dysbiosis is treated.

In another embodiment, a therapeutic composition of the instant invention in the form of a probiotic or medical food may be used to prevent or delay the onset of a dysbiosis in a subject at risk for developing a dysbiosis.

### Infection

Inflammation can be a protective response to harmful stimuli, such as invading pathogens, damaged cells, toxic compounds, or cancerous cells. However, excessive inflammatory responses to such stimuli can result in serious adverse effects, including tissue damage and even death. For example, production of pro-inflammatory cytokines such as interleukin-8 (IL-8), interleukin-6 (IL-6), interleukin-1 beta (IL-1β), and tumor necrosis factor alpha (TNFα) in response to many viral infections is one of the primary causes of the adverse symptoms associated with infection (including, in some cases, death). For example, release of inflammatory cytokines has been associated with disease severity resulting from infection by a number of viruses, including infection by coronaviruses (e.g., SARS-CoV-2, the virus that causes Coronavirus Disease 2019 (COVID-19)), influenza viruses, and respiratory syncytial viruses. For example, patients with severe COVID-19 often exhibit elevated levels of inflammatory cytokines in their lungs, which contributes to lung damage experienced by the COVID-19 patients.

In some embodiments, the methods and solid dosage forms described herein relate to the treatment or prevention of bacterial septic shock, cytokine storm and/or viral infection.

In some embodiments, the methods and solid dosage forms described herein relate to the treatment or prevention of a viral infection such as a respiratory viral infection, such as a coronavirus infection (e.g., a MERS (Middle East Respiratory Syndrome) infection, a severe acute respiratory syndrome (SARS) infection, such as a SARS-CoV-2 infection), an influenza infection, and/or a respiratory syncytial virus infection. In some embodiments, the methods and and solid dosage forms described herein provided herein are for the treatment of a coronavirus infection (e.g., a MERS infection, a severe acute respiratory syndrome (SARS) infection, such as a SARS-CoV-2 infection). In some embodiments, provided herein are methods and solid dosage forms for treating COVID-19.

In some embodiments, the methods and solid dosage forms described herein relate to the treatment or prevention of a viral infection. In some embodiments, the infection is a coronavirus infection, an influenza infection, and/or a respiratory syncytial virus infection. In some embodiments the viral infection is a SARS-CoV-2 infection.

In some embodiments, an additional therapy is administered to the subject. In some embodiments, the additional therapy comprises an antiviral medication. In some embodiments, the additional therapy comprises an antiviral medication such as ribavirin, neuraminidase inhibitor, protease inhibitor, recombinant interferons, antibodies, oseltamivir, zanamivir, peramivir or baloxavir marboxil. In some embodiments, the additional therapy comprises hydroxychloroquine and/or chloroquine. In some embodiments, the additional therapy comprises remdesivir. In some embodiments, the additional therapy comprises plasma from a subject who has recovered from infection by the same virus that is infecting the subject (e.g., plasma from a subject who has recovered from SARS-CoV-2 infection). In some embodiments, the additional therapy comprises an anti-inflammatory agent such as NSAIDs or anti-inflammatory steroids. In some embodiments, the additional therapy comprises dexamethasone.

In some embodiments, the additional therapy comprises an antibody specific for IL-6 and/or the IL-6 receptor. In some embodiments, the additional therapy comprises tocilizumab (Actemra®). In some embodiments, the additional therapy comprises sarilumab (Kevzara®).

In some embodiments, the additional therapy can comprise an anti-viral therapy. For example, the anti-viral therapy can comprise a nucleotide analog, such as remdesivir, galidesivir or clevudine; a viral RNA polymerase inhibitor such as favipiravir or galidesivir; a protease inhibitor such as ritonavir, darunavir, or danoprevir; an inhibitor of viral membrane fusion such as umifenovir; and/or anti-SARS-CoV-2 plasma.

In some embodiments, the additional therapy can comprise an anti-inflammatory therapy. For example, the anti-inflammatory therapy can comprise a corticosteroid; sirolimus; anakinra; filamod; or an antibody. In some embodiments, the antibody can comprise a GMSF inhibitor, such as lenzilumab or gimsilumab; an anti-IL1 beta inhibitor such as canakinumab; an IL-6 inhibitor such as tocilizumab or siltuximab; an IL-6R inhibitor such as sarilumab; and/or a CCR5 antagonist such as leronlimab.

In some embodiments, the additional therapy can comprise a JAK inhibitor such as baricitinib, ruxolitinib, tofacitinib, and/or pacritinib.

In some embodiments, the additional therapy can comprise a TLR7 agonist such as imiquimod or reisquimod.

In some embodiments, the additional therapy can comprise a cell based therapy. For example, the cell based therapy can comprise Remestemcel-L; bone marrow stem cell therapy, such as MultiStem or Bm-Allo-MSC; mesenchymal stromal cells; and/or adiopose derived mesenchymal stem cells such as AstroStem.

In some embodiments, the additional therapy can comprise an ACE receptor inhibitor.

In some embodiments, the additional therapy can comprise a regulator of the Sigma 1 and/or Sigma 2 receptor.

### Methods of Making Enhanced Bacteria

In certain aspects, provided herein are methods of making engineered bacteria for the production of the bacteria and/or mEVs (such as smEVs and/or pmEVs) described herein. In some embodiments, the engineered bacteria are modified to enhance certain desirable properties. For example, in some embodiments, the engineered bacteria are modified to enhance the immunomodulatory and/or therapeutic effect of the bacteria and/or mEVs (such as smEVs and/or pmEVs) (e.g., either alone or in combination with another therapeutic agent), to reduce toxicity and/or to improve bacterial and/or mEV (such as smEV and/or pmEV) manufacturing (e.g., higher oxygen tolerance, improved freeze-thaw tolerance, shorter generation times). The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, CRISPR/Cas9, or any combination thereof.

In some embodiments of the methods provided herein, the bacterium is modified by directed evolution. In some embodiments, the directed evolution comprises exposure of the bacterium to an environmental condition and selection of bacterium with improved survival and/or growth under the environmental condition. In some embodiments, the method comprises a screen of mutagenized bacteria using an assay that identifies enhanced bacterium. In some embodiments, the method further comprises mutagenizing the bacteria (e.g., by exposure to chemical mutagens and/or UV radiation) or exposing them to a therapeutic agent (e.g., antibiotic) followed by an assay to detect bacteria having the desired phenotype (e.g., an in vivo assay, an ex vivo assay, or an in vitro assay).

### EXAMPLES

### Example 1: Powder Preparation Sample Protocol

After desired level of bacterial culture growth is achieved, centrifuge cultures, discard the supernatant, leaving the pellet as dry as possible. Resuspend pellet in desired cryoprotectant solution to create a formulated cell paste. The cryoprotectant may contain, e.g., maltodextrin, sodium ascorbate, sodium glutamate, and/or calcium chloride. Load the formulated cell paste onto stainless steel trays and load into a freeze drier, e.g., operating in automated mode with defined cycle parameters. The freeze dried product is fed into a milling machine and the resulting powder is collected.

Powders are stored (e.g., in vacuum sealed bags) at 2-8 degrees C. (e.g., at 4 degrees C.). e.g., in a desiccator.

### Example 2: Gamma-Irradiation: Sample Protocol

Powders are gamma-irradiated at 17.5 kGy radiation unit at ambient temperature. Frozen biomasses are gamma-irradiated at 25 kGy radiation unit in the presence of dry ice.

### Example 3: Preparation of a Capsule Comprising Prevotella histicola

The following recipe in Table 6 is prepared.

TABLE 6
Prevotella histicola Capsule Composition

| | | Reference to | |
|---|---|---|---|
| Name of ingredient(s) | Function | standards | % w/w |
| Prevotella histicola | Active | NA | 30-50%^{#} |
| (lyophilized) powder | ingredient | | |
| Mannitol | Diluent | USP/Ph. | 50-70%^{#} |
| | | Eur. | |
| Magnesium stearate | Lubricant | USP/Ph. | 1.0 |
| | | Eur. | |
| Colloidal silicon | Glidant | USP/Ph. | 0.5 |
| dioxide | | Eur. | |
| Total Fill Weight | | | 100 |
| Capsules, Size 0 | Capsule Shell | 1 unit | 1 unit |

^{#}Adjusted based on the potency of drug substance to ensure targeted strength.

The capsule is enteric coated for release at pH 5.5.

The Prevotella histicola strain referred to above has been deposited as Prevotella histicola Strain B (NRRL accession number B 50329).

### Example 4: Preparation of a Capsule Comprising Prevotella histicola

The following recipe in Table 7 is prepared.

TABLE 7
Prevotella histicola Capsule Composition

| | | Reference to | |
|---|---|---|---|
| Name of ingredient(s) | Function | standards | % w/w |
| Prevotella histicola | Active | NA | 30-50%^{#} |
| (lyophilized) powder | ingredient | | |
| Mannitol | Diluent | USP/Ph. | 45-70%^{#} |
| | | Eur. | |
| Magnesium stearate | Lubricant | USP/Ph. | 1.0 |
| | | Eur. | |
| Colloidal silicon | Glidant | USP/Ph. | 0.5 |
| dioxide | | Eur. | |
| Total Fill Weight | | | 100 |
| Capsules, Size 0 | Capsule Shell | 1 unit | 1 unit |

^{#}Adjusted based on the potency of drug substance to ensure targeted strength.

The capsule is enteric coated for release at pH 5.5.

The Prevotella histicola strain referred to above has been deposited as Prevotella histicola Strain B (NRRL accession number B 50329).

Batches of enteric coated capsules according to this recipe have been prepared.

### Example 5: Preparation of a Capsule Comprising Prevotella histicola

Capsules according to the following recipe in Table 8 were prepared:

TABLE 8
Prevotella histicola Capsule Composition

| Name of ingredient(s) | Function | % w/w |
|---|---|---|
| Prevotella histicola | Active | 50 |
| (lyophilized) powder | ingredient | |
| Mannitol | Diluent | 48.5 |
| Magnesium Stearate | Lubricant | 1.0 |
| Colloidal Silicon | Glidant | 0.5 |
| Dioxide | | |
| Total Fill Weight | | 100 |
| Capsules^{a}, Size 0 | Capsule Shell | 1 unit |

^{a}Composed of hydroxypropyl methylcellulose and titanium dioxide.

This capsule contained 1.6×10¹¹ cells.

The Prevotella histicola strain referred to above has been deposited as Prevotella histicola Strain B (NRRL accession number B 50329).

The capsule was banded with an HPMC-based banding solution.

The banded capsule was enteric coated with a poly(methacrylic acid-co-ethyl acrylate copolymer.

### Example 6: Preparation of a Capsule Comprising Prevotella histicola

Capsules according to the recipe in Table 9 are prepared.

TABLE 9
Prevotella histicola Capsule Composition

| | | Reference to | |
|---|---|---|---|
| Name of ingredient(s) | Function | standards | % w/w |
| Prevotella histicola | Active | NA | 10-90%^{#} |
| (lyophilized) powder | ingredient | | |
| Mannitol | Diluent | USP/Ph. | 8.5-88.5%^{#} |
| | | Eur. | |
| Magnesium stearate | Lubricant | USP/Ph. | 1.0 |
| | | Eur. | |
| Colloidal silicon | Glidant | USP/Ph. | 0.5 |
| dioxide | | Eur. | |
| Total Fill Weight | | | 100 |
| Capsules, Size 0 | Capsule Shell | 1 unit | 1 unit |

^{#}Adjusted based on the potency of drug substance to ensure targeted strength.

The capsule is enteric coated for release at pH 5.5.

The Prevotella histicola strain referred to above has been deposited as Prevotella histicola Strain B (NRRL accession number B 50329).

Batches of enteric coated capsules according to this recipe have been prepared.

### Example 7: Preparation of a Capsule Comprising Prevotella histicola

Capsules according to the following recipe in Table 10 were prepared:

TABLE 10
Prevotella histicola Capsule Composition

| | | 1.6 × 10¹⁰ | 8.0 × 10¹⁰ | 1.6 × 10¹¹ |
|---|---|---|---|---|
| | | Cells | Cells | Cells |
| Name of ingredient(s) | Function | % w/w | % w/w | % w/w |
| Prevotella histicola | Active | 13.51 ^{b} | 90.22 ^{b} | 50 |
| (lyophilized) powder | ingredient | | | |
| Mannitol | Diluent | 84.99 ^{b} | 8.28 ^{b} | 48.5 |
| Magnesium Stearate | Lubricant | 1.0 | 1.0 | 1.0 |
| Colloidal Silicon Dioxide | Glidant | 0.5 | 0.5 | 0.5 |
| Total Fill Weight | | 100 | 100 | 100 |
| Capsules^{a}, Size 0 | Capsule | 1 unit | 1 unit | I unit |
| | Shell | | | |

^{a}Composed of hydroxypropyl methylcellulose and titanium dioxide.
^{b} Adjusted based on the potency of drug substance to ensure targeted strength.

The capsule was banded with an HPMC-based banding solution.

The banded capsule was enteric coated with a poly(methacrylic acid-co-ethyl acrylate copolymer.

### Example 8: Preparation of a Capsule Comprising Prevotella histicola

Capsules according to the following recipe in Table 11 were prepared:

TABLE 11
Prevotella histicola Capsule Composition

| | | 3.35 × 10¹¹ Cells |
|---|---|---|
| Name of ingredient(s) | Function | % w/w |
| Prevotella histicola | Active | 50 |
| (lyophilized) powder | ingredient | |
| Mannitol (Pearlitol SD200) | Diluent | 48.5 |
| Magnesium Stearate | Lubricant | 1.0 |
| (Ligamed MF-2-V) | | |
| Colloidal Silicon Dioxide | Glidant | 0.5 |
| (Aerosil 200P) | | |
| Total Fill Weight | | 100 |
| Capsules^{a}, Size 0 | Capsule | 1 unit |
| | Shell | |

^{a}Swedish orange Vcap capsules

The Prevotella histicola strain referred to above has been deposited as Prevotella histicola Strain B (NRRL accession number B 50329).

The capsule was banded with an HPMC-based banding solution.

The banded capsule was enteric coated with Eudragit L30-D55, a poly(methacrylic acid-co-ethyl acrylate) copolymer.

In disintegration tests, the enteric coated capsule did not disintegrate at 0.1N HCl media. In pH 6.8 buffer, the capsule disintegrated in less than 11 minutes.

### Example 9: Preparation of a Capsule Comprising Veillonella parvula

Capsules according to the following recipe in Table 12 were prepared:

TABLE 12
Veillonella parvula Capsule Composition

| | Low Dose | High Dose |
|---|---|---|
| | % w/w | % w/w |
| Veillonella Parvula | 5.0 | 60.0 |
| Strain A powder | | |
| Mannitol | 93.0 | 38.0 |
| Silicon dioxide | 0.5 | 0.5 |
| Magnesium stearate | 1.5 | 1.5 |
| Total | 100.00 | 100.00 |

The Veillonella parvula Strain A bacteria in this capsule were gamma-irradiated.

The capsule was banded with an HPMC-based banding solution. The banded capsule was enteric coated with a poly(methacrylic acid-co-ethyl acrylate copolymer.

### Example 10: Preparation of a Capsule Comprising Veillonella parvula

The following recipe in Table 13 is prepared:

TABLE 13
Veillonella parvula Capsule Composition

| | | 4.5 × 10¹⁰ | 1.5 × 10¹¹ |
|---|---|---|---|
| Name of | | Cells | Cells |
| ingredient(s) | Function | % w/w | % w/w |
| Veillonella parvula | Active | 10.6 ^{b} | 40.0 ^{b} |
| strain A powder | ingredient | | |
| (lyophilized) | | | |
| Mannitol | Diluent | 87.4 ^{b} | 58.0 ^{b} |
| Magnesium Stearate | Lubricant | 1.5 | 1.5 |
| Colloidal Silicon | Glidant | 0.5 | 0.5 |
| Dioxide | | | |
| Total Fill Weight | | 100.0 | 100.0 |
| Capsules^{a}, Size 0 | Capsule Shell | 1 unit | 1 unit |

^{a}Composed of hydroxypropyl methylcellulose, titanium dioxide, and iron oxide.
^{b} Adjusted based on the potency of drug substance to ensure targeted strength.

The capsules are banded and enteric coated after encapsulation to prevent premature capsule disintegration in the stomach.

### Example 11: Lactococcus lactis Capsule in the Absence of Mannitol

In some aspects, a solid dosage form, e.g., capsule, is prepared and no diluent is present. For example, the capsule comprises powder, lubricant and glidant. As one example, the following recipe in Table 14 is prepared:

TABLE 14
Lactococcus lactis Capsule Recipe

| Name of | | 1.5 × 10¹¹ Cells^{b,c} |
|---|---|---|
| ingredient(s) | Function | % w/w |
| Lactococcus lactis ssp. | Active | 98.5 |
| cremoris powder | ingredient | |
| (lyophilized) | | |
| Mannitol | Diluent | 0.0 |
| Magnesium Stearate | Lubricant | 1.0 |
| Colloidal Silicon | Glidant | 0.5 |
| Dioxide | | |
| Total Fill Weight | | 100.0 |
| Capsules^{a}, Size 0 | Capsule | 1 unit |
| | Shell | |

^{a}Composed of hydroxypropyl methylcellulose, titanium dioxide, and iron oxide.
^{b}TCC—Total cell count
^{c}Total fill weight is adjusted based on the potency of drug substance to ensure 100% targeted strength

### Example 12: Lactococcus lactis Capsule with Microcrystalline Cellulose

In some aspects, microcrystalline cellulose can be used as a diluent (e.g., in place of mannitol) in the solid dose forms (such as capsules) provided herein. For example, the following recipe in Table 15 is prepared:

TABLE 15
Lactococcus lactis Capsule Recipe

| Name of | | 3 × 10¹⁰ Cells ^{b,c} |
|---|---|---|
| ingredient(s) | Function | % w/w |
| Lactococcus lactis | Active | 25.1 |
| ssp. cremoris | ingredient | |
| powder | | |
| (lyophilized) | | |
| Microcrystalline | Diluent | 73.4 |
| cellulose^{c} | | |
| Magnesium Stearate | Lubricant | 1.0 |
| Colloidal Silicon | Glidant | 0.5 |
| Dioxide | | |
| Total Fill Weight | | 100.0 |
| Capsules^{a}, Size 0 | Capsule | 1 unit |
| | Shell | |

^{a}Composed of hydroxypropyl methylcellulose and titanium dioxide.
^{b} TCC—Total cell count
^{c}Total fill weight is adjusted based on the potency of drug substance to ensure 100% targeted strength

### Example 13: Representative Strains as Sources for EVs

Secreted microbial extracellular vesicles (smEVs) were isolated from the strains listed in Table J. Information on the Gram staining, cell wall structure, and taxonomic classification for each strain is also provided in Table J.

Bacteria of the taxonomic groups listed in Table J (e.g., class, order, family, genus, species or strain) can be used in the solid dosage forms described herein.

mEVs of bacteria of the taxonomic groups listed in Table J (e.g., class, order, family, genus, species or strain) can be used in the solid dosage forms described herein.

TABLE J
Strains from which extracellular vesicles (EVs) were isolated

| | | Cell | | | | |
|---|---|---|---|---|---|---|
| | Gram- | envelope | | | | |
| Strain | stain | structure | Phylum | Class | Order | Family |
| Parabacteroides | Gram- | diderm | Bacteroidota | Bacteroidia | Bacteroidales | Porphyromonadaceae |
| distasonis | stain- | | | | | |
| DRLU022118 A | negative | | | | | |
| ILEUM-6 | | | | | | |
| Parabacteroides | Gram- | diderm | Bacteroidota | Bacteroidia | Bacteroidales | Porphyromonadaceae |
| goldsteinii S4 | stain- | | | | | |
| | negative | | | | | |
| Prevotella | Gram- | diderm | Bacteroidota | Bacteroidia | Bacteroidales | Prevotellaceae |
| histicola | stain- | | | | | |
| | negative | | | | | |
| Prevotella | Grom- | diderm | Bacteroidota | Bacteroidia | Bacteroidales | Prevotellaceae |
| histicola | stain- | | | | | |
| | negative | | | | | |
| Fournierella | Grom- | monoderm | Firmicutes | Clostridia | Eubacteriales | Oscillospiraceae |
| massiliensis S10 | stain- | | | | | (formely |
| GIMucosa-297 | negative | | | | | Ruminococcacea) |
| Harryflintia | Grom- | monoderm | Firmicutes | Clostridia | Eubacteriales | Oscillospiraceae |
| acetispora S4- | stain- | | | | | |
| M5 | negative | | | | | |
| Blautia | Gram- | monoderm | Firmicutes | Clostridia | Eubacteriales | Lachnospiraceae |
| massiliensis | stain- | | | | | |
| S1046-4A5 | negative | | | | | |
| Mediterraneibacter/ | Gram- | monoderm | Firmicutes | Clostridia | Eubacteriales | Lachnospiraceae |
| [Ruminococcus] | stain- | | | | | |
| gnavus S10 | negative | | | | | |
| GIMucosa-412 | | | | | | |
| Clostridioides | Gram- | monoderm | Firmicutes | Clostridia | Eubacteriales | Peptostreptococc |
| difficile S10 | stain- | | | | | aceae |
| GImucosa-525 | positive | | | | | |
| Aminipila sp. | Gram- | monoderm | Firmicutes | Clostridia | Eubacteriales | Clostridiales |
| S16-M4 | stain- | | | | | Family |
| | positive | | | | | XIII/Incertae |
| | | | | | | sedis |
| | | | | | | 41/[Eubacteriales, |
| | | | | | | no family] |
| Megasphaera sp. | Gram- | diderm | Firmicutes | Negativicutes | Veillonellales | Veillonellaceae |
| S29-N3 | stain- | | | | | |
| | negative | | | | | |
| Megasphaera sp. | Gram- | diderm | Firmicutes | Negativicutes | Veillonellales | Veillonellaceae |
| S1007 | stain- | | | | | |
| | negative | | | | | |
| Selenomonas | Gram- | diderm | Firmicutes | Negativicutes | Selenomonadales | Selenomonadaceae |
| felix S34N-300R | stoin- | | | | | |
| | negative | | | | | |
| Veillonella | Gram- | diderm | Firmicutes | Negativicutes | Veillonellales | Veillonellaceae |
| parvula | stain- | | | | | |
| S14Ileum-201 | negative | | | | | |
| Propionispora | Gram- | diderm | Firmicutes | Negativicutes | Selenomonadales | Sporomusaceae |
| sp. DSM100705- | stain- | | | | | |
| 1A | negative | | | | | |
| Rarimicrobium | Gram- | diderm | Synergistot | Synergistia | Synergistales | Synergistaceae |
| hominis S24RS2- | stain- | | | | | |
| T2-5 | negative | | | | | |
| Cloacibacillus | Gram- | diderm | Synergistot | Synergistia | Synergistales | Synergistaceae |
| evryensis S29- | stain- | | | | | |
| M8 | negative | | | | | |
| Veillonella | Gram- | diderm | Firmicutes | Negativicutes | Veillonellales | Veillonellaceae |
| parvula S14-205 | stain- | | | | | |
| | negative | | | | | |
| Veillonella | Gram- | diderm | Firmicutes | Negativicutes | Veillonellales | Veillonellaceae |
| sp/dispar | stain- | | | | | |
| ECD01-DP-201 | negative | | | | | |
| Veillonella | Gram- | diderm | Firmicutes | Negativicutes | Veillonellales | Veillonellaceae |
| parvulal/dispar | stain- | | | | | |
| ECD01-DP-223 | negative | | | | | |
| Veillonella | Gram- | diderm | Firmicutes | Negativicutes | Veillonellales | Veillonellaceae |
| parvala S16 | stain- | | | | | |
| GIMucosa-95 | negative | | | | | |

### Example 14: Preparation of a Solid Dosage Form Comprising Prevotella histicola smEVs

Capsule of the recipes in Table 16 were prepared:

TABLE 16
Compositions of active capsules

| | Low | Medium | High |
|---|---|---|---|
| Capsules | Strength | Strength | Strength |
| Prevotella histicola smEVs | 10% (LS | 10% (HS | 90% (HS |
| (drug substance/powder) | DS) | DS) | DS) |
| Mannitol | 187.5% | 87.5% | 7.5% |
| SiO2 | 1.0% | 1.0% | 1.0% |
| Magnesium stearate | 1.5% | 1.5% | 1.5% |
| Capsule filled weight (mg) | 360 | 360 | 360 |

The Prevotella histicola smEVs in Table 16 are from strain Prevotella histicola Strain B 50329 (NRRL accession number B 50329).

HS DS: high strength drug substance.

LS DS: low strength drug substance.

LS DS was prepared by diluting HS DS 10× (using lyophilization excipients) before lyophilization.

To prepare the pharmaceutical composition capsules, wet granulation was performed on the drug substance (pharmaceutical agent) containing the smEVs. Drug substance was (i) mixed with water; (ii) dried on a fluid bed dryer; (iii) milled; (iv) then blended with the drug product excipients provided in Table 16.

The capsules were size 0.

### Example 15: Preparation of a Capsule Comprising Prevotella histicola

Capsules according to the following recipe in Table 17 were prepared:

TABLE 17
Composition of Prevotella histicola Coated
Capsules - 3.2 × 10¹¹ cells/capsule

| Name of ingredient(s) | % w/w |
|---|---|
| Prevotella histicola | 49.2^{a} |
| (lyophilized) powder | |
| Mannitol | 49.3^{a} |
| Magnesium stearate | 1.0 |
| Colloidal silicon dioxide | 0.5 |
| Total fill weight | 100.0 |
| Capsules, Size 0 | 1 unit |

^{a}Adjusted based on the potency of drug substance to ensure targeted strength.

The Prevotella histicola strain referred to above has been deposited as Prevotella histicola Strain B (NRRL accession number B 50329).

The capsule was banded with an HPMC-based banding solution.

The banded capsule was enteric coated with Eudragit L30-D55, a poly(methacrylic acid-co-ethyl acrylate) copolymer.

### Example 16: Properties of Capsules Comprising Prevotella histicola

Batch A: A batch of capsules according to the recipe for 1.6×10¹⁰ TCC/capsule in Table 6 was prepared (Batch A). Upon evaluation, the capsules had a TCC of 1.7×10¹⁰ as determined by Couter counter, and a water content of 0.99%, as determined by Karl Fisher (Ph. Eur. 2.5.32). The acceptable TCC range is set at 0.8×10¹⁰ to 2.4×10¹⁰.

Batch B: A batch of capsules according to the recipe for 8×10¹⁰ TCC/capsule in Table 6 was prepared (Batch B). Upon evaluation, the capsules had a TCC of 6.9×10¹⁰ as determined by Couter counter, and a water content of 4.0%, as determined by Karl Fisher (Ph. Eur. 2.5.32). The acceptable TCC range is set at 4×10¹⁰ to 1.2×10¹¹.

Batch C: A batch of capsules according to the recipe for 8×10¹⁰ TCC/capsule in Table 6 was prepared (Batch C). Upon evaluation, the capsules had a TCC of 7.7×10¹⁰ as determined by Couter counter, and a water content of 5.9%, as determined by Karl Fisher (Ph. Eur. 2.5.32). The acceptable TCC range is set at 4×10¹⁰ to 1.2×10¹¹.

Batch D: A batch of capsules according to the recipe for 8×10¹⁰ TCC/capsule in Table 6 was prepared (Batch D). Upon evaluation, the capsules had a TCC of 7.6×10¹⁰ as determined by Couter counter, and a water content of 3.4%, as determined by Karl Fisher (Ph. Eur. 2.5.32). The acceptable TCC range is set at 4×10¹⁰ to 1.2×10¹¹.

Batch E: A batch of capsules according to the recipe for 8×10¹⁰ TCC/capsule in Table 6 was prepared (Batch E). Upon evaluation, the capsules had a TCC of 9.4×10¹⁰ as determined by Couter counter, and a water content of 1.9%, as determined by Karl Fisher (Ph. Eur. 2.5.32). The acceptable TCC range is set at 4×10¹⁰ to 1.2×10¹¹.

Batch F: A batch of capsules according to the recipe for 3.2×10¹¹ TCC/capsule in Table 17 was prepared (Batch F). Upon evaluation, the capsules had a TCC of 3.2×10¹¹ as determined by Couter counter, and a water content of 5.2%, as determined by Karl Fisher (Ph. Eur. 2.5.32). The acceptable TCC range is set at 1.6×10¹¹ to 4.8×10¹¹.

Batch G: A batch of capsules according to the recipe for 3.2×10¹¹ TCC/capsule in Table 17 was prepared (Batch G). Upon evaluation, the capsules had a TCC of 2.9×10¹¹ as determined by Couter counter, and a water content of 3.5%, as determined by Karl Fisher (Ph. Eur. 2.5.32). The acceptable TCC range is set at 1.6×10¹¹ to 4.8×10¹¹.

### Example 17: Batch a Stability Data

The stability of Batch A capsules was assessed.

Capsule Content and Potency: Total Cells/Capsule:

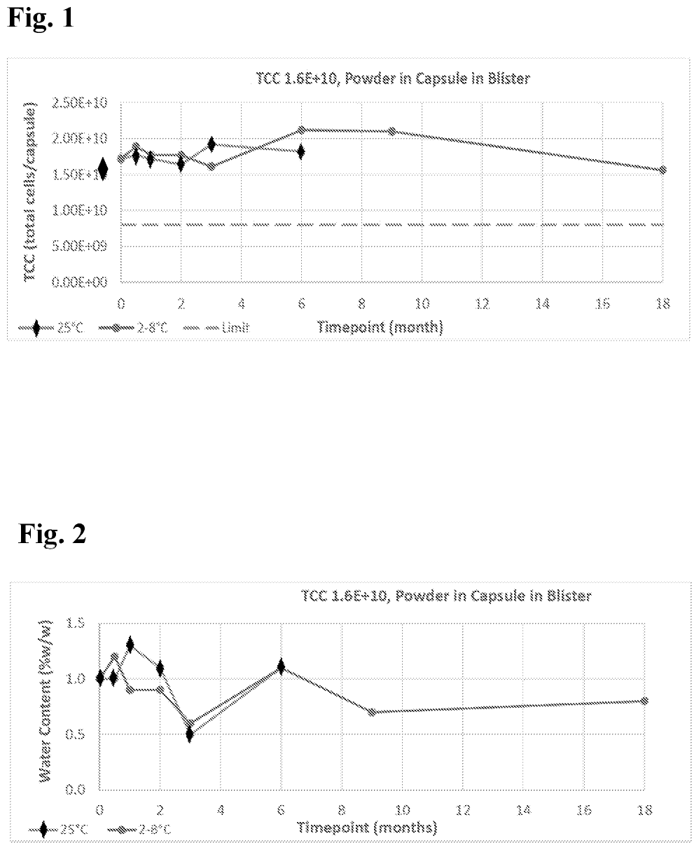

The total cells/capsule was determined for the batch for the durations shown at long-term (2-8° C.) and accelerated (25° C./60% RH (relative humidity)) storage conditions. TCC was determined by Coulter counter. The data are presented in FIG. 1. The total cells/capsule for the batch were above 50% of the target value and within stability specifications for both long-term (5° C.) and accelerated (25° C./60% RH (relative humidity)) storage conditions all time points tested.

Water Content:

The water content was determined for the batch for the durations shown for both long-term (2-8° C.) and accelerated (25° C./60% RH) storage conditions for the durations shown. The results are shown in FIG. 2. There are no apparent trends in water content on storage indicating that the capsule itself along with blister packaging configuration is providing adequate protection against water ingress.

### Example 18: Batch B Stability Data

The stability of Batch B capsules was assessed.

Capsule Content and Potency: Total Cells/Capsule:

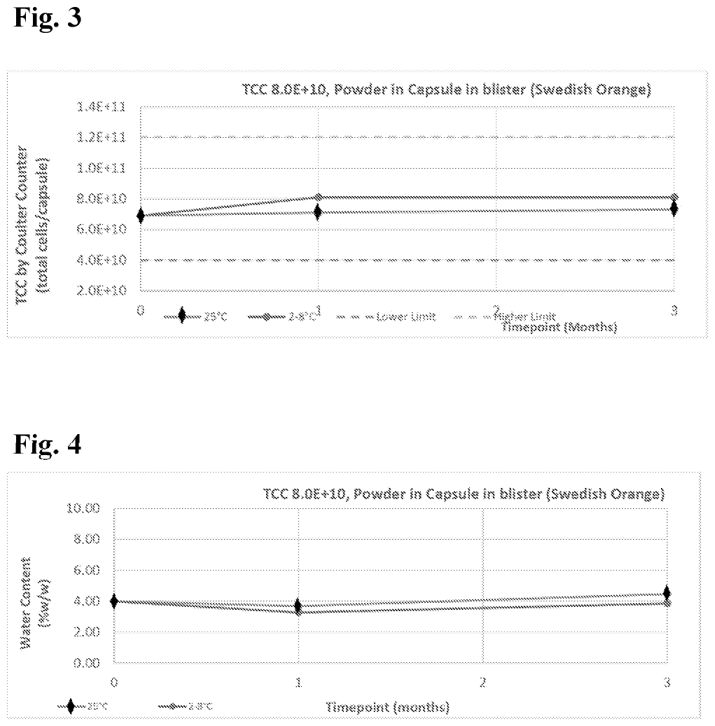

The total cells/capsule was determined for the batch for the durations shown at long-term (2-8° C.) and accelerated (25° C./60% RH (relative humidity)) storage conditions. TCC was determined by Coulter counter. The data are presented in FIG. 3. The total cells/capsule for the batch were above 50% of the target value and within stability specifications for both long-term (5° C.) and accelerated (25° C./60% RH (relative humidity)) storage conditions all time points tested.

Water Content:

The water content was determined for the batch for the durations shown for both long-term (2-8° C.) and accelerated (25° C./60% RH) storage conditions for the durations shown. The results are shown in FIG. 4. There are no apparent trends in water content on storage indicating that the capsule itself along with blister packaging configuration is providing adequate protection against water ingress.

### Example 19: Batch C Stability Data

The stability of Batch C capsules was assessed.

Capsule Content and Potency: Total Cells/Capsule:

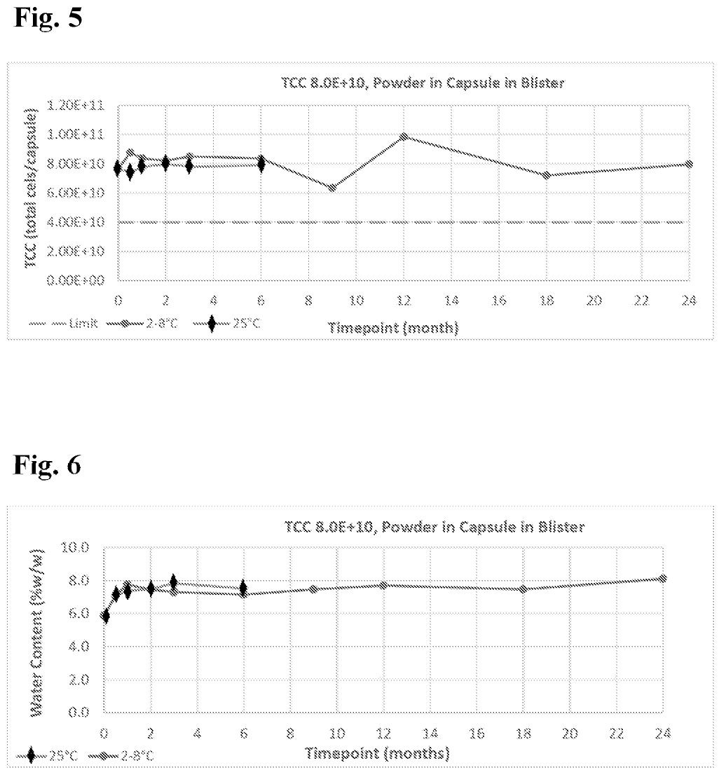

The total cells/capsule was determined for the batch for the durations shown at long-term (2-8° C.) and accelerated (25° C./60% RH (relative humidity)) storage conditions. TCC was determined by Coulter counter. The data are presented in FIG. 5. The total cells/capsule for the batch were above 50% of the target value and within stability specifications for both long-term (5° C.) and accelerated (25° C./60% RH (relative humidity)) storage conditions all time points tested.

Water Content:

The water content was determined for the batch for the durations shown for both long-term (2-8° C.) and accelerated (25° C./60% RH) storage conditions for the durations shown. The results are shown in FIG. 6. There are no apparent trends in water content on storage indicating that the capsule itself along with blister packaging configuration is providing adequate protection against water ingress.

### Example 20: Batch F Stability Data

The stability of Batch F capsules was assessed.

Capsule Content and Potency: Total Cells/Capsule:

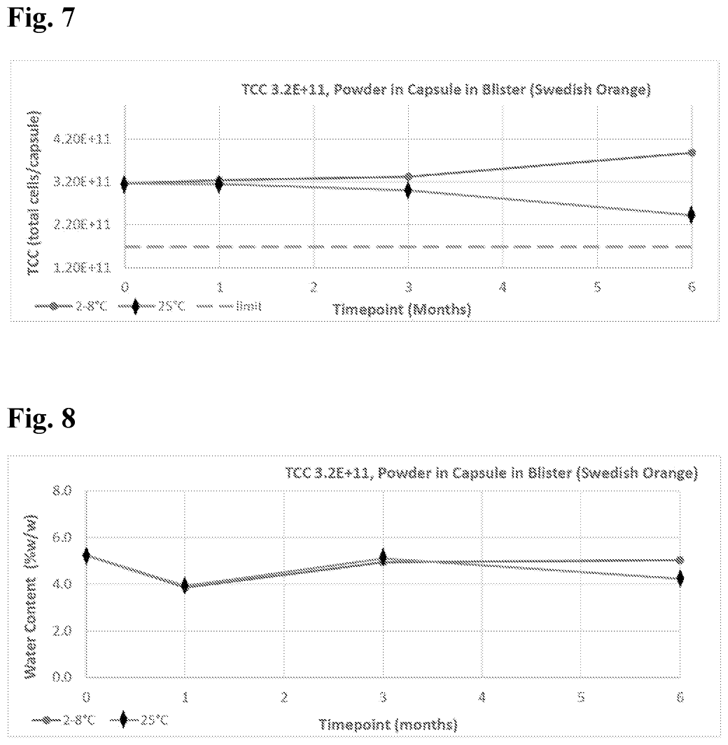

The total cells/capsule was determined for the batch for the durations shown at long-term (2-8° C.) and accelerated (25° C./60% RH (relative humidity)) storage conditions. TCC was determined by Coulter counter. The data are presented in FIG. 7. The total cells/capsule for the batch were above 50% of the target value and within stability specifications for both long-term (5° C.) and accelerated (25° C./60% RH (relative humidity)) storage conditions all time points tested.

Water Content:

The water content was determined for the batch for the durations shown for both long-term (2-8° C.) and accelerated (25° C./60% RH) storage conditions for the durations shown. The results are shown in FIG. 8. There are no apparent trends in water content on storage indicating that the capsule itself along with blister packaging configuration is providing adequate protection against water ingress.

### Example 21: Preparation of a Capsule Comprising Veillonella parvula

Capsules according to the following recipe in Table 18 were prepared:

TABLE 18
Veillonella parvula Capsule Composition

| | | 4.5 × 10¹⁰ | 1.5 × 10¹¹ |
|---|---|---|---|
| Name of | | Cells | Cells |
| ingredient(s) | Function | % w/w | % w/w |
| Veillonella parvula | Active | 10.6 ^{b} | 40.0 ^{b} |
| strain A powder | ingredient | | |
| (ly ophilized) | | | |
| Mannitol | Diluent | 87.4 ^{b} | 58.0 b |
| Magnesium Stearate | Lubricant | 1.5 | 1.5 |
| Colloidal Silicon | Glidant | 0.5 | 0.5 |
| Dioxide | | | |
| Total Fill Weight | | 100.0 | 100.0 |
| Capsules^{a}, Size 0 | Capsule Shell | 1 unit | 1 unit |

^{a}Composed of hydroxypropyl methylcellulose, titanium dioxide, and iron oxide.
^{b} Adjusted based on the potency of drug substance to ensure targeted strength.

The Veillonella parvula Strain A bacteria in these capsules were gamma-irradiated.

The capsules were banded with an HPMC-based banding solution. The banded capsules were enteric coated with a poly(methacrylic acid-co-ethyl acrylate copolymer.

The capsules were banded and enteric coated after encapsulation.

### Example 22: Properties of Capsules Comprising Veillonella parvula

4.5×10¹⁰ dose batch: A batch of capsules according to the recipe for 4.5×10¹⁰ TCC/capsule in Table 2 was prepared. Upon evaluation, the capsules had a TCC of 5×10¹⁰ as determined by Couter counter, and a water content of 1.1%, as determined by Karl Fisher (Ph. Eur. 2.5.32). The acceptable TCC range is set at 2.25×10¹⁰ to 6.75×10¹⁰.

1.5×10¹¹ dose batch: A batch of capsules according to the recipe for 1.5×10¹¹ TCC/capsule in Table 2 was prepared. Upon evaluation, the capsules had a TCC of 1.62×10¹¹ as determined by Couter counter, and a water content of 3.6%, as determined by Karl Fisher (Ph. Eur. 2.5.32). The acceptable TCC range is set at 7.5×10¹⁰ to 2.25×10¹¹.

### Example 23: Three-Month Stability Data for Low and High Dose Batches

The stability of capsules from the low and high dose batches was assessed.

Capsule Content and Potency: Total Cells/Capsule:

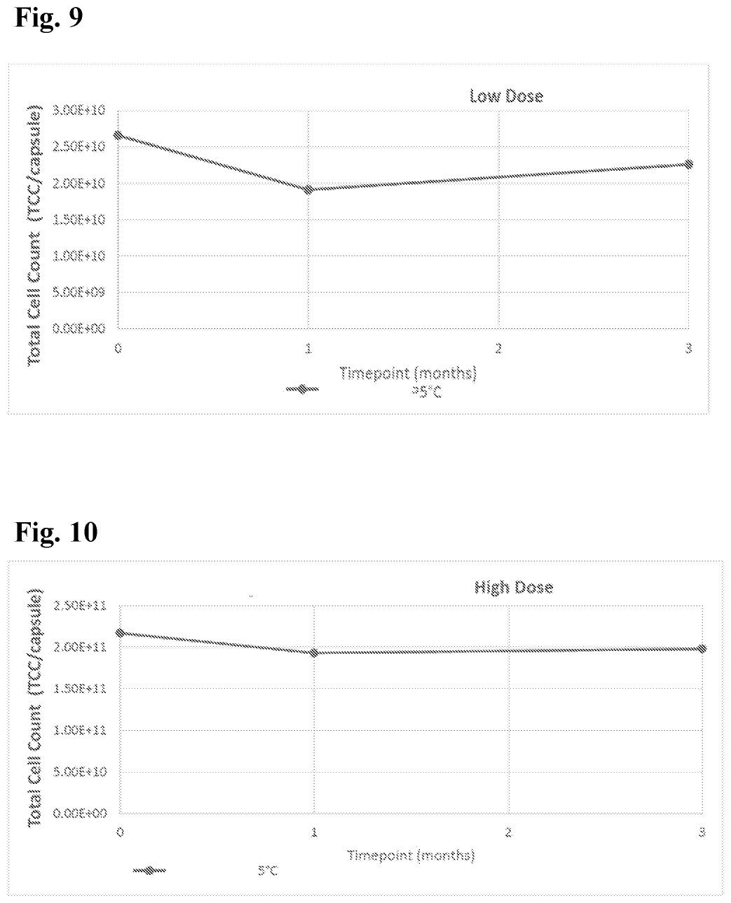

The total cells/capsule was determined for the batches for the durations shown at long-term (2-8° C.) (abbreviation: 5° C.) storage conditions. TCC was determined by Coulter counter. The data are presented in FIG. 9 (low dose batch) and FIG. 10 (high dose batch). The total cells/capsule for both batches were within the set stability specification at long term storage for the time points tested to date. For the low dose batch, the acceptance criteria was ≥2.25×10¹⁰ cells/capsule, measured as TCC (determined by Coulter counter). For the high dose batch, the acceptance criteria was ≥7.5×10¹⁰ cells/capsule, measured as TCC (determined by Coulter counter).

Water Content:

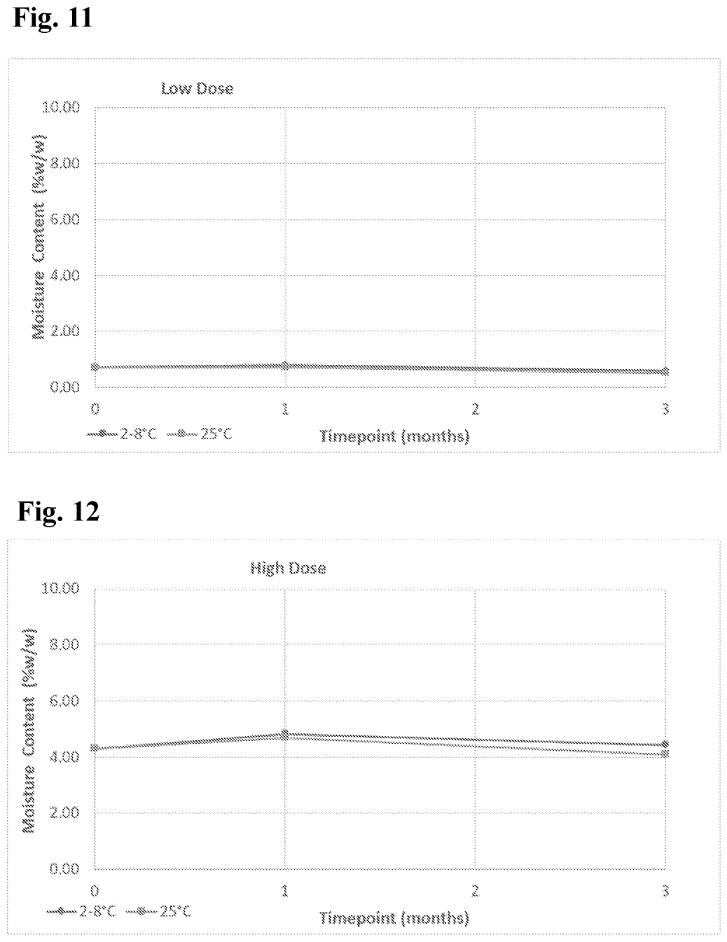

The water content was determined for the batches for the durations shown for both long-term (2-8° C.) (abbreviation: 5° C.) and accelerated (25° C. (±2° C.)/60% (±5%) RH (relative humidity)) (abbreviation: 25° C.)) storage conditions for the durations shown. The results are shown in FIG. 11 (low dose batch) and FIG. 12 (high dose batch). There are no apparent trends in water content on storage for low dose or high dose batches indicating that the blister packaging configuration is providing adequate protection against water ingress. The low and high dose drug products are shown to be stable both at regular (5° C.) and accelerated temperature (25° C.) conditions for up to 3 months.

### Example 24: Six-Month Stability Data for Low and High Dose Batches

Six-month stability data were obtained for the low and high dose batches described in Example 23.

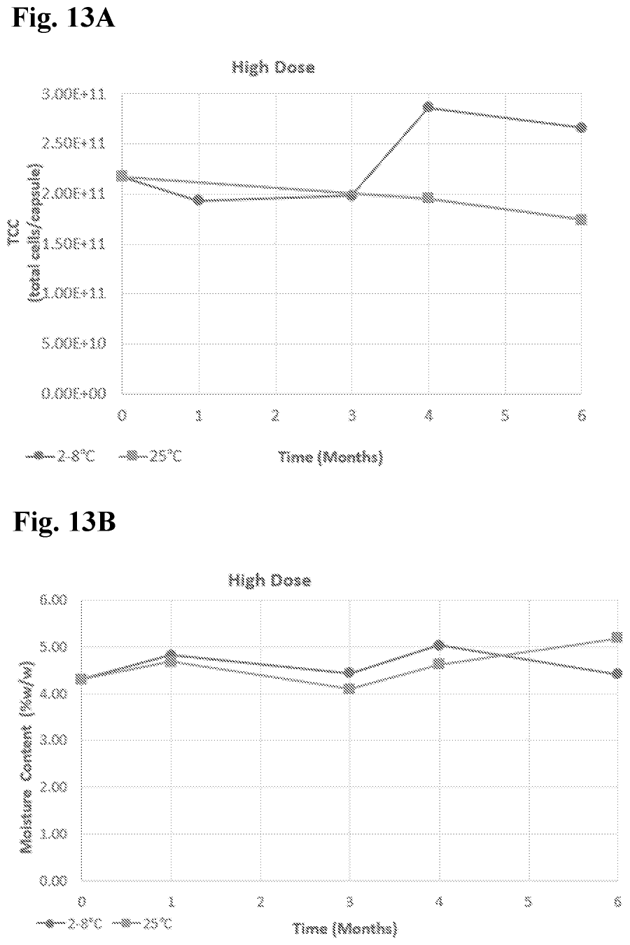

FIGS. 13A and B are graphs showing 6-month Stability Profiles for the high dose batch. FIG. 13A is a graph showing Total Cells/Capsule Stability Profile overtime long-term (2-8° C. (abbreviation: 5° C.)) and accelerated (25° C./60% RH (abbreviation: 25° C.)) storage conditions for the high dose batch. Total Cell Count (TCC) was determined by Coulter counter. FIG. 13B is a graph showing Moisture Content Stability Profile over time long-term (2-8° C.) and accelerated (25° C./60% RH (abbreviation: 25° C.)) storage conditions for the high dose batch. Moisture r content was determined by the Karl Fisher method.

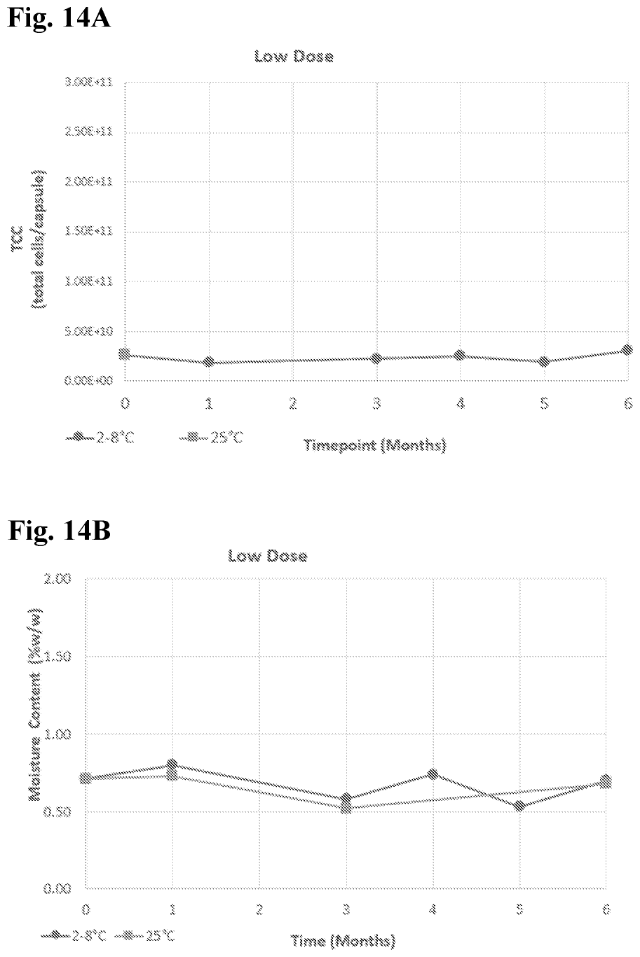

FIGS. 14A and B are graphs showing 6-month Stability Profiles for the low dose batch. FIG. 14A is a graph showing Total Cells/Capsule Stability Profile over time long-term (2-8° C. (abbreviation: 5° C.)) and accelerated (25° C./60% RH (abbreviation: 25° C.)) storage conditions for the low dose batch. Total Cell Count (TCC) was determined by Coulter counter. FIG. 14B is a graph showing Moisture Content Stability Profile over time long-term (2-8° C.) and accelerated (25° C./60% RH (abbreviation: 25° C.)) storage conditions for the low dose batch. Moisture content was determined by the Karl Fisher method.

### Example 25: Six-Month Stability Data for Low and High Dose Batches

Six-month stability data were obtained for second low and high dose batches.

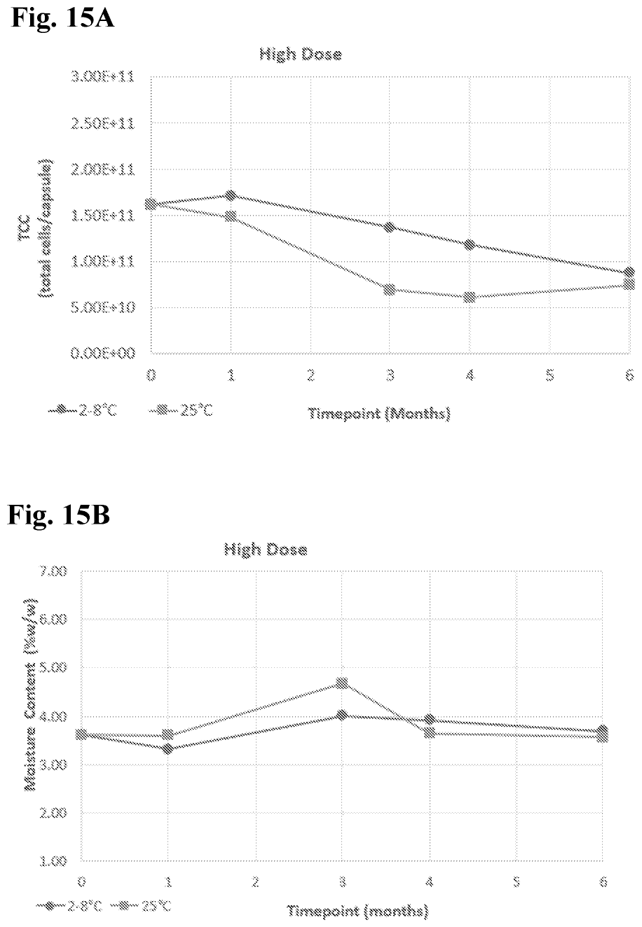

FIGS. 15A and B are graphs showing 6-month Stability Profiles for a second high dose batch. FIG. 15A is a graph showing Total Cells/Capsule Stability Profile over time long-term (2-8° C. (abbreviation: 5° C.)) and accelerated (25° C./60% RH (abbreviation: 25° C.)) storage conditions for the second high dose batch. Total Cell Count (TCC) was determined by Coulter counter. FIG. 15B is a graph showing Moisture Content Stability Profile over time long-term (2-8° C.) and accelerated (25° C./60% RH (abbreviation: 25° C.)) storage conditions for the second high dose batch. Moisture content was determined by the Karl Fisher method.

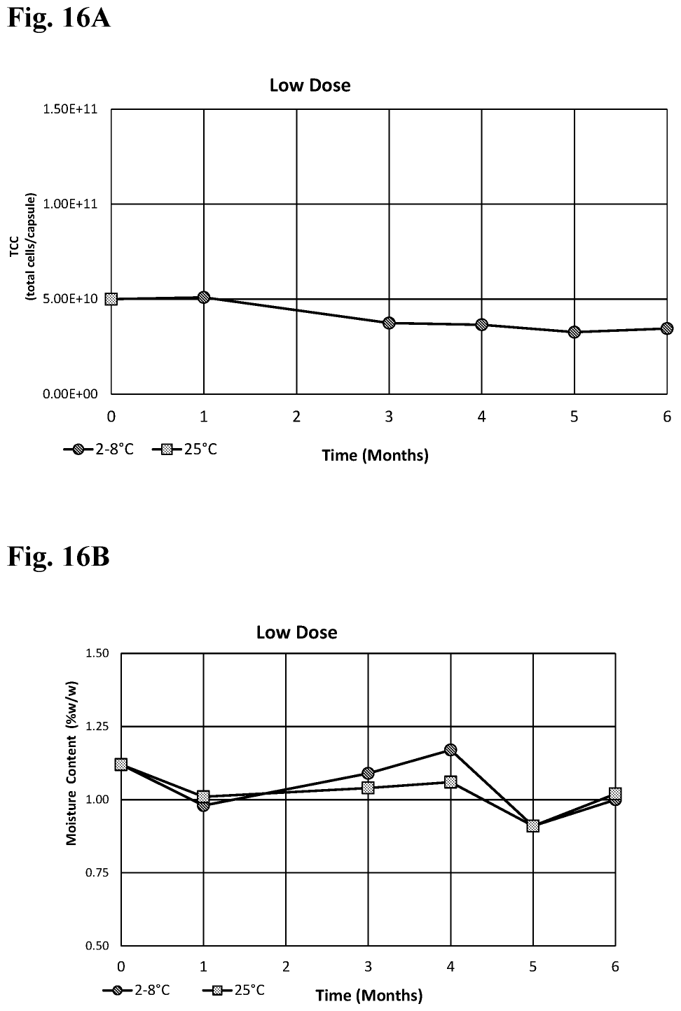

FIGS. 16A and B are graphs showing 6-month Stability Profiles for a second low dose batch. FIG. 16A is a graph showing Total Cells/Capsule Stability Profile over time long-term (2-8° C. (abbreviation: 5° C.)) and accelerated (25° C./60% RH (abbreviation: 25° C.)) storage conditions for the second dose batch. Total Cell Count (TCC) was determined by Coulter counter. FIG. 16B is a graph showing Moisture Content Stability Profile over time long-term (2-8° C.) and accelerated (25° C./60% RH (abbreviation: 25° C.)) storage conditions for the second low dose batch. Moisture content was determined by the Karl Fisher method.

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A solid dosage form of a pharmaceutical composition comprising:
a pharmaceutical agent having a total pharmaceutical agent mass that is at least 2.5% and no more than 95% of the total mass of the pharmaceutical composition, wherein the pharmaceutical agent comprises bacteria and/or microbial extracellular vesicles (mEVs) derived therefrom;
a diluent having a total mass that is at least 1% and no more than 98% of the total mass of the pharmaceutical composition;
a lubricant having a total mass that is at least 0.1% and no more than 5% of the total mass of the pharmaceutical composition; and
a glidant having a total mass that is at least 0.01% and no more than 2% of the total mass of the pharmaceutical composition.

2. The solid dosage form of claim 1, wherein the bacteria are Gram positive bacteria.

3. The solid dosage form of claim 1, wherein the bacteria are Gram negative bacteria.

4. The solid dosage form of any one of claims 1 to 3, wherein the bacteria are aerobic bacteria.

5. The solid dosage form of any one of claims 1 to 3, wherein the bacteria are anaerobic bacteria.

6. The solid dosage form of any one of claims 1 to 5, wherein the bacteria are acidophile bacteria.

7. The solid dosage form of any one of claims 1 to 5, wherein the bacteria are alkaliphile bacteria.

8. The solid dosage form of any one of claims 1 to 5, wherein the bacteria are neutralophile bacteria.

9. The solid dosage form of any one of claims 1 to 8, wherein the bacteria are fastidious bacteria.

10. The solid dosage form of any one of claims 1 to 8, wherein the bacteria are nonfastidious bacteria.

11. The solid dosage form of any one of claims 1 to 10, wherein the bacteria are from a taxonomic group (e.g., class, order, family, genus, species or strain) listed in Table 1, Table 2, or Table 3.

12. The solid dosage form of any one of claims 1 to 10, wherein the bacteria are from a bacterial strain listed in Table 1, Table 2, or Table 3.

13. The solid dosage form of any one of claims 1 to 10, wherein the bacteria are from bacteria from a taxonomic group (e.g., class, order, family, genus, species or strain) listed in Table J.

14. The solid dosage form of any one of claims 1 to 10, wherein the bacteria are from a bacterial strain listed in Table J.

15. The solid dosage form of claim 1, wherein:
the pharmaceutical agent has a total pharmaceutical agent mass that is at least 5% and no more than 95% of the total mass of the pharmaceutical composition, wherein the pharmaceutical agent comprises Prevotella histicola bacteria or microbial extracellular vesicles (mEVs) derived therefrom; and
the diluent has a total mass that is at least 1% and no more than 95% of the total mass of the pharmaceutical composition.

16. The solid dosage form of claim 15, wherein the Prevotella histicola is Prevotella histicola Strain B (NRRL accession number B 50329).

17. The solid dosage form of claim 1, wherein:
the pharmaceutical agent has a total pharmaceutical agent mass that is at least 2.5% and no more than 70% of the total mass of the pharmaceutical composition, wherein the pharmaceutical agent comprises Veillonella parvula bacteria or microbial extracellular vesicles (mEVs) derived therefrom;
the diluent has a total mass that is at least 30% and no more than 98% of the total mass of the pharmaceutical composition;
the lubricant has a total mass that is at least 0.5% and no more than 2.5% of the total mass of the pharmaceutical composition; and
the glidant has a total mass that is at least 0.1% and no more than 1% of the total mass of the pharmaceutical composition.

18. The solid dosage form of claim 17, wherein the Veillonella parvula is Veillonella parvula Strain A (ATCC Deposit Number PTA-125691).

19. The solid dosage form of any one of claims 1-18, wherein:
the pharmaceutical agent has a total pharmaceutical agent mass that is at least 4% and no more than 65% of the total mass of the pharmaceutical composition; and
the diluent has a total mass that is at least 35% and no more than 95% of the total mass of the pharmaceutical composition.

20. The solid dosage form of any one of claims 1-18, wherein:
the pharmaceutical agent has a total pharmaceutical agent mass that is about 5% to about 60% of the total mass of the pharmaceutical composition; and
the diluent has a total mass that is about 38% to 93% of the total mass of the pharmaceutical composition.

21. The solid dosage form of any one of claims 1-18, wherein:
the pharmaceutical agent has a total pharmaceutical agent mass that is at least 20% and no more than 55% of the total mass of the pharmaceutical composition; and the diluent has a total mass that is at least 45% and no more than 80% of the total mass of the pharmaceutical composition.

22. The solid dosage form of any one of claims 1-18, wherein:
the pharmaceutical agent has a total pharmaceutical agent mass that is about 8% to about 92% of the total mass of the pharmaceutical composition; and
the diluent has a total mass that is about 5% to 90% of the total mass of the pharmaceutical composition.

23. The solid dosage form of any one of claims 1-18, wherein:
the pharmaceutical agent has a total pharmaceutical agent mass that is about 20% to about 50% of the total mass of the pharmaceutical composition; and
the diluent has a total mass that is about 50% to 80% of the total mass of the pharmaceutical composition.

24. The solid dosage form of any one of claims 1-18, wherein:
the pharmaceutical agent has a total pharmaceutical agent mass that is about 30% to about 50% of the total mass of the pharmaceutical composition; and
the diluent has a total mass that is about 45% to 70% of the total mass of the pharmaceutical composition.

25. The solid dosage form of any one of claims 1-18, wherein:
the pharmaceutical agent has a total pharmaceutical agent mass that is about 50% of the total mass of the pharmaceutical composition; and
the diluent has a total mass that is about 48.5% of the total mass of the pharmaceutical composition.

26. The solid dosage form of any one of claims 1-18, wherein:
the pharmaceutical agent has a total pharmaceutical agent mass that is about 8% to about 92% of the total mass of the pharmaceutical composition; and
the diluent has a total mass that is about 5% to 90% of the total mass of the pharmaceutical composition.

27. The solid dosage form of any one of claims 1-18, wherein:
the pharmaceutical agent has a total pharmaceutical agent mass that is about 10% to about 90% of the total mass of the pharmaceutical composition; and
the diluent has a total mass that is about 8.5% to 88.5% of the total mass of the pharmaceutical composition.

28. The solid dosage form of any one of claims 1-18, wherein:
the pharmaceutical agent has a total pharmaceutical agent mass that is about 13.51% of the total mass of the pharmaceutical composition; and
the diluent has a total mass that is about 84.99% of the total mass of the pharmaceutical composition.

29. The solid dosage form of any one of claims 1-18, wherein:
the pharmaceutical agent has a total pharmaceutical agent mass that is about 90.22% of the total mass of the pharmaceutical composition; and
the diluent has a total mass that is about 8.28% of the total mass of the pharmaceutical composition.

30. The solid dosage form of any one of claims 1-18, wherein:
the pharmaceutical agent has a total pharmaceutical agent mass that is about 5% to about 50% of the total mass of the pharmaceutical composition; and
the diluent has a total mass that is about 50% to 95% of the total mass of the pharmaceutical composition.

31. The solid dosage form of any one of claims 1-18, wherein:
the pharmaceutical agent has a total pharmaceutical agent mass that is about 8% to about 45% of the total mass of the pharmaceutical composition; and
the diluent has a total mass that is about 55% to 90% of the total mass of the pharmaceutical composition.

32. The solid dosage form of any one of claims 1-18, wherein:
the pharmaceutical agent has a total pharmaceutical agent mass that is about 40% of the total mass of the pharmaceutical composition; and
the diluent has a total mass that is about 58% of the total mass of the pharmaceutical composition.

33. The solid dosage form of any one of claims 1-18, wherein:
the pharmaceutical agent has a total pharmaceutical agent mass that is about 10.6% of the total mass of the pharmaceutical composition; and
the diluent has a total mass that is about 87.4% of the total mass of the pharmaceutical composition.

34. The solid dosage form of any one of claims 1 to 33, wherein the diluent comprises mannitol.

35. The solid dosage form of any one of claims 1 to 34, wherein the lubricant comprises magnesium stearate.

36. The solid dosage form of any one of claims 1 to 35, wherein the glidant comprises colloidal silicon dioxide.

37. The solid dosage form of any one of claims 1 to 36, wherein the diluent comprises mannitol; the lubricant comprises magnesium stearate; and the glidant comprises colloidal silicon dioxide.

38. The solid dosage form of any one of claims 1 to 37, wherein the pharmaceutical agent comprises bacteria.

39. The solid dosage form of claim 38, wherein the bacteria are lyophilized bacteria.

40. The solid dosage form of any one of claims 1 to 39, wherein the pharmaceutical agent comprises isolated bacteria (e.g., from one or more strains of bacteria (e.g., bacteria of interest) (e.g., a therapeutically effective amount thereof)).

41. The solid dosage form of any one of claims 1 to 40, wherein the pharmaceutical agent comprises bacteria that have been gamma irradiated, UV irradiated, heat inactivated, acid treated, or oxygen sparged.

42. The solid dosage form of any one of claims 1 to 41, wherein the pharmaceutical agent comprises live bacteria.

43. The solid dosage form of any one of claims 1 to 41, wherein the pharmaceutical agent comprises dead bacteria.

44. The solid dosage form of any one of claims 1 to 41, wherein the pharmaceutical agent comprises non-replicating bacteria.

45. The solid dosage form of any one of claims 1 to 44, wherein the pharmaceutical agent comprises bacteria from one strain of bacteria.

46. The solid dosage form of any one of claims 1 to 45, wherein the bacteria are lyophilized (e.g., the lyophilized product further comprises a pharmaceutically acceptable excipient) (e.g., a powder form).

47. The solid dosage form of any one of claims 1 to 46, wherein the bacteria are gamma irradiated.

48. The solid dosage form of any one of claims 1 to 46, wherein the bacteria are UV irradiated.

49. The solid dosage form of any one of claims 1 to 46, wherein the bacteria are heat inactivated (e.g., at 50° C. for two hours or at 90° C. for two hours).

50. The solid dosage form of any one of claims 1 to 46, wherein the bacteria are acid treated.

51. The solid dosage form of any one of claims 1 to 46, wherein the bacteria are oxygen sparged (e.g., at 0.1 vvm for two hours).

52. The solid dosage form of any one of claims 1 to 51, wherein the pharmaceutical agent comprises microbial extracellular vesicles (mEV).

53. The solid dosage form of any one of claims 1 to 52, wherein the pharmaceutical agent comprises isolated mEVs (e.g., from one or more strains of bacteria (e.g., bacteria of interest)) (e.g., a therapeutically effective amount thereof).

54. The solid dosage form of any one of claims 1 to 53, wherein the pharmaceutical agent comprises mEVs and the mEVs comprise secreted mEVs (smEVs).

55. The solid dosage form of any one of claims 1 to 53, wherein the pharmaceutical agent comprises mEVs and the mEVs comprise processed mEVs (pmEVs).

56. The solid dosage form of any one of claims 1 to 53, wherein the pharmaceutical agent comprises pmEVs and the pmEVs are produced from bacteria that have been gamma irradiated, UV irradiated, heat inactivated, acid treated, or oxygen sparged.

57. The solid dosage form of any one of claims 1 to 53, wherein the pharmaceutical agent comprises pmEVs and the pmEVs are produced from live bacteria.

58. The solid dosage form of any one of claims 1 to 53, wherein the pharmaceutical agent comprises pmEVs and the pmEVs are produced from dead bacteria.

59. The solid dosage form of any one of claims 1 to 53, wherein the pharmaceutical agent comprises pmEVs and the pmEVs are produced from non-replicating bacteria.

60. The solid dosage form of any one of claims 1 to 59, wherein the pharmaceutical agent comprises mEVs and the mEVs are from one strain of bacteria.

61. The solid dosage form of any one of claims 1 to 60, wherein the mEVs are lyophilized (e.g., the lyophilized product further comprises a pharmaceutically acceptable excipient).

62. The solid dosage form of any one of claims 1 to 61, wherein the mEVs are gamma irradiated.

63. The solid dosage form of any one of claims 1 to 61, wherein the mEVs are UV irradiated.

64. The solid dosage form of any one of claims 1 to 61, wherein the mEVs are heat inactivated (e.g., at 50° C. for two hours or at 90° C. for two hours).

65. The solid dosage form of any one of claims 1 to 61, wherein the mEVs are acid treated.

66. The solid dosage form of any one of claims 1 to 65, wherein the mEVs are oxygen sparged (e.g., at 0.1 vvm for two hours).

67. The solid dosage form of any one of claims 1 to 51, wherein the pharmaceutical agent comprises bacteria and the dose of bacteria is about 1×10⁷ to about 2×10¹² (e.g., about 3×10¹⁰ or about 1.5×10¹¹ or about 1.5×10¹²) cells, wherein the dose is per capsule or tablet or per total number of minitablets in a capsule.

68. The solid dosage form of any one of claims 1 to 51, wherein the pharmaceutical agent comprises bacteria and the dose of bacteria is about 1×10⁹, about 3×10⁹, about 5×10⁹, about 1.5×10¹⁰, about 3×10¹⁰, about 5×10¹⁰, about 1.5×10¹¹, about 1.5×10¹², or about 2×10¹² cells, wherein the dose is per capsule or tablet or per total number of minitablets in a capsule.

69. The solid dosage form of any one of claims 1 to 68, wherein the pharmaceutical agent comprises bacteria and/or mEVs and the dose of the pharmaceutical agent (e.g., bacteria and/or mEVs) is about 10 mg to about 1500 mg, wherein the dose is per capsule or tablet or per total number of minitablets in a capsule.

70. The solid dosage form of any one of claims 1 to 68, wherein the pharmaceutical agent comprises bacteria and/or mEVs and the dose of the pharmaceutical agent (e.g., bacteria and/or mEVs) is about 30 mg to about 1300 mg (by weight of bacteria and/or mEVs) (about 25, about 30, about 35, about 50, about 75, about 100, about 120, about 150, about 250, about 300, about 350, about 400, about 500, about 600, about 700, about 750, about 800, about 900, about 1000, about 1100, about 1200, about 1250, about 1300, about 2000, about 2500, about 3000, or about 3500 mg, wherein the dose is per capsule or tablet or per total number of minitablets in a capsule.

71. The solid dosage form of any one of claims 1 to 70, wherein the pharmaceutical agent comprises bacteria and/or mEVs and the dose of the pharmaceutical agent (e.g., bacteria and/or mEVs) is about 2×10⁶ to about 2×10¹⁶ particles (e.g., wherein particle count is determined by NTA (nanoparticle tracking analysis)), wherein the dose is per capsule or tablet or per total number of minitablets in a capsule.

72. The solid dosage form of any one of claims 1 to 71, wherein the pharmaceutical agent comprises bacteria and/or mEVs and the dose of the pharmaceutical agent (e.g., bacteria and/or mEVs) is about 5 mg to about 900 mg total protein (e.g., wherein total protein is determined by Bradford assay or BCA), wherein the dose is per capsule or tablet or per total number of minitablets in a capsule.

73. The solid dosage form of any one of claims 1 to 72, wherein the solid dosage form further comprises one or more therapeutic agents.

74. The solid dosage form of any one of claims 1 to 73, wherein the solid dosage form further comprises an excipient (e.g., an excipient described herein, e.g., a diluent, a binder and/or an adhesive, a disintegrant, a lubricant and/or a glidant, a coloring agent, a flavoring agent, and/or a sweetening agent).

75. The solid dosage form of any one of claims 1 to 74, wherein the solid dosage form is a capsule.

76. The solid dosage form of claim 75, wherein the capsule comprises HPMC.

77. The solid dosage form of claim 75, wherein the capsule is banded with an HPMC-based banding solution.

78. The solid dosage form of any one of claims 1 to 77, further comprising an enteric coating.

79. The solid dosage form of any one of claims 1 to 78, wherein the enteric coating comprises a methacrylic acid ethyl acrylate (MAE) copolymer (1:1).

80. The solid dosage form of any one of claims 1 to 79, wherein the enteric coating comprises cellulose acetate phthalate (CAP), cellulose acetate trimellitate (CAT), poly(vinyl acetate phthalate) (PVAP), hydroxypropyl methylcellulose phthalate (HPMCP), a fatty acid, a wax, shellac (esters of aleurtic acid), a plastic, a plant fiber, zein, Aqua-Zein (an aqueous zein formulation containing no alcohol), amylose starch, a starch derivative, a dextrin, a methyl acrylate-methacrylic acid copolymer, cellulose acetate succinate, hydroxypropyl methyl cellulose acetate succinate (hypromellose acetate succinate), a methyl methacrylate-methacrylic acid copolymer, or sodium alginate.

81. The solid dosage form of any one of claims 1 to 80, wherein the enteric coating comprises an anionic polymeric material.

82. A method of preventing or treating a disease of a subject, the method comprising administering to the subject a solid dosage form of any one of claims 1 to 81.

83. Use of a solid dosage form of any one of claims 1 to 81 for the treatment or prevention of a disease of a subject.

84. Use of a solid dosage form of any one of claims 1 to 81 for the preparation of a medicament for treating or preventing a disease in a subject.

85. The solid dosage form of any one of claims 1 to 81 for use in the treatment or prevention of a disease of a subject.

86. A method of preventing or treating a disease of a subject, the method comprising administering to the subject a solid dosage form of any one of claims 1 to 81.

87. Use of a solid dosage form of any one of claims 1 to 81 for the treatment or prevention of a disease of a subject.

88. Use of a solid dosage form of any one of claims 1 to 81 for the preparation of a medicament for treating or preventing a disease in a subject.

89. A solid dosage form of any one of claims 1 to 81 for use in the treatment or prevention of a disease of a subject.

90. The method/solid dosage form/use of any one of claims 82 to 89, wherein the solid dosage form is orally administered (e.g., is for oral administration).

91. The method/solid dosage form/use of any one of claims 82 to 89, wherein the solid dosage form is administered on an empty stomach (e.g., one hour before eating or two hours after eating).

92. The method/solid dosage form/use of any one of claims 82 to 89, wherein the solid dosage form is administered (e.g., is for administration) 1, 2, 3, or 4 times a day.

93. The method/solid dosage form/use of any one of claims 82 to 89, wherein the solid dosage form comprises a capsule and 1, 2, 3, or 4 solid dosage forms are administered (e.g., are for administration) 1, 2, 3, or 4 times a day.

94. The method/solid dosage form/use of any one of claims 82 to 93, wherein the subject is in need of treatment (and/or prevention) of a cancer.

95. The method/solid dosage form/use of any one of claims 82 to 93, wherein the subject is in need of treatment (and/or prevention) of an autoimmune disease.

96. The method/solid dosage form/use of any one of claims 82 to 93, wherein the subject is in need of treatment (and/or prevention) of an inflammatory disease.

97. The method/solid dosage form/use of any one of claims 82 to 93, wherein the subject is in need of treatment (and/or prevention) of a metabolic disease.

98. The method/solid dosage form/use of any one of claims 82 to 97, wherein the subject is in need of treatment (and/or prevention) of a dysbiosis.

99. The method/solid dosage form/use of any one of claims 82 to 98, wherein the solid dosage form is administered in combination with a therapeutic agent.

100. A method of preparing a solid dosage form of a pharmaceutical composition, the method comprising:
(a) combining into a pharmaceutical composition:
(i) a pharmaceutical agent having a total pharmaceutical agent mass that is at least 2.5% and no more than 95% of the total mass of the pharmaceutical composition, wherein the pharmaceutical agent comprises bacteria and/or microbial extracellular vesicles (mEVs);
(ii) a diluent having a total mass that is at least 1% and no more than 98% of the total mass of the pharmaceutical composition;
(iii) a lubricant having a total mass that is at least 0.1% and no more than 5% of the total mass of the pharmaceutical composition; and
(iv) a glidant having a total mass that is at least 0.01% and no more than 2% of the total mass of the pharmaceutical composition; and
(b) loading the pharmaceutical composition into a capsule.

101. The method of claim 100, wherein:
the pharmaceutical agent has a total pharmaceutical agent mass that is at least 4% and no more than 65% of the total mass of the pharmaceutical composition; and
the diluent has a total mass that is at least 35% and no more than 95% of the total mass of the pharmaceutical composition.

102. The method of claim 100, wherein:
the pharmaceutical agent has a total pharmaceutical agent mass that is about 5% to about 60% of the total mass of the pharmaceutical composition; and
the diluent has a total mass that is about 38% to 93% of the total mass of the pharmaceutical composition.

103. The method of claim 100, wherein:
the pharmaceutical agent has a total pharmaceutical agent mass that is at least 20% and no more than 55% of the total mass of the pharmaceutical composition; and
the diluent has a total mass that is at least 45% and no more than 80% of the total mass of the pharmaceutical composition.

104. The method of claim 100, wherein:
the pharmaceutical agent has a total pharmaceutical agent mass that is about 8% to about 92% of the total mass of the pharmaceutical composition; and
the diluent has a total mass that is about 5% to 90% of the total mass of the pharmaceutical composition.

105. The method of claim 100, wherein:
the pharmaceutical agent has a total pharmaceutical agent mass that is about 20% to about 50% of the total mass of the pharmaceutical composition; and
the diluent has a total mass that is about 50% to 80% of the total mass of the pharmaceutical composition.

106. The method of claim 100, wherein:
the pharmaceutical agent has a total pharmaceutical agent mass that is about 30% to about 50% of the total mass of the pharmaceutical composition; and
the diluent has a total mass that is about 45% to 70% of the total mass of the pharmaceutical composition.

107. The method of claim 100, wherein:
the pharmaceutical agent has a total pharmaceutical agent mass that is about 50% of the total mass of the pharmaceutical composition; and
the diluent has a total mass that is about 48.5% of the total mass of the pharmaceutical composition.

108. The method of claim 100, wherein:
the pharmaceutical agent has a total pharmaceutical agent mass that is about 8% to about 92% of the total mass of the pharmaceutical composition; and
the diluent has a total mass that is about 5% to 90% of the total mass of the pharmaceutical composition.

109. The method of claim 100, wherein:
the pharmaceutical agent has a total pharmaceutical agent mass that is about 10% to about 90% of the total mass of the pharmaceutical composition; and
the diluent has a total mass that is about 8.5% to 88.5% of the total mass of the pharmaceutical composition.

110. The method of claim 100, wherein:
the pharmaceutical agent has a total pharmaceutical agent mass that is about 13.51% of the total mass of the pharmaceutical composition; and
the diluent has a total mass that is about 84.99% of the total mass of the pharmaceutical composition.

111. The method of claim 100, wherein:
the pharmaceutical agent has a total pharmaceutical agent mass that is about 90.22% of the total mass of the pharmaceutical composition; and
the diluent has a total mass that is about 8.28% of the total mass of the pharmaceutical composition.

112. The method of claim 100, wherein:
the pharmaceutical agent has a total pharmaceutical agent mass that is about 5% to about 50% of the total mass of the pharmaceutical composition; and
the diluent has a total mass that is about 50% to 95% of the total mass of the pharmaceutical composition.

113. The method of claim 100, wherein:
the pharmaceutical agent has a total pharmaceutical agent mass that is about 8% to about 45% of the total mass of the pharmaceutical composition; and
the diluent has a total mass that is about 55% to 90% of the total mass of the pharmaceutical composition.

114. The method of claim 100, wherein:
the pharmaceutical agent has a total pharmaceutical agent mass that is about 40% of the total mass of the pharmaceutical composition; and
the diluent has a total mass that is about 58% of the total mass of the pharmaceutical composition.

115. The method of claim 100, wherein:
the pharmaceutical agent has a total pharmaceutical agent mass that is about 10.6% of the total mass of the pharmaceutical composition; and
the diluent has a total mass that is about 87.4% of the total mass of the pharmaceutical composition.

116. The method of any one of claims 100 to 115, further comprising the step of enterically coating the solid dosage form to obtain an enterically coated solid dosage form.

117. The method of any one of claims 100 to 116, further comprising the step of performing wet granulation on the pharmaceutical agent prior to the combining step.

118. A method of performing wet granulation on a pharmaceutical agent comprising bacteria and/or microbial extracellular vesicles (mEVs), the method comprising:
(i) mixing the pharmaceutical agent with a granulating fluid;
(ii) drying mixed pharmaceutical agent and granulating fluid; and
(iii) milling the dried pharmaceutical agent and granulating fluid;
wherein the milled pharmaceutical agent and granulating fluid are then combined with the one or more excipients to prepare a pharmaceutical composition.

119. The method of claim 118, wherein the wet granulation comprises mixing the pharmaceutical agent with water.
